(19) United States
(12) Patent Application Publication
(10) Pub. No.: US 2026/0258063 A1
(43) Pub. Date: Sep. 3, 2026

(54) CRYSTALLINE MONOMERS FOR PREPARING ANTISENSE OLIGONUCLEOTIDES AND METHODS OF THEIR PREPARATION AND USE

(71) Applicant: EISAI R&D MANAGEMENT CO., LTD., Bunkyo-ku, Tokyo (JP)

(72) Inventors: Hyeong Wook CHOI, Andover, MA (US); Branko MITASEV, North Reading, MA (US); Francis G. FANG, Andover, MA (US); Mingde David SHAN, Andover, MA (US); Praveen Kumar VEMULA, Arlington, MA (US); Farid BENAYOUD, North Andover, MA (US); Jung Hwa LEE, Andover, MA (US)

(73) Assignee: EISAI R&D MANAGEMENT CO., LTD., Bunkyo-ku, Tokyo (JP)

(21) Appl. No.: 18/992,064

(22) PCT Filed: Jul. 6, 2023
(86) PCT No.: PCT/US2023/027033

§ 371 Date: Jan. 7, 2025

Related U.S. Application Data
(60) Provisional application No. 63/359,019, filed on Jul. 7, 2022.
(60) Provisional application No. 63/359,024, filed on Jul. 7, 2022.
(60) Provisional application No. 63/386,083, filed on Dec. 5, 2022.

(51) Int. Cl. C07F 9/6558; C07F 9/6561; C07F 9/6578; C07H 1/00; C07H 19/10; C07H 19/20

(52) U.S. Cl. CPC C07F 9/65583; C07F 9/65616; C07F 9/6578; C07H 1/00; C07H 19/10; C07H 19/20; C07B 2200/13

(57) ABSTRACT

Provided herein are stereo-encoded morpholino monomers of the general Formula (I):

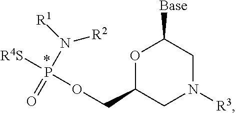

along with methods of their preparation, and methods of their activation and use to prepare stereospecific dimers and oligomers. Stereo-encoded DNA monomers and methods of their use are also provided.

### CROSS-REFERENCE TO RELATED APPLICATIONS

This application claims the benefit of U.S. Provisional Patent Application No. 63/359,019, filed on Jul. 7, 2022; U.S. Provisional Patent Application No. 63/359,024, filed on Jul. 7, 2022; and U.S. Provisional Patent Application No. 63/386,083, filed on Dec. 5, 2022. All of the preceding applications are incorporated by reference herein.

### BACKGROUND

### Field

Embodiments may relate to crystalline stable chiral monomers that may include adenine (A), cytosine (C), guanine (G), thymine (T), uracil (U) or an analog of one of those nucleosides, as well as methods of their preparation and use, including for synthesis of antisense oligonucleotides having diastereomerically pure phosphorodiamidate linkages.

### Background

Synthesis of diastereomerically pure phosphorodiamidate oligonucleotides is substantially complicated by the existence of chiral phosphorous linkages. This is contrasted with, for example, phosphodiester linkages, which do not have a chiral phosphorous.

The existence of the chiral phosphorous presents substantial challenges to synthetic routes that involve connection of a series of phosphorodiamidate nucleotides. Lack of stereochemically pure reagents (templates, subunits, building blocks) that enable stereospecific formation of phosphorodiamidate linkages leads to reaction at the stereocenter in which the phosphorus chirality of the resulting compound may not be controlled.

For sufficiently long oligonucleotides, chirally uncontrolled coupling can lead to a heterogeneous mixture of many diastereomers. This can create the need to sensitive separation techniques, and for longer oligonucleodies it may make separation into stereoisomers impossible or virtually so, while consuming raw materials to create large amounts of stereomerically undesirable compounds.

It would be useful to have stable, stereo-encoded monomers for use in preparation of antisense oligonucleotides having diastereomerically pure phosphorodiamidate linkages including stereopure PMOs.

Use of antisense oligonucleotides in drug products has been reported, for example, in Crooke, et al., “Antisense Technology: A Review,” J Biol. Chem. (2021) 296, 100416.

### SUMMARY

Embodiments as reported in this disclosure provide stereo-encoded crystalline monomers that may be useful in preparing activated stereo-encoded monomers. These activated stereo-encoded monomers may be useful, in turn, for preparing stereopure oligomers. Embodiments may include, for example, the following.

Embodiments may provide a stereo-encoded compound that is a morpholino monomer of Formula (I)

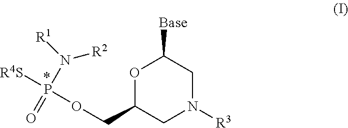

or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R³ is a protecting group for morpholino nitrogen; R⁴ is hydrogen or C1-C6 alkyl Base is selected from the group consisting of:

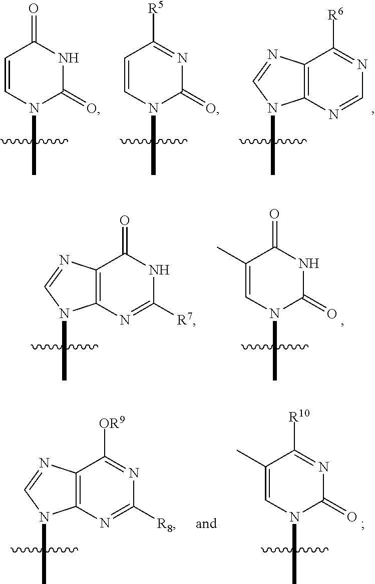

R⁵, R⁶, R⁷, R⁸, and R¹⁰ are independently —NR²⁰R²¹ or —N═CR¹²—NR¹³R¹⁴; where any of R⁵, R⁶, R⁷, R⁸, and R¹⁰ is independently —NR²⁰R²¹R²⁰ and R²¹ are independently selected from —H, —C(O)R¹¹ and —C(O)OR¹¹; R¹¹ is optionally substituted C1-C6 alkyl, optionally substituted benzyl, or aryl, optionally substituted with one or more substituents selected from halogen, nitro and alkoxyl; when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl; when any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or R¹² and R¹³ together form a 5 or 6-membered heterocycle with the adjacent C and N; and R⁹ is H, optionally substituted C1-C6 alkyl, optionally substituted benzyl, acyl, carbonate, carbamate, or silyl.

In some embodiments the compound is crystalline. In further embodiments R¹ and R² are independently optionally substituted C1-C6 alkyl. In still further embodiments R¹ and R² are methyl. In yet still further embodiments R¹ and R² together with the nitrogen they connect to form a heterocycle selected from morpholinyl, piperazinyl, pyrrolidinyl, and azetidinyl. In further embodiments R³ is selected from optionally substituted C1-C6 alkyl, trityl, benzyl, and sulfonyl. In some embodiments R³ is selected from the group consisting of trityl, p-methoxyphenyldiphenylmethyl, benzyl, methoxybenzyl, dimethoxybenzyl, diphenylmethyl, nitrobenzenesulfonyl, and dinitrobenzenesulfonyl. In some embodiments R⁴ is H or methyl. In some embodiments R¹ and R² are methyl, R³ is trityl, and R⁴ is H or methyl. In further embodiments, any of R⁵, R⁶, R⁷, R⁸ and R¹⁰ is independently: (a) —NHC(O)R¹¹, wherein R¹¹ is methyl, ethyl, isopropyl, isobutyl, phenyl, 4-methoxy phenyl, 4-bromophenyl, and 4-nitrophenyl, or (b) —NHC(O)OR¹¹, wherein R¹¹ is C1-C6 alkyl, benzyl, 2,2,2-trichloroethyl, or aryl. In some embodiments, any of R⁵, R⁶, R⁷, R⁸ and R¹⁰ is independently

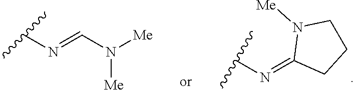 [SMILES: */N=C/N(C)C ; */N=C1/CCCN1C]

In some embodiments R⁹ is selected from the group consisting of cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-dimethylbenzyl, 3,4-dimethylbenzyl, 2,6-dimethylbenzyl, 4-methoxybenzyl, and 4-pivaloyloxy benzyl. Further embodiments provide the following compounds:

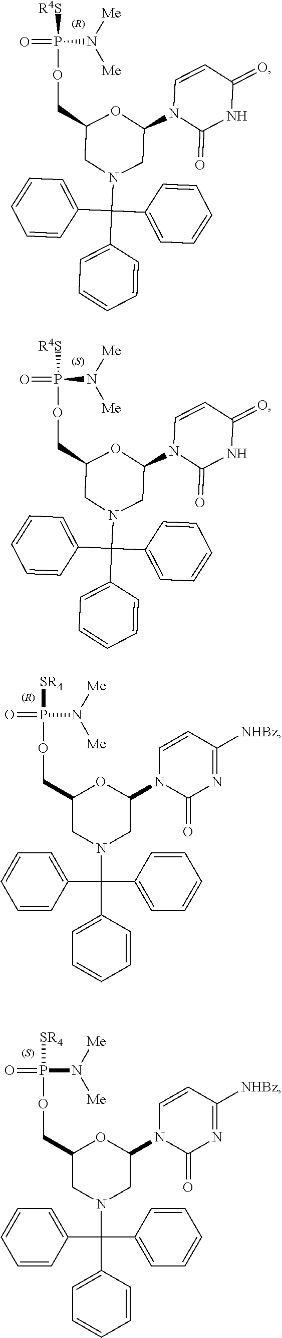

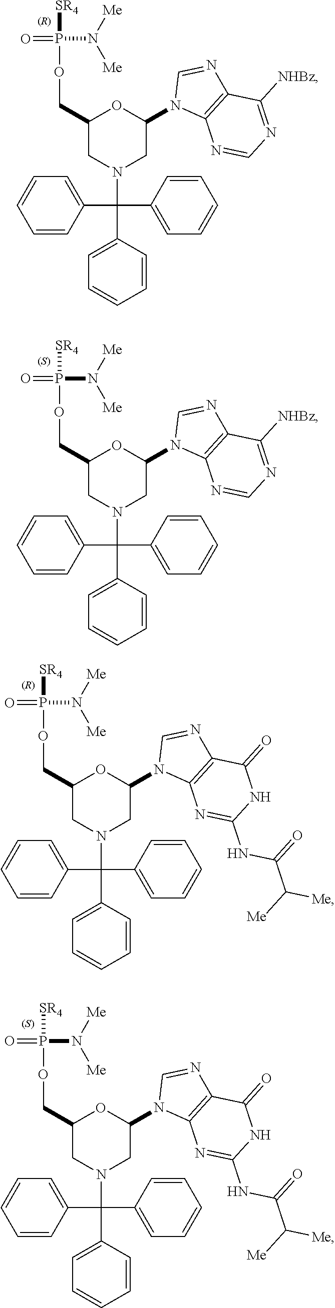

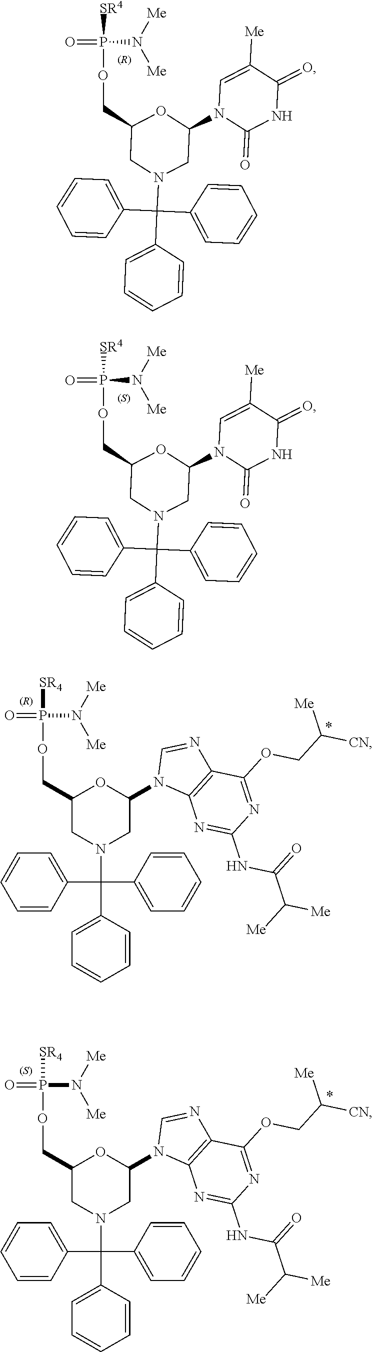

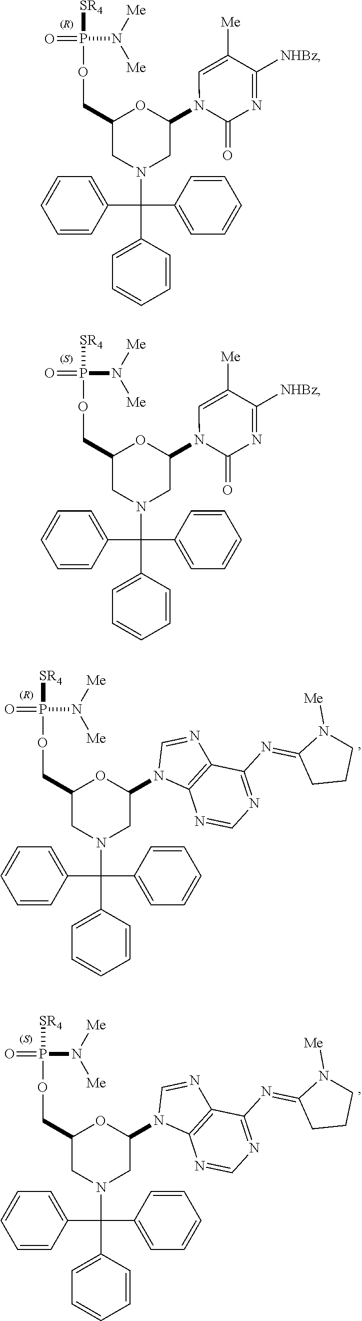

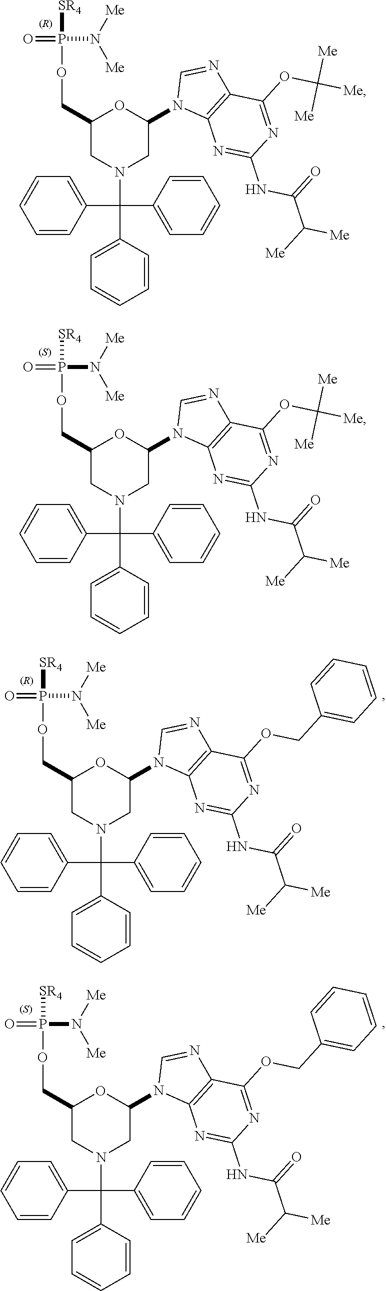

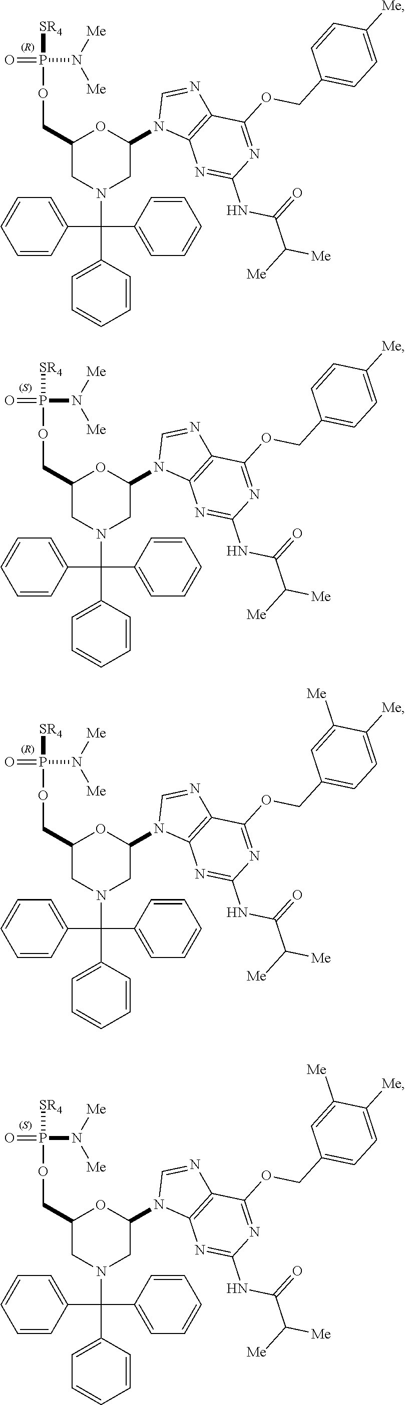

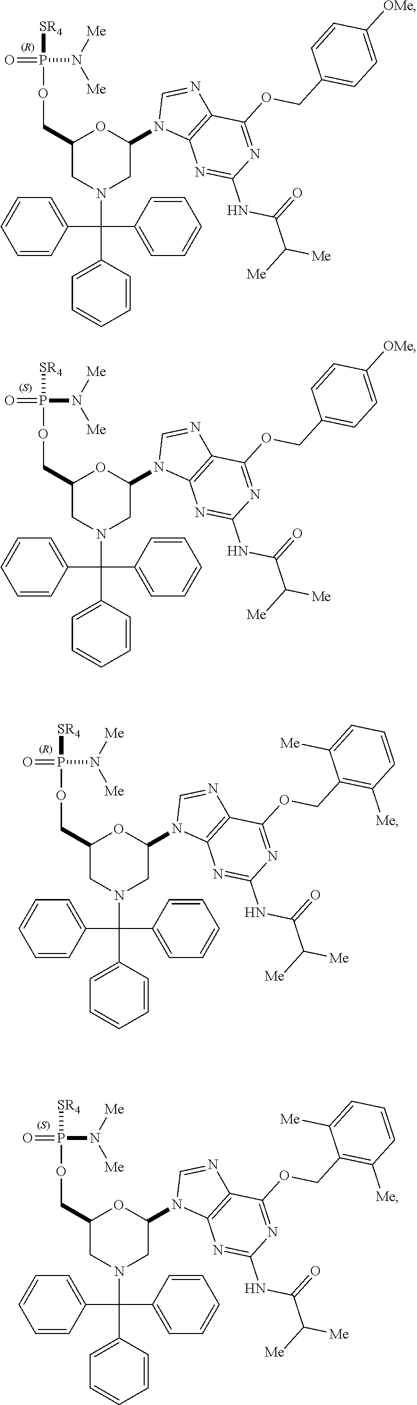

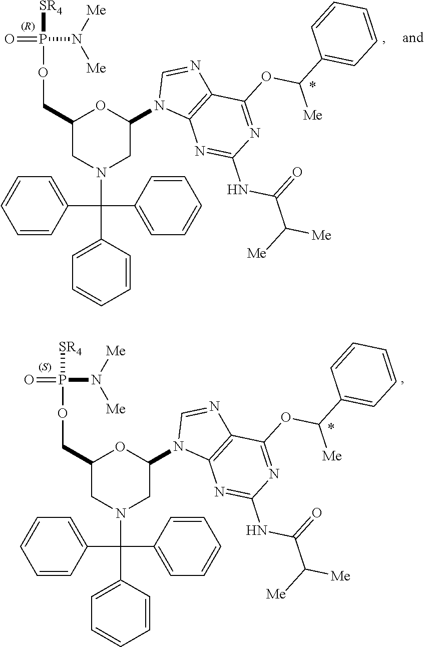

wherein * indicates either (R) or (S) stereochemistry, or a mixture thereof, and
R⁴ is H or methyl.

Some embodiments provide a method of preparing a stereo-encoded activated monomer of Formula (II)

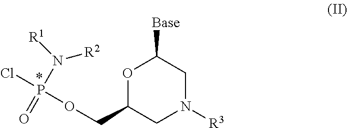

or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R³ is a protecting group for morpholino nitrogen, selected from optionally substituted C1-C6 alkyl, trityl, benzyl, or sulfonyl; Base is selected from the group consisting of:

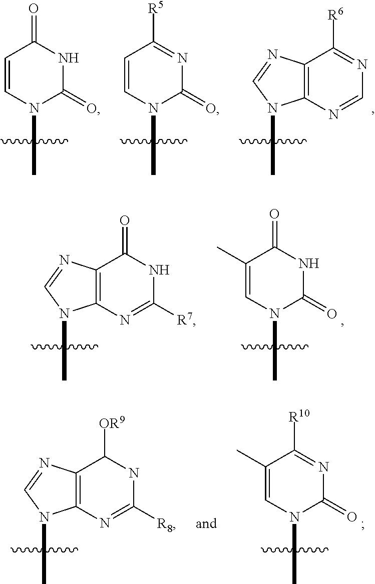

R⁵, R⁶, R⁷, R⁸, and R¹⁰ are independently —NR²⁰R²¹ or —N═CR¹²—NR¹³R¹⁴; where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹, R¹¹ is optionally substituted C1-C6 alkyl, optionally substituted benzyl, or aryl, optionally substituted with one or more substituents selected from halogen, nitro and alkoxyl; when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl; when any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or R¹² and R¹³ together form a 5 or 6-membered heterocycle with the adjacent C and N; R⁹ is H, optionally substituted C1-C6 alkyl, optionally substituted benzyl, acyl, carbonate, carbamate, or silyl;
comprising the following steps: (a) providing a stereo-encoded morpholino monomer of Formula (Ia)

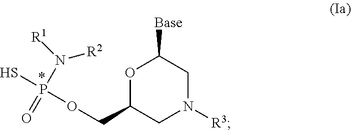

(b) optionally alkylating the sulfur of the stereo-encoded morpholino monomer of Formula (Ia) in step (a) to afford a stereo-encoded morpholino monomer of Formula (I)

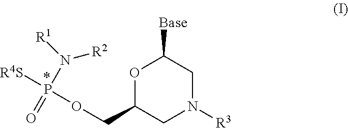

wherein R⁴ is C1-C6 alkyl; (c) reacting the stereo-encoded morpholino monomer of Formula (Ia) from step (a) or the stereo-encoded morpholino monomer of Formula (I) from step (b) with a chlorinating agent to afford the stereo-encoded activated monomer of Formula (II).

In some embodiments of the preceding method, R⁴ is methyl. In some embodiments R¹ and R² are independently C1-C6 alkyl. In some embodiments R¹ and R² are methyl. In further embodiments R¹ and R², together with the nitrogen to which they connect, form a heterocycle selected from morpholinyl, piperazinyl, pyrrolidinyl, and azetidinyl. In yet still further embodiments R³ is selected from the group consisting of trityl, p-methoxyphenyldiphenylmethyl, benzyl, methoxybenzyl, dimethoxybenzyl, diphenylmethyl, nitrobenzenesulfonyl, and dinitrobenzenesulfonyl. In still further embodiments R³ is trityl. In further embodiments R¹ and R² are methyl, R³ is trityl, and R⁴ is H or methyl. In yet still further embodiments, any of R⁵, R⁶, R⁷R⁸ and R¹⁰ is independently (a) —NHC(O)R¹¹, wherein R¹¹ is methyl, ethyl, isopropyl, isobutyl, phenyl, 4-methoxy phenyl, 4-bromophenyl, and 4-nitrophenyl, or (b) —NHC(O)OR¹¹, wherein R¹¹ is C1-C6 alkyl, benzyl, 2,2,2-trichloroethyl, or aryl. In some embodiments, any of R⁵, R⁶, R⁷ R⁸ and R¹⁰ is independently

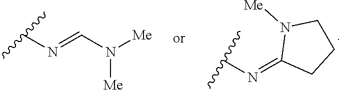 [SMILES: */N=C/N(C)C ; */N=C1/CCCN1C]

In some embodiments wherein R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-dimethylbenzyl, 3,4-dimethylbenzyl, 2,6-dimethylbenzyl, 4-methoxybenzyl, and 4-pivaloyloxy benzyl. In some embodiments the chlorinating agent is SO₂Cl₂ or tetramethyl chloroenamine. In further embodiments step (c) is conducted in the presence of a base. In still further embodiments R⁴ is H and the chlorinating agent is tetramethyl chloroenamine.

Some embodiments provide a stereo-encoded activated monomer prepared by a method recited herein.

An additional embodiment provides a method for preparing a stereo-encoded morpholino monomer of Formula (I)

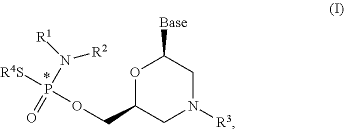

or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R³ is a protecting group for morpholino nitrogen, selected from optionally substituted C1-C6 alkyl, trityl, benzyl, or sulfonyl; R⁴ is hydrogen or C1-C6 alkyl; Base is selected from the group consisting of.

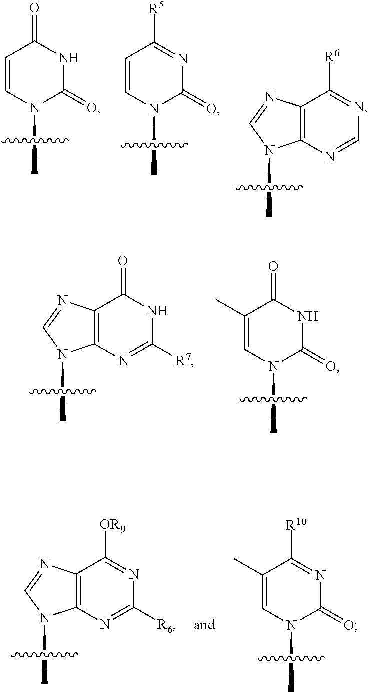

R⁵, R⁶, R⁷, R⁸, and R¹⁰ are independently —NR²⁰R²¹ or —N═CR¹²—NR¹³R¹⁴; where any of R⁵, R⁶, R⁷, R⁸, and R¹⁰ is independently —NR²⁰R²¹R²⁰ and R²¹ are independently selected from —H, —C(O)R¹¹, and —C(O)OR¹¹, and R¹¹ is optionally substituted C1-C6 alkyl, optionally substituted benzyl, or aryl, optionally substituted with one or more substituents selected from halogen, nitro and alkoxyl; when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl; when any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or R¹² and R¹³ together form a 5 or 6-membered heterocycle with the adjacent C and N; R⁹ is H, optionally substituted C1-C6 alkyl, optionally substituted benzyl, acyl, carbonate, carbamate, cyanoethyl, α-methylcyanoethyl, β-methylcyanoethyl, pivaloyloxy benzyl, or silyl;
comprising the step of reacting a compound of Formula (III)

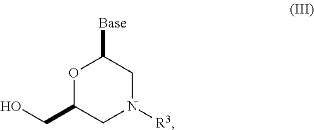

wherein R³ and Base are as defined above, with a chiral reagent of Formula (IV)

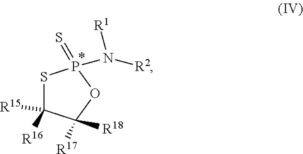

wherein R¹ and R² are as defined above; R¹⁵, ¹⁶, R¹⁷ and R¹⁸ are independently H, C1-C10 alkyl, C2-C12 alkenyl, or aryl; or any two of R¹⁵, R¹⁶, R¹⁷ and R¹⁸ together with the carbons they are attached form a cycloalkyl or heteroalkyl ring which is optionally substituted with one or more C1-C10 alkyl or C2-C12 alkenyl groups, in the presence of a base to obtain the stereo-encoded morpholino monomer of Formula (I).

In some embodiments of the preceding method, R¹ and R² are methyl. In some embodiments R¹⁵, R¹⁶, R¹⁷ and R¹⁸ are independently H, C1-C10 alkyl, or aryl. In further embodiments R¹⁵, R¹⁶, R¹⁷ and R¹⁸ are H, or R¹⁵ is C1-C10 alkyl or aryl, and R¹⁶, R¹⁷, R¹⁸ are H, or R¹⁶ is C1-C10 alkyl or aryl, and R¹⁵, R¹⁷, R¹⁸ are H; or R¹⁵ is phenyl and R¹⁶, R¹⁷, R¹⁸ are H, or R¹⁶ is phenyl, and R¹⁵, R¹⁷, R¹⁸ are H. In still further embodiments the chiral agent of Formula (IV) is selected from

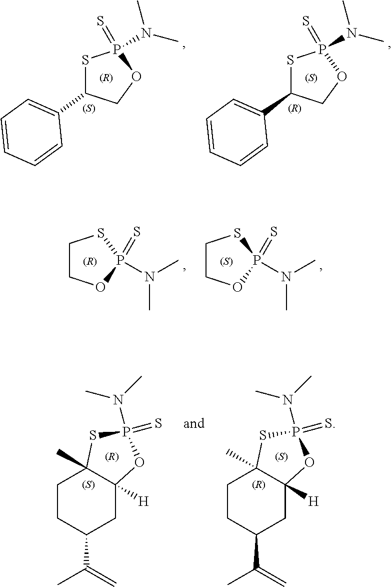

In yet still further embodiments the base is selected from the group consisting of NaH, DBU, sodium tert-amyulate, sodium tert-pentoxide, NaOtBu, KOtBu, potassium tert-pentoxide, and NaHMDS. In still further embodiments the reaction is conducted at room temperature in a polar solvent selected from the group consisting of THF, acetonitrile, 2-MeTHF, 1,6-dioxane, and DME. In further embodiments the stereo-encoded morpholino monomer of Formula (I) is crystalline. In additional embodiments the de/ee ratio/stereoselectivity is at least 90%, 95% or 98%.

A further embodiment may provide a stereo-encoded morpholino monomer of Formula (I) made by the method recited above.

A further embodiment provides a chiral reagent of Formula (IV)

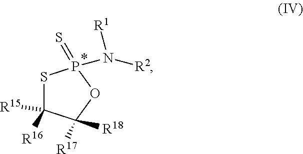

or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R¹⁵, R¹⁶, R¹⁷ and R¹⁸ are independently —H, C1-C10 alkyl, C2-C12 alkenyl, aryl, or any two of R¹⁵, R¹⁶, R¹⁷ and R¹⁸ together with the carbons they are attached form a cycloalkyl or heteroalkyl ring which is optionally substituted with one or more C1-C10 alkyl or C2-C12 alkenyl groups. In a further embodiment R¹ and R² are C1-C6 alkyl. In a still further embodiment R¹ and R² are methyl. In a yet still further embodiment R¹ and R² together with the nitrogen they connect to form a heterocycle selected from morpholinyl, piperazinyl, pyrrolidinyl, and azetidinyl. In a still further embodiment R¹⁵, R¹⁶, R¹⁷ and R¹⁸ are independently H or aryl. In another embodiment R⁵, R¹⁶, R¹⁷ and R¹⁸ are H, or R¹⁵ is C1-C10 alkyl or aryl, and R¹⁶, R¹⁷, R¹⁸ are H, or R¹⁶ is C1-C10 alkyl or aryl, and R¹⁵, R¹⁷, R¹⁸ are H In another embodiment R¹⁵ is phenyl and R¹⁶, R¹⁷, R¹⁸ are H, or R¹⁶ is phenyl, and R¹⁵, R¹⁷, R¹⁸ are H. In a further embodiment R¹⁵, R¹⁶, R¹⁷ and R¹⁸ are H. In a still further embodiment the chiral reagent of Formula (IV) is selected from the following structures

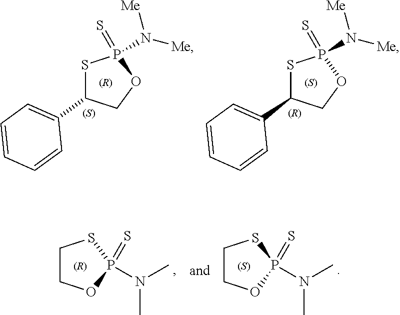

A further embodiment provides a chiral reagent wherein

R¹⁵ is methyl; R¹⁶ and R¹⁷ together form

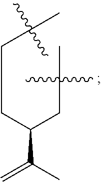 [SMILES: *CCC[C@@H](C*)C(=C)C]

and R¹⁸ is H. Yet in another embodiment, R¹⁶ is methyl; R¹⁵ and R¹⁸ together form

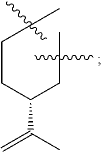 [SMILES: *CC[C@H](C*)C(=C)C]

and R¹⁷ is H. In a further embodiment the chiral reagent is

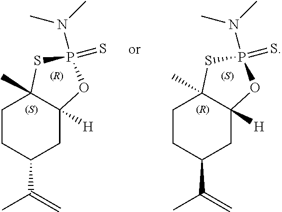

A further embodiment provides a method of preparing

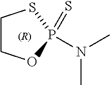

((R)-2-(dimethylamino)-1,3,2-oxathiaphospholane 2-sulfide), and/or

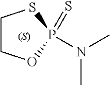

((S)-2-(dimethylamino)-1,3,2-oxathiaphospholane 2-sulfide),
comprising separating racemic 2-(dimethylamino)-1,3,2-oxathiaphospholane 2-sulfide by chiral supercritical fluid chromatography (SFC) or chiral HPLC.

A further embodiment provides a method of making a chiral reagent of formula (IV)

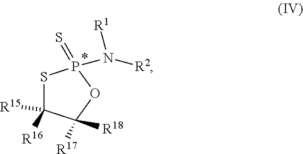

or a diastereomer or enantiomer thereof, or a salt thereof, wherein P* represents a stereocenter that is either in the (R) or (S) configuration; R¹ and R² are independently H or C1-C6 alkyl, or R¹ and R² together with the nitrogen they connect to form an optionally substituted heterocycle selected from morpholine, piperazine, pyrrolidine, and azetidine; R¹⁵, R¹⁶, R¹⁷ and R¹⁸ are independently H, C1-C10 alkyl, C2-C12 alkenyl, aryl; or any two of R¹⁵, R¹⁶, R¹⁷ and R¹⁸ together with the carbons they are attached form a cycloalkyl or heteroalkyl ring which is optionally substituted with one or more C1-C10 alkyl or C2-C12 alkenyl groups, comprising:
reacting a compound of Formula (V)

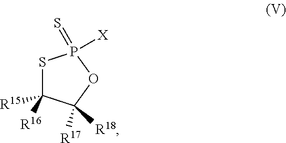

wherein X is a leaving group selected from substituted —S—Ar, —O—Ar or halide, wherein Ar is phenyl, pyridinyl, naphthyl, optionally substituted with one or more of C1-C6 alkyl, cyano, nitro or halo, with NHR¹R² in the presence of an organic base to obtain the chiral reagent of Formula (IV), wherein R¹ and R² are as defined above.

In a further embodiment of the method above, Ar is selected from phenyl, pentafluorophenyl, 4-bromophenyl, mono- or di-nitrophenyl, and 2,3,5,6-tetrafluoropyridin-4-yl. In a still further embodiment the organic base is selected from the group consisting of 1,8-diazabicyclo[5.4.0]undec-7-ene, imidazole, triethylamine, Hunig's base, lutidine, pyridine and combinations thereof. In a still further embodiment, the chiral reagent of Formula (IV) is selected from

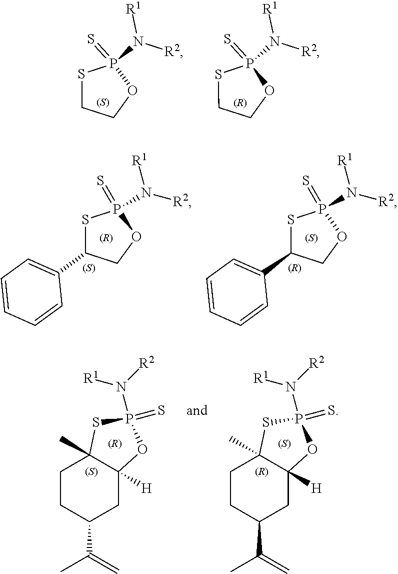

In a still further embodiment R¹ and R² are methyl. In a still further embodiment the chiral reagent of Formula (IV) is

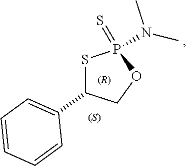

and the compound of formula (V) is

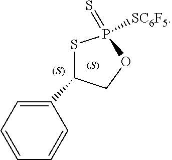

In a yet still further embodiment the chiral reagent of Formula (IV) is

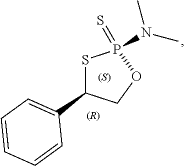

and the compound of Formula (V) is

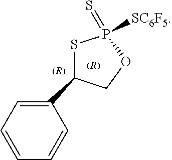

In another embodiment the chiral reagent of Formula (IV) is

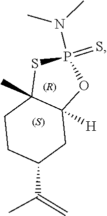

and the compound of Formula (V) is

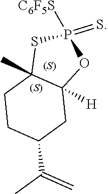

In a further embodiment the chiral reagent of Formula (IV) is

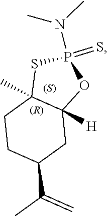

and the compound of Formula (V) is

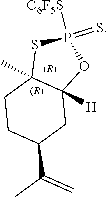

In a still further embodiment R¹ and R² are methyl.

In a still further embodiment the de/stereoselectvitiy of a method above is at least 90%, 95%, or 98%.

A further embodiment provides a chiral reagent of Formula (IV) made by a method reported above.

A further embodiment provides a method of making chiral agent of Formula (IVa)

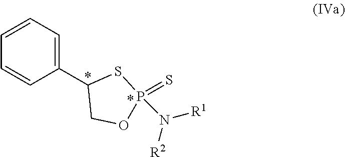

wherein * and P* each represents a stereocenter that is independently either in the (R) or (S) configuration; R¹ and R² are independently H or C1-C6 alkyl, or R¹ and R² together with the nitrogen they connect to form an optionally substituted heterocycle selected from morpholinyl, piperazinyl, pyrrolidinyl, and azetidinyl; comprising the step of reacting a compound of Formula (VI)

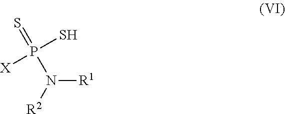

or a salt thereof, wherein X is a leaving group selected from substituted —S—Ar, —O—Ar or halide, wherein Ar is phenyl, pyridinyl, naphthyl, optionally substituted with one or more of C1-C6 alkyl, cyano, nitro or halo, with a chiral styrene oxide in the presence of chloroacetic acid to obtain the chiral reagent of Formula (IVa).

In a further embodiment as recited above, R¹ and R² are methyl. In a still further embodiment Ar is selected from phenyl, pentafluorophenyl, 4-bromophenyl, mono- or di-nitrophenyl, and 2,3,5,6-tetrafluoropyridin-4-yl. In a yet still further embodiment the chiral reagent of Formula (IVa) has the structure:

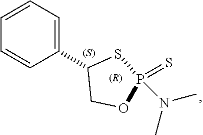

and the chiral styrene oxide is (S)-styrene oxide. In another embodiment the chiral reagent of Formula (IVa) has the structure:

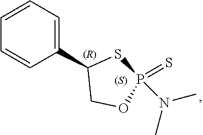

and the chiral styrene oxide is (R)-styrene oxide. In a further embodiment the salt of Formula (VI) is a triethylamine salt.

A further embodiment provides a chiral reagent of Formula (IVa) made by the method reported above.

A yet still further embodiment provides a method of making a compound of Formula (VIa)

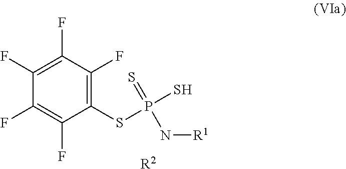

or a salt thereof, comprising the step of reacting a compound of Formula (VII)

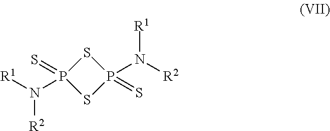

with 2,3,4,5,6-pentafluorobenzenethiol to obtain the compound of Formula (VIa) or a salt thereof.

In a further embodiment R¹ and R² are methyl. In a still further embodiment, the salt of Formula (VIa) is triethylamine salt.

Some embodiments provide a chiral compound of Formula (V)

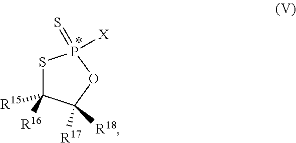

with at least 90% ee.

In Formula (V), P* indicates either (R) or (S) configuration, R¹⁵, R¹⁶, R¹⁷ and R¹⁸ are H, X is a leaving group selected from substituted —S—Ar, —O—Ar or halide, wherein Ar is phenyl, pyridinyl, naphthyl, optionally substituted with one or more of C1-C6 alkyl, cyano, nitro or halo.

In some embodiments, X is —S—Ar, and Ar is phenyl, 4-bromophenyl, 4-nitrophenyl, pentafluorophenyl, dinitrophenyl, or 2,3,5,6-tetrafluoropyridin-4-yl. In some embodiments, the chiral compound of Formula (V) is:

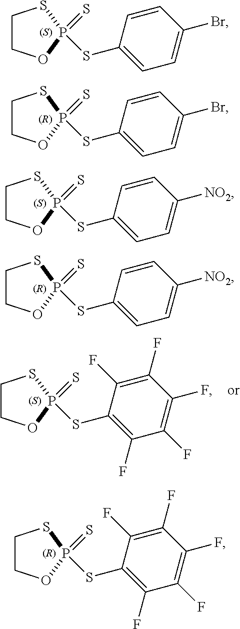

In further embodiments, the ee the chiral compound of Formula (V) is at least 92%, at least 95%, at least 98%, or at least 99%.

Also disclosed herein is a method for preparing a chiral compound of Formula (V)

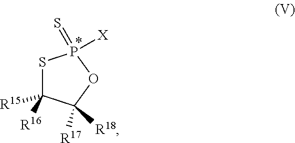

with at least 90% ee.

In Formula (V), P* indicates either (R) or (S) configuration, R¹⁵, R¹⁶, R¹⁷ and R¹⁸ are H, X is a leaving group selected from substituted —S—Ar, —O—Ar or halide, wherein Ar is phenyl, pyridinyl, naphthyl, optionally substituted with one or more of C1-C6 alkyl, cyano, nitro or halo.

In some embodiments, the method includes the step of separating a racemic mixture of

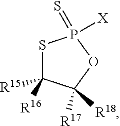

by chiral SFC or chiral HPLC,

In some embodiments, the chiral compound of Formula (V), X is —S—Ar, and Ar is phenyl, 4-bromophenyl, 4-nitrophenyl, pentafluorophenyl, dinitrophenyl, or 2,3,5,6-tetrafluoropyridin-4-yl. In some embodiments, the chiral compound of Formula (V) is:

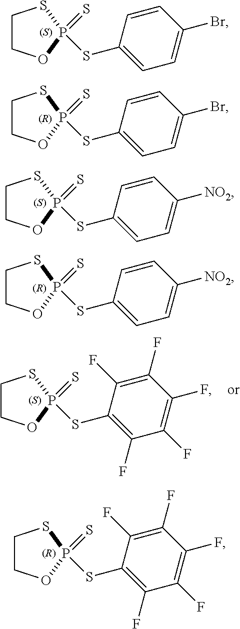

In some embodiments, the ee of the chiral compound of Formula (V) is at least 92%, at least 95%, at least 98%, or at least 99%.

A further embodiment provides a method of preparing a morpholino dimer of Formula (VIII),

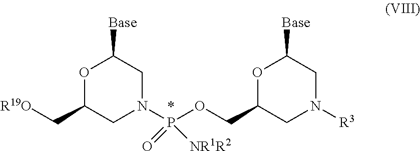

or a diastereomer or enantiomer thereof, or a salt thereof, wherein P* represents a stereocenter that is either in the (R) or (S) configuration; R¹ and R² are independently H or C1-C6 alkyl, or R¹ and R² together with the nitrogen they connect to form an optionally substituted heterocycle selected from morpholine, piperazine, pyrrolidine, and azetidine; R³ is optionally substituted alkyl, trityl, benzyl, or sulfonyl; Base is selected from the group consisting of:

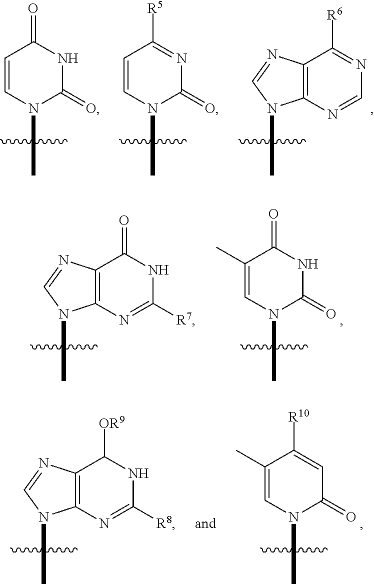

R⁵, R⁶, R⁷, R⁸, and R¹⁰ are independently —NR²⁰R²¹ or —N═CR¹²—NR¹³R¹⁴; when any of R⁵, R⁶, R⁷, R⁸, and R¹⁰ is independently —NR²⁰R²¹, R²⁰ and R²¹ are independently selected from —H, —C(O)R¹¹, and —C(O)OR¹¹, and R¹¹ is optionally substituted C1-C6 alkyl, optionally substituted benzyl, or aryl, optionally substituted with one or more substituents selected from halogen, nitro and alkoxyl; when any of R⁵, R⁶, R⁷, R⁸, and R¹⁰ is —NHC(O)R¹¹, R¹¹ is selected from methyl, ethyl, isopropyl, isobutyl, phenyl, 4-methoxyphenyl, 4-bromophenyl, and 4-nitrophenyl; when any of R⁵, R⁶, R⁷, R⁸, and R¹⁰ is —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or R¹² and R¹³ together form a 5 or 6-membered heterocycle with the adjacent C and N; R⁹ is H, optionally substituted C1-C6 alkyl, optionally substituted benzyl, acyl, carbonate, carbamate, cyanoethyl, α-methylcyanoethyl, β-methylcyanoethyl, pivaloyloxy benzyl, or silyl; and R¹⁹ is H, a protecting group or a linker to solid support;
comprising the steps of
(a) providing a stereo-encoded morpholino monomer of Formula (Ia)

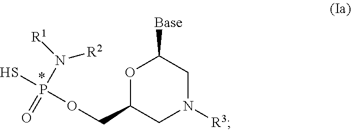

(b) optionally alkylating the sulfur of the stereo-encoded morpholino monomer of Formula (Ia) in step (a) to afford a stereo-encoded morpholino monomer of Formula (I)

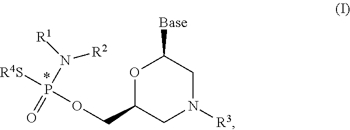

wherein R⁴ is C1-C6 alkyl; (c) reacting the stereo-encoded morpholino monomer of Formula (Ia) from step (a) or the stereo-encoded morpholino monomer of Formula (I) from step (b) with a chlorinating agent to afford a stereo-encoded activated monomer of Formula (II)

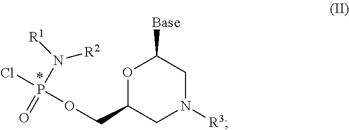

(d) optionally isolating the stereo-encoded activated compound of Formula (II) from step (c); (e) coupling the stereo-encoded activated compound of Formula (II) from step (c) or (d) with a compound of Formula (IX)

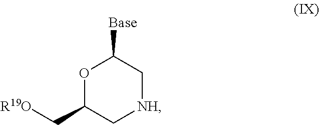

to obtain the morpholino dimer of Formula (VIII).

In a further embodiment of the preceding method, R¹ and R² are methyl. In a still further embodiment R³ is trityl. In some embodiments, R¹⁹ is selected from H or silyl, acyl, optionally substituted trityl. In a yet still further embodiment R¹⁹ is H, TBS, TBDPS, benzoyl or DMTr.

Another embodiment provides a morpholino dimer of Formula (VIII) prepared by the method reported above.

An additional embodiment provides a method of making a phosphorodiamidate morpholino oligomer of formula (X)

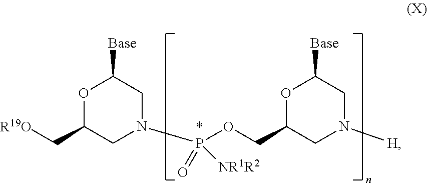

or a diastereomer or enantiomer thereof, or a salt thereof, wherein P* represents a stereocenter that is either in the (R) or (S) configuration, R¹ and R² are independently H, optionally substituted C1-C6 alkyl, optionally substituted phenyl, optionally substituted naphthyl, or R¹ and R² together with the nitrogen to which they connect to form an optionally substituted heterocycle, wherein “optionally substituted” indicates that a moiety may be substituted with one or more independently selected substitutents selected from C1-C6 alkyl, C1-C6 alkenyl, C1-C6 alkynyl, C1-C6 alkoxy, —OH, —NH₂, —NO₂, —CH₂—NH—CH₃, —OCH₂CH₂CH₃, or OCH(CH₃)₂; wherein “optionally substituted” indicates that a moiety may be substituted with one or more independently selected substitutents selected from C1-C6 alkyl, C1-C6 alkenyl, C1-C6 alkynyl, C1-C6 alkoxy, —OH, —NH₂, —NO₂, —CH₂—NH—CH₃, —OCH₂CH₂CH₃, or OCH(CH₃)₂; R¹⁹ is H or a protecting group for a hydroxyl group, or a linker to solid support comprising the following steps:
(a) providing a stereo-encoded morpholino monomer of Formula (Ia)

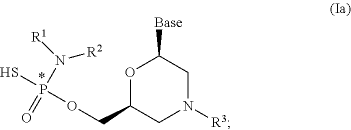

wherein R³ is optionally substituted alkyl, trityl, benzyl, or sulfonyl; (b) optionally sulfur-alkylating the stereo-encoded morpholino monomer of Formula (I) in step (a) to afford a stereo-encoded morpholino monomer of Formula (I)

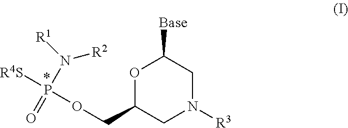

wherein R⁴ is C1-C6 alkyl; (c) reacting the stereo-encoded morpholino monomer of Formula (Ia) from step (a) or the stereo-encoded morpholino monomer of Formula (I) from (b) with a chlorinating agent to afford a stereo-encoded activated monomer of Formula (II)

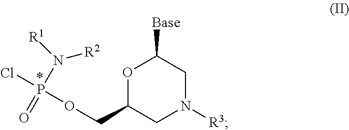

(d) optionally isolating the stereo-encoded activated compound of Formula (II) from step (c); (e) coupling the compound of Formula (II) from step (c) or (d) with a compound of Formula (IX)

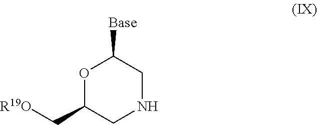

to obtain a morpholino dimer of Formula (VIII)

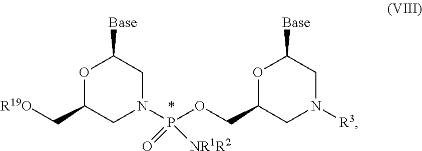

wherein R¹⁹ is a suitable protecting group for a hydroxyl group or a linker to solid support; (f) deprotecting the morpholino dimer of Formula (VIII) to obtain an intermediate oligomer of Formula (X)

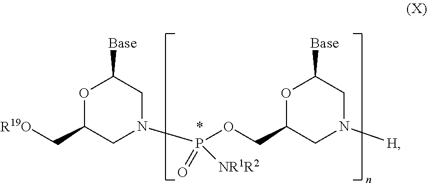

wherein n is 1; (g) repeating step (i) and step (ii) below m times, wherein m is an integer from 0 to 28 inclusive: (i) reacting the intermediate oligomer of Formula (X)

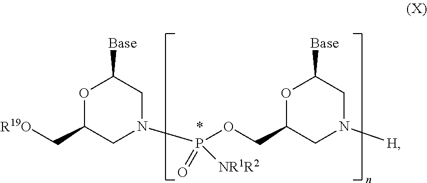

wherein n is an integer from 1 to 28 inclusive, with a stereo-encoded morpholino monomer of Formula (I)

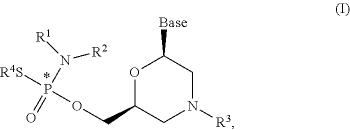

wherein R⁴ is H or C1-C6 alkyl, to provide an intermediate of formula (XI):

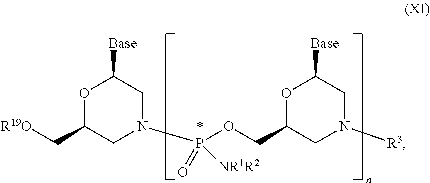

wherein n is an integer from 1 to 29 inclusive; and (ii) deprotecting the intermediate of Formula (XI) from step (i) to provide
phosphorodiamidate morpholino oligomer of Formula (X):

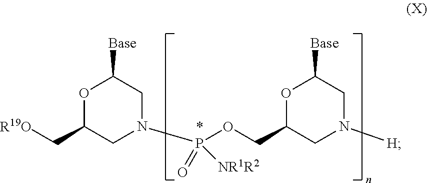

wherein n is an integer from 1 to 29 inclusive; (h) optionally removing R¹⁹ group in the intermediate oligomer of formula (X) from step (ii) of step (g), when R¹⁹ is a suitable protecting group for a hydroxyl group or a linker to solid support, to provide the phosphorodiamidate morpholino oligomer of Formula (X):

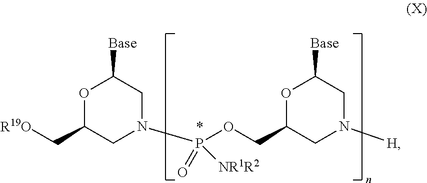

wherein R¹⁹ is H.

In a further embodiment as reported above, R¹ and R² are methyl. In a still further embodiment R³ is trityl. In a yet still further embodiment R¹⁹, as a hydroxyl protecting group in Formulas (IX), (VIII), (IX) and (X), is selected from H, TBS, TBDPS, benzoyl, or DMTr.

An embodiment provides a phosphorodiamidate morpholino oligomer made by a method as recited above.

A further embodiment provides use of a stereo-encoded morpholino monomer of Formula (I)

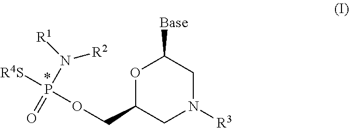

or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R³ is a protecting group for morpholino nitrogen, selected from optionally substituted C1-C6 alkyl, trityl, benzyl, or sulfonyl; R⁴ is hydrogen or C1-C6 alkyl, in the preparation of a morpholino dimer of Formula (VIII),

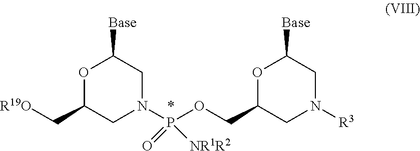

or a diastereomer or enantiomer thereof, or a salt thereof, wherein P* represents a stereocenter that is either in the (R) or (S) configuration; R¹ and R² are independently H, optionally substituted C1-C6 alkyl, optionally substituted phenyl, optionally substituted naphthyl, or R¹ and R² together with the nitrogen they connect to form an optionally substituted heterocycle; R³ is optionally substituted alkyl, trityl, benzyl, or sulfonyl; R¹⁹ is a protecting group for a hydroxyl group, wherein the stereoselectivity or diastereoselectivity is at least 90%, 95%, or 98%.

In some embodiments of the use reported above, the stereo-encoded morpholino monomer of formula (I) is selected from:

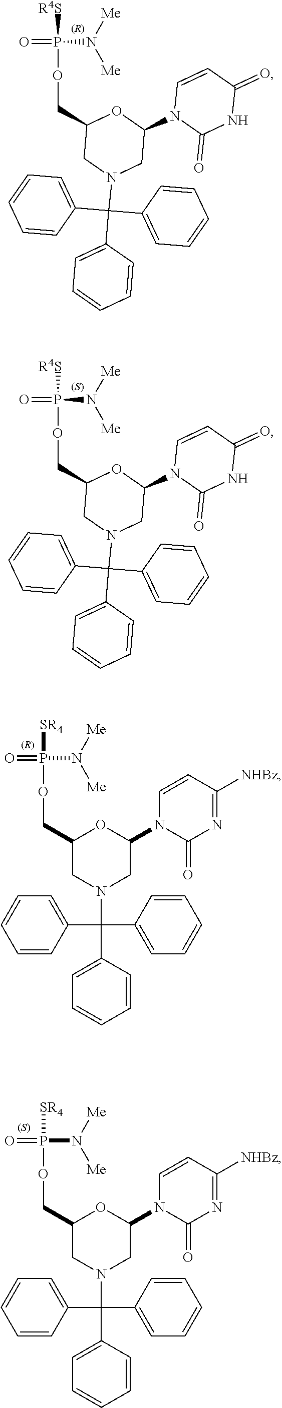

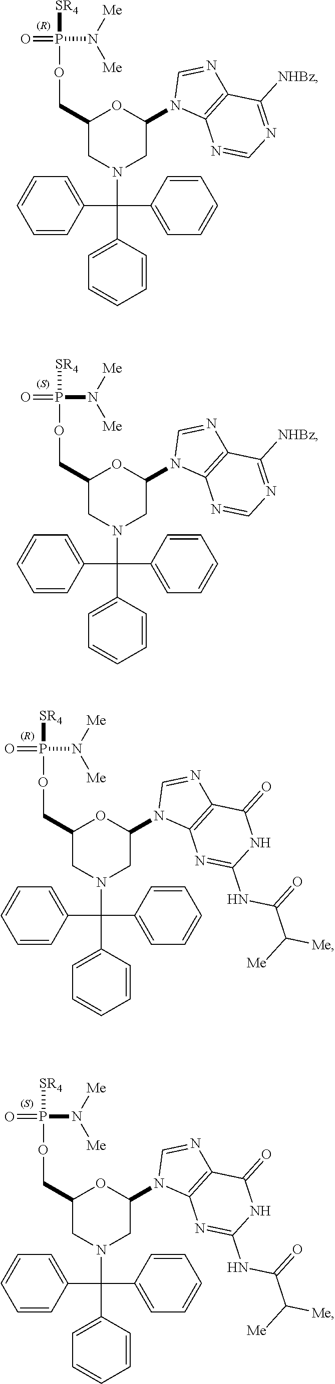

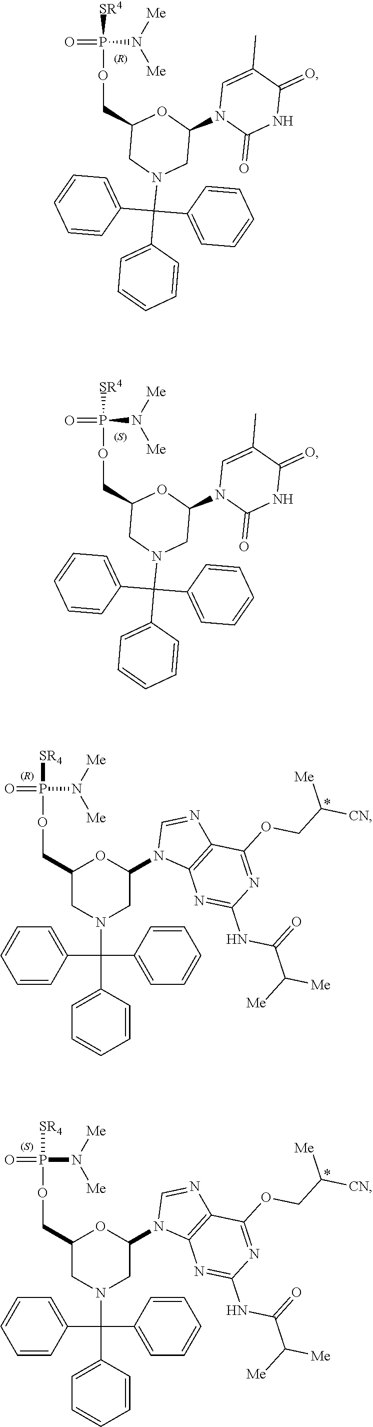

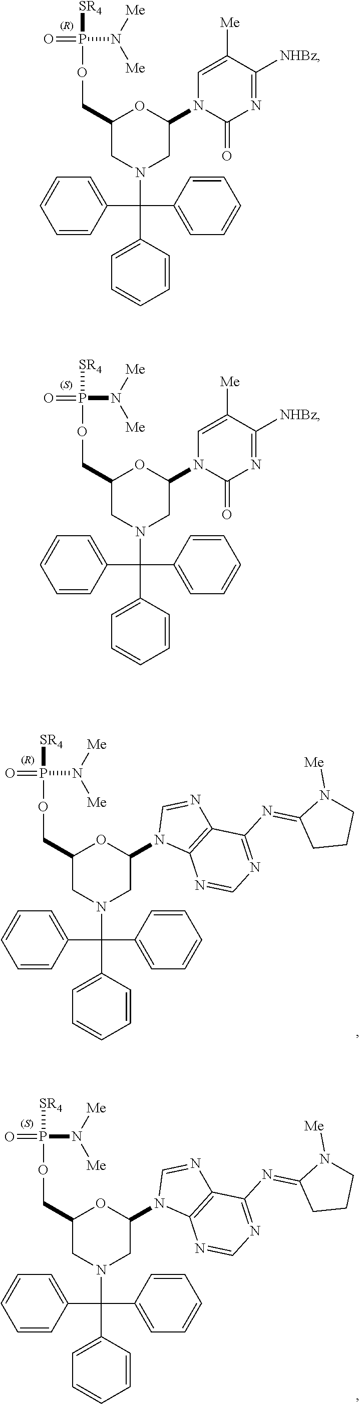

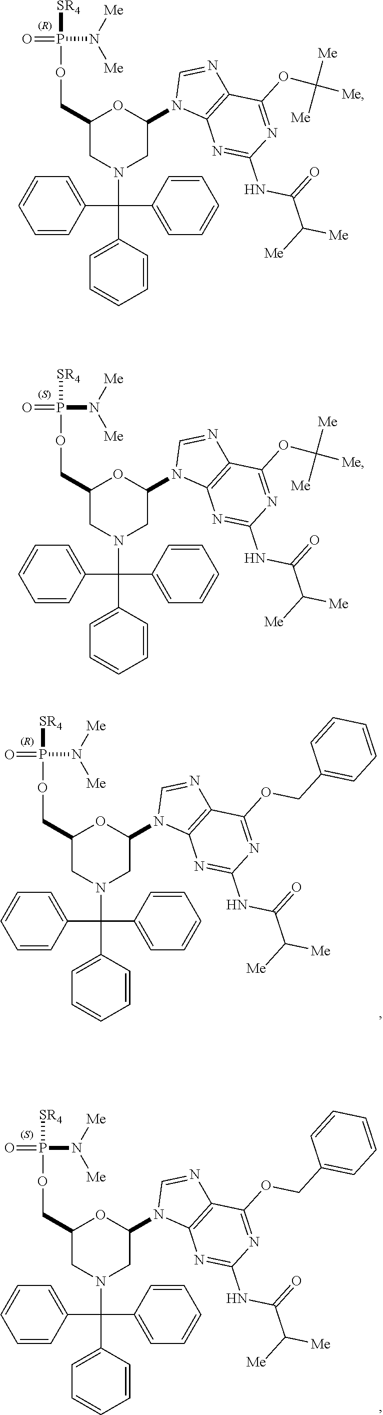

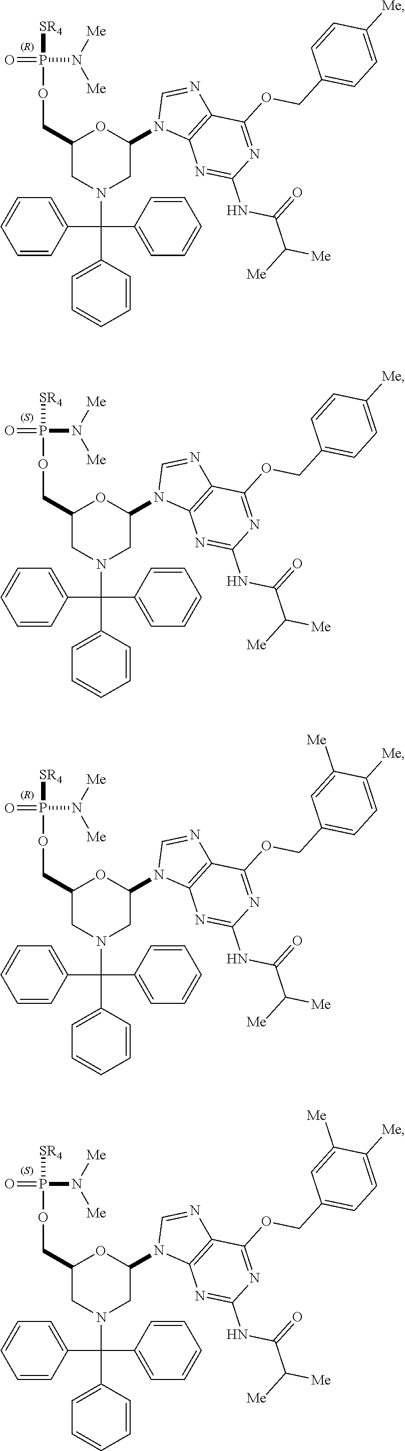

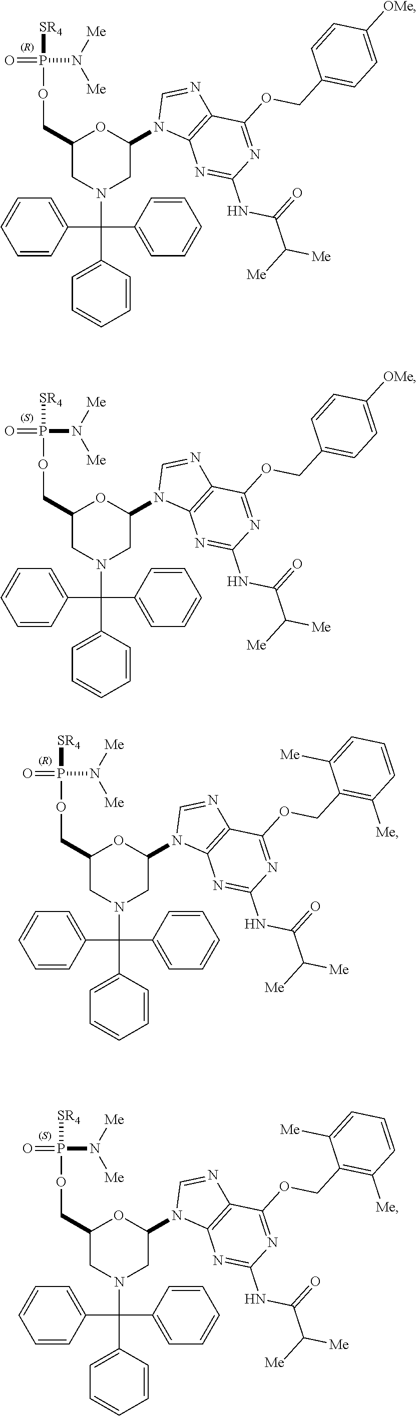

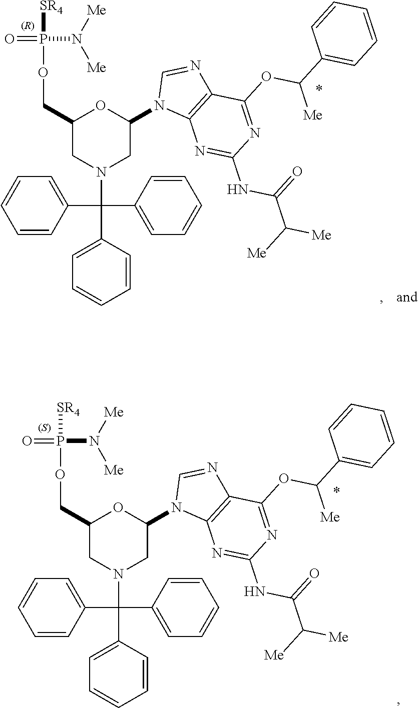

wherein * indicates either (R) or (S) stereochemistry, or a mixture thereof, and R⁴ is H or methyl.

Some embodiments provide use of a chiral reagent of Formula (IV)

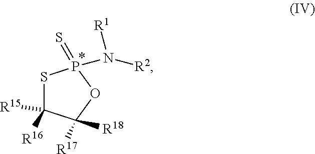

where P* represents a stereocenter that is either in the (R) or (S) configuration; R¹ and R² are independently H, optionally substituted C1-C6 alkyl, optionally substituted phenyl, optionally substituted naphthyl, or R¹ and R² together with the nitrogen they connect to form an optionally substituted heterocycle; R¹⁵, R¹⁶, R¹⁷ and R¹⁸ are independently H, C1-C10 alkyl, C2-C12 alkenyl, aryl, or any two of R¹⁵, R¹⁶, R¹⁷ and R¹⁸ together with the carbons they are attached form a cycloalkyl or heteroalkyl ring that is optionally substituted with one or more C1-C10 alkyl or C2-C12 alkenyl groups, in the preparation of a stereo-encoded morpholino monomer of formula (I),

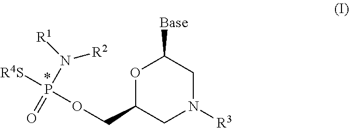

or a diastereomer or enantiomer thereof, or a salt thereof, wherein or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R³ is a protecting group for morpholino nitrogen, selected from optionally substituted C1-C6 alkyl, trityl, benzyl, or sulfonyl; R⁴ is hydrogen or C1-C6 alkyl; Base is selected from the group consisting of:

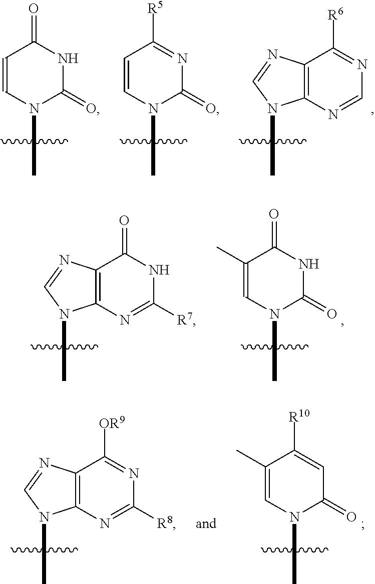

R⁵, R⁶, R⁷, R⁸, and R¹⁰ are independently —NR²⁰R²¹ or —N═CR¹²—NR¹³R¹⁴; R⁵, R⁶, R⁷, R⁸, and R¹⁰ are independently —NR²⁰R²¹, R²⁰ and R²¹ are independently selected from —H, —C(O)R¹¹, and —C(O)OR¹¹; R¹¹ is optionally substituted C1-C6 alkyl, optionally substituted benzyl, or aryl, optionally substituted with one or more substituents selected from halogen, nitro and alkoxyl; when R⁵, R⁶, R⁷, R⁸, and R¹⁰ are —NHC(O)R¹¹, R¹¹ is selected from methyl, ethyl, isopropyl, isobutyl, phenyl, 4-methoxyphenyl, 4-bromophenyl, and 4-nitrophenyl; when R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or R¹² and R¹³ together form a 5 or 6-membered heterocycle with the adjacent C and N; R⁹ is H, optionally substituted C1-C6 alkyl, optionally substituted benzyl, acyl, carbonate, carbamate, cyanoethyl, α-methylcyanoethyl, β-methylcyanoethyl, pivaloyloxy benzyl, or silyl; wherein “optionally substituted” indicates that a moiety may be substituted with one or more independently selected substitutents selected from C1-C6 alkyl, C1-C6 alkenyl, C1-C6 alkynyl, C1-C6 alkoxy, —OH, —NH₂, —NO₂, —CH₂—NH—CH₃, —OCH₂CH₂CH₃, or OCH(CH₃)₂; wherein the stereoselectivity or diastereoselectivity is at least 90%, 95%, or 98%.

In some embodiments of the use reported above, the chiral reagent is selected from

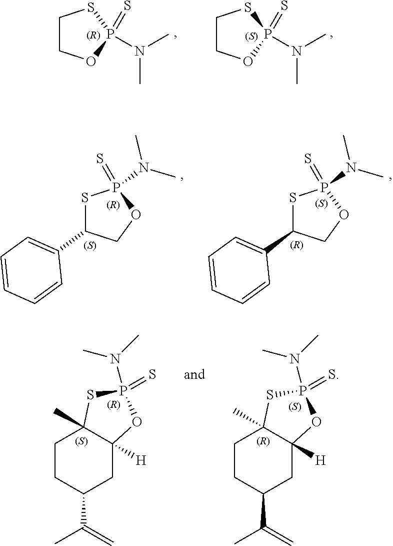

A further embodiment provides a stereo-encoded compound that is a monomer of Formula (Id)

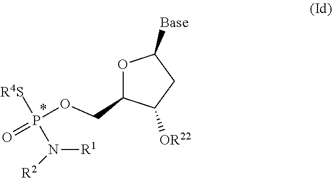

or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R²² is a protecting group for hydroxyl group; R⁴ is hydrogen or C1-C6 alkyl; Base is selected from the group consisting of:

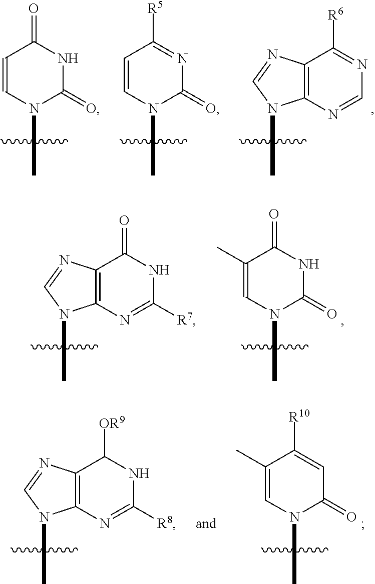

R⁵, R⁶, R⁷, R⁸, and R¹⁰ are independently —NR²⁰R²¹ or —N═CR¹²—NR¹³R¹⁴; R⁵, R⁶, R⁷, R⁸, and R¹⁰ are independently —NR²⁰R²¹, R²⁰ and R²¹ are independently selected from —H, —C(O)R¹¹, and —C(O)OR¹¹; R¹¹ is optionally substituted C1-C6 alkyl, optionally substituted benzyl, or aryl, optionally substituted with one or more substituents selected from halogen, nitro and alkoxyl; when R⁵, R⁶, R⁷, R⁸, and R¹⁰ are —NHC(O)R¹¹, R¹¹ is selected from methyl, ethyl, isopropyl, isobutyl, phenyl, 4-methoxyphenyl, 4-bromophenyl, and 4-nitrophenyl; when R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or R¹² and R¹³ together form a 5 or 6-membered heterocycle with the adjacent C and N; R⁹ is H, optionally substituted C1-C6 alkyl, optionally substituted benzyl, acyl, carbonate, carbamate, cyanoethyl, α-methylcyanoethyl, β-methylcyanoethyl, pivaloyloxy benzyl, or silyl.

In further embodiments of a compound reported above, the compound is crystalline. In a further embodiment R¹ and R² are independently optionally substituted C1-C6 alkyl. In a still further embodiment R¹ and R² are methyl. In a yet still further embodiment, R¹ and R² together with the nitrogen they connect to form a heterocycle selected from morpholinyl, piperazinyl, pyrrolidinyl, and azetidinyl. In an additional embodiment R²² is selected from the group consisting of H, trialkylsilyl, where the alkyl are the same or different and are C1-C6 alkyl, trityl, substituted trityl, p-methoxyphenyldiphenylmethyl, trityl, benzyl, 4-methoxybenzyl, 2-tetrahydropyranyl, and ethoxyethyl. R²² may be H, TBS, TBDPS, benzoyl, trityl, DMTr, p-methoxyphenyldiphenylmethyl, benzyl, 4-methoxybenzyl, 2-tetrahydropyranyl, and ethoxyethyl In a still further embodiment R²² is TBS or TBDPS. In another embodiment R⁴ is H or methyl. In a further embodiment R¹ and R² are methyl, R²² is TBS or TBDPS, and R⁴ is H or methyl. In a further embodiment, any of R⁵, R⁶, R⁷ R⁸ and R¹⁰ is independently: (a) —NHC(O)R¹¹, wherein R¹¹ is methyl, ethyl, isopropyl, isobutyl, phenyl, 4-methoxy phenyl, 4-bromophenyl, and 4-nitrophenyl, or

(b) —NHC(O)OR¹¹, wherein R¹¹ is C1-C6 alkyl, benzyl, 2,2,2-trichloroethyl, or aryl. In a yet still further embodiment, any of R⁵, R⁶, R⁷ R⁸ and R¹⁰ is independently

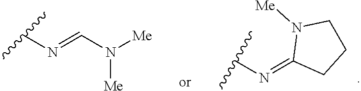 [SMILES: */N=C/N(C)C ; */N=C1/CCCN1C]

In a still further embodiment R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl. In a still further embodiment a compound as reported above is selected from the following compounds:

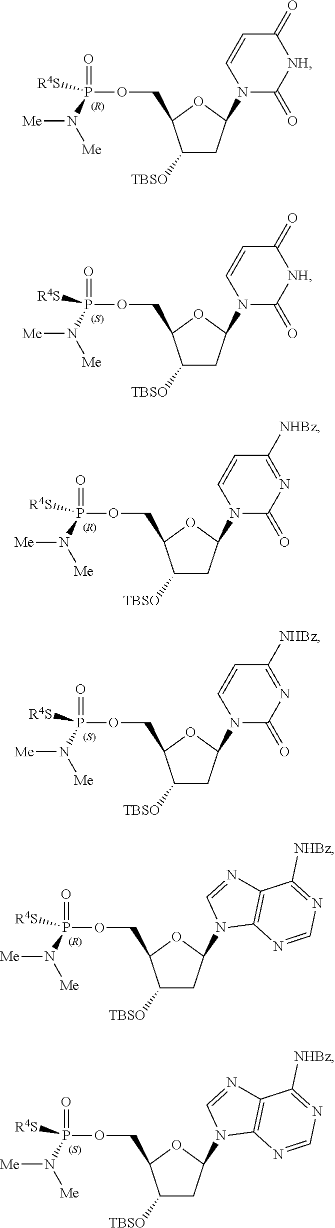

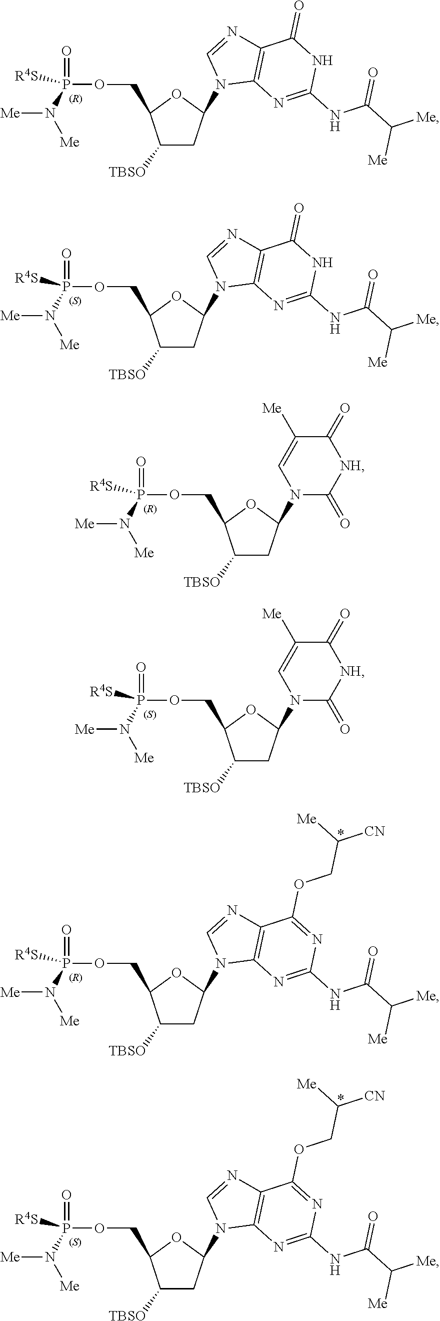

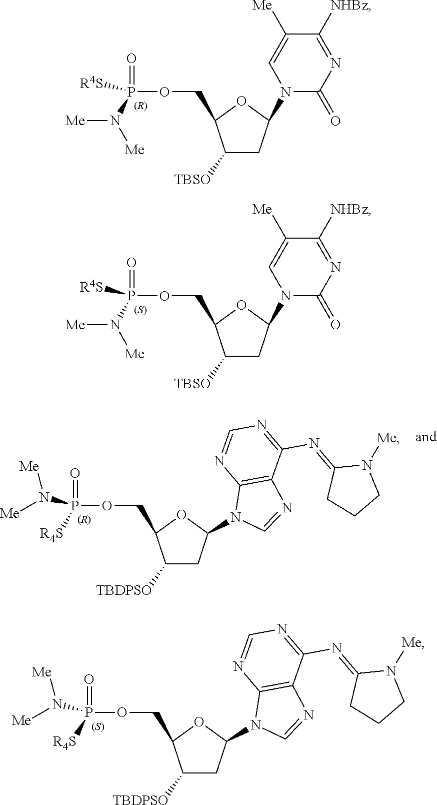

wherein * indicates either (R) or (S) stereochemistry, or a mixture thereof, and R⁴ is H or methyl.

A further embodiment provides a method of preparing a stereo-encoded activated monomer of Formula (IId)

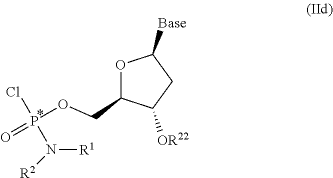

or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R²² is a protecting group for hydroxyl oxygen; Base is selected from the group consisting of:

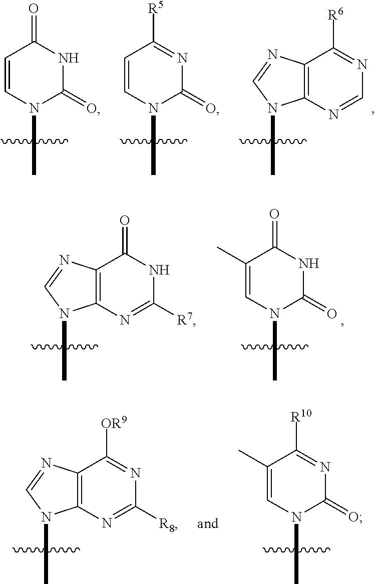

R⁵, R⁶, R⁷, R⁸, and R¹⁰ are independently —NR²⁰R²¹ or —N═CR¹²—NR¹³R¹⁴; R⁵, R⁶, R⁷, R⁸, and R¹⁰ are independently —NR²⁰R²¹, R²⁰ and R²¹ are independently selected from —H, —C(O)R¹¹, and —C(O)OR¹¹; when R⁵, R⁶, R⁷, R⁸, and R¹⁰ are —NHC(O)R¹¹, R¹¹ is selected from methyl, ethyl, isopropyl, isobutyl, phenyl, 4-methoxyphenyl, 4-bromophenyl, and 4-nitrophenyl; when R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or R¹² and R¹³ together form a 5 or 6-membered heterocycle with the adjacent C and N; R⁹ is H, optionally substituted C1-C6 alkyl, optionally substituted benzyl, acyl, carbonate, carbamate, cyanoethyl, α-methylcyanoethyl, β-methylcyanoethyl, pivaloyloxy benzyl, or silyl;
comprising the following steps:
(a) providing a stereo-encoded monomer of Formula (Iad)

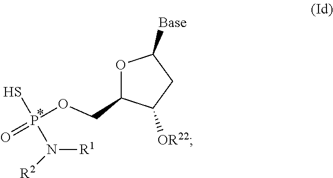

(b) optionally alkylating the sulfur of the stereo-encoded monomer of Formula (Iad) in step (a) to afford a stereo-encoded monomer of Formula (Id)

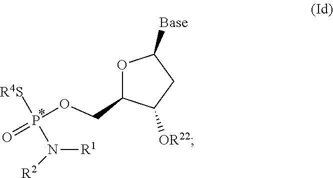

wherein R⁴ is C1-C6 alkyl; (c) reacting the stereo-encoded monomer of Formula (Ia) from step (a) or the stereo-encoded monomer of Formula (I) from step (b) with a chlorinating agent to afford the stereo-encoded activated monomer of Formula (IId).

In further embodiments of the method reported above, R⁴ is methyl. In a further embodiment R¹ and R² are independently C1-C6 alkyl. In a still further embodiment R¹ and R² are methyl. In a yet still further embodiment R¹ and R², together with the nitrogen to which they connect, form a heterocycle selected from morpholinyl, piperazinyl, pyrrolidinyl, and azetidinyl. In a further embodiment R²² is selected from the group consisting of H, TBS, TBDPS, benzoyl, trityl, DMTr, p-methoxyphenyldiphenylmethyl, benzyl, 4-methoxybenzyl, 2-tetrahydropyranyl, and ethoxyethyl. In a further embodiment R²² is TBS or TBDPS. In a further embodiment R¹ and R² are methyl, R²² is TBS or TBDPS, and R⁴ is H or methyl. In a still further embodiment, any of R⁵, R⁶, R⁷ R⁸ and R¹⁰ is independently (a) —NHC(O)R¹¹, wherein R¹¹ is methyl, ethyl, isopropyl, isobutyl, phenyl, 4-methoxy phenyl, 4-bromophenyl, and 4-nitrophenyl, or (b) —NHC(O)OR¹¹, wherein R¹¹ is C1-C6 alkyl, benzyl, 2,2,2-trichloroethyl, or aryl. In a further embodiment, any of R⁵, R⁶, R⁷ R⁸ and R¹⁰ is independently

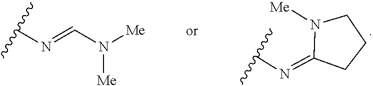 [SMILES: */N=C/N(C)C ; */N=C1/CCCN1C]

In a still further embodiment wherein R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl. In a yet still further embodiment the chlorinating agent is SO₂Cl₂. In another embodiment step (b) further includes reacting with 2,4,6-collidine. A still further embodiment provides wherein R⁴ is H and the chlorinating agent is tetramethyl chloroenamine.

An embodiment may provide a stereo-encoded activated monomer prepared by a method reported above.

A further embodiment may provide a method for preparing a stereo-encoded monomer of Formula (Id)

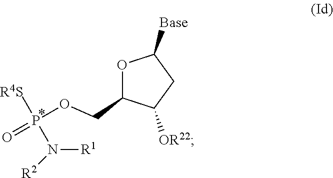

or a diastereomer or enantiomer thereof, or a salt thereof, wherein P* represents a stereocenter that is either in the (R) or (S) configuration; R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle; R²² is a protecting group for hydroxyl oxygen; R⁴ is hydrogen or C1-C6 alkyl; Base is selected from the group consisting of:

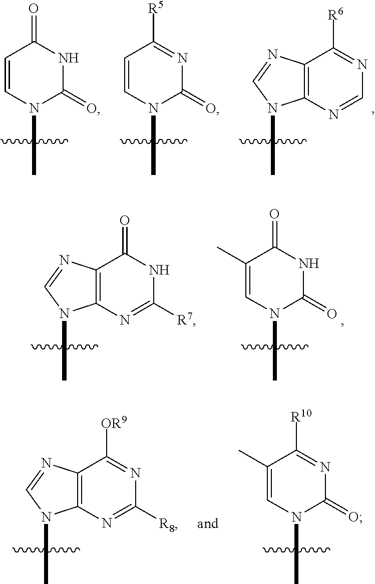

R⁵, R⁶, R⁷, R⁸, and R¹⁰ are independently —NR²⁰R²¹ or —N═CR¹²—NR¹³R¹⁴; where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹; when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl; when any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or R¹² and R¹³ together form a 5 or 6-membered heterocycle with the adjacent C and N; R⁹ is H, optionally substituted C1-C6 alkyl, optionally substituted benzyl, acyl, carbonate, carbamate, cyanoethyl, α-methylcyanoethyl, β-methylcyanoethyl, pivaloyloxy benzyl, or silyl; comprising the step of
reacting a compound of Formula (IIId)

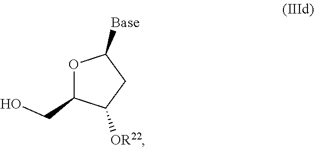

wherein R²² and Base are as defined above, with a chiral reagent of Formula (IV)

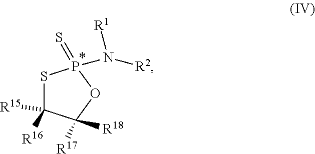

wherein R¹ and R² are as defined above; R¹⁵, R¹⁶, R¹⁷ and R¹⁸ are independently H, C1-C10 alkyl, C2-C12 alkenyl, aryl,
or any two of R¹⁵, R¹⁶, R¹⁷ and R¹⁸ together with the carbons they are attached form a cycloalkyl or heteroalkyl ring which is optionally substituted with one or more C1-C10 alkyl or C2-C12 alkenyl groups,
in the presence of a base to obtain the stereo-encoded monomer of Formula (Id).

In a further embodiment of a method reported above, R¹ and R² are methyl. In a still further embodiment R¹⁵, R¹⁶, R¹⁷ and R¹⁸ are independently H, C1-C10 alkyl, or aryl. In a still further embodiment R¹⁵, R¹⁶, R¹⁷ and R¹⁸ are H; or R¹⁵ is C1-C10 alkyl or aryl, and R¹⁶, R¹⁷, R¹⁸ are H; or R¹⁶ is C1-C10 alkyl or aryl, and R¹⁵, R¹⁷, R¹⁸ are H; or R¹⁵ is phenyl and R¹⁶, R¹⁷, R¹⁸ are H; or R¹⁶ is phenyl, and R¹⁵, R¹⁷, R¹⁸ are H. In a still further embodiment the chiral agent of Formula (IV) is selected from

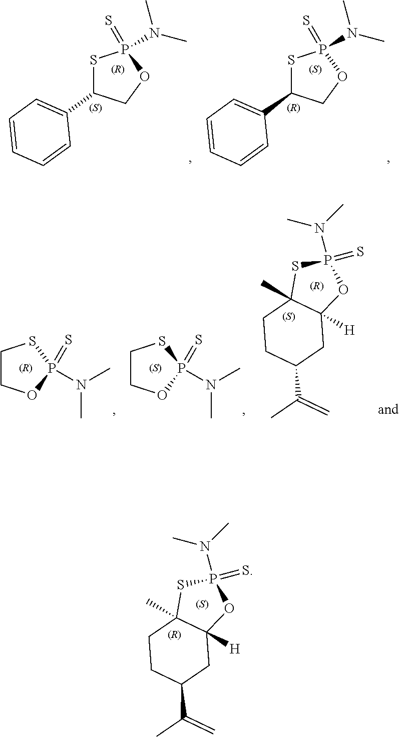

In further embodiments of the preceding method, the base is selected from the group consisting of NaH, DBU, sodium tert-amylate, sodium tert-pentoxide, NaOtBu, KOtBu, potassium tert-pentoxide, and NaHMDS. In further embodiments the reaction is conducted at room temperature in a polar solvent selected from the group consisting of THF, acetonitrile, 2-MeTHF, 1,6-dioxane, and DME. In further embodiments the stereo-encoded monomer of Formula (Id) is crystalline. In still further embodiments the de/ee ratio/stereoselectivity is at least 90%, 95% or 98%.

Further embodiments provide a stereo-encoded monomer of Formula (Id) prepared as reported above.

Some embodiments provide a method of making a phosphorodiamidate morpholino-DNA oligomer of formula (Xd)

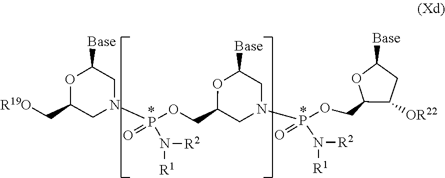

or a diastereomer or enantiomer thereof, or a salt thereof, wherein each P* represents a stereocenter that is either in the (R) or (S) configuration, n is an integer between 1 and 7 inclusive, R¹ and R² are independently H, optionally substituted C1-C6 alkyl, optionally substituted phenyl, optionally substituted naphthyl, or R¹ and R² together with the nitrogen to which they connect to form an optionally substituted heterocycle, and wherein each Base may be the same or different and is selected from the group consisting of

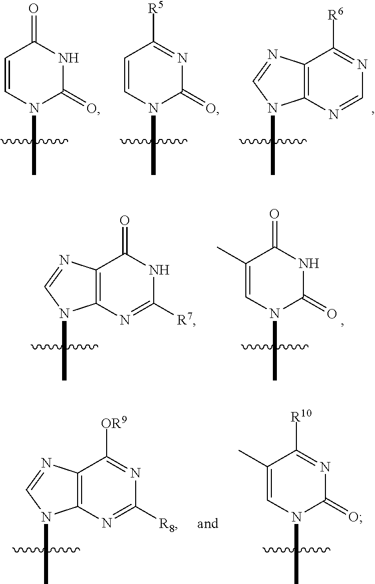

wherein R⁵, R⁶, R⁷, R, and R¹⁰ are independently —NR²⁰R²¹ or —N═CR¹²—NR¹³R¹⁴.
wherein any of R⁵, R⁶, R⁷, R⁸, and R¹⁰ is independently —NR²⁰R²¹, R²⁰ and R²¹ are independently selected from —H, —C(O)R¹¹, and —C(O)OR¹¹; where R⁵, R⁶, R⁷, R⁸, and R¹⁰ are —NHC(O)R¹¹, R¹¹ is methyl, ethyl, isopropyl, isobutyl, phenyl, 4-methoxyphenyl, 4-bromophenyl, or 4-nitrophenyl; or where R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl; or wherein any of R⁵, R⁶, R⁷, R⁸, and R¹⁰ is independently —N═CR¹²—NR¹³R¹⁴; or wherein R¹², R¹³, and R¹⁴ are independently C1-C6 alkyl, or R¹² and R¹³ together form a 5 or 6-membered heterocycle with the adjacent C and N; and wherein R⁹ is selected from the group consisting of H, optionally substituted C1-C6 alkyl, optionally substituted benzyl, acyl, carbonate, carbamate, and silyl; R¹⁹ and R²² are the same or different and are independently H or a protecting group for a hydroxyl group, or a linker to solid support, comprising the following steps:
   (a) providing a stereo-encoded morpholino monomer of Formula (Ia)

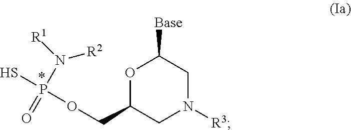

wherein R³ is optionally substituted alkyl, trityl, benzyl, or sulfonyl; (b) optionally sulfur-alkylating the stereo-encoded morpholino monomer of Formula (Ia) in step (a) to afford a stereo-encoded morpholino monomer of Formula (I)

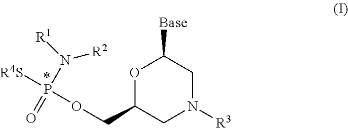

wherein R⁴ is C1-C6 alkyl; (c) reacting the stereo-encoded morpholino monomer of Formula (Ia) from step (a) or the stereo-encoded morpholino monomer of Formula (I) from (b) with a chlorinating agent to afford a stereo-encoded activated monomer of Formula (II)

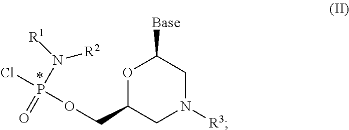

(d) optionally isolating the stereo-encoded activated compound of Formula (II) from step (c); (e) coupling the compound of Formula (II) from step (c) or (d) with a compound of Formula (IX)

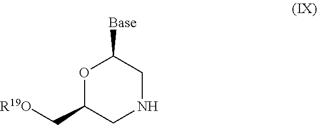

to obtain a morpholino dimer of Formula (VIII)

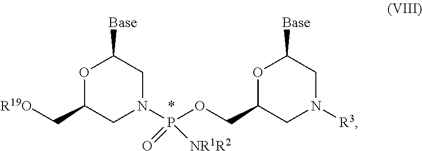

wherein R¹⁹ is a suitable protecting group for a hydroxyl group; (f) deprotecting the morpholino dimer of Formula (VIII) to obtain an intermediate oligomer of Formula (X)

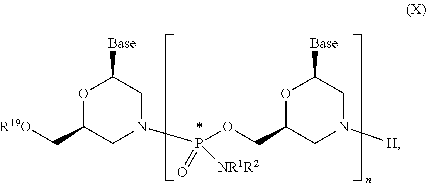

wherein n is 1; (g) repeating step (i) and step (ii) below m times, wherein m is an integer from 0 to 6 inclusive:
(i) reacting the intermediate oligomer of Formula (X)

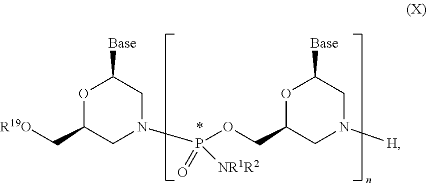

wherein n is an integer from 1 to 7 inclusive, with a stereo-encoded activated compound of Formula (II) prepared from a stereo-encoded morpholino monomer of Formula (I) by steps (a) to (d)

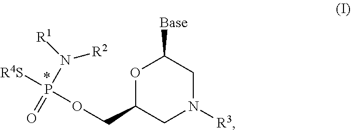

wherein R⁴ is H or C1-C6 alkyl, to provide an intermediate of formula (XI):

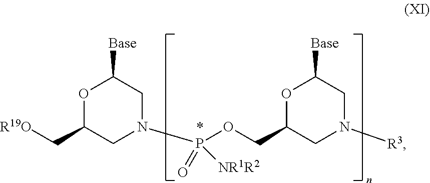

wherein n is an integer from 1 to 7 inclusive; and (ii) deprotecting the intermediate of Formula (XI) from step (i) to provide a phosphorodiamidate morpholino oligomer of Formula (X):

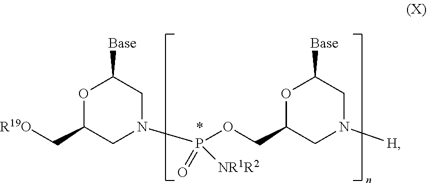

wherein n is an integer from 1 to 7 inclusive;
(h) providing a stereo-encoded DNA monomer of Formula (Iad)

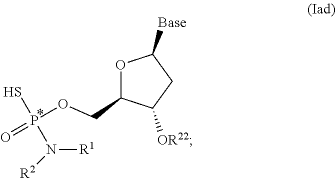

wherein R²² is a protecting group for hydroxyl group; (j) optionally sulfur-alkylating the stereo-encoded DNA monomer of Formula (Iad) in step (h) to afford a stereo-encoded DNA monomer of Formula (Id)

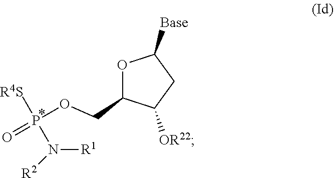

wherein R⁴ is C1-C6 alkyl; (k) reacting the stereo-encoded DNA monomer of Formula (Iad) from step (h) or the stereo-encoded DNA monomer of Formula (Id) from (j) with a chlorinating agent to afford a stereo-encoded activated monomer of Formula (IId)

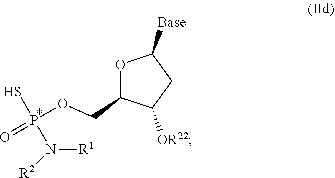

(I) optionally isolating the stereo-encoded activated compound of Formula (IId) from step (k); (m) coupling the compound of Formula (IId) from step (k) or (l) with a compound of Formula (X) from step (g):

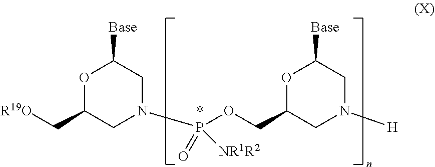

wherein n is an integer from 1 to 7 inclusive; to provide a phosphorodiamidate morpholino-DNA oligomer of formula (Xd):

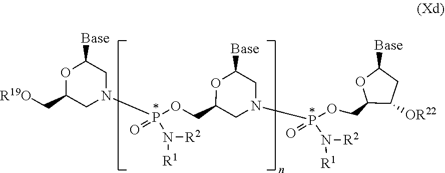

(n) optionally, removing the R²² group and/or the R¹⁹ group in the phosphorodiamidate morpholino-DNA oligomer of formula (Xd), to provide a PMO-DNA hetero-oligomer of Formula (Xd):

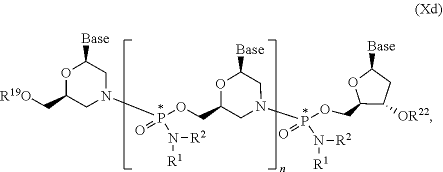

wherein n is from 1 to 7 inclusive, and R¹⁹ and R²² are independently H or a hydroxyl protecting groups, or a linker to solid support, that may be the same or different.

In further embodiments of the method above R¹ and R² are methyl. In still further embodiments R³ is trityl. In still further embodiments wherein R¹⁹ and R²² are independently selected from H, TBS, TBDPS, benzoyl, trityl, DMTr, p-methoxyphenyldiphenylmethyl, benzyl, 4-methoxybenzyl, 2-tetrahydropyranyl, and ethoxyethyl, or a linker to solid support.

Further embodiments may provide a phosphorodiamidate morpholino-DNA hetero-oligomer made by a method reported above.

### DETAILED DESCRIPTION OF EMBODIMENTS

Provided herein are crystalline stable stereo-encoded (that is, chiral) monomers that may include one of adenine (A), cytosine (C), guanine (G), uracil (U), and thymine (T), or their analogs, as well as methods of their preparation and use, including for synthesis of stereopure polymorpholino oligomers. Embodiments further provide methods of stereo-specific activated monomers. Some embodiments provide crystalline stable, chiral Morpholino monomers. Other embodiments further provide crystalline stable stereo-encoded DNA monomers.

### A. Definitions

Compounds useful as active agents in accordance with the present disclosure include those described generally above and below, and are further illustrated by the embodiments, sub-embodiments, and species disclosed herein. As used herein, the following definitions shall apply unless otherwise indicated.

An “activated monomer” as used herein is a monomer that has been stereo-encoded and prepared to use for synthesis of a dimer or oligomer.

A “stereo-encoded” or “stereomerically encoded” monomer as used herein is a monomer that, when used according to methods presented in this disclosure, will prepare a stereo-specific dimer or oligomer.

As described herein, compounds of the invention may optionally be substituted with one or more substituents, such as those illustrated generally herein, or as exemplified by particular classes, subclasses, and species of the invention. In general, the term “substituted” refers to the replacement of hydrogen in a given structure with a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable compounds.

An “optionally substituted” moiety is a moiety that may be unsubstituted, or that may be substituted. If no specific options for optional substitution are indicated, then it will be understood that the compound may be substituted with one or more of C1-C6 alkyl, C2-C8 alkenyl, C2-C6 alkynyl, C1-C6 alkoxy (such as, for example, but not limited to, —OCH₂CH₂CH₃, or —OCH(CH₃)₂), C1-C8 amino, C3-C10 heterocyclyl, C5-C15 aryl, —OH, —NH₂, —F, —Cl, —Br, —SO₃, —CN, —NO₂, —CF₃, —CH₂—NH—CH₃. Any of the hydrogen adjacent to a carbon or nitrogen in the optional substituents may be further substituted by a group such as —OH, —NH₂, —F, —Cl, —Br, —SO₃, —CN, —NO₂, —CF₃, —CH₂—NH—CH. If the optionally substituted moiety is cyclic, then the optional substitution may be an alkylene bridge between two atoms in the ring. A description such as “optionally substituted Group 1, Group 2, and Group 3” means all Groups are optionally substituted, i.e., optionally substituted Group 1, optionally substituted Group 2, and optionally substituted Group 3.

“Stable” as used herein refers to chemically feasible compound is one that is not substantially altered when kept at a temperature of 40° C. or less, in the absence of moisture or other chemically reactive conditions, for at least a week.

As would be understood by those of skill in the art, as used herein “H” is hydrogen, “C” is carbon, “N” is nitrogen, “S” is sulfur, and “O” is oxygen.

“Alkyl” or “alkyl group,” as used herein, means a straight-chain (i.e., unbranched), or branched hydrocarbon chain that is completely saturated. In some embodiments, the alkyl has 1, 2, 3, 4, 5, 6, 7, 8, 9, 10 or more carbon atoms. In certain embodiments, alkyl groups contain 1-10 carbon atoms (C1-C10 alkyl). In certain embodiments, alkyl groups contain 1-6 carbon atoms (C1-C6 alkyl). In certain embodiments, alkyl groups contain 1-4 carbon atoms (C1-C4 alkyl). In certain embodiments, alkyl groups contain 1-3 carbon atoms (C1-C3 alkyl). In still other embodiments, alkyl groups contain 2-3 carbon atoms (C2-C3 alkyl), and in yet other embodiments alkyl groups contain 1-2 carbon atoms (C1-C2 alkyl).

“Alkenyl” or “alkenyl group,” as used herein, refers to a straight-chain (i.e., unbranched), or branched hydrocarbon chain that has one or more double bonds. In some embodiments, the alkenyl has 2, 3, 4, 5 or 6 carbon atoms. In certain embodiments, alkenyl groups contain 2-12 carbon atoms (C2-C12 alkenyl). In certain embodiments, alkenyl groups contain 2-6 carbon atoms (C2-C6 alkenyl). In still other embodiments, alkenyl groups contain 3-4 carbon atoms (C3-C4 alkenyl), and in yet other embodiments alkenyl groups contain 2-3 carbon atoms (C2-C3 alkenyl). According to another aspect, the term alkenyl refers to a straight chain hydrocarbon having two double bonds, also referred to as “diene.” Non-limiting examples of exemplary alkenyl groups include —CH═CH₂, —CH₂CH═CH₂, —CH═CHCH₃, —CH₂CH₂CH═CH₂, —CH₂CH═CHCH₃, —CH═CHCH₂CH₃, and —CH═CHCH═CH₂.

“Alkynyl” or “alkynyl group” as used herein refers to a straight-chain (i.e., unbranched), or branched hydrocarbon chain that has one or more triple bonds. In some embodiments, the alkynyl has 2, 3, 4, 5 or 6 carbon atoms. In certain embodiments, alkynyl groups contain 2-8 carbon atoms (C2-C8 alkynyl). In certain embodiments, alkynyl groups contain 2-6 carbon atoms (C2-C6 alkynyl). In still other embodiments, alkynyl groups contain 3-4 carbon atoms (C3-C4 alkynyl), and in yet other embodiments alkynyl groups contain 2-3 carbon atoms (C2-C3 alkynyl).

“Ar” or “aryl” refer to an aromatic carbocyclic moiety having one or more closed rings. Examples include, without limitation, phenyl, naphthyl, anthracenyl, phenanthracenyl, biphenyl, and pyrenyl. Unless otherwise stated, “Ar” or “aryl” encompasses heteroaryl as described below.

“Acyl” refers to a moiety derived from the removal of one or more hydroxyl groups from an oxoacid. Examples include, without limitation, formyl, acetyl, propionyl, butyryl, acrylyl, crotonyl, and benzoyl.

“Halo” refers to chloro (Cl), fluoro (F), bromo (Br) or iodo (I). Reference to “halogen” substituents includes substitution with a “halo.”

“Halide” refers to a halogen leaving group.

“Heteroaryl” refers to a heterocyclic moiety having one or more closed rings, with one or more heteroatoms (oxygen, nitrogen or sulfur) in at least one of the rings, wherein at least one of the rings is aromatic, and wherein the ring or rings may independently be fused, and/or bridged. Examples include, without limitation, quinolinyl, isoquinolinyl, indolyl, furyl, pyridinyl, thienyl, pyrazolyl, quinoxalinyl, pyrrolyl, indazolyl, thieno[2,3-c]pyrazolyl, benzofuryl, pyrazolo[1,5-a]pyridyl, thiophenylpyrazolyl, benzothienyl, benzothiazolyl, thiazolyl, 2-phenylthiazolyl, imidazolyl, pyrrolyl, furanyl, thiophenyl, pyridinyl, pyrimidinyl, pyrazinyl, indolyl, quinolinyl, and isoxazolyl.

“—OR” or “oxy” refers to an R group appended to the parent molecular moiety through an oxygen atom, wherein R is H, alkyl, alkenyl, alkynyl, and the like.

“Sulfonyl” refers to a group for the general formula R—S(═O)₂—R′. Examples of sulfonyl groups include, without limitation, p-toluenesulfonyl, p-bromobenzenesulfonyl, o-nitrobenzenesulfonyl, p-nitrobenzenesulfonyl, methanesulfonyl, triflyuoromethanesulfonyl, 2,2,2-trifluoroethyl-1-sulfonyl, and 5-(dimethylamino)naphthalene-1-sulfonyl.

“Alkoxy” refers to an alkyl group, as herein defined, attached to the principal carbon chain through an oxygen (“alkoxy”) atom. Representative examples of “alkoxy” include, but are not limited to, methoxy, ethoxy, propoxy, phenoxy, 2-propoxy, butoxy, tert-butoxy, pentyloxy, hexyloxy and the like.

“Hydroxy” or “hydroxyl” refers to an —OH group.

“Carbonyl” is a group having a carbon atom double-bonded to an oxygen atom (C═O), often depicted in chemical formulae as C(O).

An “amine” or “amino” refers to a group —NH₂, wherein none, one or two of the hydrogens may replaced by a suitable substituent as described herein, such as alkyl, alkenyl, alkynyl, and the like.

An “amide” or “amido” refers to a group having a carbonyl bonded to a nitrogen atom, such as —C(O)NH₂, wherein none, one or two of the hydrogens may replaced by a suitable substituent as described herein, such as alkyl, alkenyl, alkynyl, and the like.

“Benzyl” is a benzene ring attached to a methylene group, i.e. C₆H₅CH₂—

“Cycloalkyl” as used herein, refers to a saturated cyclic hydrocarbon group containing from 3 to 8 carbons or more. Representative examples of cycloalkyl include, but are not limited to, cyclopropyl, cyclobutyl, cyclopentyl, cyclohexyl, cycloheptyl, and cyclooctyl.

“Cycloalkenyl” as used herein, refers to an unsaturated cyclic hydrocarbon group containing from 3 to 8 carbons or more and having one or more double bonds.

“Cycloalkynyl” as used herein, refers to an unsaturated cyclic hydrocarbon group containing from 3 to 8 carbons or more and having one or more triple bonds.

“Alpha-beta-unsaturated amide” or “unsaturated amide” as used herein refers to an amide comprising an alkene or alkyne bonded directly to the amide carbonyl group and is represented by the structure

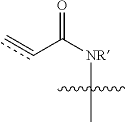

wherein R′ is hydrogen or alkyl.

“Heteroatom” refers to O, S or N.

“Heterocycle” or “heterocyclyl” as used herein, means a monocyclic heterocycle, a bicyclic heterocycle, or a tricyclic heterocycle containing at least one heteroatom in the ring.

A monocyclic heterocycle is a 3-, 4-, 5-, 6-, 7, or 8-membered ring containing at least one heteroatom independently selected from the group consisting of O, N, and S. In some embodiments, the heterocycle is a 3- or 4-membered ring containing one heteroatom selected from the group consisting of O, N and S. In some embodiments, the heterocycle is a 5-membered ring containing zero, one or two double bond and one, two. three, or four heteroatoms selected from the group consisting of O, N and S. In some embodiments, the heterocycle is a 6-, 7-, or 8-membered ring containing zero, one, two, three or four double bonds and one, two, three or four heteroatoms selected from the group consisting of O, N and S. Representative examples of monocyclic heterocycles include, but are not limited to, azetidinyl, azepanyl, aziridinyl, diazepanyl, 1,3-dioxanyl, 1,3-dioxolanyl, dihydropyranyl (including 3,4-dihydro-2H-pyran-6-yl), 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydropyranyl (including tetrahydro-2H-pyran-4-yl), tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

Bicyclic heterocycles may be exemplified by a monocyclic heterocycle fused to an aryl group, or a monocyclic heterocycle fused to a monocyclic cycloalkyl, or a monocyclic heterocycle fused to a monocyclic cycloalkenyl, or a monocyclic heterocycle fused to a monocyclic heterocycle. Representative examples of bicyclic heterocycles include, but are not limited to, 3,4-dihydro-2H-pyranyl, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, 3,4-dihydroquinolin-2(1H)-one and 1,2,3,4-tetrahydroquinolinyl.

The tricyclic heterocycle is a bicyclic heterocycle fused to an aryl group, or a bicyclic heterocycle fused to a monocyclic cycloalkyl, or a bicyclic heterocycle fused to a monocyclic cycloalkenyl, or a bicyclic heterocycle fused to a monocyclic heterocycle. Representative examples of tricyclic heterocycles include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b, d]thienyl.

In the above heteroaryl and heterocycles the nitrogen or sulfur atoms can be optionally oxidized to various oxidation states. In a specific example, the group S(O)₀₋₂ refers to —S— (sulfide), —S(O)— (sulfoxide), and —SO₂— (sulfone) respectively. For convenience, nitrogens, particularly but not exclusively, those defined as annular aromatic nitrogens, are meant to include those corresponding N-oxide forms.

“Salt” as used herein refer to acid addition salts or base addition salts of the compounds in the present disclosure. Salts include, but are not limited to, metal complexes and salts of both inorganic and carboxylic acids. Salts also include metal salts such as aluminum, calcium, iron, magnesium, manganese and complex salts. In addition, salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycolic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Salts may be derived from amino acids including, but not limited to, cysteine. Methods for producing compounds as salts are known to those of skill in the art (see, e.g., Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977).

Unless indicated otherwise, nomenclature used to describe chemical groups or moieties as used herein follow the convention where, reading the name from left to right, the point of attachment to the rest of the molecule is at the right-hand side of the name. For example, the group “arylC1-C6alkyl” is attached to the rest of the molecule at the alkyl end.

Unless indicated otherwise, where a chemical group is described by its chemical formula, including a terminal bond moiety indicated by “−,” it will be understood that the attachment is read from left to right. For example, —C(O)C1-C6alkyl is attached to the rest of the molecule at the carbonyl end.

Unless otherwise stated, structures depicted herein are also meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. In addition, unless otherwise stated, all rotamer forms of the compounds of the invention are within the scope of the invention. Unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

“Isomers” refer to compounds having the same number and kind of atoms and hence the same molecular weight, but differing with respect to the arrangement or configuration of the atoms. It will be understood, however, that some isomers or racemates or others mixtures of isomers may exhibit more activity than others. “Stereoisomers” refer to isomers that differ only in the arrangement of the atoms in space. “Diastereoisomers” or “diastereomers” refer to stereoisomers that are not mirror images of each other. “Enantiomers” refers to stereoisomers that are non-superimposable mirror images of one another.

In some embodiments, enantiomeric compounds taught herein may be “enantiomerically pure” isomers that comprise substantially a single enantiomer, for example, greater than or equal to 90%, 92%, 95%, 98%, or 99%, or equal to 100% of a single enantiomer.

In some embodiments, enantiomeric compounds taught herein may be stereomerically pure. “Stereomerically pure” as used herein means a compound or composition thereof that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of diastereomers, and substantially free of the opposite enantiomer, of the compound. A typical stereomerically pure compound comprises greater than about 80% by weight of one stereoisomer of the compound and less than about 20% by weight of the other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. See, e.g., U.S. Pat. No. 7,189,715.

“R” and “S” as terms describing isomers are descriptors of the stereochemical configuration at an asymmetrically substituted carbon atom. The designation of an asymmetrically substituted carbon atom as “R” or “S” is done by application of the Cahn-Ingold-Prelog priority rules, as are well known to those skilled in the art, and described in the International Union of Pure and Applied Chemistry (IUPAC) Rules for the Nomenclature of Organic Chemistry. Section E, Stereochemistry.

“Enantiomeric excess” (ee) of an enantiomer is [(the mole fraction of the major enantiomer) minus (the mole fraction of the minor enantiomer)]×100.

### B. Chiral Reagents of Formula (IV)

Embodiments may provide a chiral reagent of Formula (IV), which may be useful in a stereoselective synthesis described further herein:

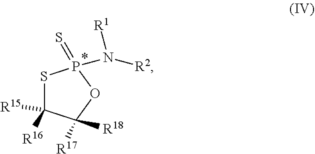

Embodiments may also provide a diastereomer or enantiomer thereof, or a salt thereof. In Formula (IV), P* represents a stereocenter that is either in the (R) or (S) configuration. R¹ and R² are independently selected from —H, optionally substituted C1-C6 alkyl, optionally substituted phenyl, and optionally substituted naphthyl, or R¹ and R² together with the nitrogen they connect to form an optionally substituted heterocycle. Example of the optionally substituted heterocycle includes morpholinyl, piperazinyl, pyrrolidinyl, and azetidinyl. In some embodiments both R¹ and R² are C1-C6 alkyl. In some further embodiments, both R¹ and R² are methyl.

R¹⁵, R¹⁶, R¹⁷, and R¹⁸ may be independently H, C1-C10 alkyl, C2-C12 alkenyl, or aryl. Any two of R¹⁵, R¹⁶, R¹⁷, and R¹⁸ together with the carbons they are attached may form a cycloalkyl or heteroalkyl ring, which is optionally substituted with one or more C1-C10 alkyl or C2-C12 alkenyl groups.

In some embodiments, R¹⁵, R¹⁶, R¹⁷, R¹⁸ are H. In some embodiments, R¹⁵ is C1-C10 alkyl or aryl, and R¹⁶, R¹⁷, R¹⁸ are H, or R¹⁶ is C1-C10 alkyl or aryl, and R¹⁵, R¹⁷, R¹⁸ are H. In some embodiments, R¹⁵ is phenyl and R¹⁶, R¹⁷, and R¹⁸ are H, or R¹⁶ is phenyl, and R¹⁵, R¹⁷, and R¹⁸ are H. In some embodiments, R¹⁵ and R¹⁶ are methyl or form a cyclohexyl ring, and R¹⁷ and R¹⁸ are H.

In further embodiments, R¹⁵ is methyl; R¹⁶ and R¹⁷ together form

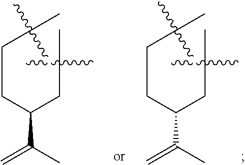

### and R¹⁸ is H.

Non-limiting examples of compounds of Formula (IV) are show in Table 1, below.

TABLE 1

| Compound Number | Compound |
|---|---|
| 1 | 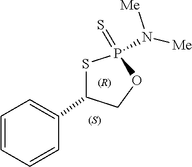 |
| 2 | 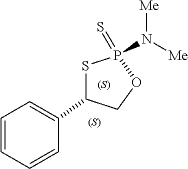 |
| 3 | 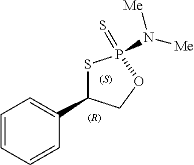 |
| 4 | 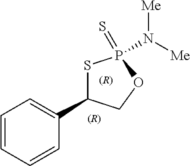 |
| 5 | 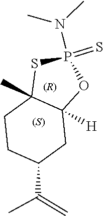 |
| 6 | 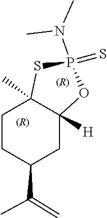 |
| 7 | 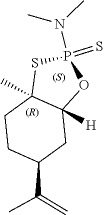 |
| 8 | 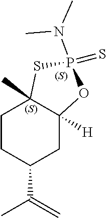 |
| 9 | 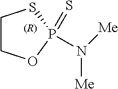 |
| 10 | 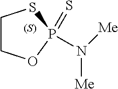 |

### C. Methods of Preparing Chiral Reagents of Formula (IV)

### C1. One Method of Preparing Chiral Reagents of Formula (IV)

Embodiments may provide a method of making a chiral reagent of formula (IV):

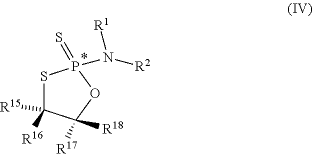

or a diastereomer or enantiomer thereof, or a salt thereof. P* represents a stereocenter that is either in the (R) or (S) configuration. R¹ and R² are independently H or C1-C6 alkyl, or R¹ and R² together with the nitrogen they connect to form an optionally substituted heterocycle selected from morpholinyl, piperazinyl, pyrrolidinyl, and azetidinyl. In some embodiments both R¹ and R² are C1-C6 Alkyl. In some further embodiments, both R¹ and R² are methyl.

R¹⁵, R¹⁶, R¹⁷, and R¹⁸ may be independently H, C1-C10 alkyl, C2-C12 alkenyl, or aryl. In some embodiments, any two of R¹⁵, R¹⁶, R¹⁷, and R¹⁸ together with the carbons they are attached may form a cycloalkyl or heteroalkyl ring, which is optionally substituted with one or more C1-C10 alkyl or C2-C12 alkenyl groups.

In some embodiments, R¹⁵, R¹⁶, R¹⁷, R¹⁸ are H. In some embodiments, R¹⁵ is C1-C10 alkyl or aryl, and R¹⁶, R¹⁷, R¹⁸ are H, or R¹⁶ is C1-C10 alkyl or aryl, and R¹⁵, R¹⁷, R¹⁸ are H. In some embodiments, R¹⁵ is phenyl and R¹⁶, R¹⁷, and R¹⁸ are H, or R¹⁶ is phenyl, and R¹⁵, R¹⁷, and R¹⁸ are H.

In further embodiments, R¹⁵ is methyl; R¹⁶ and R¹⁷ together form

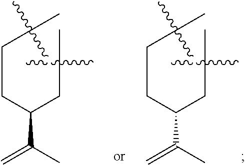

### and R¹⁸ is H.

In one embodiment the method of preparation of a compound of Formula (IV) includes the steps of reacting a compound of Formula (V)

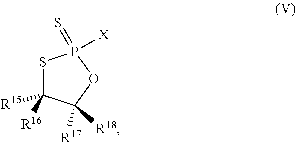

wherein X is a leaving group selected from substituted —S-aryl, —O-aryl or halide, wherein aryl is phenyl, naphthyl, optionally substituted with one or more of alkyl, cyano, nitro or halo, with NHR¹R² in the presence of an organic base to obtain the chiral reagent of Formula (IV).

In some embodiments, the leaving group X is —S—Ar or —O—Ar, wherein Ar is one of phenyl, pentafluorophenyl, 4-bromophenyl, mononitrophenyl, or dinitrophenyl.

In some embodiments, the organic base is 1,8-Diazabicyclo[5.4.0]undec-7-ene, commonly known as DBU, imidazole, triethylamine, Hunig's base, lutidine, pyridine and any combination there of.

Nonlimiting examples of compounds that may be made according to this method are those show in Table 1, above.

### C2. Another Method of Preparing Chiral Reagents of Formula (IV)

An additional method of making a subgenus of chiral reagents of Formula (IV), referred to as Formula (IVa) is provided below.

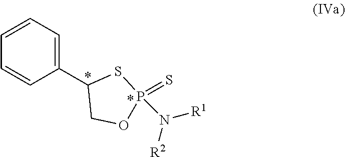

wherein C* and P* each represents a stereocenter that is independently either in the (R) or (S) configuration. R¹ and R² are independently H or C1-C6 alkyl, or R¹ and R² together with the nitrogen they connect to form an optionally substituted heterocycle selected from morpholinyl, piperazinyl, pyrrolidinyl, and azetidinyl.

This embodiment includes the step of reacting a compound of Formula (VI)

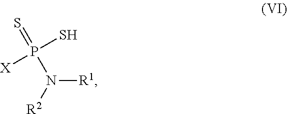

or a salt thereof, with a chiral styrene oxide (i.e. S-styrene oxide or R-styrene oxide) in the presence of chloroacetic acid to obtain the chiral reagent of Formula (IVa). In some embodiments, X is a leaving group, for example, substituted —S—Ar, —O—Ar or halide. In some embodiments, Ar is phenyl, pyridinyl, naphthyl, optionally substituted with one or more of C1-C6 alkyl, cyano, nitro or halo. In some embodiments Ar is a pentafluorophenyl or 4-bromophenyl group. In some embodiments the compound of Formula (VI) is a salt. In some embodiments the compound of Formula (VI) is a salt of triethylamine or Hunig's base; i.e., the cation may be, for example, triethylamine or Hunig's base.

A method of making a compound of Formula (VIa)

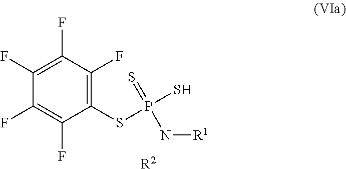

or a salt thereof, such as a trimethylamine salt, includes the step of reacting a compound of Formula (VII)

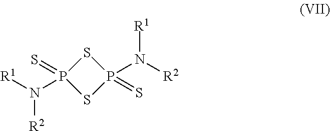

with 2,3,4,5,6-pentafluorobenzenethiol. In some embodiments, R¹ and R² are methyl.

### C3. A Further Method of Preparing Chiral Reagents of Formula (IV)

Provided herein according to some embodiments for preparing a chiral reagent of Formula (IV) from a chiral compound of Formula (V)

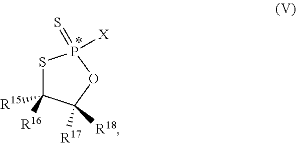

(V), with at least 90% ee.

In formulate (V), P* indicates either (R) or (S) configuration, R¹⁵, R¹⁶, R¹⁷ and R¹⁸ are H, X is a leaving group selected from substituted —S—Ar, —O—Ar or halide, wherein Ar is phenyl, pyridinyl, naphthyl, optionally substituted with one or more of C1-C6 alkyl, cyano, nitro or halo.

For example, X can be —S—Ar, and Ar is phenyl, 4-bromophenyl, 4-nitrophenyl, pentafluorophenyl, dinitrophenyl, or 2,3,5,6-tetrafluoropyridin-4-yl. In some embodiments, the chiral compound of Formula (V) is:

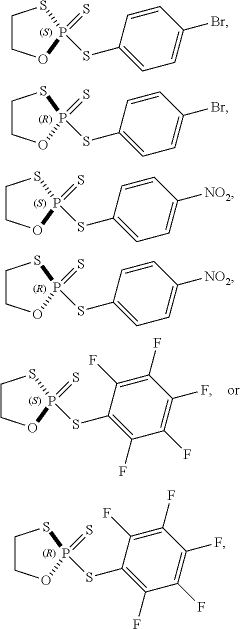

The ee the chiral compound of Formula (V) may be at least 92%, at least 95%, at least 98%, or at least 99%.

Also disclosed herein is a method for preparing a chiral compound of Formula (V)

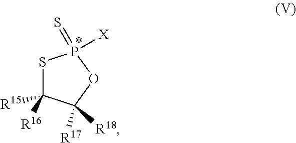

with at least 90% ee.

In Formula (V), P* indicates either (R) or (S) configuration, R¹⁵, R¹⁶, R¹⁷ and R¹⁸ are H, X is a leaving group selected from substituted —S—Ar, —O—Ar or halide, wherein Ar is phenyl, pyridinyl, naphthyl, optionally substituted with one or more of C1-C6 alkyl, cyano, nitro or halo.

Disclosed is a method to preparing a chiral reagent of Formula (V), including the step of separating a racemic mixture of

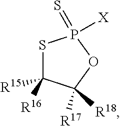

by chiral SFC or chiral HPLC, For the chiral compound of Formula (V), X is —S—Ar, and Ar is phenyl, 4-bromophenyl, 4-nitrophenyl, pentafluorophenyl, dinitrophenyl, or 2,3,5,6-tetrafluoropyridin-4-yl. For example, the chiral compound of Formula (V) can be:

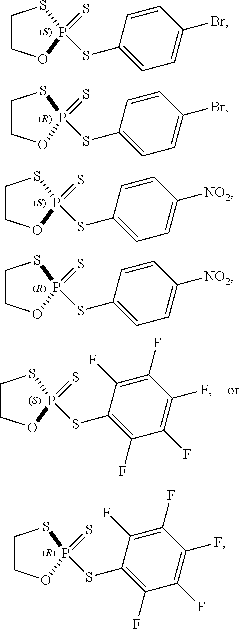

The ee of the chiral compound of Formula (V) may be at least 92%, at least 95%, at least 98%, or at least 99%.

### D. Morpholino Monomer Compounds of Formula (I) and DNA Monomer Compounds of Formula (Id)

### 1. Morpholino Monomer Compounds of Formula (I)

Provided herein according to some embodiments is a stereo-encoded morpholino monomer of Formula (I):

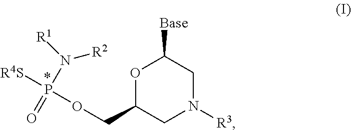

or a diastereomer or enantiomer thereof, or a salt thereof.

In Formula (I), P* represents a phosphorus stereocenter that is either in the (R) or (S) configuration. R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle. In some embodiments, R¹ and R² are both methyl. In some embodiments the optionally substituted heterocycle formed by R¹ and R² together is an optionally substituted morpholinly, piperazinyl, pyrrolidinyl, and azetidinyl.

R³ is a protecting group for morpholino nitrogen. Suitable protecting groups for morpholino nitrogen include, for example, but are not limited to, optionally substituted C1-C6 alkyl, trityl, substituted trityl, including MMTr (p-methoxyphenyldiphenylmethyl), benzyl, 4-methoxybenzyl (PMB or MPM), 3,4-dimethoxybenzyl, diphenylmethyl (DPM), or sulfonyl. Sulfonyl may be, for example, 2-nitrobenzenesulfonyl, 4-nitrobenzenesulfonyl, or 2,4-dinitrobenzenesulfonyl. In some embodiments, R³ is trityl.

R⁴ is hydrogen or C1-C6 alkyl. In some particular embodiments R⁴ is methyl.

Base is selected from the group consisting of.

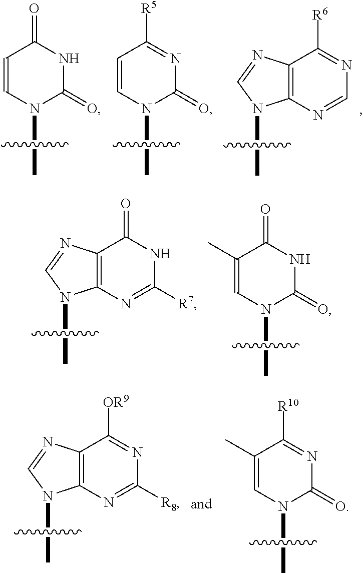

Wherein R⁵, R⁶, R⁷, R⁸, and R¹⁰ are independently —NR²⁰R²¹ or —N═CR¹²—NR¹³R¹⁴.

Where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹. R¹¹ is optionally substituted C1-C6 alkyl, optionally substituted benzyl, or aryl, optionally substituted with one or more substituents selected from halogen, nitro and alkoxyl.

In some embodiments where any of R⁵, R⁶, R⁷, R⁸, and R¹⁰ is independently —NHC(O)R¹¹, R¹¹ is methyl, ethyl, isopropyl, isobutyl, phenyl, 4-methoxyphenyl, 4-bromophenyl, or 4-nitrophenyl. In some embodiments where any of R⁵, R⁶, R⁷, R, and R¹⁰ is independently —NHC(O)OR¹¹, R¹¹ is C1-C6 alkyl, benzyl, 2,2,2-trichloroethyl, or optionally substituted aryl.

Where any of R⁵, R⁶, R⁷, R⁸, and R¹⁰ is independently —N═CR¹²—NR¹³R¹⁴ in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

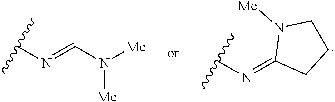 [SMILES: */N=C/N(C)C ; */N=C1/CCCN1C]

R⁹ is H, optionally substituted C1-C6 alkyl, optionally substituted benzyl, acyl, carbonate, carbamate, or silyl. Examples of acyl substituents include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl.

In some embodiments, the compound of Formula (I), its diastereomer, enantiomer, or salt thereof is crystalline.

Some embodiments of compounds of Formula (I) are shown below in Table 2.

TABLE 2

| Compound Number | Compound* |
|---|---|
| 11 | 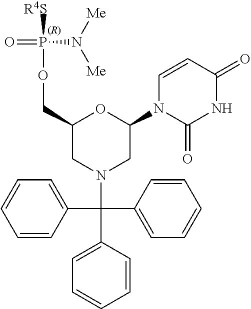 |
| 12 | 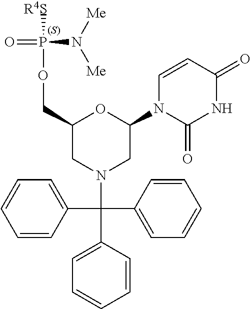 |
| 13 | 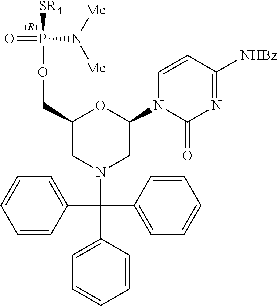 |
| 14 | 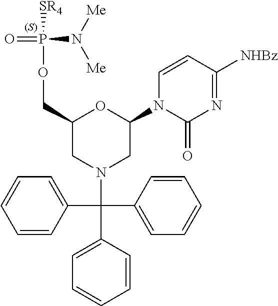 |
| 15 | 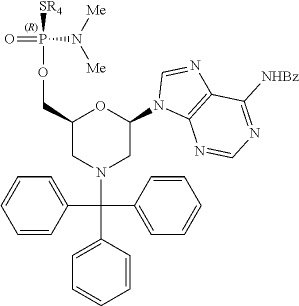 |
| 16 | 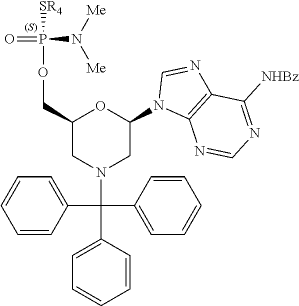 |
| 17 | 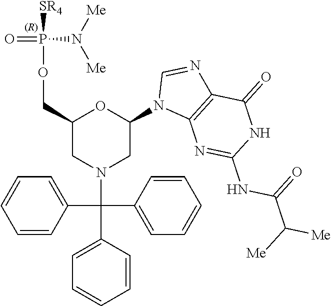 |
| 18 | 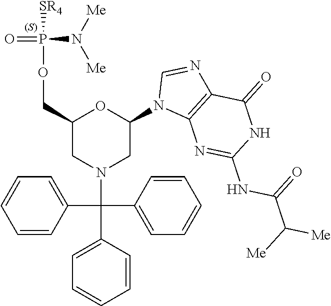 |
| 19 | 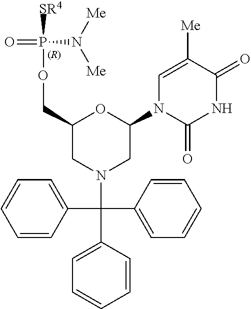 |
| 20 | 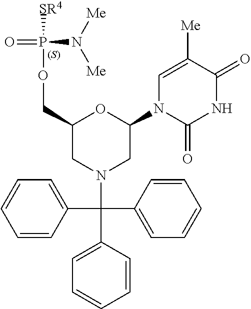 |
| 21 | 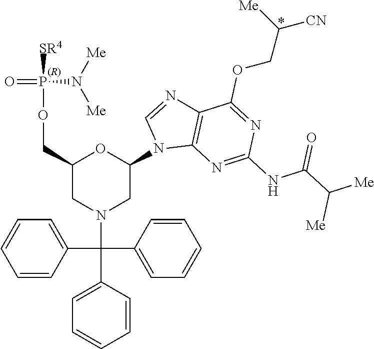 |
| | Chiral center marked with * could be R, S or mixture of R and S. |
| 22 | 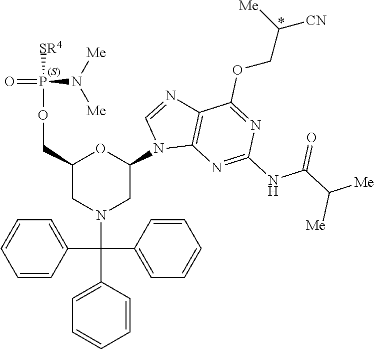 |
| | Chiral center marked with * could be R, S or mixture of R and S. |
| 23 | 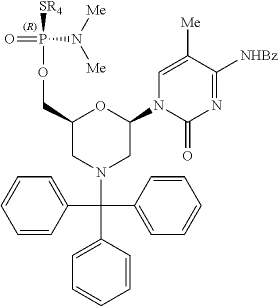 |
| 24 | 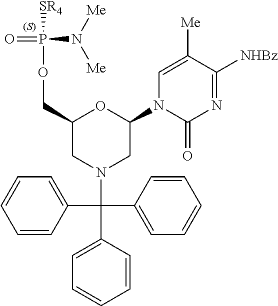 |
| 25 | 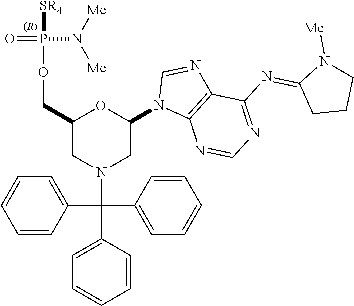 |
| 26 | 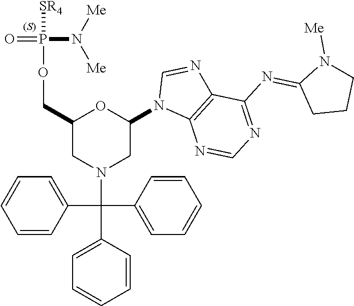 |
| 27 | 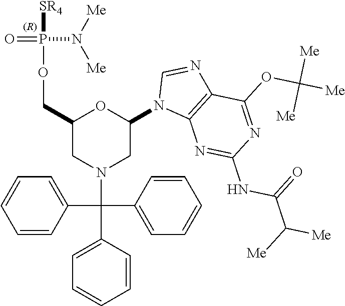 |
| 28 | 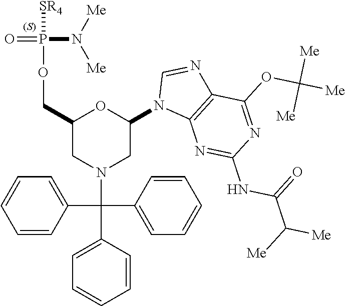 |
| 29 | 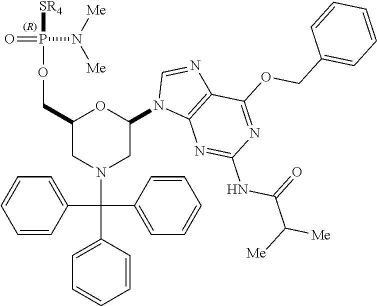 |
| 30 | 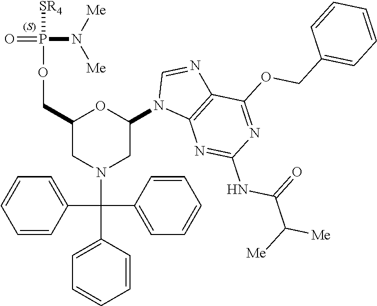 |
| 31 | 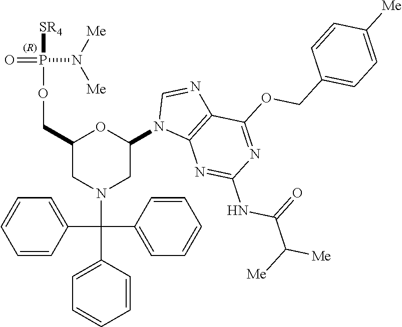 |
| 32 | 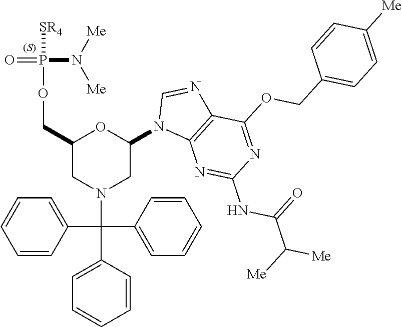 |
| 33 | 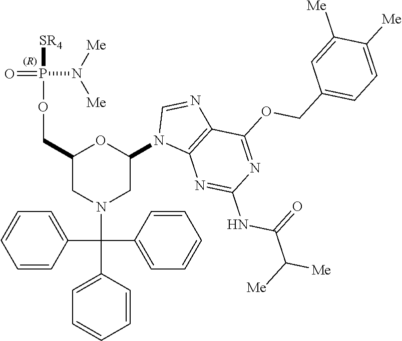 |
| 34 | 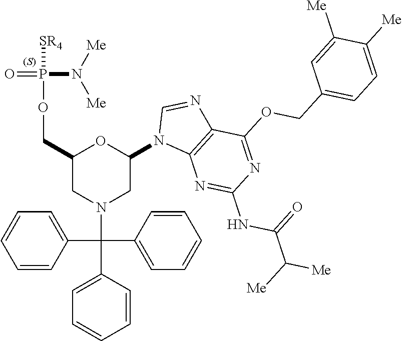 |
| 35 | 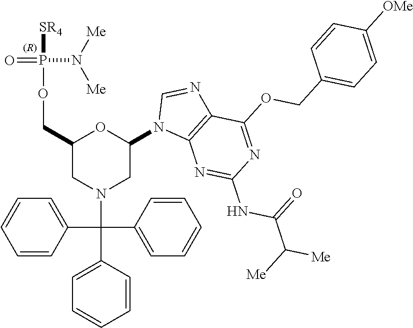 |
| 36 | 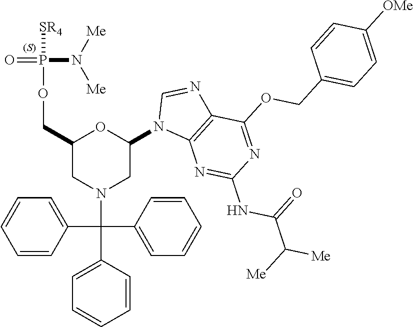 |
| 37 | 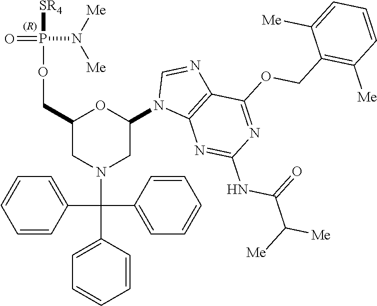 |
| 38 | 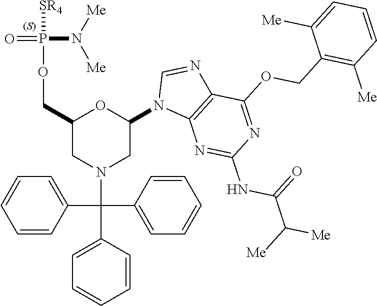 |
| 39 | 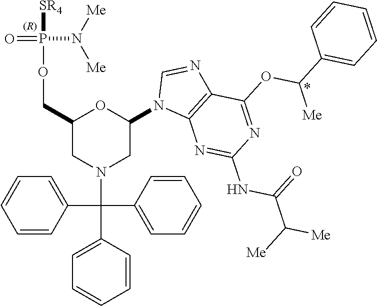 |
| 40 | 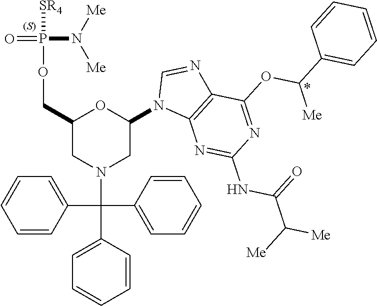 |

(R⁴ = H or Methyl, chiral center marked with * could be R, S or mixture of R and S.)

### 2. DNA Monomer Compounds of Formula (Id)

Provided herein according to some embodiments is a stereo-encoded DNA monomer of Formula (Id):

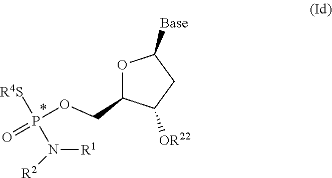

or a diastereomer or enantiomer thereof, or a salt thereof.

In Formula (Id), P* represents a phosphorus stereocenter that is either in the (R) or (S) configuration. R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle. In some embodiments, R¹ and R² are both methyl. In some embodiments the optionally substituted heterocycle formed by R¹ and R² together is an optionally substituted morpholinly, piperazinyl, pyrrolidinyl, and azetidinyl.

R²² is a protecting group for hydroxyl oxygen. Suitable protecting groups for hydroxyl oxygen include, for example, but are not limited to, trialkylsilyl (where the alkyl are the same or different and are C1-C6 alkyl, such as tert-butyldimethylsilyl (TBS)), tert-butyldiphenylsilyl, triphenylsilyl, trityl, substituted trityl, including MMTr (p-methoxyphenyldiphenylmethyl) and DMTr (4,4′-dimethoxytriphenylmethyl), benzyl, 4-methoxybenzyl (PMB or MPM), and acid labile protecting groups, including, for example, 2-tetrahydropyranyl or 1-ethoxyethyl.

R⁴ is hydrogen or C1-C6 alkyl. In some particular embodiments R⁴ is methyl.

Base is selected from the group consisting of:

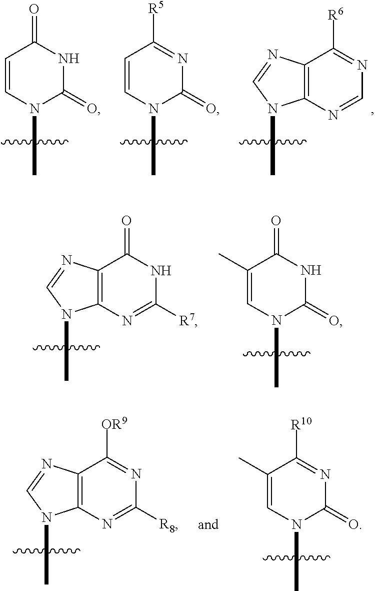

Wherein R⁵, R⁶, R⁷, R⁸, and R¹⁰ are independently —NR²⁰R²¹ or —N═CR¹²—NR¹³R¹⁴.

Where R⁵, R⁶, R⁷, R⁸, and R¹⁰ are independently —NR²⁰R²¹, R²⁰ and R²¹ are independently selected from —H, —C(O)R¹¹, and —C(O)OR¹¹. R¹¹ is optionally substituted C1-C6 alkyl, optionally substituted benzyl, or aryl, optionally substituted with one or more substituents selected from halogen, nitro and alkoxyl. In some embodiments where R⁵, R⁶, R⁷, R⁸, and R¹⁰ are —NHC(O)R¹¹, R¹¹ is methyl, ethyl, isopropyl, isobutyl, phenyl, 4-methoxyphenyl, 4-bromophenyl, or 4-nitrophenyl. In some embodiments where R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl.

Where R⁵, R⁶, R⁷, R⁸, and R¹⁰ are independently —N═CR¹²—NR¹³R¹⁴, in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

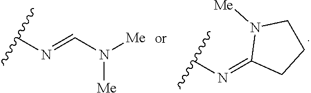 [SMILES: */N=C/N(C)C ; */N=C1/CCCN1C]

R⁹ is H, optionally substituted C1-C6 alkyl, optionally substituted benzyl, acyl, carbonate, carbamate, or silyl. Examples of acyl substituents include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) β-methylcyanoethyl, isobutyl, t-butyl, benzyl, u-methylbenzyl, 4-methylbenzyl, 2,4-, 3,4-, and 2,6-dimethylbenzyl, 4-methoxybenzyl, and 4-pivaloyloxy benzyl.

In some embodiments, the compound of Formula (Id), its diastereomer, enantiomer, or salt thereof is crystalline.

Some embodiments of compounds of Formula (Id) are shown below in Table 3.

TABLE 3

| Compound Number | Compound* |
|---|---|
| 41 | 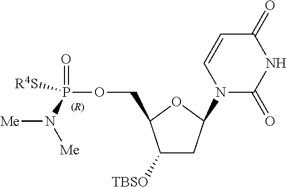 |
| 42 | 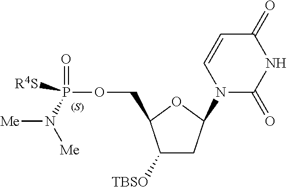 |
| 43 | 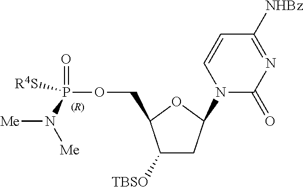 |
| 44 | 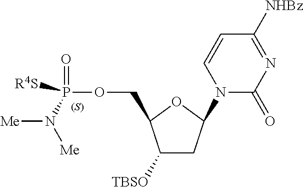 |
| 45 | 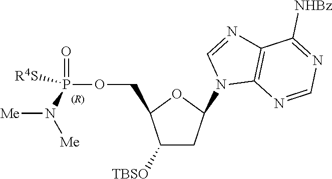 |
| 46 | 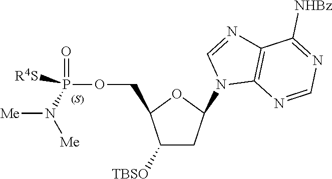 |
| 47 | 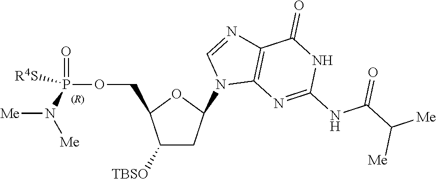 |
| 48 | 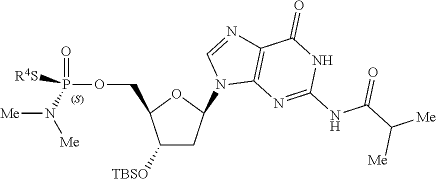 |
| 49 | 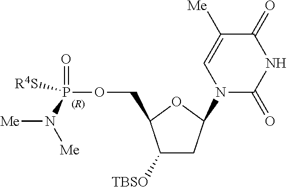 |
| 50 | 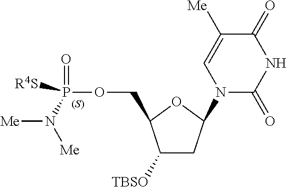 |
| 51 | 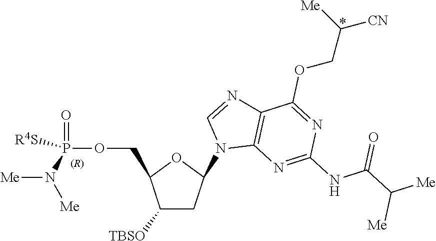 |
| | Chiral center marked with * could be R, S or mixture of R and S. |
| 52 | 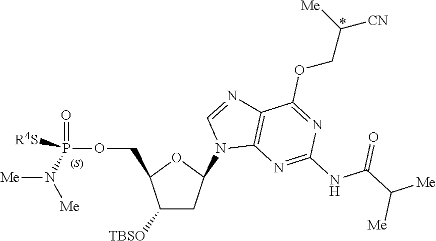 |
| | Chiral center marked with * could be R, S or mixture of R and S. |
| 53 | 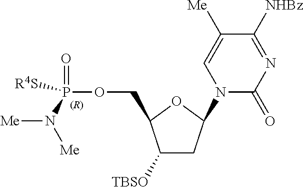 |
| 54 | 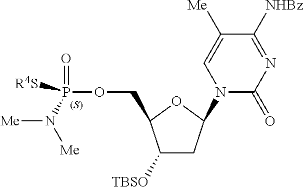 |
| 55 | 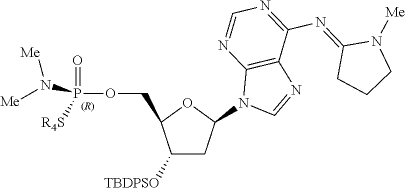 |
| 56 | 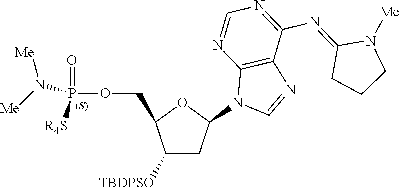 |

(R⁴ = H or Methyl)

### E. Methods of Preparing Crystalline Morpholino Monomers of Formula (I) and DNA Monomers of Formula (Id)

### 1. Methods of Preparing Crystalline Morpholino Monomers of Formula (I)

Still further embodiments provide a method for preparing a stereo-encoded morpholino monomer of Formula (I):

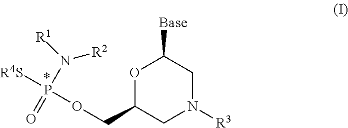

or a diastereomer or enantiomer thereof, or a salt thereof.

In Formula (I), P* represents a stereocenter that is either in the (R) or (S) configuration. R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle. In some embodiments, R¹ and R² are both methyl. In some embodiments the optionally substituted heterocycle formed by R¹ and R² together is an optionally substituted morpholinly, piperazinyl, pyrrolidinyl, and azetidinyl.

R³ is a protecting group for morpholino nitrogen. Suitable protecting groups for morpholino nitrogen include, for example, but are not limited to, optionally substituted C1-C6 alkyl, trityl, substituted trityl, including MMTr (p-methoxyphenyldiphenylmethyl), benzyl, 4-methoxybenzyl (PMB or MPM), 3,4-dimethoxybenzyl, diphenylmethyl (DPM), or sulfonyl. Sulfonyl may be, for example, 2-nitrobenzenesulfonyl, 4-nitrobenzenesulfonyl, or 2,4-dinitrobenzenesulfonyl. In some embodiments, R³ is trityl.

R⁴ is hydrogen or C1-C6 alkyl. In some particular embodiments R⁴ is methyl.

Base is selected from the group consisting of:

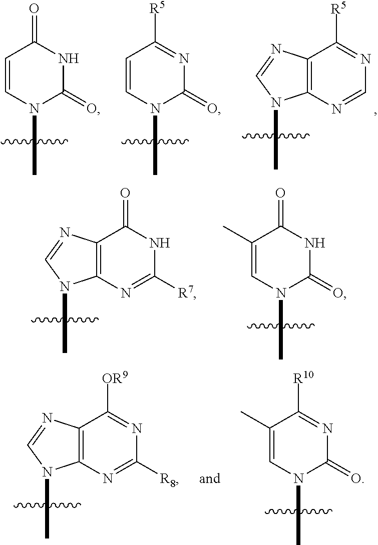

Wherein R⁵, R⁶, R⁷, R⁸, and R¹⁰ are independently —NR²⁰R²¹ or —N═CR¹²—NR¹³R¹⁴

Where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹. R¹¹ is optionally substituted C1-C6 alkyl, optionally substituted benzyl, or aryl, optionally substituted with one or more substituents selected from halogen, nitro and alkoxyl. In some embodiments where any of R⁵, R⁶, R⁷, R⁸, and R¹⁰ is independently —NHC(O)R¹¹, R¹¹ is methyl, ethyl, isopropyl, isobutyl, phenyl, 4-methoxyphenyl, 4-bromophenyl, or 4-nitrophenyl. In some embodiments where any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl.

Where any of R⁵, R⁶, R⁷, R⁸, and R¹⁰ is independently —N═CR¹²—NR¹³R¹⁴, in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

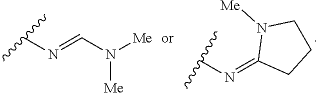 [SMILES: */N=C/N(C)C ; */N=C1/CCCN1C]

R⁹ is H, optionally substituted C1-C6 alkyl, optionally substituted benzyl, acyl, carbonate, carbamate, or silyl. Examples of acyl substituents include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl.

The method may provide the following steps:
reacting a compound of Formula (III):

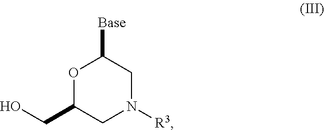

wherein R³ and Base are as defined above, with a chiral reagent having the structure of Formula (IV):

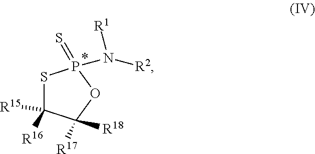

wherein R¹ and R² are as defined above; R¹⁵, R¹⁶, R¹⁷ and R¹⁸ are independently H, C1-C10 alkyl, C2-C12 alkenyl, aryl, or any two of R¹⁵, R¹⁶, R¹⁷, and R¹⁸, together with the carbons they are attached form a cycloalkyl or heteroalkyl ring which is optionally substituted with one or more C1-C10 alkyl or C2-C12 alkenyl groups, in the presence of a base to obtain the stereo-encoded morpholino monomer of Formula (I).

In some embodiments, R¹⁵, R¹⁶, R¹⁷ and R¹⁸ are H. In some embodiments, R¹⁵ is C1-C10 alkyl or aryl, and R¹⁶, R¹⁷, R¹⁸ are H. In some embodiments, R¹⁶ is C1-C10 alkyl or aryl, and R¹⁵, R¹⁷, R¹⁸ are H. In certain embodiments, R¹⁵ is phenyl and R¹⁶, R¹⁷, R¹⁸ are H. In some embodiments, R¹⁶ is phenyl, and R¹⁵, R¹⁷, R¹⁸ are H. Without limitation, the chiral reagent of Formula (IV) may be one of the examples in Table 1. In some embodiments the stereo-encoded morpholino monomer of Formula (I) is crystalline.

The base in the presence of which the reaction is conducted may be, for example, any of sodium hydroxide, sodium hydride (NaH), 1,8-Diazabicyclo[5.4.0]undec-7-ene (DBU), sodium amylate, potassium tert-butoxide, potassium tert-pentoxide, and sodium bis(trimethylsilyl)amide (NaHMDS).

In one aspect the reactions reported above are conducted at room temperature. In one aspect they are conducted in a polar solvent. That polar solvents may be, for example, tetrahydrofuran (THF), acetonitrile, 2-MeTHF, 1,6-dioxane, and DME.

In some embodiments, the diastereomeric excess/enantiomeric excess ratio (de/ee ratio/stereoselectivity) of the resulting stereo-encoded morpholino monomer is at least 90%, at least 95%, at least 98%, or at least 99%.

### 2. Methods of Preparing DNA Monomers of Formula (Id)

Still further embodiments provide a method for preparing a stereo-encoded DNA monomer of Formula (Id):

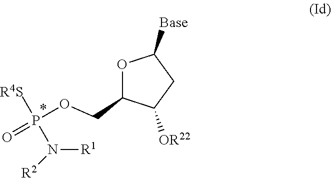

or a diastereomer or enantiomer thereof, or a salt thereof.

In Formula (Id), P* represents a stereocenter that is either in the (R) or (S) configuration. R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle. In some embodiments, R¹ and R² are both methyl. In some embodiments the optionally substituted heterocycle formed by R¹ and R² together is an optionally substituted morpholinly, piperazinyl, pyrrolidinyl, and azetidinyl.

R²² is a protecting group for hydroxyl oxygen. Suitable protecting groups for hydroxyl oxygen include, for example, but are not limited to, trialkylsilyl (where the alkyl are the same or different and are C1-C6 alkyl, such as tert-butyl(dimethyl)silyl (TBS)), tert-butyldiphenylsilyl, triphenylsilyl, trityl, substituted trityl, including MMTr (p-methoxyphenyldiphenylmethyl) and DMTr (4,4′-dimethoxytriphenylmethyl), benzyl, 4-methoxybenzyl (PMB or MPM), and acid labile protecting groups, including, for example, 2-tetrahydropyranyl or 1-ethoxyethyl.

R⁴ is hydrogen or C1-C6 alkyl. In some particular embodiments R⁴ is methyl.

Base is selected from the group consisting of:

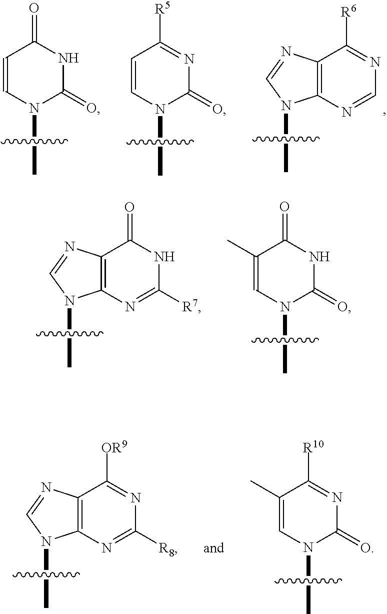

Wherein R⁵, R⁶, R⁷, R⁸, and R¹⁰ are independently —NR²⁰R²¹ or —N═CR¹²—NR¹³R¹⁴.

Where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹, and R¹¹ is optionally substituted C1-C6 alkyl, optionally substituted benzyl, or aryl, optionally substituted with one or more substituents selected from halogen, nitro and alkoxyl. In some embodiments where any of R⁵, R⁶, R⁷, R⁸, and R¹⁰ is independently —NHC(O)R¹¹, R¹¹ is methyl, ethyl, isopropyl, isobutyl, phenyl, 4-methoxyphenyl, 4-bromophenyl, or 4-nitrophenyl. In some embodiments where any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl.

Where any of R⁵, R⁶, R⁷, R⁸, and R¹⁰ is independently —N═CR¹²—NR¹³R¹⁴, in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

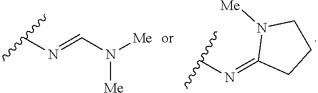 [SMILES: */N=C/N(C)C ; */N=C1/CCCN1C]

R⁹ is H, optionally substituted C1-C6 alkyl, optionally substituted benzyl, acyl, carbonate, carbamate, or silyl. Examples of acyl substituents include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) β-methylcyanoethyl, isobutyl, t-butyl, benzyl, u-methylbenzyl, 4-methylbenzyl, 2,4-, 3,4-, and 2,6-dimethylbenzyl, 4-methoxybenzyl, and 4-pivaloyloxy benzyl.

The method may provide the following steps:
reacting a compound of Formula (IIId):

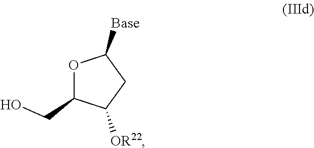

wherein R²² and Base are as defined above, with a chiral reagent having the structure of Formula (IV):

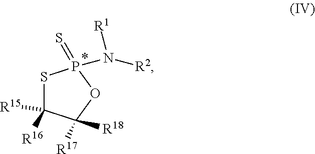

wherein R¹ and R² are as defined above; R¹⁵, R¹⁶, R¹⁷ and R¹⁸ are independently H, C1-C10 alkyl, C2-C12 alkenyl, aryl,
or any two of R¹⁵, R¹⁶, R¹⁷, and R¹⁸, together with the carbons they are attached form a cycloalkyl or heteroalkyl ring which is optionally substituted with one or more C1-C10 alkyl or C2-C12 alkenyl groups, in the presence of a base to obtain the stereo-encoded DNA monomer of Formula (Id).

In some embodiments, R¹⁵, R¹⁶, R¹⁷ and R¹⁸ are H. In some embodiments, R¹⁵ is C1-C10 alkyl or aryl, and R¹⁶, R¹⁷, R¹⁸ are H. In some embodiments, R¹⁶ is C1-C10 alkyl or aryl, and R¹⁵, R¹⁷, R¹⁸ are H. In certain embodiments, R¹⁵ is phenyl and R¹⁶, R¹⁷, R¹⁸ are H. In some embodiments, R¹⁶ is phenyl, and R¹⁵, R¹⁷, R¹⁸ are H. Without limitation, the chiral reagent of Formula (IV) may be one of the examples in Table 1. In some embodiments the stereo-encoded DNA monomer of Formula (Id) is crystalline.

The base in the presence of which the reaction is conducted may be, for example, any of sodium hydroxide, sodium hydride (NaH), 1,8-Diazabicyclo[5.4.0]undec-7-ene (DBU), sodium amylate, potassium tert-butoxide, potassium tert-pentoxide, and sodium bis(trimethylsilyl)amide (NaHMDS).

In one aspect the reactions reported above are conducted at room temperature. In one aspect they are conducted in a polar solvent. That polar solvent may be, for example, tetrahydrofuran (THF), acetonitrile, 2-MeTHF, 1,6-dioxane, and DME.

In some embodiments, the diastereomeric excess/enantiomeric excess ratio (de/ee ratio/stereoselectivity) of the resulting stereo-encoded DNA monomer is at least 90%, at least 95%, at least 98%, or at least 99%.

### F. Applications of Chiral Reagents of Formula (V) and Other Reagents in Preparing Other Monomers

The chiral reagent of Formula (V)

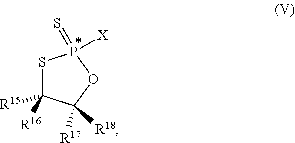

may be useful in the synthesis of of stereopure monomers and stereopure oligonucleotides having phosphorothioates linkages.

In other embodiments, a chiral reagent having the formula of (V′)

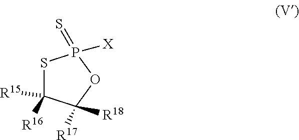

may also be useful in the synthesis of monomers and oligonucleotides having phosphodiester linkages.

In Formulas (V) and (V′), * indicates a stereocenter in either (R) or (S) configuration, X is a leaving group selected from substituted —S-aryl, —O-aryl or halide. For example, aryl can be phenyl, naphthyl, optionally substituted with one or more of alkyl, cyano, nitro or halo. R¹⁵, R¹⁶, R¹⁷ and R¹⁸ are H,

The chiral monomers made from chiral reagent of Formulas (V) and (V′) may include, for example, the compounds shown below:

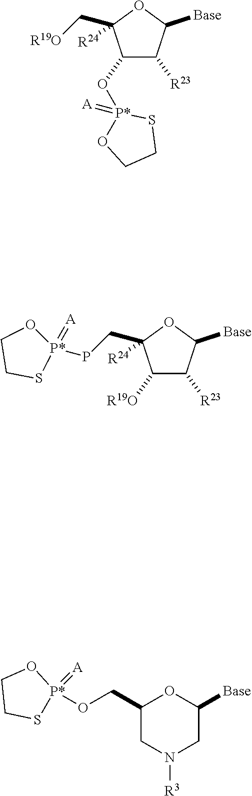

and diastereomers and enantiomers thereof.

A is O or S; * indicates stereocenter in either an (R) or (S) configuration when A is S; R³ is H or a protecting group for morpholino nitrogen, selected from optionally substituted C1-C6 alkyl, trityl, benzyl, and sulfonyl; R¹⁹ is H or a protecting group for a hydroxyl group.

In some embodiments, R²³ is F, or OR²⁵, wherein R²⁵ is C1-C6 alkyl, (C1-C6 alkoxy)alkyl or a protecting group for hydroxyl group, such as methyl or —OCH₂CH₂OMe; and R24 is H. In other embodiments, R²³ and R²⁴ together create an optionally substituted ring to form a locked nucleic acid (LNA), 2′-amino-LNA, 2′-S-constrained ethyl (cEt) or 2′-O, 4′-C-ethylene-bridged nucleic acid (ENA).

Base may be, for example, selected from:

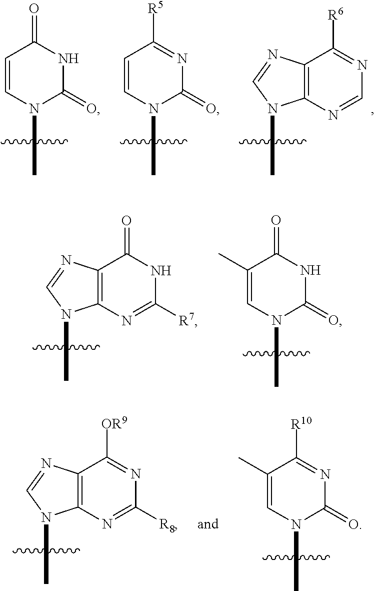

R⁵, R⁶, R⁷, R⁸, and R¹⁰ are independently —NR²⁰R²¹ or —N═CR¹²—NR¹³R¹⁴; where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹, and R¹¹ is optionally substituted C1-C6 alkyl, optionally substituted benzyl, or aryl, optionally substituted with one or more substituents selected from halogen, nitro and alkoxyl;
when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl;
when any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl; R¹², R¹³, and R¹⁴ are independently C1-C6 alkyl, or
R¹² and R¹³ together form a 5 or 6-membered heterocycle with the adjacent C and N; R⁹ is selected from the group consisting of H, cyanoethyl, (R) and/or (S) α-methylcyanoethyl, (R) and/or (S) β-methylcyanoethyl, isobutyl, t-butyl, benzyl, u-methylbenzyl, 4-methylbenzyl, 2,4-, 3,4-, and 2,6-dimethylbenzyl, 4-methoxybenzyl, and 4-pivaloyloxy benzyl.

### G. Methods of Preparing Stereo-Encoded Activated Monomers of Formula (II) and Stereo-Encoded Activated Monomers of Formula (IId)

### 1. Methods of Preparing Stereo-Encoded Activated Monomers of Formula (II)

Further embodiments provide a method of preparing a stereo-encoded activated monomer of Formula (II):

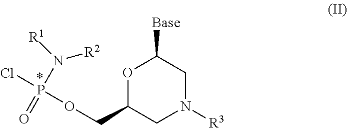

or a diastereomer or enantiomer thereof, or a salt thereof. In Formula (II) P* represents a stereocenter that is either in the (R) or (S) configuration. R¹ and R² are independently H, optionally substituted C1-C6 alkyl, optionally substituted phenyl, optionally substituted naphthyl, or R¹ and R² together with the nitrogen to which they connect, form an optionally substituted heterocycle. In some embodiments, R¹ and R² are methyl.

R³ is a protecting group for morpholino nitrogen. Suitable protecting groups for morpholino nitrogen include, for example, but are not limited to, optionally substituted alkyl, benzyl, trityl, and sulfonyl. Substituted trityl may be, for example, including MMTr (p-methoxyphenyldiphenylmethyl). Substituted benzyl may be 4-methoxybenzyl (PMB or MPM), 3,4-dimethoxybenzyl, or diphenylmethyl (DPM). Sulfonyl may be, for example, 2-nitrobenzenesulfonyl, 4-nitrobenzenesulfonyl, or 2,4-dinitrobenzenesulfonyl. In some embodiments, R³ is trityl.

Base is selected from the group consisting of:

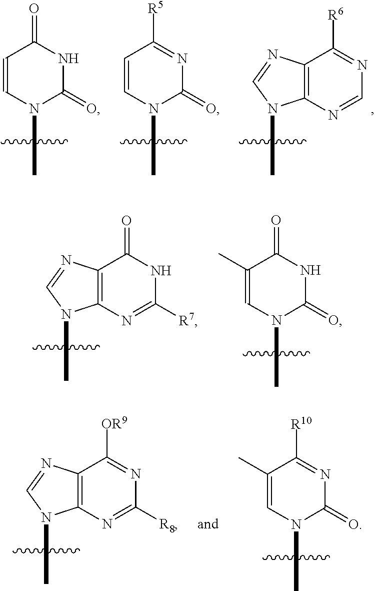

Wherein R⁵, R⁶, R⁷, R⁸, and R¹⁰ are independently —NR²⁰R²¹ or —N═CR¹²—NR¹³R¹⁴.

Where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹, and R¹¹ is optionally substituted C1-C6 alkyl, optionally substituted benzyl, or aryl, optionally substituted with one or more substituents selected from halogen, nitro and alkoxyl. In some embodiments where any of R⁵, R⁶, R⁷, R⁸, and R¹⁰ is independently —NHC(O)R¹¹, R¹¹ is methyl, ethyl, isopropyl, isobutyl, phenyl, 4-methoxyphenyl, 4-bromophenyl, or 4-nitrophenyl. In some embodiments where R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl.

Where any of R⁵, R⁶, R⁷, R⁸, and R¹⁰ is independently —N═CR²—NR¹³R¹⁴, in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

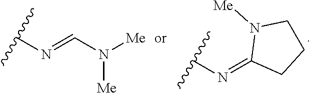 [SMILES: */N=C/N(C)C ; */N=C1/CCCN1C]

R⁹ is H, optionally substituted C1-C6 alkyl, optionally substituted benzyl, acyl, carbonate, carbamate, or silyl. Examples of acyl substituents include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl.

The reaction may include the following steps:
(a) providing a stereo-encoded morpholino monomer of Formula (Ia)

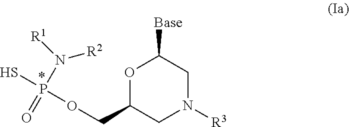

(b) optionally alkylating the sulfur of the stereo-encoded morpholino monomer of Formula (Ia) in step (a) to afford a stereo-encoded morpholino monomer of Formula (I)

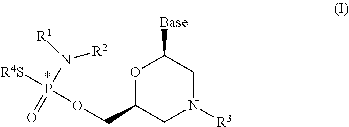

wherein R⁴ is C1-C6 alkyl;
(c) reacting the stereo-encoded morpholino monomer of Formula (Ia) from step (a) or the stereo-encoded morpholino monomer of Formula (I) from step (b) with a chlorinating agent to afford the stereo-encoded activated monomer of Formula (II).

In some embodiments alkylation is conducted by addition of triethylamine and methyl iodide to a compound of Formula (Ia). In some embodiments the chlorinating agent is sulfuryl chloride. In embodiments where Formula (Ia) is used, the chlorinating agent may be tetramethyl chloroenamine. In some embodiments, step (c) is conducted in the presence of a base. The base can be collidine, such as 2,4,6-trimethylpyridine (also known as 2,4,6-collidine) is included with the chlorinating agent.

### 2. Methods of Preparing Stereo-Encoded Activated Monomers of Formula (IId)

Further embodiments provide a method of preparing a stereo-encoded activated monomer of Formula (IId):

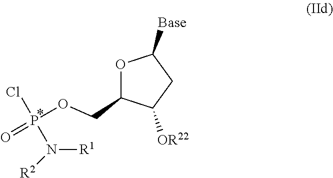

or a diastereomer or enantiomer thereof, or a salt thereof. In Formula (IId) P* represents a stereocenter that is either in the (R) or (S) configuration. R¹ and R² are independently H, optionally substituted C1-C6 alkyl, optionally substituted phenyl, optionally substituted naphthyl, or R¹ and R² together with the nitrogen to which they connect, form an optionally substituted heterocycle.

R²² is a protecting group for hydroxyl oxygen. Suitable protecting groups for hydroxyl oxygen include, for example, but are not limited to, trialkylsilyl (where the alkyl are the same or different and are C1-C6 alkyl, such as tert-butyl(dimethyl)silyl (TBS)), tert-butyldiphenylsilyl, triphenylsilyl, trityl, substituted trityl, including MMTr (p-methoxyphenyldiphenylmethyl) and DMTr (4,4′-dimethoxytriphenylmethyl), benzyl, 4-methoxybenzyl (PMB or MPM), and acid labile protecting groups, including, for example, 2-tetrahydropyranyl or 1-ethoxyethyl.

Base is selected from the group consisting of:

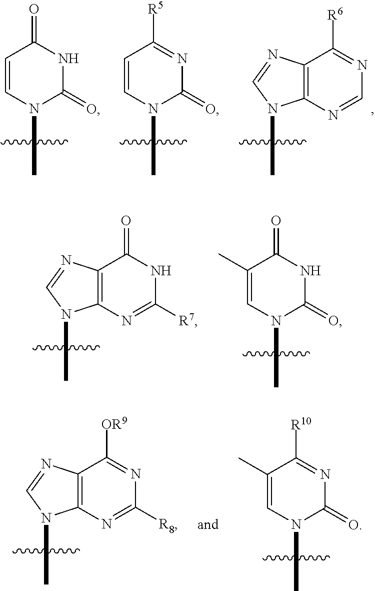

Wherein R⁵, R⁶, R⁷, R⁸, and R¹⁰ are independently —NR²⁰R²¹ or —N═CR¹²—NR¹³R¹⁴.

Where R⁵, R⁶, R⁷, R⁸, and R¹⁰ are independently —NR²⁰R²¹, R²⁰ and R²¹ are independently selected from —H, —C(O)R¹¹, and —C(O)OR¹¹. R¹¹ is optionally substituted C1-C6 alkyl, optionally substituted benzyl, or aryl, optionally substituted with one or more substituents selected from halogen, nitro and alkoxyl. In some embodiments where R⁵, R⁶, R⁷, R⁸, and R¹⁰ are —NHC(O)R¹¹, R¹¹ is methyl, ethyl, isopropyl, isobutyl, phenyl, 4-methoxyphenyl, 4-bromophenyl, or 4-nitrophenyl. In some embodiments where R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl.

Where R⁵, R⁶, R⁷, R, and R¹⁰ are independently —N═CR²—NR¹³R¹⁴, in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

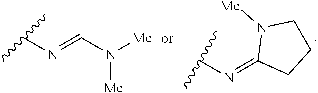 [SMILES: */N=C/N(C)C ; */N=C1/CCCN1C]

R⁹ is H, optionally substituted C1-C6 alkyl, acyl, carbonate, carbamate, benzyl or silyl. Examples of acyl substituents include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl.

The reaction may include the following steps:
(a) providing a stereo-encoded DNA monomer of Formula (Iad)

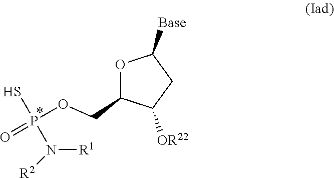

wherein R²² is a protecting group for hydroxyl oxygen. Suitable protecting groups for hydroxyl oxygen include, for example, but are not limited to, trialkylsilyl (where the alkyl are the same or different and are C1-C6 alkyl, such as tert-butyl(dimethyl)silyl (TBS)), tert-butyldiphenylsilyl, triphenylsilyl, trityl, substituted trityl, including MMTr (p-methoxyphenyldiphenylmethyl) and DMTr (4,4′-dimethoxytriphenylmethyl), benzyl, 4-methoxybenzyl (PMB or MPM), and acid labile protecting groups, including, for example, 2-tetrahydropyranyl or 1-ethoxyethyl.
(b) optionally alkylating the sulfur of the stereo-encoded DNA monomer of Formula (Iad) in step
(a) to afford a stereo-encoded DNA monomer of Formula (Id)

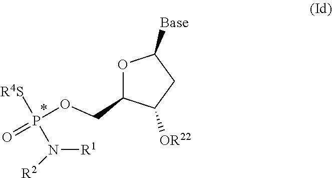

wherein R⁴ is C1-C6 alkyl;
(c) reacting the stereo-encoded DNA monomer of Formula (Iad) from step (a) or the stereo-encoded DNA monomer of Formula (Id) from step (b) with a chlorinating agent to afford the stereo-encoded activated monomer of Formula (IId).

In some embodiments alkylation is conducted by addition of triethylamine and methyl iodide to a compound of Formula (Iad). In some embodiments the chlorinating agent is sulfuryl chloride. In embodiments where Formula (Iad) is used, the chlorinating agent may be tetramethyl chloroenamine. In some embodiments 2,4,6-trimethylpyridine (also known as collidine) is included with the chlorinating agent.

### H. Methods for Preparing Morpholino Dimers From Activated Morpholino Monomers

Embodiments may provide a method of preparing a morpholino dimer of Formula (VIII),

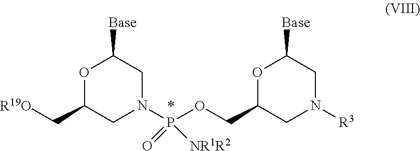

or a diastereomer or enantiomer thereof, or a salt thereof, using stereo-encoded activated monomers as reported herein.

In Formula (VIII), P* represents a stereocenter that is either in the (R) or (S) configuration; R¹ and R² are independently H, optionally substituted C1-C6 alkyl, optionally substituted phenyl, optionally substituted naphthyl, or R¹ and R², together with the nitrogen to which they connect, form an optionally substituted heterocycle, such as morpholine, piperazine, pyrrolidine, and azetidine. In some embodiments, R¹ and R² are methyl. R³ is optionally substituted alkyl, trityl, benzyl, or sulfonyl. In some embodiments, R³ is trityl.

Base is selected from the group consisting of.

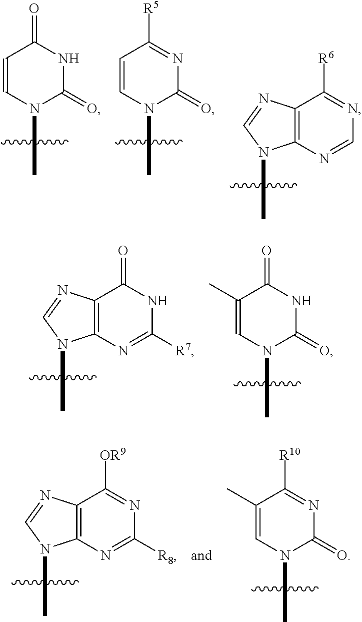

Wherein R⁵, R⁶, R⁷, R⁸, and R¹⁰ are independently —NR²⁰R²¹ or —N═CR¹²—NR¹³R¹⁴.

Where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹. R¹¹ is optionally substituted C1-C6 alkyl, optionally substituted benzyl, or aryl, optionally substituted with one or more substituents selected from halogen, nitro and alkoxyl. In some embodiments where R⁵, R⁶, R⁷, R⁸, and R¹⁰ are —NHC(O)R¹¹, R¹¹ is methyl, ethyl, isopropyl, isobutyl, phenyl, 4-methoxyphenyl, 4-bromophenyl, or 4-nitrophenyl. In some embodiments where R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl.

Where any of R⁵, R⁶, R⁷, R⁸, and R¹⁰ is independently —N═CR¹²—NR¹³R¹⁴, in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

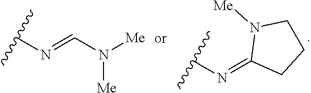 [SMILES: */N=C/N(C)C ; */N=C1/CCCN1C]

R⁹ is H, optionally substituted C1-C6 alkyl, optionally substituted benzyl, acyl, carbonate, carbamate, or silyl. Examples of acyl groups include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) 3-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl.

R¹⁹ is a suitable protecting group for a hydroxyl group. In some embodiments the protecting group is selected from silyl, acyl, and optionally substituted trityl.

In specific embodiments R¹⁹ may be, for example, H, TBS, TBDPS, benzoyl, trityl, DMTr, p-methoxyphenyldiphenylmethyl, benzyl, 4-methoxybenzyl, 2-tetrahydropyranyl, and ethoxyethyl.

In one aspect the method as disclosed includes at least the following steps:
(a) providing a stereo-encoded morpholino monomer of Formula (Ia)

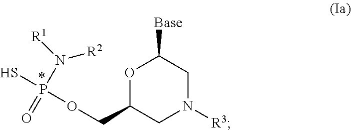

(b) optionally alkylating the sulfur of the stereo-encoded morpholino monomer of Formula (Ia) in step (a) to afford a stereo-encoded morpholino monomer of Formula (I)

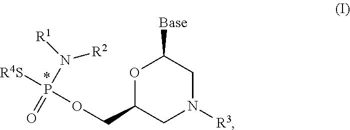

wherein R⁴ is C1-C6 alkyl;
(c) reacting the stereo-encoded morpholino monomer of Formula (Ia) from step (a) or the stereo-encoded morpholino monomer of Formula (I) from step (b) with a chlorinating agent to afford a stereo-encoded activated monomer of Formula (II):

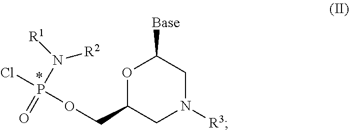

(d) optionally isolating the stereo-encoded activated compound of Formula (II) from step (c);
(e) coupling the stereo-encoded activated compound of Formula (II) from step (c) or (d) with a compound of Formula (IX)

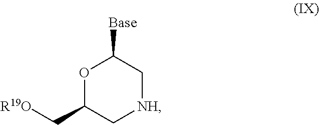

to obtain the morpholino dimer of Formula (VIII). R¹⁹ may be, for example, H, TBS, TBDPS, benzoyl, trityl, DMTr, p-methoxyphenyldiphenylmethyl, benzyl, 4-methoxybenzyl, 2-tetrahydropyranyl, and ethoxyethyland may be a “linker” which is generally used to connect morpholino monomer derivatives to a solid support/resin. Examples include succinyl, succinyl-3-aminopropanoyl, sarcosyl, and succinyl sarcosyl. An example of a morpholino dimer of Formula (VIII) is shown below:

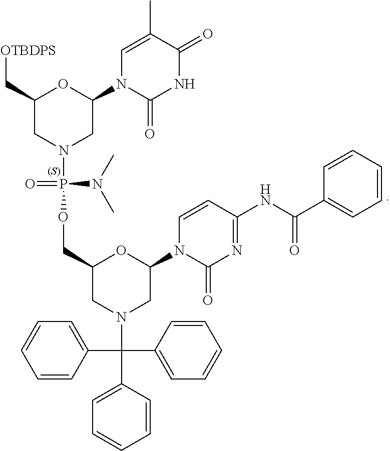

In a further embodiment, a PMO-DNA hetero-dimer may be prepared by coupling the stereo-encoded activated compound of Formula (IId)

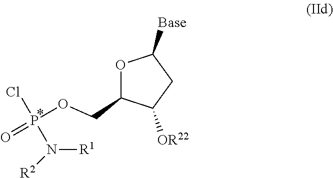

with chiral reagent of Formula (IX):

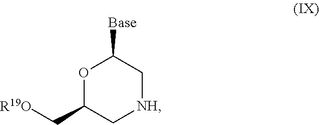

The stereo-encoded activated compound of Formula (IId) may be prepared by the following steps, from a stereo-encoded DNA monomer of Formula (Iad).
(a) providing a stereo-encoded DNA monomer of Formula (Iad)

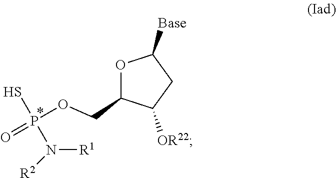

wherein R²² is a protecting group for hydroxyl oxygen. Suitable protecting groups for hydroxyl oxygen include, for example, but are not limited to, trialkylsilyl (where the alkyl are the same or different and are C1-C6 alkyl, such as tert-butyl(dimethyl)silyl (TBS)) or tert-butyldiphenylsilyl, triphenylsilyl, trityl, substituted trityl, including MMTr (p-methoxyphenyldiphenylmethyl) and DMTr (4,4′-dimethoxytriphenylmethyl), benzyl, 4-methoxybenzyl (PMB or MPM), and acid labile protecting groups, including, for example, 2-tetrahydropyranyl or 1-ethoxyethyl; (b) optionally sulfur-alkylating the stereo-encoded DNA monomer of Formula (Iad) in step (a) to afford a stereo-encoded DNA monomer of Formula (Id)

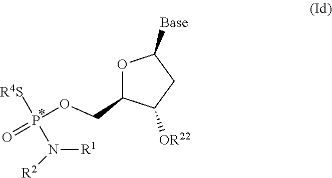

wherein R⁴ is C1-C6 alkyl;
(c) reacting the stereo-encoded DNA monomer of Formula (Iad) from step (a) or the stereo-encoded DNA monomer of Formula (Id) from (b) with a chlorinating agent to afford a stereo-encoded activated monomer of Formula (IId)

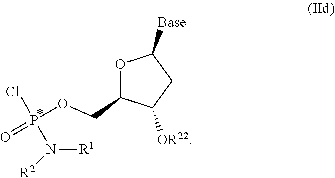

(d) optionally isolating the stereo-encoded activated compound of Formula (IId) from step (c); The activated monomer of Formula (IId) then can be coupled with a compound of Formula (IX):
(e) coupling the stereo-encoded activated compound of Formula (IId) from step (c) or (d) with a compound of Formula (IX)

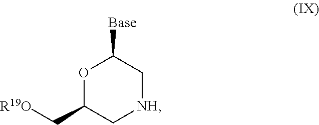

to obtain the PMO-DNA hetero-dimer (XIId).

R¹⁹ may be, for example TBS, TBDPS, benzoyl, trityl, DMTr, p-methoxyphenyldiphenylmethyl, benzyl, 4-methoxybenzyl, 2-tetrahydropyranyl, and ethoxyethyland may be a “linker” which is generally used to connect morpholino monomer derivatives to a solid support/resin. Examples include succinyl, succinyl-3-aminopropanoyl, sarcosyl, and succinyl sarcosyl.

An example of a PMO-DNA hetero-dimer of Formula (XIId) is shown below:

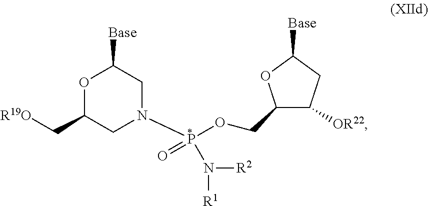

where Base, R¹¹, and R² are defined as above, R¹⁹, R²² can be independently protection groups, or a linker to solid support, as defined above, or H after the protection group is removed or the linker is cleaved from solid support under condition known in the art.
   I. Methods for Preparing Oligomers from Morpholino Dimers
   1. Preparing Oligomers from Morpholino Dimers

Aspects as reported herein may provide a method of making a phosphorodiamidate morpholino oligomer of formula (X)

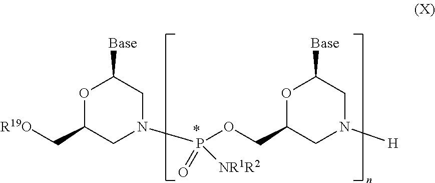

or a diastereomer or enantiomer thereof, or a salt thereof. In the compound of Formula (X), P* represents a stereocenter that is either in the (R) or (S) configuration. R¹ and R² are independently H, optionally substituted C1-C6 alkyl, optionally substituted phenyl, optionally substituted naphthyl, or R¹ and R², together with the nitrogen to which they connect, form an optionally substituted heterocycle.

Base is selected from the group consisting of

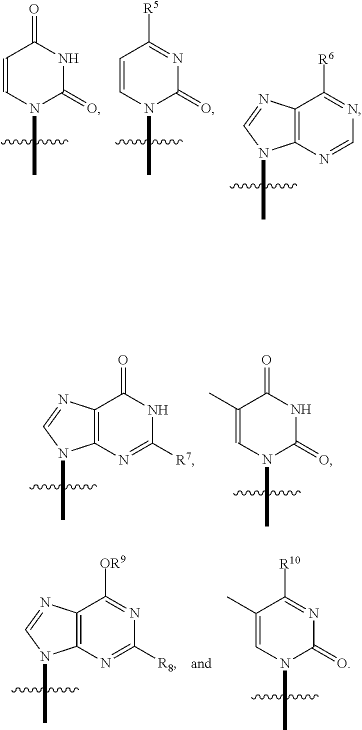

Wherein R⁵, R⁶, R⁷, R⁸, and R¹⁰ are independently —NR²⁰R²¹ or —N═CR¹²—NR¹³R¹⁴.

Where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹. R¹¹ is optionally substituted C1-C6 alkyl, optionally substituted benzyl, or aryl, optionally substituted with one or more substituents selected from halogen, nitro and alkoxyl. In some embodiments where R⁵, R⁶, R⁷, R⁸, and R¹⁰ are —NHC(O)R¹¹, R¹¹ is methyl, ethyl, isopropyl, isobutyl, phenyl, 4-methoxyphenyl, 4-bromophenyl, or 4-nitrophenyl. In some embodiments where R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl.

Where any of R⁵, R⁶, R⁷, R⁸, and R¹⁰ is independently —N═CR¹²—NR¹³R¹⁴, in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

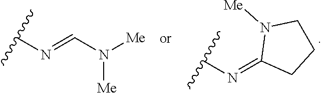 [SMILES: */N=C/N(C)C ; */N=C1/CCCN1C]

R⁹ is H, optionally substituted C1-C6 alkyl, optionally substituted benzyl, acyl, carbonate, carbamate, or silyl. Examples of acyl substituents include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) β-methylcyanoethyl, isobutyl, t-butyl, benzyl, u-methylbenzyl, 4-methylbenzyl, 2,4-, 3,4-, and 2,6-dimethylbenzyl, 4-methoxybenzyl, and 4-pivaloyloxy benzyl.

In some embodiments, R¹⁹ is H or a suitable protecting group for a hydroxyl group. Suitable protecting groups, such as, but are not limited to TBS, TBDPS, benzoyl, or DMTr. In some embodiments, R¹⁹ may be a “linker” to a linker to solid support which is generally used to connect morpholino monomer derivatives to solid support/resin. Examples of a linker to solid support include succinyl, succinyl-3-aminopropanoyl, sarcosyl, and succinyl sarcosyl.

The method includes the following steps:
(a) providing a stereo-encoded morpholino monomer of Formula (Ia)

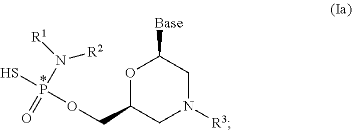

wherein R³ is optionally substituted C1-C6 alkyl, trityl, benzyl, or sulfonyl;
(b) optionally sulfur-alkylating the stereo-encoded morpholino monomer of Formula (Ia) in step
(a) to afford a stereo-encoded morpholino monomer of Formula (I)

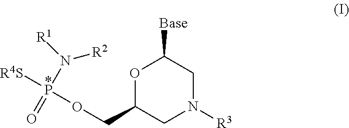

wherein R⁴ is C1-C6 alkyl;
(c) reacting the stereo-encoded morpholino monomer of Formula (Ia) from step (a) or the stereo-encoded morpholino monomer of Formula (I) from (b) with a chlorinating agent to afford a stereo-encoded activated monomer of Formula (II)

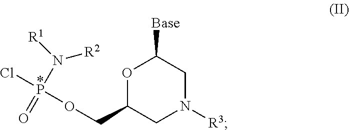

(d) optionally isolating the stereo-encoded activated compound of Formula (II) from step (c);
(e) coupling the compound of Formula (II) from step (c) or (d) with a compound of Formula (IX)

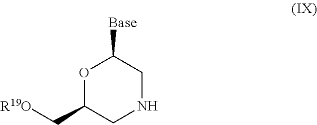

to obtain a morpholino dimer of Formula (VIII)

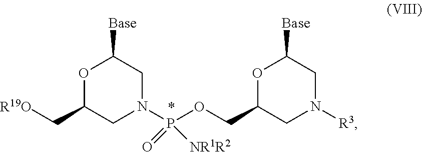

wherein R¹⁹ is H or a suitable protecting group for a hydroxyl group; or R¹⁹ may be a “linker” which is generally used to connect morpholino monomer derivatives to solid support/resin. Examples include 3-aminopropyl, succinyl, 2,2′-diethanosulfonyl and a long chain alkyl amino group.
(f) deprotecting the morpholino dimer of Formula (VIII) to obtain an intermediate oligomer of Formula (X)

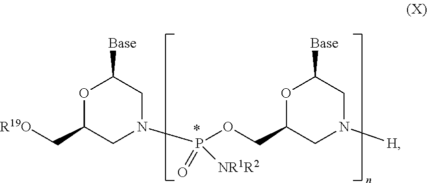

wherein n is 1;
(g) repeating step (i) and step (ii) below m times, wherein m is an integer from 0 to 28 inclusive:
   (i) reacting the intermediate oligomer of Formula (X)

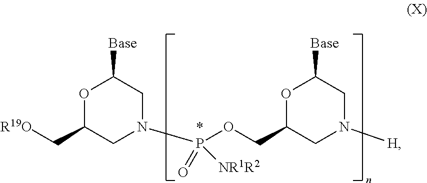

wherein n is an integer from 1 to 28 inclusive,
with a stereo-encoded activated compound of Formula (II)

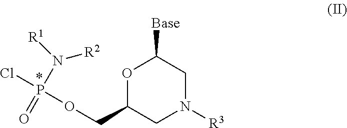

prepared from a stereo-encoded morpholino monomer of Formula (I) by steps (a) to (d)

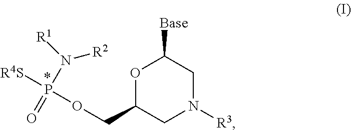

wherein R⁴ is H or C1-C6 alkyl,
to provide an intermediate of formula (XI):

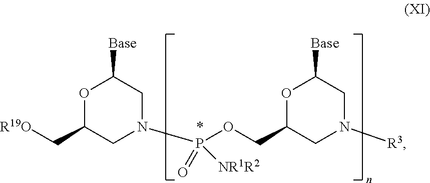

wherein n is an integer from 1 to 29 inclusive; and
   (ii) deprotecting the intermediate of Formula (XI) from step (i) to provide a phosphorodiamidate morpholino oligomer of Formula (X):

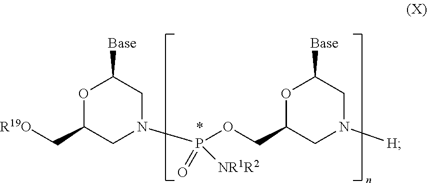

wherein n is an integer from 1 to 29 inclusive;
(h) optionally, removing the R¹⁹ group in the intermediate oligomer of Formula (X) from step (ii) of step (g), when R¹⁹ is a suitable protecting group for a hydroxyl group, or when R¹⁹ is a linker connecting to a solid support/resin. This provides a phosphorodiamidate morpholino oligomer of Formula (XIII):

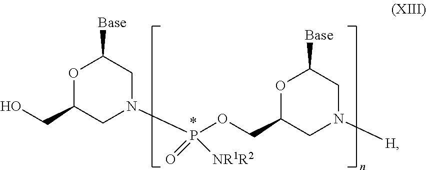

where n is from 1 to 29 inclusive.

The R¹⁹ protecting group or linker maybe removed by standard deprotecting condition, for example, 7N ammonia in methanol or aqueous ammonia with water, methanol, ethnaol, isopropanol and a mixture of these solvents. at 50~65° C.

### 2. Methods for Preparing PMO-DNA Hetero-Oligomers

Aspects as reported herein may provide a method of making a PMO-DNA hetero-oligomer of Formula (Xd):

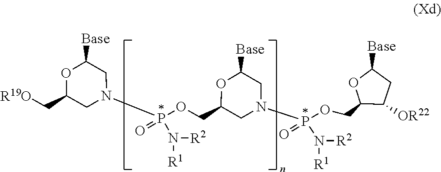

or a diastereomer or enantiomer thereof, or a salt thereof. In the compound of Formula (Xd), P* represents a stereocenter that is either in the (R) or (S) configuration, and each P* does not need to be the same. In Formula (Xd), n is an integer between 1 and 7 inclusive. R¹ and R² are independently H, optionally substituted C1-C6 alkyl, optionally substituted phenyl, optionally substituted naphthyl, or R¹ and R², together with the nitrogen to which they connect, form an optionally substituted heterocycle.

Each Base may be the same or different and is selected from the group consisting of:

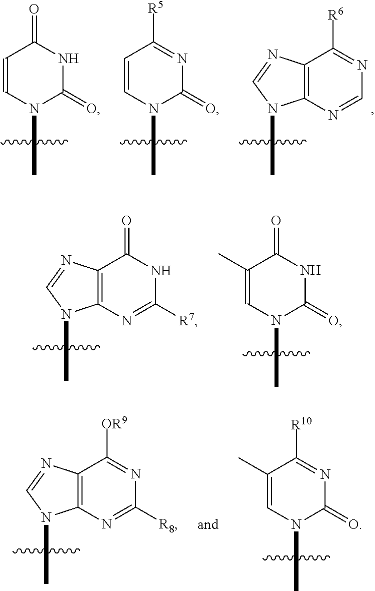

Wherein R⁵, R⁶, R⁷, R⁸, and R¹⁰ are independently —NR²⁰R²¹ or —N═CR¹²—NR¹³R¹⁴.

Where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹. R¹¹ is optionally substituted C1-C6 alkyl, optionally substituted benzyl, or aryl, optionally substituted with one or more substituents selected from halogen, nitro and alkoxyl. In some embodiments where R⁵, R⁶, R⁷, R⁸, and R¹⁰ are —NHC(O)R¹¹, R¹¹ is methyl, ethyl, isopropyl, isobutyl, phenyl, 4-methoxyphenyl, 4-bromophenyl, or 4-nitrophenyl. In some embodiments where R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl.

Where any of R⁵, R⁶, R⁷, R⁸, and R¹⁰ is independently —N═CR¹²—NR¹³R¹⁴, in some embodiments R¹², R¹³, and R¹⁴ are independently C1-C6 alkyl. In other embodiments R¹² and R¹³ together form a 5 or 6-membered heterocycle with the adjacent C and N. For example, when shown with the adjacent nitrogen R¹², R¹³, and R¹⁴ may be one of the following:

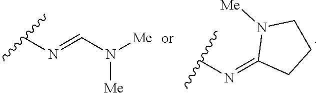 [SMILES: */N=C/N(C)C ; */N=C1/CCCN1C]

R⁹ is H, optionally substituted C1-C6 alkyl, optionally substituted benzyl, acyl, carbonate, carbamate, or silyl. Examples of acyl substituents include, for example, formyl, acetyl, propionyl, butyryl, acrylyl, crotonyl, and benzoyl. In some embodiments, R⁹ is selected from the group consisting of H, cyanoethyl, (R) and/or (S) α-methylcyanoethyl, (R) and/or (S) β-methylcyanoethyl, isobutyl, t-butyl, benzyl, u-methylbenzyl, 4-methylbenzyl, 2,4-, 3,4-, and 2,6-dimethylbenzyl, 4-methoxybenzyl, and 4-pivaloyloxy benzyl.

R¹⁹ is H or a suitable protecting group for a hydroxyl group, or a linker to solid support. Suitable protecting groups include but are not limited to TBS, TBDPS, benzoyl, trityl, DMTr, p-methoxyphenyldiphenylmethyl, benzyl, 4-methoxybenzyl, 2-tetrahydropyranyl, and ethoxyethyl.

R²² is H, a suitable protecting group for hydroxyl group, or a linker to solid support. Suitable protecting groups for hydroxyl group include, for example, but are not limited to, trialkylsilyl (where the alkyl are the same or different and are C1-C6 alkyl, such as tert-butyl(dimethyl)silyl (TBS)), tert-butyldiphenylsilyl, triphenylsilyl, trityl, substituted trityl, including MMTr (p-methoxyphenyldiphenylmethyl) and DMTr (4,4′-dimethoxytriphenylmethyl), benzyl, 4-methoxybenzyl (PMB or MPM), and acid labile protecting groups, including, for example, 2-tetrahydropyranyl or 1-ethoxyethyl.

The method includes the following steps:
(a) providing a stereo-encoded morpholino monomer of Formula (Ia)

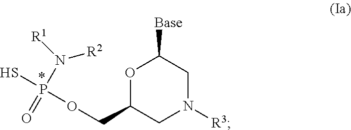

wherein R³ is optionally substituted C1-C6 alkyl, trityl benzyl, or sulfonyl;
(b) optionally sulfur-alkylating the stereo-encoded morpholino monomer of Formula (Ia) in step
(a) to afford a stereo-encoded morpholino monomer of Formula (I)

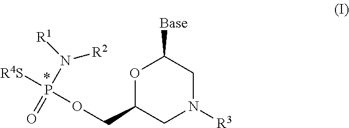

wherein R⁴ is C1-C6 alkyl;
(c) reacting the stereo-encoded morpholino monomer of Formula (Ia) from step (a) or the stereo-encoded morpholino monomer of Formula (I) from (b) with a chlorinating agent to afford a stereo-encoded activated monomer of Formula (II)

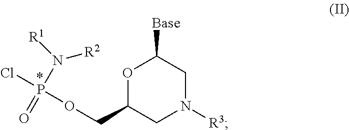

(d) optionally isolating the stereo-encoded activated compound of Formula (II) from step (c);
(e) coupling the compound of Formula (II) from step (c) or (d) with a compound of Formula (IX)

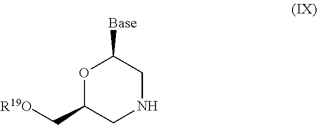

to obtain a morpholino dimer of Formula (VIII)

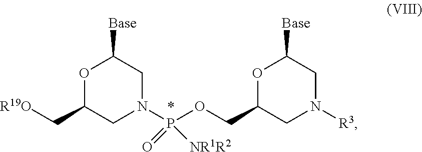

wherein R¹⁹ is a suitable protecting group for a hydroxyl group; and may be a “linker” which is generally used to connect morpholino monomer derivatives to solid support/resin. Examples include 3-aminopropyl, succinyl, 2,2′-diethanosulfonyl and a long chain alkyl amino group;
(f) deprotecting the morpholino dimer of Formula (VIII) to obtain an intermediate oligomer of Formula (X)

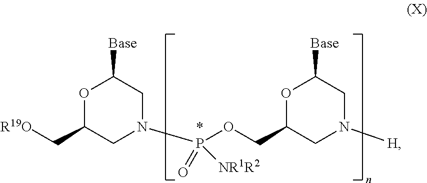

wherein n is 1;
(g) repeating step (i) and step (ii) below m times, wherein m is an integer from 0 to 6 inclusive:
   (i) reacting the intermediate oligomer of Formula (X)

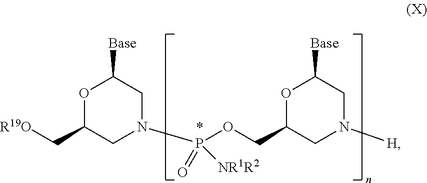

wherein n is an integer from 1 to 7 inclusive,
with a stereo-encoded activated compound of Formula (II) prepared from a stereo-encoded morpholino monomer of Formula (I) by steps (a) to (d)

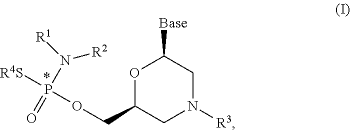

wherein R⁴ is H or C1-C6 alkyl,
to provide an intermediate of formula (XI):

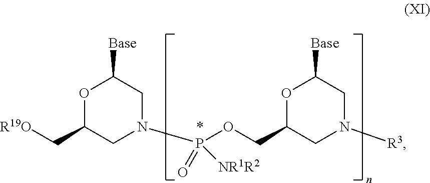

wherein n is an integer from 1 to 4 inclusive; and
   (ii) deprotecting the intermediate of Formula (XI) from step (i) to provide a phosphorodiamidate morpholino oligomer of Formula (X):

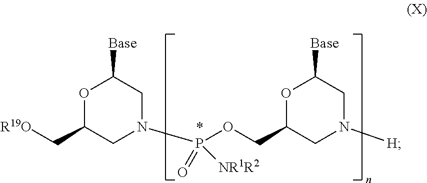

wherein n is an integer from 1 to 7 inclusive;
(h) providing a stereo-encoded DNA monomer of Formula (Iad)

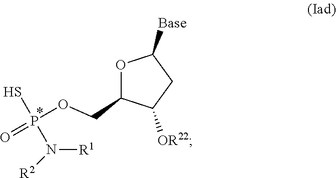

wherein R²² is a protecting group for hydroxyl oxygen. Suitable protecting groups for hydroxyl oxygen include, for example, but are not limited to, trialkylsilyl (where the alkyl are the same or different and are C1-C6 alkyl, such as tert-butyl(dimethyl)silyl (TBS)), tert-butyldiphenylsilyl, triphenylsilyl, trityl, substituted trityl, including MMTr (p-methoxyphenyldiphenylmethyl) and DMTr (4,4′-dimethoxytriphenylmethyl), benzyl, 4-methoxybenzyl (PMB or MPM), and acid labile protecting groups, including, for example, 2-tetrahydropyranyl or 1-ethoxyethyl; (j) optionally sulfur-alkylating the stereo-encoded DNA monomer of Formula (Iad) in step (h) to afford a stereo-encoded DNA monomer of Formula (Id)

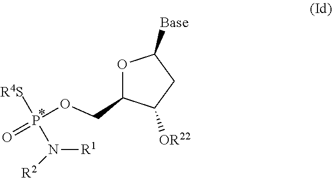

wherein R⁴ is C1-C6 alkyl;
(k) reacting the stereo-encoded DNA monomer of Formula (Iad) from step (h) or the stereo-encoded DNA monomer of Formula (Id) from (j) with a chlorinating agent to afford a stereo-encoded activated monomer of Formula (IId)

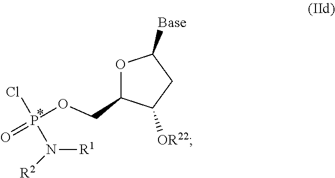

(l) optionally isolating the stereo-encoded activated compound of Formula (IId) from step (k);
(m) coupling the compound of Formula (IId) from step (k) or (1) with a compound of Formula (X) from step (g):

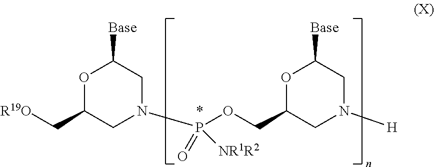

wherein n is an integer from 1 to 7 inclusive;
to provide an intermediate of formula (Xd):

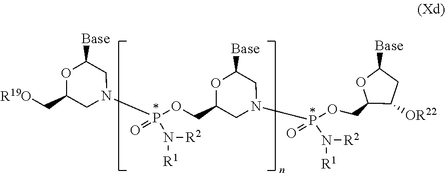

(n) optionally, removing the R²² group and/or the R¹⁹ group, to provide a PMO-DNA hetero-oligomer of Formula (Xd):

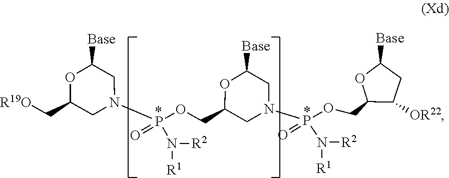

wherein n is an integer from 1 to 7 inclusive. P* represents a stereocenter that is either in the (R) or (S) configuration, and each P* does not need to be the same.
R¹¹, R², Base are as defined above. R¹⁹, R²² can be independently protection groups as defined above, or H after the protection group or linker is removed under condition known in the art. When R¹⁹ is a linker, it may be cleaved by condition known in the art.

So that the disclosure may be more fully understood, the following examples are included. These examples are for illustrative purposes only and are not to be construed as limiting.

### EXAMPLES

Definitions: The following abbreviations have the indicated meanings:
ACN: Acetonitrile
aq.: Aqueous
Boc₂O: Di-tert-butyl dicarbonate
Brettphos: 2-(Dicyclohexylphosphino)-3,6-dimethoxy-2′,4′,6′-triisopropyl-1,1′-biphenyl
tBuONa: Sodium tert-butoxide
Bz: Benzoyl
CH₂Cl₂: Dichloromethane
CH₃I: Iodomethane
Cs₂CO₃: Cesium carbonate
DCC: N,N′-dicyclohexylcarbodiimide
DCM: Dichloromethane
DIEA: N,N-diisopropylethylamine
DIPEA: N,N-diisopropylethylamine
DMAP: 4-(Dimethylamino)pyridine
DME: Dimethoxyethane
DMF: Dimethylformamide
DMSO: Dimethyl sulfoxide
EGTA: Ethylene glycol tetraacetic acid
ESI-MS: Electrospray ionization-mass spectrometry
EtOH: Ethanol
EtOAc: Ethyl acetate
h: Hours
HATU: 1-[Bis(dimethylamino)methylene]-1H-1,2,3-trizolo[4,5-b]pyridinium 3-oxid hexafluorophosphate
H₂SO₄: Sulfuric acid
Hunig's base: N,N-Diisopropylethylamine
iPrOH: Isopropanol
K₂CO₃: Potassium carbonate
KHMDS: Potassium bis(trimethylsilyl)amide
KOH: Potassium hydroxide
LCMS: Liquid chromatography—mass spectrometry
MeOH: Methanol
MgSO₄: Magnesium sulfate
min: Minutes
MsCl: Methansulfonyl chloride
NaBH₃CN: Sodium cyanoborohydride
NaBH(OAc)₃: Sodium triacetoxyborohydride
NaHCO₃: Sodium bicarbonate
NH₄Cl: Ammonium chloride
NH₄HCO₃: Ammonium bicarbonate
NaI Sodium iodide
NaNO₃: Sodium nitrate
NaOAc: Sodium acetate
nBuOH: n-Butanol
prep-HPLC: Preparative high-performance liquid chromatography
prep-TLC: Preparative thin layer chromatography
TBAF: Tetrabutylammonium fluoride
TBDMS-CL: tert-Butyldimethylsilyl chloride
TBSCl: tert-Butyldimethylsilyl chloride
TBSOTf: tert-Butyldimethylsilyl trifluoromethanesulfonate
TEA: Triethylamine
TESCl: Chlorotriethylsilane
TFA: Trifluoroacetic acid
THF: Tetrahydrofuran
Ti(OⁱPr)₄: Titanium isopropoxide
TLC: Thin-layer chromatography
PPTS: Pyridinium p-toluenesulfonate
PE: Petroleum ether
PEG: Poly(ethylene glycol)
PtO₂: platinum dioxide
Pd/C: Palladium (0) on carbon
Pd₂(dba)₃: Tris(dibenzylideneacetone) dipalladium(0)
Pd(dppf)₂Cl₂: [1,1′-Bis(diphenylphosphino)ferrocene]dichloropalladium(II)
RT: room temperature
Ruphos: 2-Dicyclohexylphosphino-2′,6′-diisopropoxybiphenyl
sat.: Saturated
SFC: supercritical fluid chromatography
Xantphos: 4,5-Bis(diphenylphosphino)-9,9-dimethylxanthene

### Example 1—Preparation of (2R,4S)-2-(dimethylamino)-4-phenyl-1,3,2-oxathiaphospholane 2-sulfide

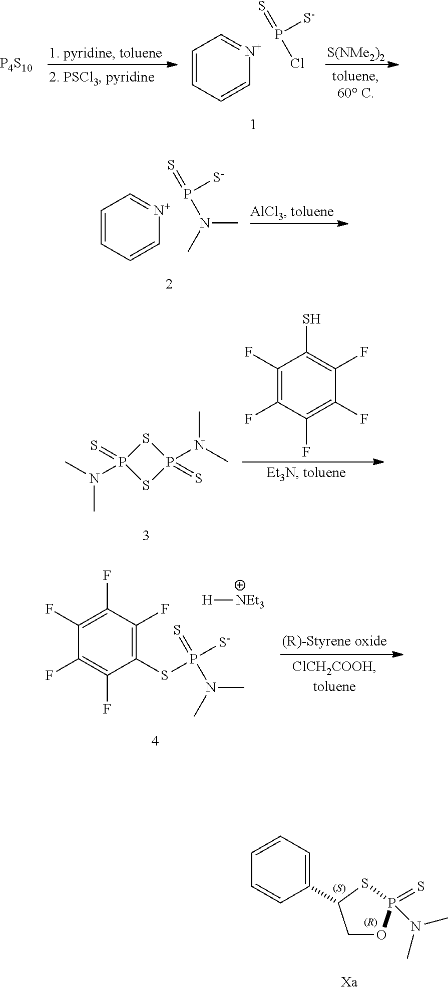

To 2,4-bis(dimethylamino)-1,3,2,4-dithiadiphosphetane 2,4-disulfide (3) (prepared using literature procedure: Z. anorg. Allg. Chemie. 1973, 397, 225-320 and Eur. J. Inorg. Chem. 2004, 3842-3845, both of which are incorporated by reference herein) (1.00 g, 3.59 mmol) in toluene (20 mL) was added 2,3,4,5,6-pentafluorobenzenethiol (1.44 g, 7.18 mmol). The reaction flask was made inert by flusing nitrogen for 10 min. Triethylamine (2.0 mL, 14 mmol) was added over a period of 30 min. The solution was stirred at room temperature overnight (³¹P NMR spectrum analysis indicated that starting material was consumed). The reaction mixture was concentrated under vacuum to give salt 4 as viscous liquid, which was used for next step directly without further purification.

Salt 4 (prepared above) was suspended in toluene (20 mL) and treated with (R)-styrene oxide (0.65 g, 5.4 mmol) followed by chloroacetic acid (1.69 g, 18.0 mmol). The mixture was stirred overnight at room temperature. The reaction mixture was poured into water (20 mL) and diluted with EtOAc (30 mL). After layer separation, the organic layer was washed with an aqueous solution of 10% K₂HPO₄, dried over Na₂SO₄, filtered, and concentrated in vacuo. The residue was dissolved in EtOAc (5 mL), and then treated with n-heptane (30 mL) to precipitatet the compound Xa as a yellow solid. ¹H and ³¹P NMR spectrum of compound Xa matched those of previously prepared compound Xa.

### Example 1A—Alternative Preparation of (2R,4S)-2-(dimethylamino)-4-phenyl-1,3,2-oxathiaphospholane 2-sulfide

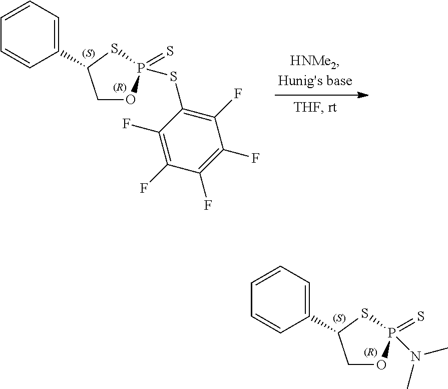

Synthesis: To a solution of (2S,4S)-2-((perfluorophenyl)thio)-4-phenyl-1,3,2-oxathiaphospholane 2-sulfide (30.0 g, 72.4 mmol) in anhydrous THF (400 mL) were added dimethylamine (91.0 mL, 181 mmol) (2.0 M in THF) followed by Hunig's base (12.7 mL, 72.4 mmol) at 0° C. and was stirred at room temperature for overnight. The reaction mixture was concentrated approximately to 100 mL and then diluted with aq. NaHCO₃ (200 mL) and EtOAc (300 mL). The organic layer was separated, washed with aq. NaHCO₃ (200 mL) and brine (2×200 mL), dried over MgSO₄, filtered and concentrated. The crude material was redissolved in EtOAc (200 mL), and filtered through a celite pad to remove any solids. The filtrate was concentrated and purified by silica gel column chromatography (CH₂Cl₂/n-heptane=0% to 100%) to give viscous liquid.

Precipitation: The above purified compound was dissolved in EtOAc (50 mL), slowly treated with n-heptane (200 mL), and then stirred at room temperature for 2 h. The resulting solid was filtered, washed with n-heptane (50 mL), and dried under vacuum for 18 h to give (2R,4S)-2-(dimethylamino)-4-phenyl-1,3,2-oxathiaphospholane 2-sulfide (12.4 g, 66% yield) as a white solid.

¹H NMR (400 MHz, CDCl₃) δ 7.36-7.24 (m, 5H), 5.12 (dt, J=4.9, 8.3 Hz, 1H), 4.62-4.49 (m, 1H), 4.22 (ddd, J=8.3, 9.8, 14.5 Hz, 1H), 2.87 (s, 3H), 2.84 (s, 3H); ³¹P NMR (162 MHz, CDCl₃) δ 102.9; ESI-MS m/z: Calculated for [C₁₀H₁₄NOPS₂+H]⁺ 260.03; Found 260.08.

### Example 1C—Preparation of (2S,4R)-2-(dimethylamino)-4-phenyl-1,3,2-oxathiaphospholane 2-sulfide

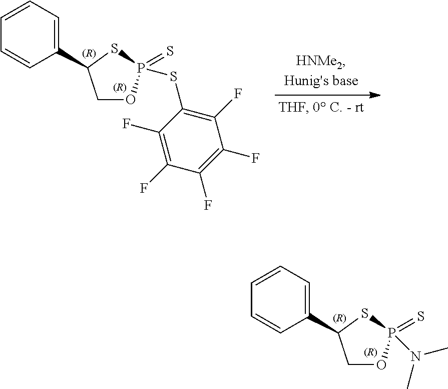

Using the same procedure and same equivalents as for the preparation of (2R,4S)-2-(dimethylamino)-4-phenyl-1,3,2-oxathiaphospholane 2-sulfide in Example 1, 20.0 g of (2R,4R)-2-((perfluorophenyl)thio)-4-phenyl-1,3,2-oxathiaphospholane 2-sulfide afforded the title compound (8.4 g, 67%) as a white solid.

¹H NMR (400 MHz, CDCl₃) δ 7.35-7.22 (m, 5H), 5.14-5.08 (m, 1H), 4.59-4.48 (m, 1H), 4.32 (ddd, J=8.5, 9.6, 14.7 Hz, 1H), 2.85 (s, 3H), 2.82 (s, 3H); ³¹P NMR (162 MHz, CDCl₃) δ 102.9; ESI-MS m/z: Calculated for [C₁₀H₁₄NOPS₂+H]⁺ 260.03; Found 260.17.

### Example 2—Preparation of Crystalline Morpholino Monomer O-(((2S,6R)-6-(4-benzamido-2-oxopyrimidin-1(2H)-yl)-4-tritylmorpholin-2-yl)methyl)S-hydrogen (S)-dimethylphosphoramidothioate

### Example 2A: Using Styrene-PSI—NMe₂

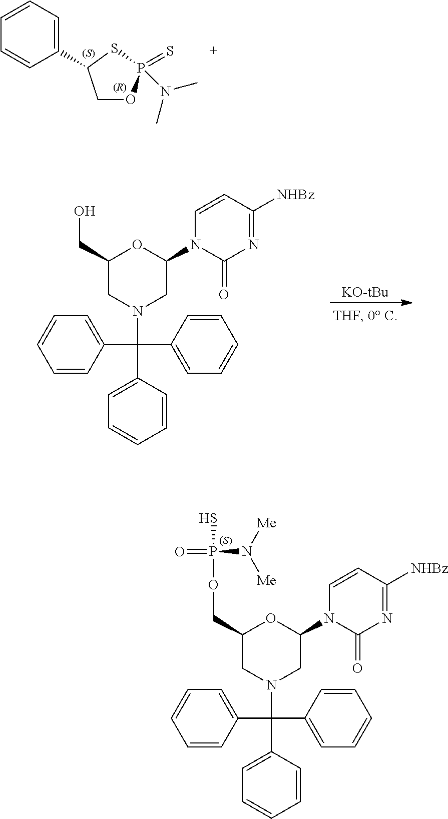

A solution of N-(1-((2R,6S)-6-(hydroxymethyl)-4-tritylmorpholin-2-yl)-2-oxo-1,2-dihydropyrimidin-4-yl)benzamide (1.00 g, 1.74 mmol) and (2R,4S)-2-(dimethylamino)-4-phenyl-1,3,2-oxathiaphospholane 2-sulfide (0.68 g, 2.62 mmol) in THF (17.5 mL) was cooled to 0° C. using ice-bath. Potassium tert-butoxide (5.2 mL, 5.24 mmol) (1.0 M in THF) was added in dropwise over 20 min. After stirring at 0° C. for 2 h, the reaction was quenched by addition of AcOH (0.6 mL, 10.5 mmol) and stirred at room temperature for 1 h. The reaction mixture was diluted with 10% aq. NaH₂PO₄ (20 mL) and EtOAc (30 mL). The organic layer was separated, washed with brine (3×20 mL), dried over MgSO₄, filtered and concentrated to final approximate volume of 5 mL. The residue was diluted with n-heptane (25 mL), and stirred for 30 min to precipitate the thioate as a white solid. The solid was filtered, washed with n-heptane (10 mL), and dried under vacuum for overnight to give the title compound (950 mg, 78%).

A small quantity of compound was purified by silica gel column chromatography (THF/n-heptane=0% to 100%) to record spectral data.

¹H NMR (400 MHz, CDCl₃) δ 11.59 (br s, 1H), 8.01 (br d, J=7.3 Hz, 2H), 7.65-7.38 (m, 10H), 7.36-7.14 (m, 10H), 6.12 (br d, J=8.5 Hz, 1H), 4.47-4.31 (m, 1H), 3.99-3.85 (m, 1H), 3.65-3.54 (m, 1H), 3.49 (br d, J=11.0 Hz, 1H), 3.15-3.03 (m, 1H), 3.03-2.94 (m, 6H), 2.74-2.66 (m, 1H), 1.37-1.13 (m, 2H); ³¹P NMR (162 MHz, CDCl₃) δ 65.1; ESI-MS m/z: Calculated for [C₃₇H₃₈N₅O₅PS+H]⁺ 696.24; Found 696.30.

### Example 2B: Using chiral 2-(dimethylamino)-1,3,2-oxathiaphospholane 2-sulfide

### Synthesis of 2-(dimethylamino)-1,3,2-oxathiaphospholane 2-sulfide

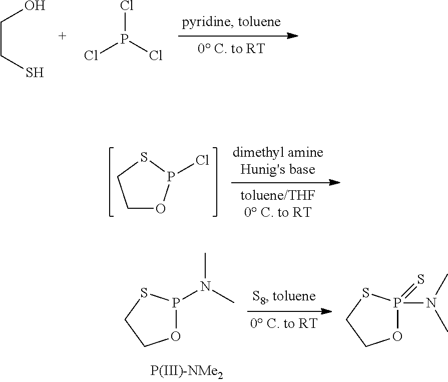

Into a solution of trichlorophosphane (33.5 mL, 384 mmol) in toluene (400 mL) was added pyridine (62.1 mL, 768 mmol) at 0° C. After 20 min, 2-mercaptoethan-1-ol (30.0 g, 384 mmol) was added to the reaction mixture in dropwise at 0° C. for 30 min (Caution: maintain the reaction temperature under 0° C. with vigorous stirring. Pyridinium chloride was precipitated as a white solids). After completion of addition, the reaction mixture was warmed to room temperature and continued stirring for additional 1 h. Filtered the solids, washed toluene (100 mL), and the filtrate was concentrated under reduced pressure to an approximate volume of 300 mL (maintain the water bath temperature 30° C. at 20 mm of Hg).

To the residual toluene solution was added Hunig's base (101 mL, 575 mmol) followed by dimethylamine (384 mL, 767 mmol, 2.0 M in THF) at 0° C. After stirring at room temperature for 6 h, diisopropylamine hydrochloride solids were removed by filtration, solvent was evaporated under reduced pressure, and the product was distilled (105-110° C. at house vacuum).

Into a solution of P(III)-NMe₂ in toluene (200 mL) was added molecular sulfur (18.47 g, 576 mmol) at 0° C. The reaction mixture was stirred at 0° C. for 1 h, at RT for overnight. The reaction was filtered through a pad of Celite, washed with EtOAc (100 mL), the filtrate was concentrated and the crude material was purified using silica gel column chromatography (EA/n-heptane=0 to 50%) to give 2-(dimethylamino)-1,3,2-oxathiaphospholane 2-sulfide (23.5 g, 128 mmol, 33% yield) as pale yellow oil.

¹H NMR (400 MHz, DICHLOROMETHANE-d₂) δ 4.42-4.58 (m, 1H), 4.29 (tdd, J=10.22, 10.22, 5.69, 4.88 Hz, 1H), 3.50-3.58 (m, 1H), 3.38-3.48 (m, 1H), 2.87-2.90 (m, 3H), 2.83-2.85 (m, 3H); ³¹P NMR (162 MHz, DICHLOROMETHANE-d₂) δ 102.05 (s, 1 P).

The racemic mixture 2-(dimethylamino)-1,3,2-oxathiaphospholane 2-sulfide was subjected to the following chiral SFC conditions to isolate the (R) and (S)-stereoisomers of the product:

### Analytical Conditions:

Column: ChiralCel OD-H 4.6×100 mm
Mobile Phase: 10% Isopropanol in CO2
Flow Rate: 2.5 m/min
Sample: 1.0 mg/mL
Injection: 5 uL
Detection: 220 nm

### Preparative Conditions:

Column: ChiralCel OD-H 21×250 mm
Mobile Phase: 10% Isopropanol in CO2
Flow Rate: 60 mE/min
Sample: 500 mg was dissolved in 16.6 mL Isopropanol+16.6 mL
   Dichloromethane
Injection: 1.0 mL
Detection: 220 nm

### (R)-2-(dimethylamino)-1,3,2-oxathiaphospholane 2-sulfide

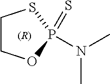

Peak 1 (Retention time=1.48 min)

¹H NMR (400 MHz, DICHLOROMETHANE-d₂) δ 4.43-4.60 (m, 1H), 4.25-4.35 (m, 1H), 3.49-3.59 (m, 1H), 3.39-3.49 (m, 1H), 2.87-2.92 (m, 3H), 2.81-2.85 (m, 3H); ³¹P NMR (162 MHz, DICHLOROMETHANE-d₂) δ 102.05 (s, 1 P).

### (S)-2-(dimethylamino)-1,3,2-oxathiaphospholane 2-sulfide

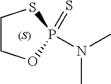

Peak 2 (Retention time=1.81 min)

¹H NMR (400 MHz, DICHLOROMETHANE-d₂) δ 4.43-4.60 (m, 1H), 4.25-4.35 (m, 1H), 3.49-3.59 (m, 1H), 3.39-3.49 (m, 1H), 2.87-2.92 (m, 3H), 2.81-2.85 (m, 3H); ³¹P NMR (162 MHz, DICHLOROMETHANE-d₂) δ 102.05 (s, 1 P).

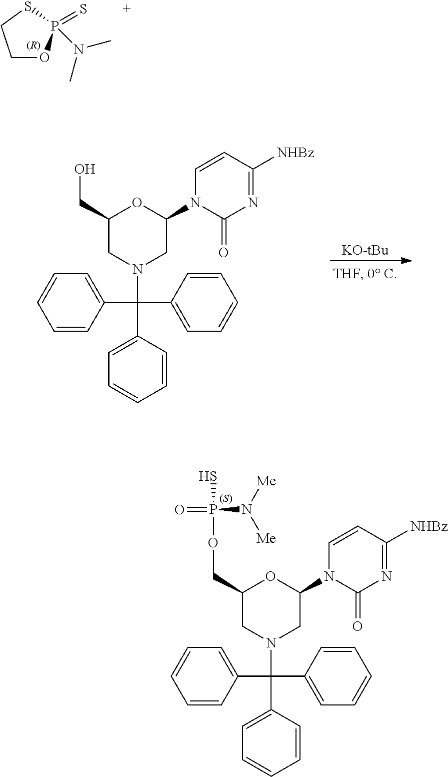

A solution of N-(1-((2R,6S)-6-(hydroxymethyl)-4-tritylmorpholin-2-yl)-2-oxo-1,2-dihydropyrimidin-4-yl)benzamide (100 mg, 0.17 mmol) and 2-(dimethylamino)-1,3,2-oxathiaphospholane 2-sulfide (48 mg, 0.26 mmol) (isolated as peak 1 from chiral SFC of 2-(dimethylamino)-1,3,2-oxathiaphospholane 2-sulfide) in THF (1.8 mL) was cooled to 0° C. using ice-bath. Potassium tert-butoxide (0.52 mL, 0.52 mmol) (1.0 M in THF) was added in dropwise over 5 min. After stirring at 0° C. for 1 h, the reaction was quenched by addition of AcOH (0.06 mL, 1.05 mmol) and stirred at room temperature for 30 min. The reaction mixture was diluted with 10% aq. NaH₂PO₄ (5 mL) and EtOAc (10 mL). The organic layer was separated, washed with brine (2×5 mL), dried over Na₂SO₄, filtered and concentrated. The crude residue was dissolved on EtOAc (2 ml), slowly diluted with n-heptane (8 mL), and stirred for 30 min to precipitate the thioate as a white solid. The solid was filtered, washed with n-heptane (10 mL), and dried under vacuum for overnight to give the title compound (94 mg, 77%).

### Example 3—Preparation of Crystalline Morpholino Monomer O-(((2S,6R)-6-(4-benzamido-2-oxopyrimidin-1(2H)-yl)-4-tritylmorpholin-2-yl)methyl)S-hydrogen (R)-dimethylphosphoramidothioate

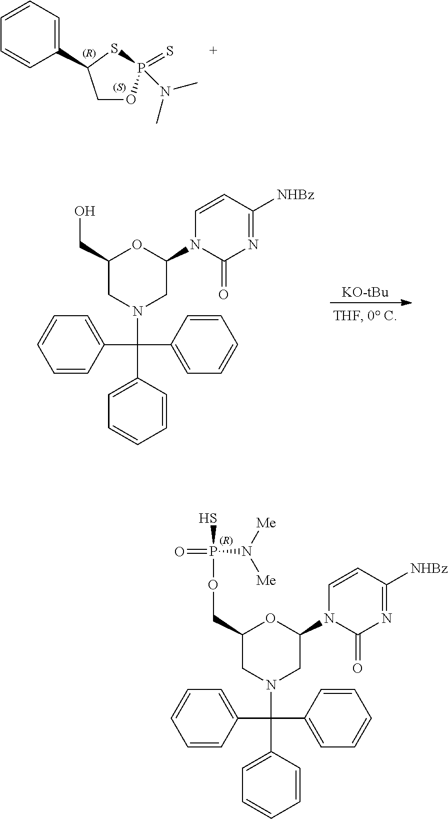

According to the procedure reported for O-(((2S,6R)-6-(4-benzamido-2-oxopyrimidin-1(2H)-yl)-4-tritylmorpholin-2-yl)methyl)S-hydrogen (S)-dimethylphosphoramidothioate (in Example 4), using N-(1-((2R,6S)-6-(hydroxymethyl)-4-tritylmorpholin-2-yl)-2-oxo-1,2-dihydropyrimidin-4-yl)benzamide (0.5 g, 0.87 mmol) and (2S,4R)-2-(dimethylamino)-4-phenyl-1,3,2-oxathiaphospholane 2-sulfide (0.34 g, 1.31 mmol) afforded the title compound (0.53 g, 87%) as a white solid.

¹H NMR (400 MHz, CDCl₃) δ 8.85 (br s, 1H), 8.00 (br d, J=7.5 Hz, 2H), 7.72 (br d, J=7.5 Hz, 1H), 7.62-7.37 (m, 9H), 7.33-7.05 (m, 10H), 6.16 (br. d, J=8.7 Hz, 1H), 4.42 (br s, 1H), 4.21-4.00 (m, 2H), 3.53 (br d, J=10.8 Hz, 1H), 3.14 (br d, J=11.5 Hz, 1H), 2.81 (s, 3H), 2.80 (s, 3H), 1.59-1.40 (m, 1H), 1.37-1.25 (m, 2H); ³¹P NMR (162 MHz, CDCl₃) δ 63.3; ESI-MS m/z: Calculated for [C₃₇H₃₈N₅O₅PS+H]⁺ 696.24; Found 696.15.

### Example 4—Preparation of Crystalline Morpholino Monomer O-(((2S,6R)-6-(5-methyl-2,4-dioxo-3,4-dihydropyrimidin-1(2H)-yl)-4-tritylmorpholin-2-yl)methyl)S-hydrogen (S)-dimethylphosphoramidothioate

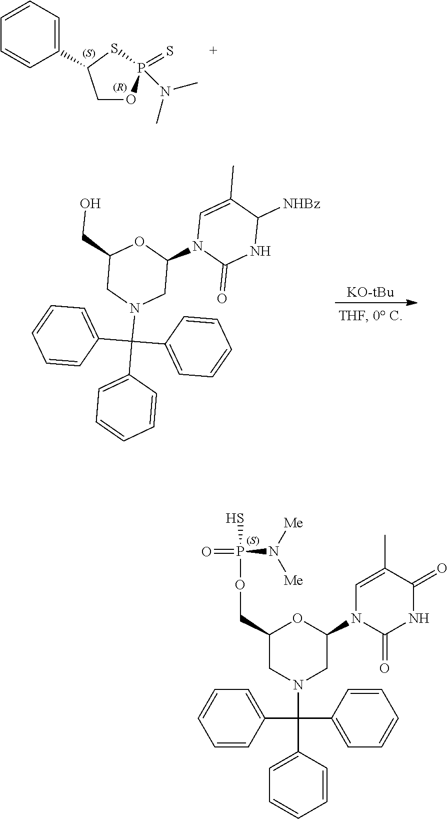

A solution of 1-((2R,6S)-6-(hydroxymethyl)-4-tritylmorpholin-2-yl)-5-methylpyrimidine-2,4(1H,3H)-dione (0.50 g, 1.03 mmol) (2R,4S)-2-(dimethylamino)-4-phenyl-1,3,2-oxathiaphospholane 2-sulfide (0.40 g, 1.55 mmol) in THF (8.0 mL) was cooled to 0° C. and added potassium tert-butoxide (3.1 mL, 3.10 mmol) (1.0 M in THF) in dropwise over 20 min. After stirring at 0° C. for 2 h, the reaction mixture was quenched by the addition of AcOH (0.35 mL, 6.21 mmol) and stirred at room temperature for 1 h. The reaction mixture was diluted with 10% aq. NaH₂PO₄ (20 mL) and EtOAc (30 mL). The organic layer was separated, washed with brine (3×20 mL), dried over MgSO₄, filtered and concentrated to final approximate volume of 5 mL. The residue was diluted with n-heptane (25 mL), and stirred for 30 min to precipitate the thioate as a white solid. The solids were filtered, washed with n-heptane (10 mL), and dried under vacuum for overnight to give the title compound (0.54 g, 87%)

A small quantity of compound was purified by silica gel column chromatography (THF/n-heptane=0% to 100%) to record spectral data.

1H NMR (400 MHz, CDCl₃) δ 9.58-9.31 (br s, 1H), 7.55-7.41 (m, 6H), 7.38-7.25 (m, 6H), 7.25-7.12 (m, 3H), 6.12 (d, J=9.3 Hz, 1H), 4.45-4.37 (m, 1H), 4.22-4.12 (m, 1H), 4.12-4.00 (m, 1H), 3.77 (t, J=6.3 Hz, 1H), 3.38 (d, J=10.5 Hz, 1H), 3.14 (d, J=11.3 Hz, 1H), 2.74 (s, 3H), 2.70 (s, 3H), 1.90-1.85 (m, 1H), 1.83 (s, 3H), 1.54-1.41 (m, 1H), 1.36-1.25 (m, 1H); ³¹P NMR (CDCl₃, 162 MHz) δ 65.0; ESI-MS m/z: Calculated for [C₃₁H₃₅N₄O₅PS+Na]⁺ 629.19; Found 629.4.

### Example 5—Preparation of Crystalline Morpholino Monomer O-(((2S,6R)-6-(6-benzamido-9H-purin-9-yl)-4-tritylmorpholin-2-yl)methyl)S-hydrogen (S)-dimethylphosphoramidothioate

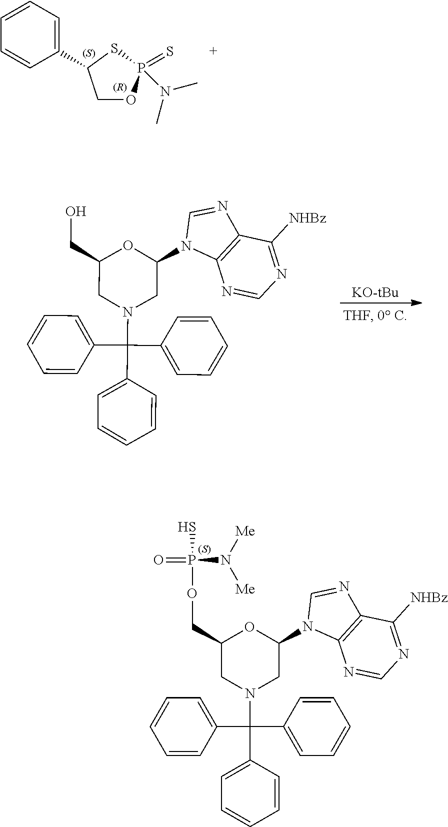

A solution of N-(9-((2R,6S)-6-(hydroxymethyl)-4-tritylmorpholin-2-yl)-9H-purin-6-yl)benzamide (0.50 g, 0.84 mmol) and (2R,4S)-2-(dimethylamino)-4-phenyl-1,3,2-oxathiaphospholane 2-sulfide (0.26 g, 1.01 mmol) in THF (8.4 mL) was cooled to 0° C. and, treated with potassium tert-butoxide (2.51 mL, 2.51 mmol) (1.0 M in THF) in dropwise for 10 min, and stirred at the same temperature for 2 h. The reaction was quenched with AcOH (0.3 mL, 5.03 mmol), and stirred at 0° C. for 30 min and at RT for 30 min. The reaction mixture was diluted with 1.0 N aq. NaH₂PO₄ solution (20 mL) and EtOAc (30 mL). The organic layer was separated, washed with brine (3×15 mL), dried over MgSO₄, filtered and concentrated under vacuum to a final approximate volume of 5 mL. The residue was diluted with n-heptane (25 mL), and stirred for 30 min to precipitate the thioate as a white solid. The solids were filtered, washed with n-heptane (10 mL), dried under vacuum for overnight to give the title compound (0.48 g, 80%).

¹H NMR (THF-d8, 400 MHz) δ 10.02-10.33 (m, 1H), 8.54-8.68 (m, 1H), 8.15-8.28 (m, 1 H), 7.96-8.08 (m, 2H), 7.42-7.71 (m, 9H), 7.10-7.41 (m, 10H), 6.43-6.53 (m, 1H), 4.47-4.61 (m, 1H), 3.89-4.12 (m, 2H), 3.80-3.89 (m, 1H), 3.31-3.42 (m, 1H), 2.57-2.58 (m, 6 H), 1.99-2.11 (m, 1H), 1.63-1.71 (m, 1H); ³¹P NMR (THF-d8, 162 MHz) δ 71.9; ESI-MS m/z: Calculated for [C₃₈H₃₈N₇O₄PS+H]⁺ 720.25; Found 720.21.

### Example 6—Preparation of Crystalline Morpholino Monomer O-(((2S,6R)-6-(2-isobutyramido-6-oxo-1,6-dihydro-9H-purin-9-yl)-4-tritylmorpholin-2-yl)methyl)S-hydrogen (S)-dimethylphosphoramidothioate

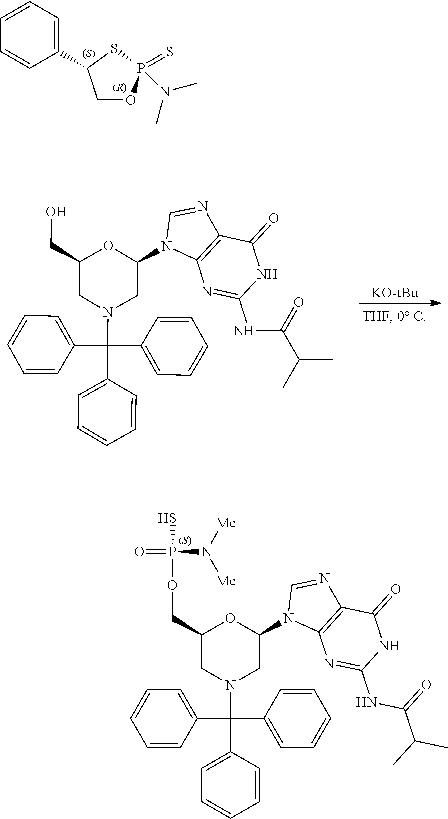

A solution of N-(9-((2R,6S)-6-(hydroxymethyl)-4-tritylmorpholin-2-yl)-6-oxo-6,9-dihydro-1H-purin-2-yl)isobutyramide (0.50 g, 0.86 mmol) and (2R,4S)-2-(dimethylamino)-4-phenyl-1,3,2-oxathiaphospholane 2-sulfide (0.45 g, 1.72 mmol) in THF (8.6 mL) was cooled to 0° C., treated with potassium tert-butoxide (3.5 mL, 3.46 mmol) (1.0 M in THF) in dropwise over 20 min, and stirred at the same temperature for 2 h. The reaction was quneched with AcOH (0.4 mL, 6.91 mmol), and stirred at 0° C. for 30 min and at room temperature for 30 min. The reaction mixture was diluted with 1.0 N aq. NaH₂PO₄ solution (20 mL) and EtOAc (30 mL). The organic layer was separated, washed with brine (3×15 mL), dried over MgSO₄, filtered and concentrated under vacuum to a final approximate volume of 5 mL. The residue was diluted with n-heptane (25 mL) and stirred for 30 min to precipitate the thioate as a white solid. The solids were filtered, washed with n-heptane (10 mL), and dried under vacuum for overnight to give the title compound (0.38 g, 64%).

1H NMR (CDCl_{3,} 400 MHz) δ 12.41-12.13 (m, 1H), 10.27-9.93 (m, 1H), 7.86-7.66 (m, 1H), 7.55-7.36 (m, 5H), 7.36-7.23 (m, 10H), 6.07-5.84 (m, 1H), 4.51-4.34 (m, 1H), 4.32-4.12 (m, 1H), 4.11-3.91 (m, 1H), 3.48-3.29 (m, 1H), 3.22-3.06 (m, 1H), 2.93-2.70 (m, 7H), 2.69-2.57 (m, 1H), 1.92-1.66 (m, 1H), 1.65-1.48 (m, 1H), 1.34-1.07 (m, 6H); ³¹p NMR (CDCl₃, 162 MHz) δ 64.0; ESI-MS m/z: Calculated for [C₃₅H₄₀N₇O₅PS+H]⁺ 702.26; Found 702.18.

### Example 7—Preparation of ((2S,6R)-6-(4-benzamido-2-oxopyrimidin-1(2H)-yl)-4-tritylmorpholin-2-yl)methyl (R)-dimethylphosphoramidochloridate

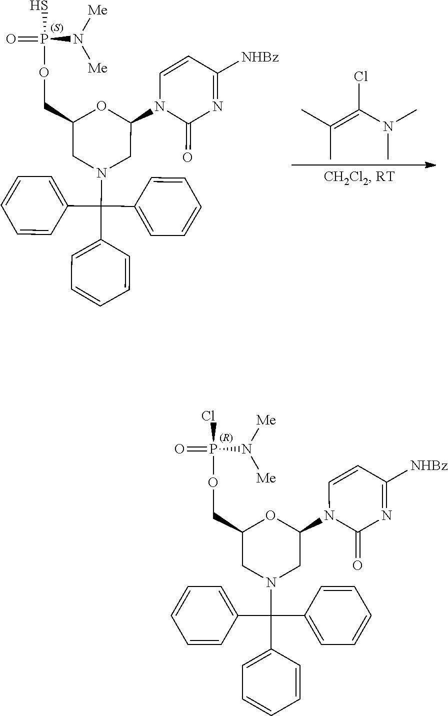

A solution of O-(((2S,6R)-6-(4-benzamido-2-oxopyrimidin-1(2H)-yl)-4-tritylmorpholin-2-yl)methyl)S-hydrogen (S)-dimethylphosphoramidothioate (0.65 g, 0.93 mmol) in anhydrous CH₂Cl₂ (10 mL) was cooled to 0° C. 1-chloro-N,N,2-trimethylprop-1-en-1-amine (0.25 mL, 1.86 mmol) was added and the mixture was stirred at room temperature for 1 h. The reaction was quenched with aq. sat. NaHCO₃ (20 mL), and diluted with CH₂Cl₂ (30 mL). Two layers were separated and the aqueous layer was extracted with CH₂Cl₂(3×20 mL). The combined organic layers were dried over MgSO₄, filtered, concentrated, and purified using silica gel column chromatography (EtOAc/n-heptane=10% to 100%) to give ((2S,6R)-6-(4-benzamido-2-oxopyrimidin-1(2H)-yl)-4-tritylmorpholin-2-yl)methyl (R)-dimethylphosphoramidochloridate (0.43 g, 67% yield) as a off-white solid.

1H NMR (CDCl₃, 400 MHz) δ 8.75-8.65 (m, 1H), 7.93-7.86 (m, 2H), 7.78-7.74 (m, 1H), 7.66-7.29 (m, 16H), 7.27-7.16 (m, 3H), 6.30 (dd, J=9.1, 1.9 Hz, 1H), 4.50-4.42 (m, 1H), 4.25-4.10 (m, 2H), 3.63 (br d, J=11.3 Hz, 1H), 3.19 (br d, J=11.7 Hz, 1H), 2.68 (s, 3H), 2.65 (s, 3H), 1.62-1.51 (m, 1H), 1.37-1.24 (m, 1H); ³¹P NMR (CDCl₃, 162 MHz) δ 18.4 (Sp isomer), 18.1 (Rp isomer); ESI-MS m/z: Calculated for [C₃₇H₃₇C1N5O5P+H]⁺698.23; Found 698.22.

Diastereomeric ratio: >200 (Rp isomer): 1 (Sp isomer) (³¹P NMR).

### Example 8—Preparation of ((2S,6R)-6-(4-benzamido-2-oxopyrimidin-1(2H)-yl)-4-tritylmorpholin-2-yl)methyl (S)-dimethylphosphoramidochloridate

### Example 8A: One Step Procedure Using SO₂Cl₂ 2,4,6-Collidine

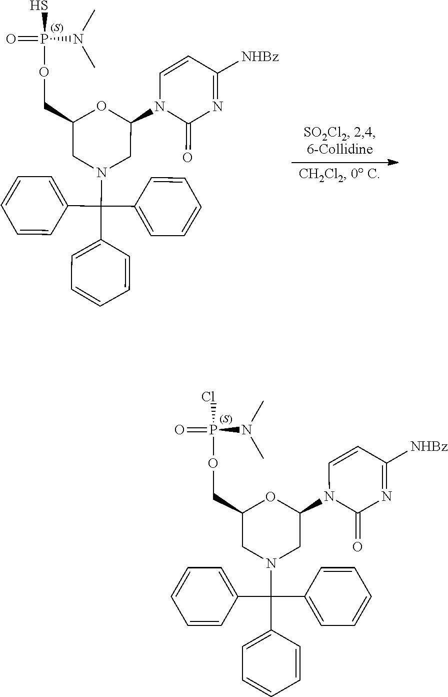

To a solution of O-(((2S,6R)-6-(4-benzamido-2-oxopyrimidin-1(2H)-yl)-4-tritylmorpholin-2-yl)methyl)S-hydrogen (R)-dimethylphosphoramidothioate (50 mg, 0.07 mmol) in anhydrous CH₂Cl₂ (0.5 mL) was added 2,4,6-trimethylpyridine (87 mg, 0.72 mmol) at 0° C. After 10 min, sulfuryl dichloride (30 mg, 0.22 mmol) (0.5 M in toluene) was added and stirred at 0° C. for 30 min. The reaction was quenched by the addition of aq. NaHCO₃ (2.0 mL), warmed to room temperature, diluted with CH₂Cl₂(2.0 mL). The aqueous layer was extracted with CH₂Cl₂ (3×2 mL). The combined organic layers were dried over Na₂SO₄, filtered, concentrated and purified by using silica gel column chromatography (EtOAc/n-heptane=10% to 100%) to yield title compound (33 mg, 66%).

¹H NMR (CDCl₃, 400 MHz) δ 8.67-8.42 (m, 1H), 7.89-7.79 (m, 1H), 7.70-7.60 (m, 1H), 7.60-7.47 (m, 1H), 7.44-7.27 (m, 8H), 7.27-7.17 (m, 6H), 7.17-7.07 (m, 3H), 6.28-6.16 (m, 1H), 4.42-4.28 (m, 1H), 4.12-3.90 (m, 2H), 3.57-3.49 (m, 1H), 3.19-3.09 (m, 1H), 2.59 (s, 3H), 2.56 (s, 3H), 1.51-1.41 (m, 1H), 1.34-1.17 (m, 1H); ³¹P NMR (CDCl₃, 162 MHz) δ 18.4 (Sp isomer), 18.1 (Rp isomer); ESI-MS m/z: Calculated for [C₃₇H₃₇ClN₅O₅P+H]⁺ 698.23; Found 698.29.

Diastereomeric ratio: 5 (Sp isomer): 1 (Rp isomer) (³¹P NMR integration).

### Example 8B: Two Step Procedure

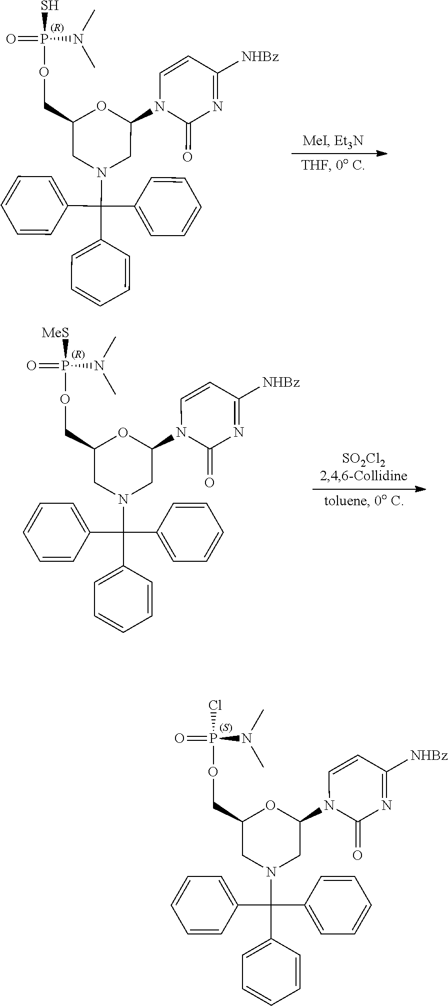

O-(((2S,6R)-6-(4-benzamido-2-oxopyrimidin-1(2H)-yl)-4-tritylmorpholin-2-yl)methyl)S-hydrogen (R)-dimethylphosphoramidothioate (1.0 g, 1.43 mmol) was dissolved in THF (10.0 mL).

To the suspension triethylamine (0.41 mL, 2.87 mmol) followed by methyl iodide (0.18 mL, 2.87 mmol) were added. The reaction was quenched with water, extract with EtOAc (30 mL). The organic layer was washed with brine (20 mL), dried over MgSO₄, concentrated and purified by silicagel chromatography (MeOH/DCM=0% to 20%) to afford O-(((2S,6R)-6-(4-benzamido-2-oxopyrimidin-1(2H)-yl)-4-tritylmorpholin-2-yl)methyl)S-methyl (R)-dimethylphosphoramidothioate.

¹H NMR (CDCl₃, 400 MHz) δ 9.13-8.92 (m, 1H), 8.01-7.90 (m, 2H), 7.86-7.79 (m, 1H), 7.67-7.44 (m, 10H), 7.36-7.29 (m, 6H), 7.24-7.16 (m, 3H), 6.32-6.23 (m, 1H), 4.49-4.38 (m, 1H), 4.14-3.99 (m, 2H), 3.65-3.57 (m, 1H), 3.22-3.16 (m, 1H), 2.71 (s, 3H), 2.68 (s, 3H), 2.23-2.17 (d, J=12.0 Hz, 3H), 1.62-1.50 (m, 1H), 1.38-1.28 (m, 1H); ³¹P NMR (CDCl₃, 162 MHz) δ 37.7 (Sp isomer), 37.4 (Rp isomer); ESI-MS m/z: Calculated for [C₃₈H₄₀N₅O₅P+H]⁺ 710.25; Found 710.05.

Diastereomeric ratio: 1 (Sp isomer): 10 (Rp isomer) (³¹P NMR integration)

A flask was charged with O-(((2S,6R)-6-(4-benzamido-2-oxopyrimidin-1(2H)-yl)-4-tritylmorpholin-2-yl)methyl)S-methyl (R)-dimethylphosphoramidothioate (0.1 g, 0.14 mmol) in toluene (2.0 mL) and cooled to 0° C. The reaction mixture was treated with 2,4,6-trimethylpyridine (0.2 mL, 1.41 mmol) followed by sulfuryl chloride (0.34 mL, 0.17 mmol) (0.5 M solution in toluene) and stirred at the same temperature for 30 min. The reaction mixture was quenched with aq. NaHCO₃ (5 mL). The aqueous layer was extracted with EtOAc (3×5 mL) and the combined organic layers were dried over Na₂SO₄, filtered, concentrated.

³¹P NMR (CDCl₃, 162 MHz) δ 18.5 (Sp isomer), 18.1 (Rp isomer); ESI-MS m/z: Calculated for [C₃₇H₃₇ClN₅O₅P+H]⁺ 698.23; Found 698.01.

Diastereomeric ratio: 9 (Sp isomer): 1 (Rp isomer) (³¹P NMR integration)

### Example 9—Preparation of ((2S,6R)-6-(5-methyl-2,4-dioxo-3,4-dihydropyrimidin-1(2H)-yl)-4-tritylmorpholin-2-yl)methyl (R)-dimethylphosphoramidochloridate

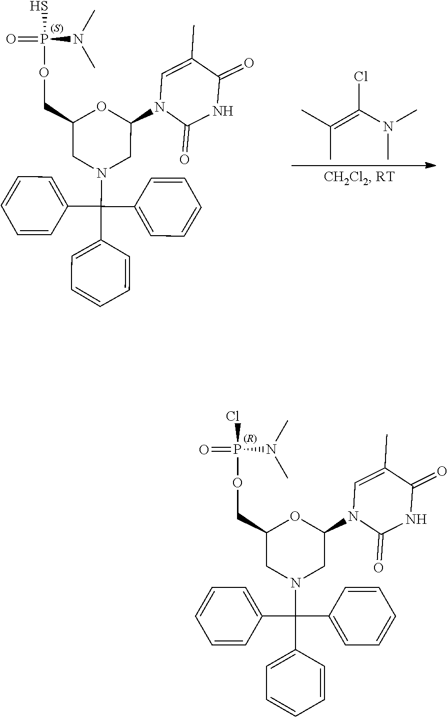

To a solution of O-(((2S,6R)-6-(5-methyl-2,4-dioxo-3,4-dihydropyrimidin-1(2H)-yl)-4-tritylmorpholin-2-yl)methyl)S-hydrogen (S)-dimethylphosphoramidothioate (0.65 g, 1.07 mmol) in CH₂Cl₂ (10.0 mL) was added 1-chloro-N,N,2-trimethylpropenylamine (0.21 mL, 1.61 mmol) at 0° C. After 1 h stirring at the same temperature, the reaction mixture was quenched with aq. sat. NaHCO₃ (20 mL), then diluted with CH₂Cl₂ (30 mL). The two layers were separated, and the aqueous layer was extracted with CH₂Cl₂ (3×20 mL). The combined organic layers were dried over MgSO₄, filtered, concentrated and purified using silica gel column chromatography (EtOAc/n-heptane=10% to 100%) to give pure ((2S,6R)-6-(5-methyl-2,4-dioxo-3,4-dihydropyrimidin-1(2H)-yl)-4-tritylmorpholin-2-yl)methyl (R)-dimethylphosphoramidochloridate (0.36 g, 55% yield) as a white solid.

¹H NMR (CDCl₃, 400 MHz) δ 8.06-8.02 (m, 1H), 7.53-7.44 (m, 5H), 7.36-7.29 (m, 6H), 7.25-7.18 (m, 3H), 7.07 (s, 1H), 6.15 (dd, J=9.5, 2.3 Hz, 1H), 4.46-4.38 (m, 1H), 4.17-4.10 (m, 2H), 3.42-3.36 (m, 1H), 3.21-3.15 (m, 1H), 2.69 (s, 3H), 2.66 (s, 3H), 1.85 (s, 3H), 1.56-1.38 (m, 2H); ³¹P NMR (CDCl₃, 162 MHz) δ 18.4 (Sp isomer), 18.1 (Rp isomer); ESI-MS m/z: Calculated for [C₃₁H₃₄ClN₄O₅P+Na]⁺ 631.18; Found 631.17.

Diastereomeric ratio: 1 (Sp isomer): 116 (Rp isomer) (³¹P NMR integration).

### Example 10A—Preparation of ((2S,6R)-6-(6-benzamido-9H-purin-9-yl)-4-tritylmorpholin-2-yl)methyl (R)-dimethyl-phosphoramidochloridate

### One Step Protocol:

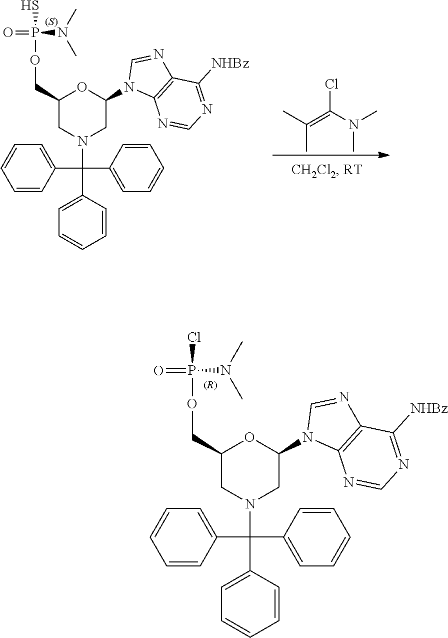

To a solution of O-(((2S,6R)-6-(6-benzamido-9H-purin-9-yl)-4-tritylmorpholin-2-yl)methyl)S-hydrogen (S)-dimethylphosphoramidothioate (0.1 g, 0.14 mmol) in CH₂Cl₂ (2.0 mL) was added 1-chloro-N,N,2-trimethylpropenylamine (30 μL, 0.21 mmol) at 0° C. After 1 h stirring at the same temperature, the reaction was quenched with aq. sat. NaHCO₃ (2 mL), then diluted with CH₂Cl₂ (5 mL). Two layers were separated and the aqueous layer was extracted with CH₂Cl₂(3×5 mL). The combined organic layers were dried over MgSO₄, filtered, concentrated and purified using silica gel column chromatography (EtOAc/n-heptane=0% to 100%) to give pure ((2S,6R)-6-(6-benzamido-9H-purin-9-yl)-4-tritylmorpholin-2-yl)methyl (R)-dimethylphosphoramidochloridate (68 mg, 67% yield) as white solid.

¹H NMR (CDCl₃, 400 MHz) δ 9.12-8.97 (m, 1H), 8.84-8.81 (m, 1H), 8.07-8.01 (m, 3H), 7.66-7.59 (m, 1H), 7.57-7.48 (m, 7H), 7.37-7.31 (m, 6H), 7.27-7.21 (m, 3H), 6.46-6.41 (m, 1H), 4.59-4.50 (m, 1H), 4.19-4.12 (m, 2H), 3.61-3.53 (m, 1H), 3.31-3.25 (m, 1H), 2.67 (s, 3H), 2.64 (s, 3H), 1.89-1.81 (m, 1H), 1.72-1.60 (m, 1H); ³¹P NMR (CDCl₃, 162 MHz) δ 18.5 (Sp isomer),18.1 (Rp isomer); ESI-MS m/z: Calculated for [C₃₈H₃₇ClN₇O₄P+H]⁺ 723.18; Found 724.61.

Diastereomeric ratio: 1 (Sp isomer): 20 (Rp isomer) (³¹P NMR).

### Two Step Protocol

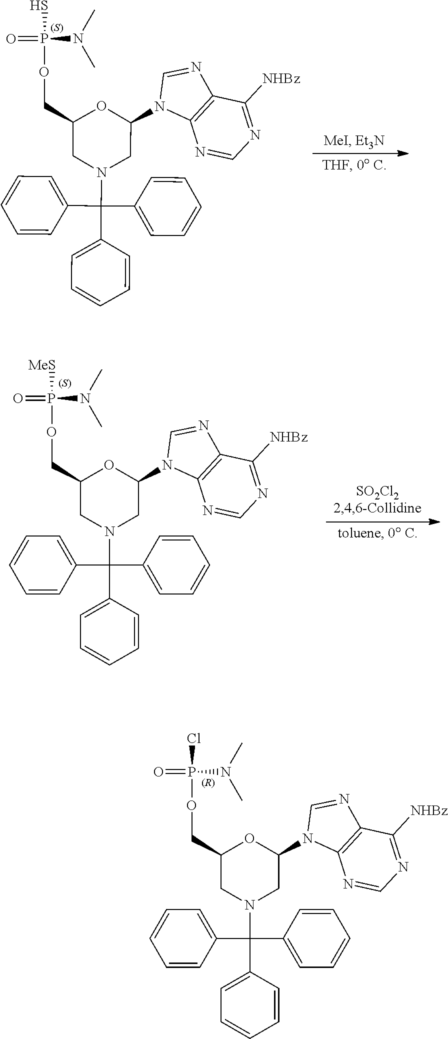

To a solution of O-(((2S,6R)-6-(6-benzamido-9H-purin-9-yl)-4-tritylmorpholin-2-yl)methyl)S-hydrogen (S)-dimethylphosphoramidothioate (100 mg, 0.14 mmol) in THF (2.0 mL) at 0° C. was added triethylamine (60 μL, 0.42 mmol) and iodomethane (20 μL 0.21 mmol). After stirring at 0° C. for 30 min, the reaction was quenched with aq. NH₄Cl (2 mL) and diluted with EtOAc (5 mL). Two layers were separated and the aqueous layer was extracted with EtOAc (2×5 mL). The organic layers were combined, washed with brine (5 mL), dried over Na₂SO₄, concentrated and purified by silica gel column chromatography (THF/n-heptane=0% to 100%) to afford O-(((2S,6R)-6-(6-benzamido-9H-purin-9-yl)-4-tritylmorpholin-2-yl)methyl)S-methyl (S)-dimethylphosphoramidothioate (63 mg, 62% yield).

¹H NMR (CDCl₃, 400 MHz) δ 8.98-8.88 (m, 1H), 8.71 (s, 1H), 7.98-7.88 (m, 3H), 7.55-7.34 (m, 8H), 7.31-7.20 (m, 6H), 7.16-7.07 (m, 3H), 6.34 (dd, J=9.6, 1.9 Hz, 1H), 4.47-4.37 (m, 1H), 3.99-3.92 (m, 2H), 3.70-3.63 (m, 1H), 3.45 (br d, J=11.3 Hz, 1H), 3.18 (br d, J=11.8 Hz, 1H), 2.56 (s, 3H), 2.54 (s, 3H), 2.08 (d, J=14.5 Hz, 3H), 1.81-1.69 (m, 1H), 1.59-1.50 (m, 1H); ³¹P NMR (CDCl₃,162 MHz) δ 37.6 (Sp isomer), 37.4 (Rp isomer); ESI-MS m/z: Calculated for [C₃₉H₄₀N₇O₄PS+H]⁺ 734.27; Found 734.41.

Diastereomeric ratio: 24 (Sp isomer): 1 (Rp isomer) (³¹P NMR).

A solution of O-(((2S,6R)-6-(6-benzamido-9H-purin-9-yl)-4-tritylmorpholin-2-yl)methyl) S-methyl (S)-dimethylphosphoramidothioate (0.068 g, 0.09 mmol) in anhydrous toluene (1.0 mL) was cooled to 0° C. using ice-bath. The solution was treated with sulfuryl chloride (0.28 mL, 0.14 mmol) (0.5M in toluene) followed by 2,4,6-trimethylpyridine (0.13 mL, 0.93 mmol), and stirred at 0° C. for 30 min. The reaction was quenched with aq. NaHCO₃ (2.0 mL) and diluted with EtOAc (5.0 mL). The organic layer was separated, dried over Na₂SO₄, filtered, concentrated and purified by silica gel column chromatography (EtOAc/n-heptane=0% to 100%) to give ((2S,6R)-6-(6-benzamido-9H-purin-9-yl)-4-tritylmorpholin-2-yl)methyl (R)-dimethylphosphoramido-chloridate (48 mg, 72% yield).

¹H NMR (CDCl₃, 400 MHz) δ 8.98-8.88 (m, 1H), 8.74-8.70 (m, 1H), 7.96-7.88 (m, 3H), 7.56-7.47 (m, 1H), 7.47-7.37 (m, 7H), 7.29-7.21 (m, 6H), 7.17-7.10 (m, 3H), 6.38-6.32 (m, 1H), 4.50-4.39 (m, 1H), 4.13-4.02 (m, 2H), 3.52-3.44 (m, 1H), 3.22-3.15 (m, 1H), 2.56 (s, 3H), 2.53 (s, 3H), 1.82-1.73 (m, 1H), 1.63-1.53 (m, 1H); ³¹P NMR (CDCl₃,162 MHz) δ 18.4 (Sp isomer), 18.1 (Rp isomer); ESI-MS m/z: Calculated for [C₃₈H₃₇N₇O₄P+H]⁺ 723.18; Found 723.52.

Diastereomeric ratio: 10 (Rp isomer): 1 (Sp isomer) (³¹P NMR).

### Example 10B—Preparation of ((2S,6R)-6-(6-(((E)-1-methylpyrrolidin-2-ylidene)amino)-9H-purin-9-yl)-4-tritylmorpholin-2-yl)methyl (R)-dimethylphosphoramidochloridate and ((2S,6R)-6-(6-(((E)-1-methylpyrrolidin-2-ylidene)amino)-9H-purin-9-yl)-4-tritylmorpholin-2-yl)methyl (S)-dimethylphosphoramidochloridate

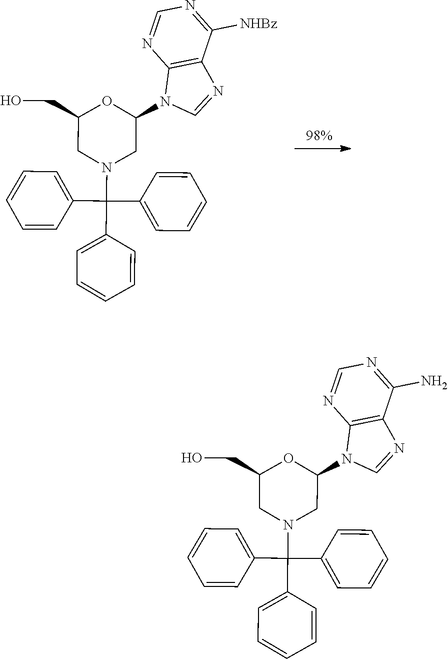

### ((2S,6R)-6-(6-amino-9H-purin-9-yl)-4-tritylmorpholin-2-yl)methanol

To a suspension of N-(9-((2R,6S)-6-(hydroxymethyl)-4-tritylmorpholin-2-yl)-9H-purin-6-yl)benzamide (5.00 g, 8.38 mmol) in MeOH (50.0 mL) was added ammonium hydroxide aqueous solution (50.0 mL, 372 mmol). The mixture was stirred with with a 55° C. sand bath for 1 day, and then cooled down to rt. The solid was collected by filtration, and rinsed with water three times. The solid was dried under vacuum to afford product (4.04 g, 98%)

¹H NMR (400 MHz, CD₃OD) δ 8.20 (s, 1H), 8.07 (s, 1H), 7.51 (m, 6H), 7.29 (m, 6H), 7.17 (m, 3H), 6.30 (d, J=9.6 Hz, 1H), 4.29 (m, 1H), 3.53 (m, 2H), 3.42 (br d, J=11.2 Hz, 1H), 3.24 (br d, J=12.0 Hz, 1H), 1.92 (dd, J=10.8, 10.4 Hz, 1H), 1.65 (dd, J=11.2, 11.2 Hz, 2H); ESI-MS m/z: Calculated for [C₂₉H₂₈N₆O₂+H]⁺ 493.2; Found 493.3.

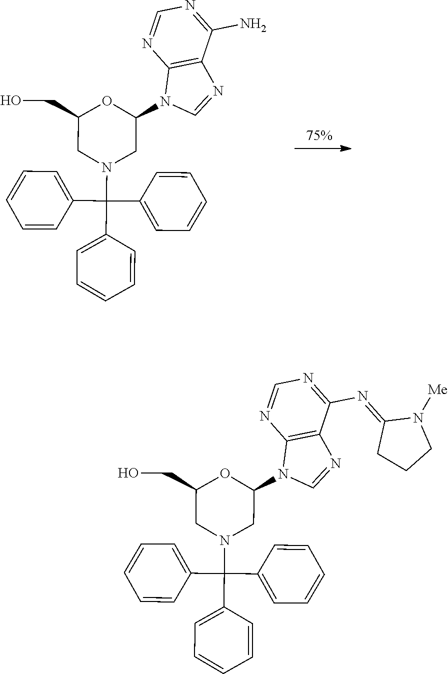 [SMILES: Nc1ncnc2c1ncn2[C@H]1CN(C(c2ccccc2)(c2ccccc2)c2ccccc2)C[C@@H](CO)O1 ; CN1CCC/C1=N\c1ncnc2c1ncn2[C@H]1CN(C(c2ccccc2)(c2ccccc2)c2ccccc2)C[C@@H](CO)O1]

### ((2S,6R)-6-(6-(((E)-1-methylpyrrolidin-2-ylidene)amino)-9H-purin-9-yl)-4-tritylmorpholin-2-yl)methanol

A solution of ((2S,6R)-6-(6-amino-9H-purin-9-yl)-4-tritylmorpholin-2-yl)methanol (4.50 g, 9.14 mmol) in N,N-Dimethylacetamide (22.5 ml, 242.763 mmol) was subjected to azeotropic evaporation on rotavap with anhydrous toluene (50 mL×3) before it was treated with 2,2-diethoxy-1-methylpyrrolidine (2.30 g, 13.3 mmol) at RT overnight. The reaction was quenched with water (0.45 ml, 25 mmol), and it was stirred for 10 min before more water (45 mL) was added. The suspension was sonicated and then stirred for 30 min. The solid was collected by filtration, rinsed subsequently with water twice, and MeCN twice to afford the product (3.92 g, 75%).

¹H NMR (400 MHz, CDCl₃) δ 8.60 (s, 1H), 7.83 (s, 1H), 7.51 (m, 6H), 7.32 (m, 6H), 7.21 (m, 3H), 6.41 (d, J=9.2 Hz, 1H), 4.37 (m, 1H), 3.71-3.56 (m, 2H), 3.54-3.42 (m, 3H), 3.18 (m, 1H), 3.15 (s, 3H), 2.94 (m, 2H), 2.06 (m, 2H), 1.94 (m, 1H), 1.64 (m, 1H); ESI-MS m/z: Calculated for [C₃₄H₃₅N₇O₂+H]⁺ 574.3; Found 574.3.

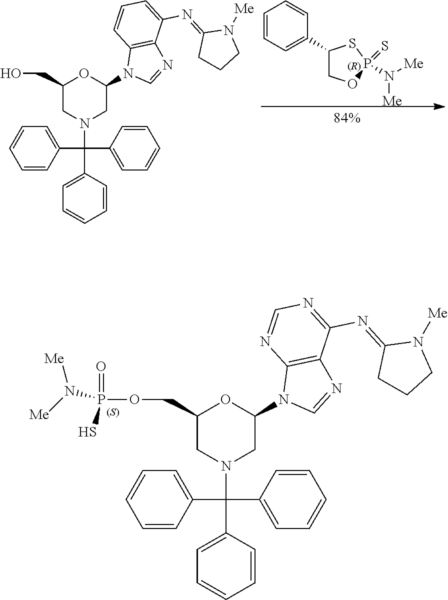

### O-(((2S,6R)-6-(6-(((E)-1-methylpyrrolidin-2-ylidene)amino)-9H-purin-9-yl)-4-tritylmorpholin-2-yl)methyl)S-hydrogen (S)-dimethylphosphoramidothioate

A mixture of ((2S,6R)-6-(6-(((E)-1-methylpyrrolidin-2-ylidene)amino)-9H-purin-9-yl)-4-tritylmorpholin-2-yl)methanol (1.0 g, 1.7 mmol) and (2R,4S)-2-(dimethylamino)-4-phenyl-1,3,2-oxathiaphospholane 2-sulfide (0.678 g, 2.62 mmol) was dried by azeotropic evaporation with anhydrous THF/MeCN, then with anhydrous MeCN before it was dissolved in THF (10.0 mL), into which was added a solution of potassium tert-butoxide in THF (1M, 5.22 mL) dropwise at 0° C. over 5 min. It was kept stirring with ice bath for 1 h before it was diluted with EtOAc and quenched at 0° C. with acetic acid (0.599 mL, 10.5 mmol), followed by addition of sodium dihydrogen phosphate aqueous solution (50.2 mL, 10 wt %). The mixture was extracted with EtOAc (150 mL×2). The combined organic layers were washed subsequently with water twice, half sat. brine, dried over Na₂SO₄, and concentrated. The resulted residue was then redissolved in a mixture of THF (33 mL) and EtOAc (33 mL), into which was added n-heptane (330 mL). The precipitate was collected by filtration, rinsed with a solution of EtOAc (20 mL) in n-heptane (100 mL). The solid was dried under vacuum with a string of N₂ overnight to afford product (1.0 g, 84%).

¹H NMR (400 MHz, THF-d8) δ 8.46 (s, 1H), 7.97 (s, 1H), 7.57 (m, 6H), 7.30 (m, 6H), 7.17 (m, 3H), 6.40 (d, J=10.0 Hz, 1H), 4.54 (m, 1H), 4.05-3.95 (m, 1H), 3.86-3.78 (m, 1H), 3.51 (m, 3H), 3.34 (m, 1H), 3.09 (s, 3H), 2.97 (m, 2H), 2.65 (s, 3H), 2.62 (s, 3H), 2.10-1.90 (m, 3H), 1.65 (dd, J=11.2, 10.8 Hz, 1H); ³¹P NMR (162 MHz, THF-d₈) δ 73.3; ESI-MS m/z: Calculated for [C₃₆H₄₁N₈O₃PS+H]⁺ 697.3; Found 697.5.

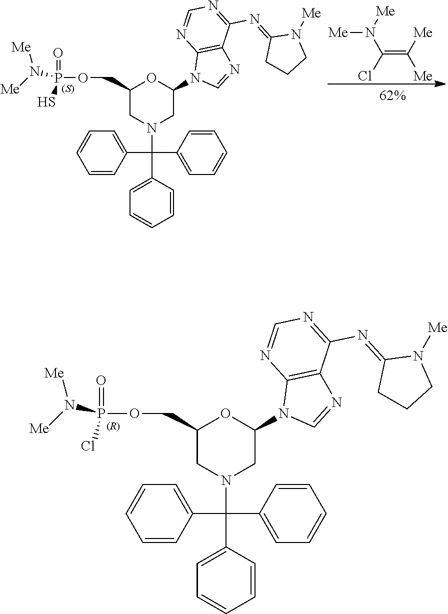

### ((2S,6R)-6-(6-(((E)-1-methylpyrrolidin-2-ylidene)amino)-9H-purin-9-yl)-4-tritylmorpholin-2-yl)methyl (R)-dimethylphosphoramidochloridate

To a solution of O-(((2S,6R)-6-(6-(((E)-1-methylpyrrolidin-2-ylidene)amino)-9H-purin-9-yl)-4-tritylmorpholin-2-yl)methyl)S-hydrogen (S)-dimethylphosphoramidothioate (232 mg, 0.333 mmol) in DCM (3000 μL) was added 1-Chloro-N,N,2-trimethylpropenylamine (176 μL, 1.33 mmol) at 0° C. After addition, the reaction mixture was stirred at 0° C. for 2 h before it was diluted with DCM (50 mL), and then quenched with sat. NaHCO₃ aqueous solution (50 mL). It was extracted with DCM twice, and the combined DCM layers (125 mL) were washed with half sat. brine (100 mL), dried over Na₂SO₄, concentrated. The crude material was purified by silica gel column chromatography, eluting with 0% to 100% THF in EtOAc to afford product (144 mg, 62%). The diastereomeric ratio was determined by ³¹P NMR to be 99:1.

¹H NMR (400 MHz, CDCl₃) δ 8.51 (s, 1H), 7.78 (s, 1H), 7.41 (m, 6H), 7.23 (m, 6H), 7.12 (m, 3H), 6.31 (d, J=9.6 Hz, 1H), 4.43 (m, 1H), 4.05 (m, 2H), 3.42 (m, 3H), 3.16 (d, J=11.6 Hz, 1H), 3.06 (s, 3H), 2.86 (m, 2H), 2.56 (s, 3H), 2.53 (s, 3H), 2.04-1.84 (m, 3H), 1.54 (dd, J=11.2, 11.2 Hz, 1H); ³¹P NMR (162 MHz, CDCl₃) δ 18.1; ESI-MS m/z: Calculated for [C₃₆H₄₀ClN₈O₃P+H]⁺ 699.3; Found 699.5.

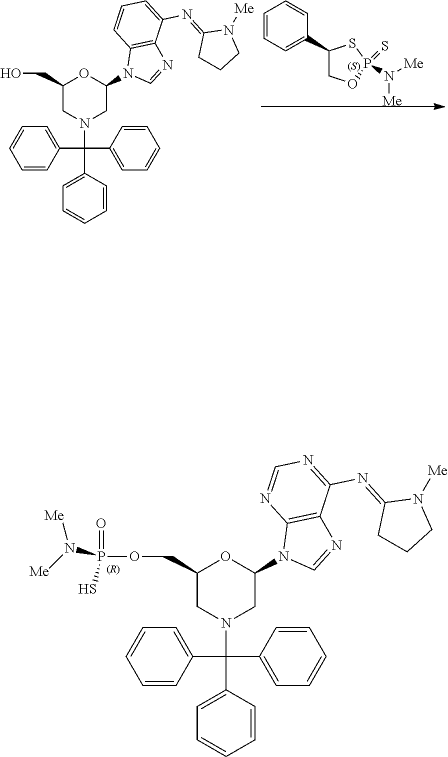

### O-(((2S,6R)-6-(6-(((E)-1-methylpyrrolidin-2-ylidene)amino)-9H-purin-9-yl)-4-tritylmorpholin-2-yl)methyl)S-hydrogen (R)-dimethylphosphoramidothioate

A mixture of ((2S,6R)-6-(6-(((E)-1-methylpyrrolidin-2-ylidene)amino)-9H-purin-9-yl)-4-tritylmorpholin-2-yl)methanol (53.2 mg, 0.093 mmol) and (2S,4R)-2-(dimethylamino)-4-phenyl-1,3,2-oxathiaphospholane 2-sulfide (74.5 mg, 0.287 mmol) was dried by co-evaporation with THF/MeCN, then with MeCN once before it was dissolved in THF (1000 μl, 12.204 mmol). To the solution was added a solution of potassium tert-butoxide in THF (278 μl, 0.278 mmol) dropwise at 0° C. After addition, the reaction mixture was stirred at 0° C. for 1.5 h before additional potassium tert-butoxide solution (93 μL, 0.093 mmol) was added. It was kept stirring at 0° C. for another 1 h before it was quenched with acetic acid (42.5 μl, 0.742 mmol). To the mixture was added aqueous solution of sodium phosphate, dibasic (6.58 mL, 10%), and it was then extracted with EtOAc twice. The organic layers were combined and washed subsequently with water, half sat. brine, dried over Na₂SO₄, and concentrated. The residue was redissolved in a mixture of THF (2 mL) and EtOAc (2 mL), into which was then added n-heptane (20 mL). The solid was collected by filtration, rinsed with a mixture of n-heptane and EtOAc (12 mL, 5:1 v/v), and dried under vacuum at rt with a string of N₂ overnight to afford the title product (56.4 mg, 87%).

¹H NMR (400 MHz, CDCl₃) δ 8.84 (s, 1H), 7.86 (s, 1H), 7.51 (m, 6H), 7.34 (m, 6H), 7.22 (m, 3H), 6.31 (d, J=9.2 Hz, 1H), 4.67 (m, 1H), 4.10-3.90 (m, 2H), 3.61 (m, 2H), 3.47 (m, 1H), 3.25 (m, 1H), 3.14 (s, 3H), 3.05-2.85 (m, 2H), 2.66 (s, 3H), 2.63 (s, 3H), 2.26-2.02 (m, 2H), 1.68 (dd, J=10.8, 10.0 Hz, 1H), 1.55 (dd, J=11.2, 10.8 Hz, 1H); ³¹P NMR (162 MHz, CDCl₃) δ 66.7; ESI-MS m/z: Calculated for [C₃₆H₄₁N₈O₃PS+H]⁺ 697.3; Found 697.1.

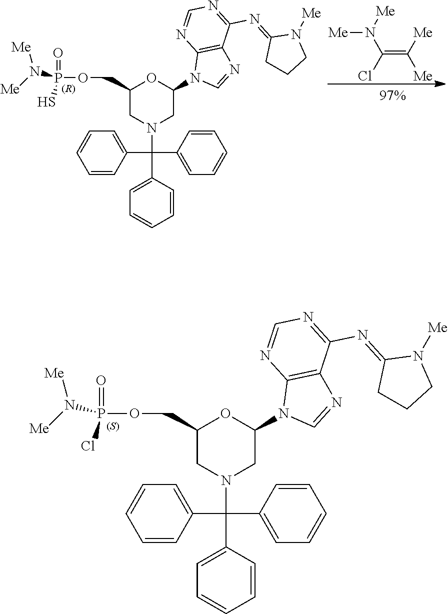

### ((2S,6R)-6-(6-(((E)-1-methylpyrrolidin-2-ylidene)amino)-9H-purin-9-yl)-4-tritylmorpholin-2-yl)methyl (S)-dimethylphosphoramidochloridate

To a solution of O-(((2S,6R)-6-(6-(((E)-1-methylpyrrolidin-2-ylidene)amino)-9H-purin-9-yl)-4-tritylmorpholin-2-yl)methyl)S-hydrogen (R)-dimethylphosphoramidothioate (56.4 mg, 0.081 mmol) in DCM (729 μL, 11.326 mmol) was added 1-Chloro-N,N,2-trimethylpropenylamine (53.5 μL, 0.405 mmol) at 0° C. The reaction mixture was stirred at 0° C. for 2 h before it was diluted with DCM, and then quenched with sat. NaHCO₃ aqueous solution. After phase separation, it was back extracted with DCM once. The combined DCM layers were washed with half sat. brine, dried over Na₂SO₄, concentrated. The crude material was purified by silica gel column chromatography, eluting with 0% to 100% THF in EtOAc to afford the title product (55 mg, 97%). The diastereomeric ratio was determined to be 99.4:0.6 by ³¹P NMR.

¹H NMR (400 MHz, CDCl₃) δ 8.53 (s, 1H), 7.80 (s, 1H), 7.41 (m, 6H), 7.23 (m, 6H), 7.12 (m, 3H), 6.32 (d, J=9.2 Hz, 1H), 4.44 (m, 1H), 4.04 (m, 2H), 3.41 (m, 3H), 3.16 (d, J=12.0 Hz, 1H), 3.05 (s, 3H), 2.87 (m, 2H), 2.54 (s, 3H), 2.51 (s, 3H), 2.04-1.84 (m, 3H), 1.52 (dd, J=11.2, 10.8 Hz, 1H); ³¹P NMR (162 MHz, CDCl₃) δ 18.5; ESI-MS m/z: Calculated for [C₃₆H₄₀ClN₈O₃P+H]⁺ 699.3; Found 699.3.

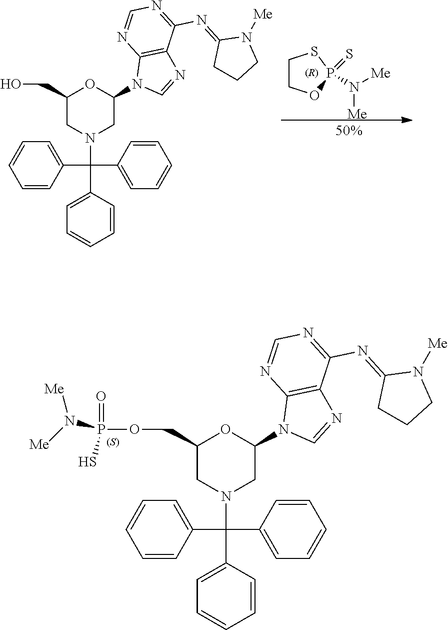

### O-(((2S,6R)-6-(6-(((E)-1-methylpyrrolidin-2-ylidene)amino)-9H-purin-9-yl)-4-tritylmorpholin-2-yl)methyl)S-hydrogen (S)-dimethylphosphoramidothioate

The mixture of ((2S,6R)-6-(6-(((E)-1-methylpyrrolidin-2-ylidene)amino)-9H-purin-9-yl)-4-tritylmorpholin-2-yl)methanol (500 mg, 0.872 mmol) and (R)-2-(dimethylamino)-1,3,2-oxathiaphospholane 2-sulfide (188 mg, 1.03 mmol) was dried by coevaporation with anhydrous MeCN before it was suspended in THF (5000 μL), into which was then added Sodium-tert-amoxide (784 μL, 2.62 mmol) dropwise at 0° C. over 10 min. The reaction mixture was stirred at 0° C. for 1 h before additional (R)-2-(dimethylamino)-1,3,2-oxathiaphospholane 2-sulfide (43 mg, 0.23 mmol) was added, and it was kept stirring at 0° C. for 1 h. It was diluted with EtOAc at 0° C., and quenched with NaH₂PO₄ solution (aq., 10%). After phase separation, it was back extracted with EtOAc once. The combined organic layers (100 mL) were washed subsequently with water (20 mL) and half sat. brine (20 mL), dried over Na₂SO₄, and concentrated. The crude material was redissolved in a mixture of THF (10 mL) and EtOAc (10 mL), into which was then added n-heptane (100 mL). The resulted solid was collected by filtration, rinsed with a solution of EtOAc (10 mL) in n-heptane (50 mL), dried under vacuum at rt with a string of N₂ overnight to afford the title product (306 mg, 50%).

¹H NMR (400 MHz, CDCl₃) δ 8.61 (s, 1H), 7.93 (s, 1H), 7.52 (m, 6H), 7.34 (m, 6H), 7.23 (m, 3H), 6.17 (d, J=9.2 Hz, 1H), 4.57 (m, 1H), 4.45-4.35 (m, 1H), 3.90-3.80 (m, 1H), 3.75-3.58 (m, 2H), 3.51 (d, J=11.2 Hz, 1H), 3.18 (m, 1H), 3.16 (s, 3H), 3.14-3.02 (m, 1H), 2.88-2.73 (m, 1H), 2.70 (s, 3H), 2.67 (s, 3H), 2.19 (m, 2H), 1.55 (m, 2H); ³¹P NMR (162 MHz, CDCl₃) δ 67.7; ESI-MS m/z: Calculated for [C₃₆H₄₁N₈O₃PS+H]⁺ 697.3; Found 697.4.

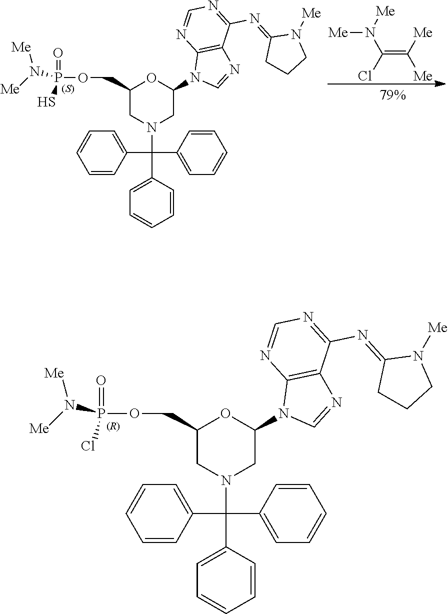

### ((2S,6R)-6-(6-(((E)-1-methylpyrrolidin-2-ylidene)amino)-9H-purin-9-yl)-4-tritylmorpholin-2-yl)methyl (R)-dimethylphosphoramidochloridate

O-(((2S,6R)-6-(6-(((E)-1-methylpyrrolidin-2-ylidene)amino)-9H-purin-9-yl)-4-tritylmorpholin-2-yl)methyl)S-hydrogen (S)-dimethylphosphoramidothioate (128 mg, 0.184 mmol) was dried by coevaporation with anhydrous MeCN once before it was dissolved in DCM (1.65 mL). To the solution was then added 1-Chloro-N,N,2-trimethylpropenylamine (97 μL, 0.74 mmol) at 0° C. The reaction mixture was stirred at 0° C. for 2 h before it was diluted with EtOAc, and then quenched with sat. NaHCO₃(aq.). After phase separation, it was back extracted with EtOAc once. The combined organic layers (60 mL) were washed with half sat. brine, dried over Na₂SO₄, and concentrated. The crude material was purified by silica gel column chromatography, eluting with 0% to 100% THF in EtOAc to afford the title product (101 mg, 79%). The diastereomeric ratio was determined to be 99.4:0.6 by ³¹P NMR.

¹H NMR (400 MHz, CDCl₃) δ 8.50 (s, 1H), 7.76 (s, 1H), 7.41 (m, 6H), 7.23 (m, 6H), 7.12 (m, 3H), 6.30 (dd, J=10.0, 2.4 Hz, 1H), 4.42 (m, 1H), 4.05 (m, 2H), 3.41 (m, 3H), 3.16 (d, J=112.0 Hz, 1H), 3.06 (s, 3H), 2.85 (m, 2H), 2.56 (s, 3H), 2.53 (s, 3H), 2.04-1.84 (m, 3H), 1.54 (dd, J=11.2, 11.2 Hz, 1H); ³¹P NMR (162 MHz, CDCl₃) δ 18.1; ESI-MS m/z: Calculated for [C₃₆H₄₀ClN₈O₃P+H]⁺ 699.3; Found 699.1.

### Example 11A—Preparation of ((2S,6R)-6-(2-isobutyramido-6-oxo-1,6-dihydro-9H-purin-9-yl)-4-tritylmorpholin-2-yl)methyl (R)-dimethylphosphoramidochloridate

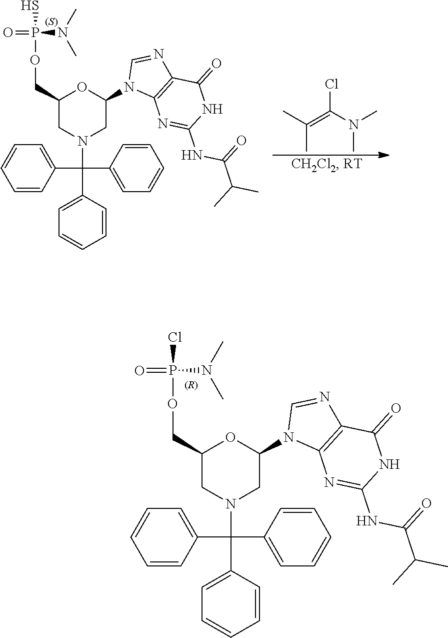

To a solution of O-(((2S,6R)-6-(2-isobutyramido-6-oxo-1,6-dihydro-9H-purin-9-yl)-4-tritylmorpholin-2-yl)methyl)S-hydrogen (S)-dimethylphosphoramidothioate (0.500 g, 0.71 mmol) in CH₂Cl₂ (7.0 mL) was added 1-chloro-N,N,2-trimethylpropenylamine (0.19 mL, 1.43 mmol) at 0° C. After stirring at room temperature for 1 h, the reaction was quenched with aq. sat. NaHCO₃ (10 mL) and diluted with CH₂Cl₂ (15 mL). Two layers were separated and the aqueous layer was extracted with CH₂Cl₂ (3×10 mL). The combined organic layers were dried over MgSO₄, filtered, concentrated and purified using silica gel column chromatography to (EtOAc/n-heptane=0% to 100% then MeOH/CH₂Cl₂=0% to 10%) give ((2S,6R)-6-(2-isobutyramido-6-oxo-1,6-dihydro-9H-purin-9-yl)-4-tritylmorpholin-2-yl)methyl (R)-dimethylphosphoramidochloridate (230 mg, 46% yield).

¹H NMR (CDCl₃, 400 MHz) δ 12.0 (s, 1H), 9.59-9.38 (m, 1H), 7.60 (m, 1H), 7.57-7.37 (m, 3H), 7.37-7.15 (m, 10H), 7.16-7.09 (m, 2H), 6.09-5.95 (m, 1H), 4.42-4.34 (m, 1H), 4.09-4.02 (m, 1H), 3.40-3.34 (m, 1H), 3.19-3.12 (m, 1H), 2.68-2.60 (m, 1H), 2.58 (s, 3H), 2.54 (s, 3H), 1.69-1.61 (m, 1H), 1.53-1.43 (m, 1H), 1.17 (d, J=6.5 Hz, 3H), 1.05 (d, J=6.7 Hz, 3H); ³¹P NMR (CDCl₃, 162 MHz) δ 18.6 (Sp isomer), 18.3 (Rp isomer); ESI-MS m/z: Calculated for [C₃₅H₃₉ClN₇O₅P+H]⁺ 704.25; Found 723.52.

Diastereomeric ratio: 14 (Rp isomer):1 (Sp isomer) (³¹P NMR).

### Example 11B—Preparation of Doubly Protected PMO-G Activated Monomers

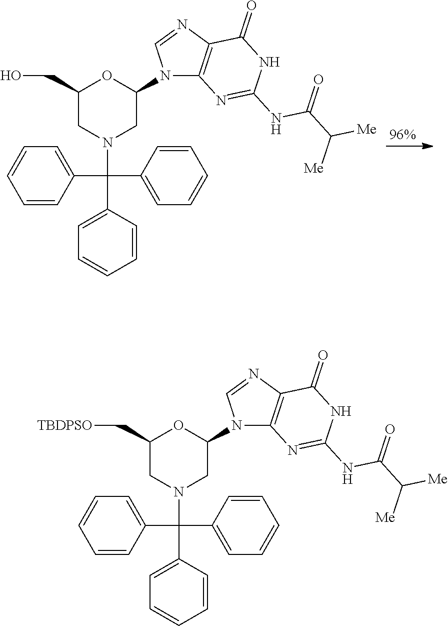

### N-(9-((2R,6S)-6-(((tert-butyldiphenylsilyl)oxy)methyl)-4-tritylmorpholin-2-yl)-6-oxo-6,9-dihydro-1H-purin-2-yl)isobutyramide

To a suspension of N-(9-((2R,6S)-6-(hydroxymethyl)-4-tritylmorpholin-2-yl)-6-oxo-6,9-dihydro-1H-purin-2-yl)isobutyramide (5.0 g, 8.6 mmol) and imidazole (3.5 g, 51 mmol) in DMF (50 mL) was added TBDPS-Cl (4.44 mL, 17.3 mmol) at rt. The reaction mixture was stirred at rt for 2.5 h before it was diluted with MTBE, and then quenched with water (100 mL) at rt. The mixture was extracted with EtOAc twice. The combined organic layers (350 mL) was washed subsequently with saturated NaHCO₃ aqueous solution (80 mL), saturated brine (50 mL), dried over Na₂SO₄, and concentrated. The crude material was purified by silica gel column chromatography, eluting with 55% to 85% EtOAc in n-heptane to afford the title product (6.77 g, 96%).

¹H NMR (400 MHz, CDCl₃) δ 11.87 (s, 1H), 7.87 (brs, 1H), 7.45-7.60 (m, 10H), 7.45-7.38 (m, 3H), 7.38-7.29 (m, 10H), 7.26-7.19 (m, 2H), 6.99 (d, J=9.2 Hz, 1H), 4.30 (m, 1H), 3.77 (dd, J=10.8, 4.8 Hz, 1H), 3.61 (dd, J=10.4, 5.6 Hz, 1H), 3.43 (br d, J=11.6 Hz, 1H), 3.33 (br d, J=11.6 Hz, 1H), 2.64 (m, 1H), 1.75 (dd, J=10.8, 10.4 Hz, 1H), 1.58 (dd, J=11.2, 10.8 Hz, 1H), 1.35 (d, J=7.2 Hz, 1H), 1.34 (d, J=6.8 Hz, 1H), 0.97 (s, 9H); ESI-MS m/z: Calculated for [C₄₉H₅₂N₆O₄Si+H]⁺ 817.4; Found 817.2.

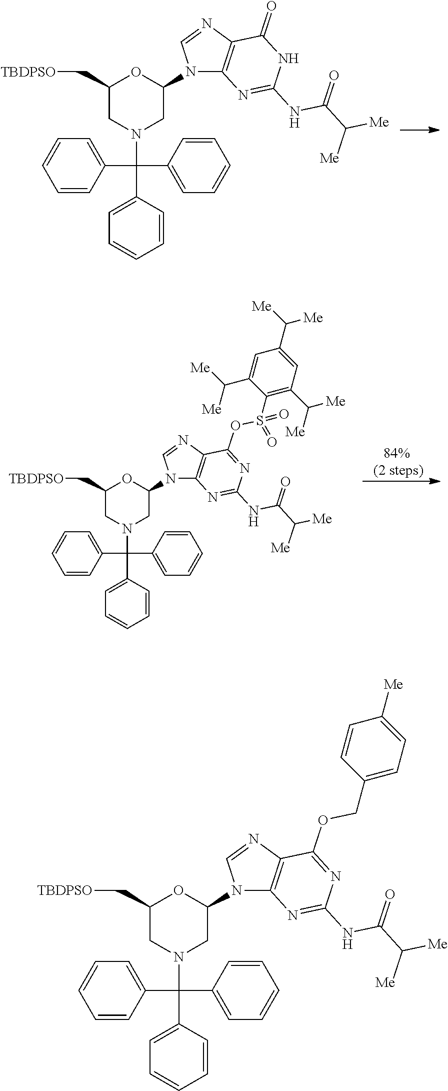

### N-(9-((2R,6S)-6-(((tert-butyldiphenylsilyl)oxy)methyl)-4-tritylmorpholin-2-yl)-6-((4-methylbenzyl)oxy)-9H-purin-2-yl)isobutyramide

To a solution of N-(9-((2R,6S)-6-(((tert-butyldiphenylsilyl)oxy)methyl)-4-tritylmorpholin-2-yl)-6-oxo-6,9-dihydro-1H-purin-2-yl)isobutyramide (6.77 g, 8.29 mmol) in DCM (95 mL) was added DMAP (0.202 g, 1.66 mmol), triethylamine (4.16 mL, 29.8 mmol), and 2,4,6-triisopropylbenzenesulfonyl chloride (4.52 g, 14.9 mmol) at rt. The reaction mixture was stirred at rt for 1 day before it was cooled in ice bath, and then quenched with sodium dihydrogen phosphate aqueous solution (252 mL, 10%). It was extracted with DCM twice. The combined organic layers were washed with half saturated brine (250 mL), dried over Na₂SO₄, and concentrated to give crude intermediate.

One third of the crude intermediate was then dried by coevaporation with toluene three times before it was dissolved in DCM (30 mL). Into the solution was added (4-methylphenyl)methanol (0.677 g, 5.54 mmol), DBU (1.25 mL, 8.31 mmol) and N-methylpyrrolidine (0.432 mL, 4.15 mmol) at rt. The reaction mixture was stirred at rt for 1 day before it was diluted with EtOAc, and then quenched with sodium dihydrogen phosphate aqueous solution (100 mL, 10%) at rt. It was extracted with EtOAc twice, and the combined organic layers were washed subsequently with sat. NaHCO₃ aqueous solution, sat. brine, dried over Na₂SO₄, and concentrated. The crude material was purified by silica gel column chromatography, eluting with 25% to 40% EtOAc in n-heptane to afford title product (2.44 g, 84% over 2 steps).

¹H NMR (400 MHz, CDCl₃) δ 7.79 (brs, 1H), 7.66 (s, 1H), 7.60-7.45 (m, 10H), 7.45-7.37 (m, 5H), 7.36-7.27 (m, 10H), 7.25-7.18 (m, 2H), 7.16 (d, J=8.0 Hz, 2H), 6.21 (dd, J=10.0, 2.4 Hz, 1H), 5.62 (d, J=12.0 Hz, 1H), 5.55 (d, J=12.0 Hz, 1H), 4.33 (m, 1H), 3.77 (dd, J=10.8, 4.8 Hz, 1H), 3.60 (dd, J=10.4, 5.6 Hz, 1H), 3.42 (br d, J=11.2 Hz, 1H), 3.34 (br d, J=11.6 Hz, 1H), 3.31 (m, 1H), 2.35 (s, 3H), 1.71 (dd, J=10.8, 10.4 Hz, 1H), 1.59 (dd, J=11.6, 11.6 Hz, 1H), 1.36 (d, J=6.8 Hz, 1H), 1.34 (d, J=6.8 Hz, 1H), 0.97 (s, 9H); ESI-MS m/z: Calculated for [C₅₇H₆₀N₆O₄Si+H]⁺ 921.4; Found 921.2.

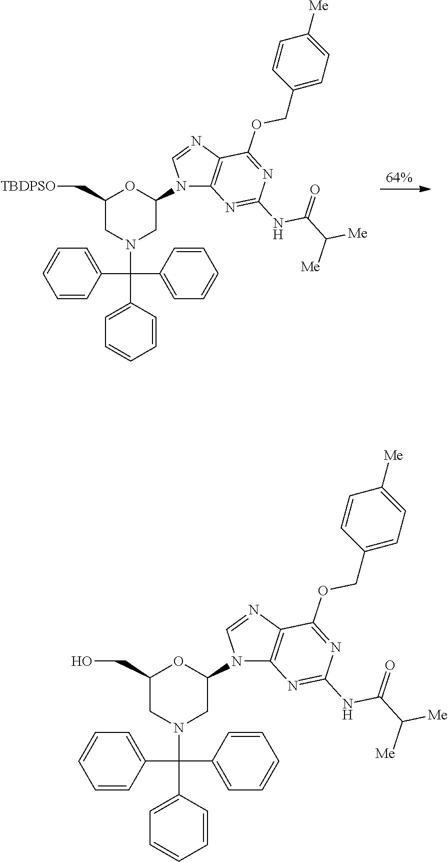

### N-(9-((2R,6S)-6-(hydroxymethyl)-4-tritylmorpholin-2-yl)-6-((4-methylbenzyl)oxy)-9H-purin-2-yl)isobutyramide

To a solution of N-(9-((2R,6S)-6-(((tert-butyldiphenylsilyl)oxy)methyl)-4-tritylmorpholin-2-yl)-6-((4-methylbenzyl)oxy)-9H-purin-2-yl)isobutyramide (2.44 g, 2.65 mmol) in DCM (23.9 mL) was added pyridine (12.0 mL, 148 mmol), triethylamine (24.0 mL, 172 mmol), and triethylamine trihydrofluoride (3.02 mL, 18.5 mmol) dropwise at RT. The reaction mixture was stirred at rt overnight before additional triethylamine (4.8 mL, 34.4 mmol), and triethylamine trihydrofluoride (0.61 mL, 3.7 mmol) was added. It was stirred at rt for another day before trimethylmethoxysilane (14.6 mL, 106 mmol) was added at rt. It was stirred at rt for one more day before it was concentrated to give crude material. It was then redissolved in a mixture of THF (20 mL) and EtOAc (20 mL), and some insoluble particle was removed by filtration, washed with EtOAc (2 mL). Into the filtrate was then added n-heptane (500 mL), and the resulted solid was collected by filtration, rinsed with n-heptane twice, and then dried under vacuum over a stream of N₂ to give the title product (1.15 g, 64%).

¹H NMR (400 MHz, CD₃OD) δ 8.08 (s, 1H), 7.66 (s, 1H), 7.58-7.45 (m, 6H), 7.40-7.35 (m, 2H), 7.34-7.26 (m, 6H), 7.20-7.12 (m, 5H), 6.56 (dd, J=9.6, 2.0 Hz, 1H), 5.55 (m, 2H), 4.58 (brs, 1H), 4.30 (m, 1H), 3.53 (m, 2H), 3.45 (m, 1H), 3.22 (m, 1H), 2.90 (m, 1H), 2.30 (s, 3H), 1.82 (dd, J=10.8, 10.4 Hz, 1H), 1.62 (dd, J=11.6, 10.4 Hz, 1H), 1.28 (d, J=6.8 Hz, 6H); ESI-MS m/z: Calculated for [C₄₁H₄₂N₆O₄+H]⁺ 683.3; Found 683.1.

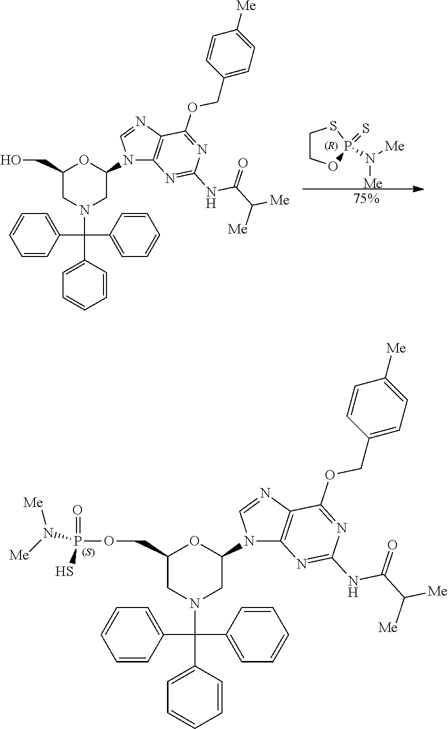

### O-(((2S,6R)-6-(2-isobutyramido-6-((4-methylbenzyl)oxy)-9H-purin-9-yl)-4-tritylmorpholin-2-yl)methyl)S-hydrogen (S)-dimethylphosphoramidothioate

A mixture of N-(9-((2R,6S)-6-(hydroxymethyl)-4-tritylmorpholin-2-yl)-6-((4-methylbenzyl)oxy)-9H-purin-2-yl)isobutyramide (120 mg, 0.176 mmol) and (R)-2-(dimethylamino)-1,3,2-oxathiaphospholane 2-sulfide (48.7 mg, 0.266 mmol) was dried by coevaporation with anhydrous MeCN once before it was suspended in THF (1.20 mL, 14.6 mmol), into which was added sodium-tert-amoxide solution in toluene (158 μL, 0.527 mmol) at 0° C. The reaction mixture was stirred at 0° C. for 5 h before it was quenched with acetic acid (60 μL, 1.0 mmol) at 0° C., diluted with EtOAc. Into the mixture was added sodium dihydrogen phosphate aq. solution (10.1 mL, 10%), and it was then extracted with EtOAc (50 mL×2). Combined organic layers were washed with half sat. brine (20 mL), dried over Na₂SO₄, and concentrated. The residue was redissolved in EtOAc (5 mL), into which was added n-heptane (20 mL). The resulted solid was collected by filtration, rinsed with a mixture of n-heptane-EtOAc (25 mL, 4:1 v/v), and dried under vacuum with a stream of N₂ at rt over weekend to give the title product (106 mg, 75%).

¹H NMR (400 MHz, CDCl₃) δ 8.30 (brs, 1H), 7.90 (brs, 1H), 7.58-7.39 (m, 6H), 7.39-7.34 (m, 2H), 7.34-7.22 (m, 6H), 7.22-7.06 (m, 5H), 6.16 (d, J=9.6 Hz, 1H), 5.57 (m, 2H), 4.37 (brs, 1H), 4.16 (m, 1H), 4.00 (m, 1H), 3.46 (d, J=10.4 Hz, 1H), 3.17 (d, J=11.2 Hz, 2H), 2.66 (s, 3H), 2.63 (s, 3H), 2.50 (m, 1H), 2.35 (s, 3H), 1.76 (dd, J=10.8, 10.4 Hz, 1H), 1.57 (dd, J=11.2, 10.8 Hz, 1H), 1.37 (d, J=6.8 Hz, 3H), 1.34 (d, J=6.8 Hz, 3H); ³¹P NMR (162 MHz, CDCl₃) δ 65.3; ESI-MS m/z: Calculated for [C₄₃H₄₇N₇O₅PS+H]⁺ 806.3; Found 806.1.

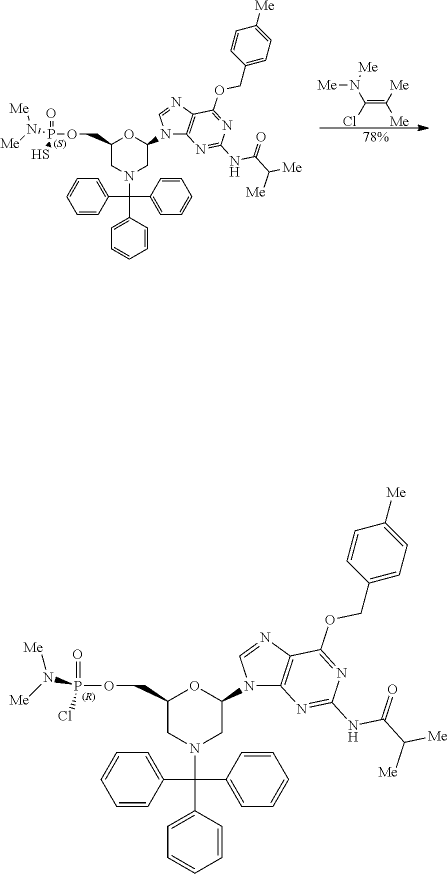

### ((2S,6R)-6-(2-isobutyramido-6-((4-methylbenzyl)oxy)-9H-purin-9-yl)-4-tritylmorpholin-2-yl)methyl (R)-dimethylphosphoramidochloridate

O-(((2S,6R)-6-(2-isobutyramido-6-((4-methylbenzyl)oxy)-9H-purin-9-yl)-4-tritylmorpholin-2-yl)methyl)S-hydrogen (S)-dimethylphosphoramidothioate (90 mg, 0.11 mmol) was dried by coevaporation with anhydrous MeCN once before it was dissolved in DCM (1.0 mL), into which was then added 1-Chloro-N,N,2-trimethylpropenylamine (59 μL, 0.45 mmol) at 0° C. The reaction mixture was kept stirring at 0° C. for 3 h before it was diluted with EtOAc and then quenched with saturated sodium bicarbonate aqueous solution (7.5 mL) at 0° C. The mixture was extracted with EtOAc twice, and the combined organic layers (80 mL) were washed with half saturated brine, dried over Na₂SO₄, concentrated. The crude material was purified by column chromatography, eluting with 70% to 100% EtOAc in n-heptane to afford the title product (70 mg, 78%). The diastereomeric ratio was determined to 95.9:4.1 by ³¹P NMR.

¹H NMR (400 MHz, CDCl₃) δ 7.79 (s, 2H), 7.58-7.42 (m, 6H), 7.39 (d, J=8.0 Hz, 2H), 7.36-7.29 (m, 6H), 7.25-7.19 (m, 3H), 7.16 (d, J=8.0 Hz, 2H), 6.25 (dd, J=10.0, 2.4 Hz, 1H), 5.58 (m, 2H), 4.50 (m, 1H), 4.13 (m, 2H), 3.49 (br d, J=11.2 Hz, 1H), 3.35-3.20 (m, 2H), 2.67 (s, 3H), 2.63 (s, 3H), 2.65-2.60 (m, 1H), 2.35 (s, 3H), 1.78 (dd, J=10.4, 10.0 Hz, 1H), 1.60 (dd, J=11.6, 10.8 Hz, 1H), 1.37 (d, J=6.8 Hz, 3H), 1.35 (d, J=6.8 Hz, 3H); ³¹P NMR (162 MHz, CDCl₃) δ 18.1; ESI-MS m/z: Calculated for [C₄₃H₄₆ClN₇O₅P+H]⁺ 808.3; Found 808.0.

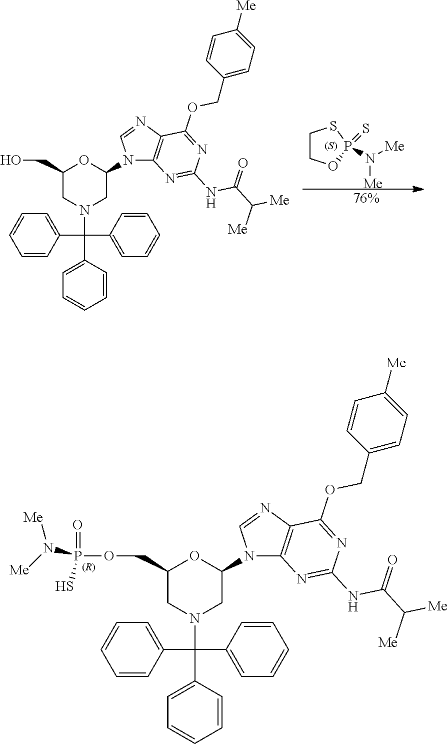

### O-(((2S,6R)-6-(2-isobutyramido-6-((4-methylbenzyl)oxy)-9H-purin-9-yl)-4-tritylmorpholin-2-yl)methyl)S-hydrogen (R)-dimethylphosphoramidothioate

N-(9-((2R,6S)-6-(hydroxymethyl)-4-tritylmorpholin-2-yl)-6-((4-methylbenzyl)oxy)-9H-purin-2-yl)isobutyramide (120 mg, 0.176 mmol) and (S)-2-(dimethylamino)-1,3,2-oxathiaphospholane 2-sulfide (48.4 mg, 0.264 mmol) was dried by coevaporation with anhydrous MeCN once before it was dissolved in THF (1.20 mL), into which was added Sodium-tert-amoxide (158 μL, 0.527 mmol) solution in toluene. The reaction mixture was stirred at 0° C. for 7 h before additional (S)-2-(dimethylamino)-1,3,2-oxathiaphospholane 2-sulfide (12 mg, 0.066 mmol) was added. The mixture was stirred at 0° C. for 1 h before it was quenched with acetic acid (60.4 μL, 1.05 mmol) at 0° C. The mixture was diluted with EtOAc, and treated with sodium dihydrogen phosphate aqueous solution (10.0 mL, 10%) before it was extracted with EtOAc (50 mL×2). The combined organic layers were washed with half sat. brine (20 mL), dried over Na₂SO₄, and concentrated. The crude material was dissolved in EtOAc (5 mL), into which was then added n-heptane (20 mL). The solid was collected by filtration, rinsed with a mixture of n-heptane-EtOAc (25 mL, 4:1 v/v), and dried under vacuum with a stream of N₂ at rt over weekend to give the title product (107 mg, 76%).

¹H NMR (400 MHz, CDCl₃) δ 8.67 (brs, 1H), 7.84 (s, 1H), 7.56-7.39 (m, 6H), 7.38-7.33 (m, 2H), 7.33-7.24 (m, 6H), 7.24-7.16 (m, 3H), 7.13 (d, J=8.0 Hz, 2H), 6.20 (d, J=8.0 Hz, 1H), 5.58 (m, 2H), 4.32 (m, 1H), 4.14 (m, 1H), 4.05 (m, 1H), 3.41 (d, J=11.2 Hz, 1H), 3.21 (m, 1H), 3.12 (d, J=11.6 Hz, 1H), 2.64 (s, 3H), 2.613 (s, 3H), 2.56 (m, 1H), 2.34 (s, 3H), 1.77 (dd, J=10.8, 10.8 Hz, 1H), 1.60 (dd, J=11.2, 11.2 Hz, 1H), 1.35 (d, J=6.8 Hz, 3H), 1.32 (d, J=6.8 Hz, 3H); ³¹P NMR (162 MHz, CDCl₃) δ 65.2; ESI-MS m/z: Calculated for [C₄₃H₄₇N₇O₅PS+H]⁺ 806.3; Found 806.0.

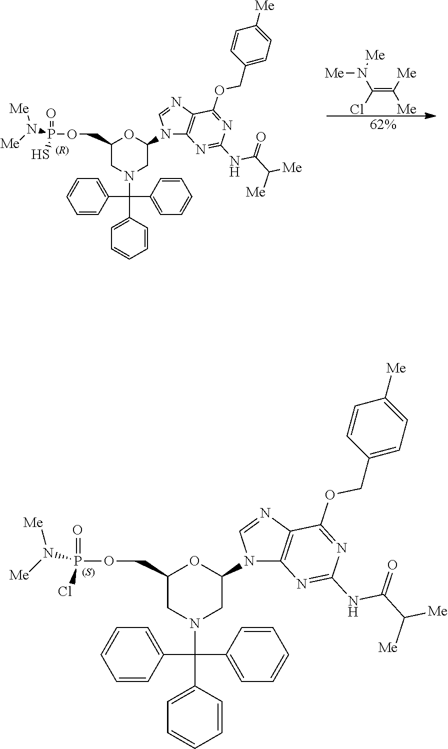

### ((2S,6R)-6-(2-isobutyramido-6-((4-methylbenzyl)oxy)-9H-purin-9-yl)-4-tritylmorpholin-2-yl)methyl (S)-dimethylphosphoramidochloridate

O-(((2S,6R)-6-(2-isobutyramido-6-((4-methylbenzyl)oxy)-9H-purin-9-yl)-4-tritylmorpholin-2-yl)methyl)S-hydrogen (R)-dimethylphosphoramidothioate (90 mg, 0.11 mmol) was dried by coevaporation with anhydrous MeCN once before it was dissolved in DCM (1.0 mL), into which was added 1-Chloro-N,N,2-trimethylpropenylamine (59 μL, 0.45 mmol) at 0° C. The reaction mixture was stirred at 0° C. for 2 h before it was quenched with saturated sodium bicarbonate aqueous solution (0.75 mL) at 0° C., and diluted with EtOAc (10 mL). The mixture was extracted with EtOAc twice, and the combined organic layers (80 mL) were washed with half sat. brine, dried over Na₂SO₄, concentrated. The crude material was purified by column chromatography, eluting with 70% to 100% EtOAc in n-heptane to give the title product (56 mg, 62%). The diastereomeric ratio was determined to be 95.5:4.5 by ³¹P NMR.

¹H NMR (400 MHz, CDCl₃) δ 7.80 (brs, 1H), 7.78 (s, 1H), 7.58-7.42 (m, 6H), 7.39 (d, J=8.0 Hz, 2H), 7.36-7.29 (m, 6H), 7.25-7.19 (m, 3H), 7.16 (d, J=8.0 Hz, 2H), 6.26 (dd, J=9.6, 2.4 Hz, 1H), 5.58 (m, 2H), 4.50 (m, 1H), 4.13 (m, 2H), 3.48 (br d, J=11.2 Hz, 1H), 3.36-3.18 (m, 2H), 2.68-2.63 (m, 1H), 2.65 (s, 3H), 2.61 (s, 3H), 2.35 (s, 3H), 1.77 (dd, J=10.4, 10.4 Hz, 1H), 1.60 (dd, J=11.6, 10.8 Hz, 1H), 1.37 (d, J=7.2 Hz, 3H), 1.35 (d, J=6.8 Hz, 3H); ³¹P NMR (162 MHz, CDCl₃) δ 18.5; ESI-MS m/z: Calculated for [C₄₃H₄₆ClN₇O₅P+H]⁺ 808.3; Found 808.1.

### Example 12—Solid Phase Synthesis of Stereopure PMOs Using Peptide Synthesizer

### Example 12A—Deprotection of Fmoc on Sar-Wang Resin

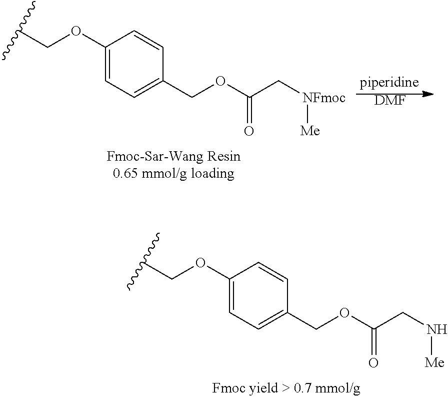

Allow Fmoc-SAR-Wang resin (1.0 mmol, purchased from Aapptec, RWG103, Lot #9953380, 0.65 mmol/g, 110-200 mesh) to swell in DMF (8 vol) for 2 h and drain DMF. Treat the resin with 20% piperidine in DMF (6 vol), shake for 3 min, collect the solvent and dry for 5 min under N₂ gas (repeat the same sequence for 4 times). Finally, wash the resin with DMF (5 vol×5 times), with CH₂Cl₂ (5 vol×5 times) and dry under vacuum using N₂ gas atmosphere for overnight.

Calculation of resin loading: Add 20% piperidine in DMF to the above collected piperidine solution to make an approximate final volume of 40 vol. Then, take 0.1 mL of solution and dilute to 100 volumes with DMF and measure UV absorbance at 301 nm of the Fmoc group per gram.

### Conditions for UV Measurement

Solvent: 20% piperidine in DMF
Wave length: 301 nm
ε=7800

### Example 12B—General Procedure for Solid-Phase Synthesis of PMOs

(a) Synthetic Plan for all-Sp-PMOs

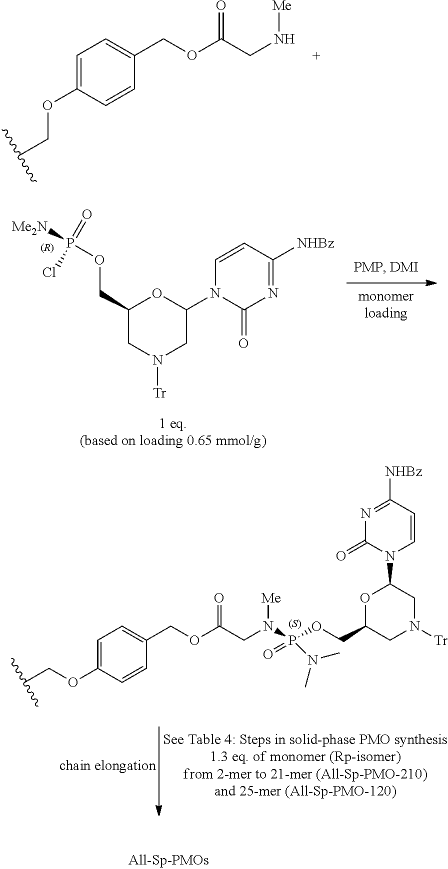

(b) Synthetic Plan for all-Rp-PMOs

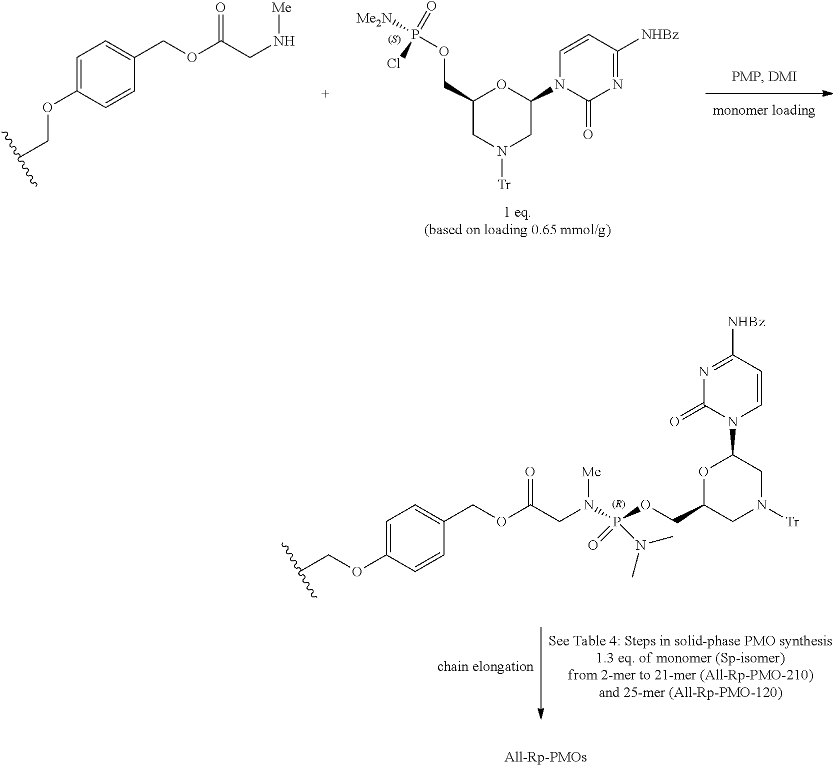

Transfer Fmoc deprotected resin (1 mmol) in to a peptide synthesizer reaction vessel, wash with CH₂Cl₂(20 vol×5 times), with acetonitrile (20 vol×5 times), and dry the resin. Add stereopure cytosine dimethylphosphoramidochloridate (1 mmol), anhydrous 1,3-dimethyl-2-imidazolidinone (DMI, 5.0 vol), and 1,2,2,6,6-pentamethyl-piperidine (PMP, 10.0 vol) to the resin flask and shake at rt for 20 h. Record LCMS of aliquot to confirm complete loading of monomer on resin. Then, perform steps 5-9, Table 4 as in order.

TABLE 4
Steps in solid-phase PMO synthesis

| Step | Reaction | Reagent | | |
|---|---|---|---|---|
| 1 | Detritylation | 2% 3-cyanipyridine-TFA, 0.9% EtOH, 20% | 7 | cycles |
| | | TFE/DCM then CH₂Cl₂ wash | | |
| 2 | Neutralization | 10% Hunig's base in NMP | 4 | times |
| 3 | Wash | 1,3-dimethyl-2-imidazolidinone wash (2 times) | | |
| | | MeCN wash (10 times), | | |
| | | CH₂Cl₂ wash (15 times) | | |
| 4 | Coupling | Monomer (1.3 eq), DMI (0.05M), PMP (10 eq.) | 20 | h |
| 5 | Wash | With MeCN (10 times), | | |
| | | With CH₂Cl₂ (10 times) | | |
| 6 | Capping | 2,6-Lutidine (30% in MeCN) and Ac₂O (20% in | 3 | times |
| | | MeCN) | | |
| 7 | Wash | With CH₂Cl₂ (10 times) | | |
| 8 | Ac₂O removal | 10% Morpholine in NMP | 4 | times |

| | | |
|---|---|---|
| 9 | Wash | With CH₂Cl₂ (10 times) |

After the first monomer loading on resin, repeat the synthetic cycle (deprotection, neutralization, coupling and capping) as shown Table 4 to obtain the required oligonucleotide length. In each synthetic cycle, add the required monomer (confirm the purities of monomers by HPLC-Mass before use) to obtain the titled nucleotide sequence.

In each synthetic cycle, after coupling reaction (step 4, Table 4), treat a small amount of resin to cleavage conditions (0.1 mL of 7 N NH₃/MeOH), and record RP HPLC-Mass to record the coupling efficiency.

### Example 12C—Cleavage from the Resin and Base Deprotection

After completion of desired length of oligonucleotide, dry the resin then transfer into a centrifugal bottle, add 7 N NH₃/MeOH (100 vol) and stir at 50-55° C. Filter the solids, and wash the resin with methanol. Concentrate the filtrate under reduced pressure to ¼th of initial volume, filter through 0.45 micron membrane filter, concentrate and dry the product. Redissolve the crude product in 1/1 (v/v) mixture of aq. Et₃NHOAc/MeCN (1/1) and doppe with Et₃N (0.1%). Purify the crude product using reversed phase HPLC column conditions.

### Example 12D—Final Detritylation

Cool the flask containing 3′-N-Tr-PMO (1 eq.) to 0° C., add freshly prepared 0.1 M aq. phosphoric acid (20 eq.) and stir the mixture at room temperature. Upon reaction completion (confirmed by RP-LCMS), basify the reaction mixture with 28% ammonium hydroxide (40 eq.), stir at room temperature for 2 hours, filter the solids through membrane filter (0.45 μ), wash with water. Purify the compound using reverse phase HPLC. Analyze the each HPLC fraction, concentrate the product containing fractions, and freeze-dry to get the desired oligonucleotide.

### Example 13—Dimer Synthesis

### ((2S,6R)-6-(4-benzamido-2-oxopyrimidin-1(2H)-yl)-4-tritylmorpholin-2-yl)methyl (S)-P-((2S,6R)-2-(((tert-butyldiphenylsilyl)oxy)methyl)-6-(5-methyl-2,4-dioxo-3,4-dihydropyrimidin-1(2H)-yl)morpholino)-N,N-dimethylphosphonamidate

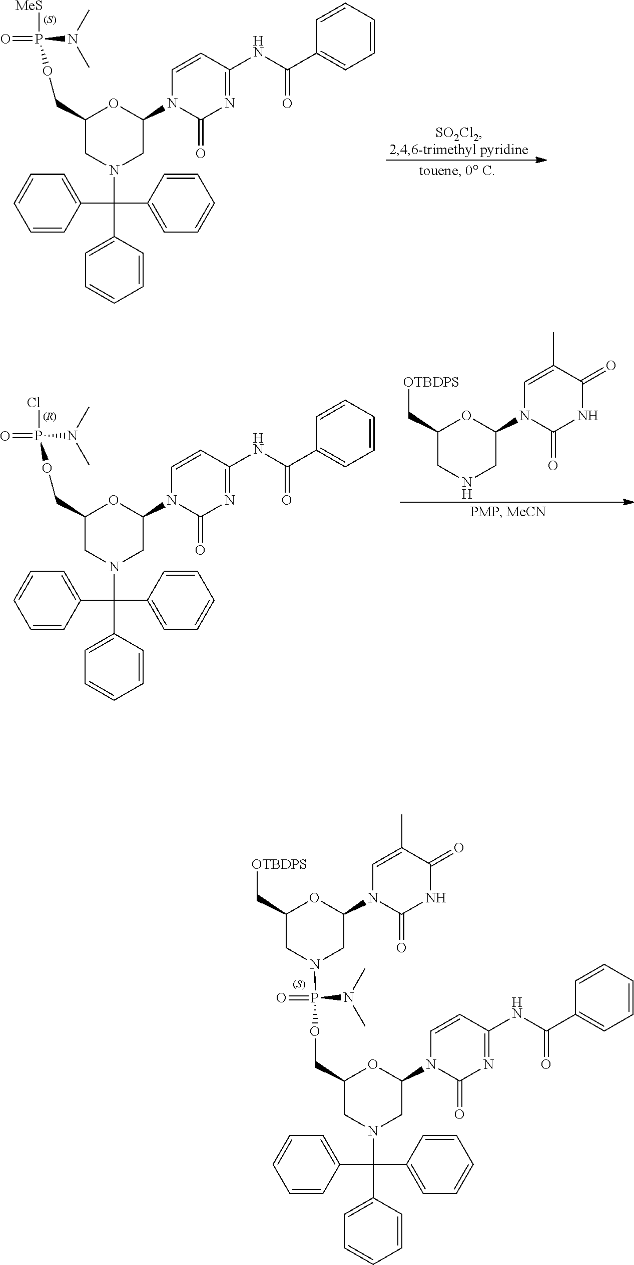

Flask charged with O-(((2S,6R)-6-(4-benzamido-2-oxopyrimidin-1(2H)-yl)-4-tritylmorpholin-2-yl)methyl)S-methyl (S)-dimethylphosphoramidothioate (0.060 g, 0.085 mmol), then added toluene (1.2 mL). The reaction formed a clear solution after 10 min stirring at room temperature. The reaction mixture was cooled to 0° C., treated with 2,4,6-trimethylpyridine (0.11 mL, 0.84 mmol) and sulfuryl chloride (0.17 mL, 0.085 mmol) (0.5 M solution in toluene), and stirred at 0° C. for 1 h.

Another flask was charged with 1-((2R,6S)-6-(((tert-butyldiphenylsilyl)oxy)methyl) morpholin-2-yl)-5-methylpyrimidine-2,4(1H,3H)-dione (0.049 g, 0.10 mmol), acetonitrile (0.6 mL) and 1,2,2,6,6-pentamethylpiperidine (0.066 g, 0.42 mmol). The mixture was treated with the solution of chloride (prepared above) and stirred at room temperature for 2 h. The reaction mixture was concentrated under vacuum and purified using silica gel column chromatography (MeOH/EtOAc=0% to 10%) to yield the titled product (48 mg, 50% yield). ³¹P NMR (CDCl₃, 162 MHz) δ 16.1 (desired), 16.4 (undesired); ESI-MS m/z: Calculated for [C₆₃H₆₉N₈O₉PSi+H]⁺ 1141.47; Found 1141.2.

¹³P NMR showed ~1:6 diastereoselectivity (undesired vs desired).

In situ coupling:

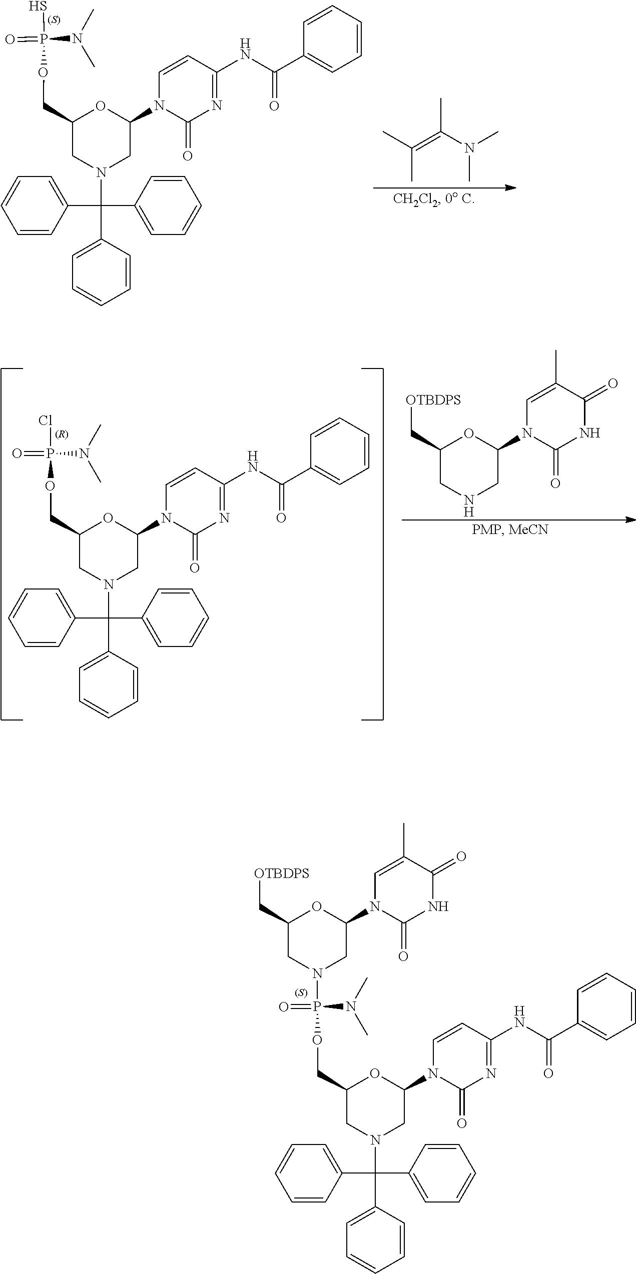

O-(((2S,6R)-6-(4-benzamido-2-oxopyrimidin-1(2H₁)-yl)-4-tritylmorpholin-2-yl)methyl)S-hydrogen (S)-dimethylphosphoramidothioate (100 mg, 0.14 mmol) was dissolved in anhydrous CH₂Cl₂ (1.5 mL) and cooled to 0° C. The mixture was treated with 1-chloro-N,N,2-trimethylprop-1-en-1-amine (29 mg, 0.26 mmol) and stirred at 0° C. for 1 h. After completion of activation (monitored by LCMS), the mixture was treated with a solution of 1-((2R,6S)-6-(((tert-butyldiphenylsilyl)oxy)methyl)morpholin-2-yl)-5-methylpyrimidine-2,4(1H,3H)-dione (62 mg, 0.13 mmol) and 1,2,2,6,6-pentamethylpiperidine (0.26 mL, 1.4 mmol) in MeCN (1.5 mL), and stirred at room temperature for 4 h. The reaction was quenched with aq. 10% citric acid (5 mL) and diluted with EtOAc (5 mL). Two layers were separated and the aqueous layer was extracted with EtOAc (2×5 mL). The combined organic layers were washed with brine (5 mL), dried over Na₂SO₄, filtered, concentrated and purified using silica gel column chromatography (EtOAc/n-heptane=20% to 100% then MeOH/DCM=0% to 20%) to yield the title product (106 mg, 72% yield).

³¹P NMR (CDCl_{3,} 162 MHz) δ 16.1 (desired), 16.4 (undesired); ESI-MS m/z: Calculated for [C₆₃H₆₉N₈O₉PSi+H]⁺ 1141.47; Found 1141.2.

¹³P NMR showed >99:1 dr.

### Example 14—Preparation of 2′-Deoxyribonucleoside Activated Monomers

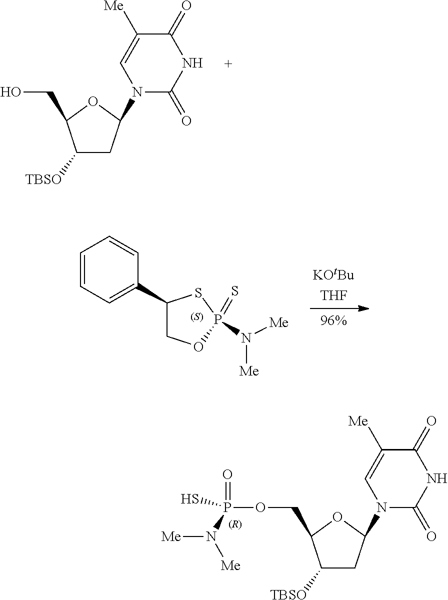

### O-(((2R,3S,5R)-3-((tert-butyldimethylsilyl)oxy)-5-(5-methyl-2,4-dioxo-3,4-dihydropyrimidin-1(2H)-yl)tetrahydrofuran-2-yl)methyl)S-hydrogen (R)-dimethylphosphoramidothioate

To a solution of 1-((2R,4S,5R)-4-((tert-butyldimethylsilyl)oxy)-5-(hydroxymethyl)tetrahydrofuran-2-yl)-5-methylpyrimidine-2,4(1H,3H)-dione (1.0 g, 2.8 mmol) in THF (14 mL) was added potassium tert-butoxide (8.4 mL in THF, 8.4 mmol) at 0° C., followed by addition of (2S,4R)-2-(dimethylamino)-4-phenyl-1,3,2-oxathiaphospholane 2-sulfide (1.1 g, 4.2 mmol). The reaction mixture was stirred at 0° C. for 2 h before additional (2S,4R)-2-(dimethylamino)-4-phenyl-1,3,2-oxathiaphospholane 2-sulfide (0.51 g, 1.9 mmol) was added followed by addition of potassium tert-butoxide (2.8 mL in THF, 2.8 mmol) at 0° C. The mixture was kept stirring for another 30 min before additional potassium tert-butoxide (0.7 mL in THF, 0.7 mmol) at 0° C. It was stirred at 0° C. for another 20 min, and the reaction was quenched with acetic acid (1.4 mL, 24 mmol). The reaction was worked up with EtOAc (100 mL) and aq. sodium dihydrogen phosphate (50 mL, 10%). The aq. layer was back extracted with EtOAc (100 mL). Combined organic layers were combined, washed subsequently with water (50 mL) and sat. brine (50 mL), dried over Na₂SO₄, and concentrated to ca. 25 mL, into which was added n-heptane (150 mL), and the supernatant was decanted. The sticky stuff was redissolved in EtOAc (20 mL), and into the solution was then added n-heptane (120 mL). The resulted suspension was centrifuged, and the solid was rinsed with a mixture of EtOAc/n-heptane (v/v 1:6, 49 mL). The solid was dried by coevaporation with a mixture of THF and MeCN to give the title compound (1.29 g, 96%).

¹H NMR (400 MHz, CHLOROFORM-d) δ ppm 8.76-8.89 (m, 1H), 7.36-7.45 (m, 1H), 6.13-6.27 (m, 1H), 4.33-4.43 (m, 2H), 4.22-4.32 (m, 1H), 3.98-4.07 (m, 1H), 2.86 (s, 3H), 2.84 (s, 3H), 2.20-2.28 (m, 2H), 1.95 (s, 3H), 0.89 (s, 9H), 0.09 (s, 6H)

³¹P NMR (162 MHz, CHLOROFORM-d) δ ppm 65.03 (br s, 1 P)

MS (ESI) m/z: [M+H]⁺ Calcd for C18H34N306PSSi 480.2; Found 480.0

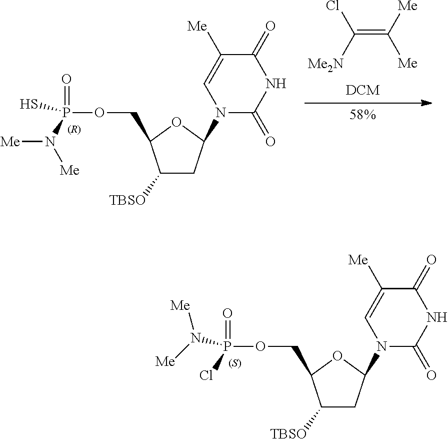

### ((2R,3S,5R)-3-((tert-butyldimethylsilyl)oxy)-5-(5-methyl-2,4-dioxo-3,4-dihydropyrimidin-1(2H)-yl)tetrahydrofuran-2-yl)methyl (S)-dimethylphosphoramidochloridate

To a solution of O-(((2R,3S,5R)-3-((tert-butyldimethylsilyl)oxy)-5-(5-methyl-2,4-dioxo-3,4-dihydropyrimidin-1(2H)-yl)tetrahydrofuran-2-yl)methyl)S-hydrogen (R)-dimethylphosphoramidothioate (1.29 g, 2.69 mmol) in DCM (13.5 mL) was added 1-Chloro-N,N,2-trimethylpropenylamine (0.53 mL, 4.0 mmol) dropwise at 0° C. After addition, the reaction mixture was stirred at 0° C. for 30 min before more 1-Chloro-N,N,2-trimethylpropenylamine (0.18 mL, 1.3 mmol) was added. The mixture was stirred at 0° C. for another 20 min before it was diluted up with EtOAc (100 mL), and then quenched with sat. NaHCO₃(aq.) at 0° C. After phase separation, aqueous layer was back extracted with EtOAc (100 mL). Combined organic layers were washed with half sat. brine, dried over Na₂SO₄, and concentrated. The crude material was purified by silica gel column chromatography, eluting with 60% EtOAc in n-heptane to afford the title compound (0.75 g, 58%), and the diastereomeric ratio was determined by HPLC to be 99.5:0.5.

¹H NMR (400 MHz, CHLOROFORM-d) δ ppm 8.23 (s, 1H), 7.32 (s, 1H), 6.24-6.32 (m, 1H), 4.44 (dt, J=6.4, 3.1 Hz, 1H), 4.22-4.37 (m, 2H), 4.04-4.10 (m, 1H), 2.77 (s, 3H), 2.73 (s, 3H), 2.23-2.41 (m, 1H), 1.99-2.08 (m, 1H), 1.94 (s, 3H), 0.89 (s, 9H), 0.10 (s, 6H)

³¹P NMR (162 MHz, CHLOROFORM-d) δ ppm 19.04 (s, 1 P)

MS (ESI) m/z: [M+H]⁺ Calcd for C18H33ClN3O6PSi 482.2; Found 482.0

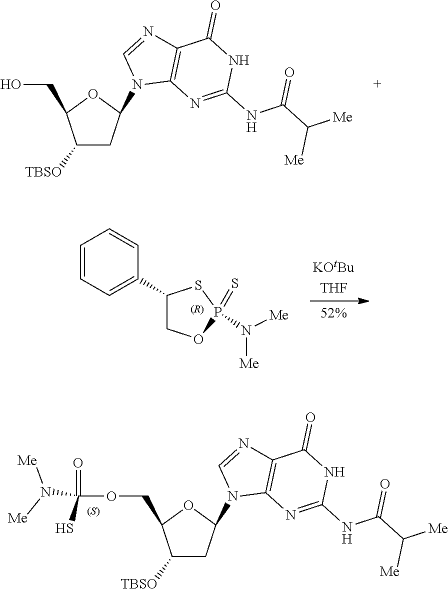

### O-(((2R,3S,5R)-3-((tert-butyldimethylsilyl)oxy)-5-(2-isobutyramido-6-oxo-1,6-dihydro-9H-purin-9-yl)tetrahydrofuran-2-yl)methyl)S-hydrogen (S)-dimethylphosphoramidothioate

To a solution of N-(9-((2R,4S,5R)-4-((tert-butyldimethylsilyl)oxy)-5-(hydroxymethyl)tetrahydrofuran-2-yl)-6-oxo-6,9-dihydro-1H-purin-2-yl)isobutyramide (1.0 g, 2.3 mmol) and (2R,4S)-2-(dimethylamino)-4-phenyl-1,3,2-oxathiaphospholane 2-sulfide (0.89 g, 3.4 mmol) in THF (11.3 mL) was added potassium tert-butoxide (6.8 mL in THF, 6.8 mmol) dropwise at 0° C. The reaction mixture was stirred at 0° C. for 40 min before additional (2R,4S)-2-(dimethylamino)-4-phenyl-1,3,2-oxathiaphospholane 2-sulfide (0.45 g, 1.7 mmol) was added. It was kept stirring with ice bath overnight, and the reaction was quenched with acetic acid (0.78 mL, 13.6 mmol) at 0° C. It was worked up with EtOAc (100 mL) and aq. sodium dihydrogen phosphate (40 mL, 10%). After phase separation, the aqueous layer was back extracted with EtOAc (150 mL×3). Organic layers were combined, washed with half sat. brine (150 mL), dried over Na₂SO₄, and concentrated to a residue. The crude material was redissolved in EtOAc (40 mL), into the solution was then added n-heptane (320 mL). The suspension was then filtered, and rinsed with a mixture of EtOAc/n-heptane (v/v 1:8, 90 mL). The solid was dried by coevaporation with a mixture of THF/MeCN to afford title compound (0.67 g, 52%).

¹H NMR (400 MHz, CHLOROFORM-d) δ ppm 12.30 (s, 1H), 11.34 (s, 1H), 7.61 (s, 1H), 5.87-6.01 (m, 1H), 4.56-4.72 (m, 2H), 4.42-4.56 (m, 1H), 4.00-4.11 (m, 1H), 2.99 (s, 3H), 2.97 (s, 3H), 2.84-3.11 (m, 2H), 1.91-2.07 (m, 1H), 1.37 (d, J=8.0 Hz, 3H), 1.22 (d, J=4.0 Hz, 3 H), 0.89 (m, 9H), 0.14 (s, 3H), 0.09 (s, 3H)

³¹P NMR (162 MHz, CHLOROFORM-d) δ ppm 64.31 (s, 1 P).

MS (ESI) m/z: [M+H]⁺ Calcd for C22H39N6O6PSSi 575.2; Found 575.1

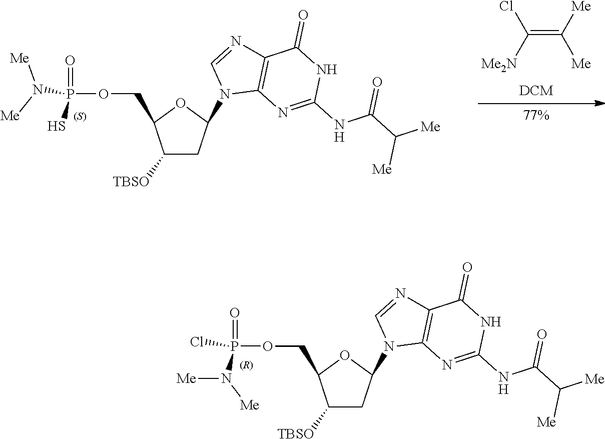

### ((2R,3S,5R)-3-((tert-butyldimethylsilyl)oxy)-5-(2-isobutyramido-6-oxo-1,6-dihydro-9H-purin-9-yl)tetrahydrofuran-2-yl)methyl (R)-dimethylphosphoramidochloridate

To a solution of O-(((2R,3S,5R)-3-((tert-butyldimethylsilyl)oxy)-5-(2-isobutyramido-6-oxo-1,6-dihydro-9H-purin-9-yl)tetrahydrofuran-2-yl)methyl)S-hydrogen (S)-dimethylphosphoramidothioate (0.67 g, 1.2 mmol) in DCM (6.7 mL) was added 1-Chloro-N,N,2-trimethylpropenylamine (0.39 mL, 2.9 mmol) dropwise at 0° C. The reaction mixture was stirred at 0° C. for 45 min before it was diluted with EtOAc (100 mL), and then quenched with sat. NaHCO₃(aq.) at 0° C. After phase separation, the aqueous layer was back extracted with EtOAc (100 mL). Combined organic layers were washed with half sat. brine, dried over Na₂SO₄, and concentrated. The crude material was purified by silica gel column chromatography, eluting with EtOAc to afford the title compound (0.52 g, 77%), and the diastereomeric ratio was determined by HPLC to be 93.4:6.6.

¹H NMR (400 MHz, CHLOROFORM-d) δ ppm 12.16 (s, 1H), 10.23 (s, 1H), 7.66 (s, 1H), 6.18-6.25 (m, 1H), 5.19-5.29 (m, 1H), 4.82-4.90 (m, 1H), 4.26-4.37 (m, 1H), 4.04-4.17 (m, 1 H), 3.15-3.28 (m, 1H), 2.80 (s, 3H), 2.76 (s, 3H), 2.66-2.75 (m, 1H), 2.27-2.38 (m, 1H), 1.19-1.30 (m, 6H), 0.90 (s, 9H), 0.13 (s, 3H), 0.12 (s, 3H)

³¹P NMR (162 MHz, CHLOROFORM-d) δ ppm 19.67 (s, 1 P)

MS (ESI) m/z: [M+H]⁺ Calcd for C22H39ClN6O6PSi 577.2; Found 577.1

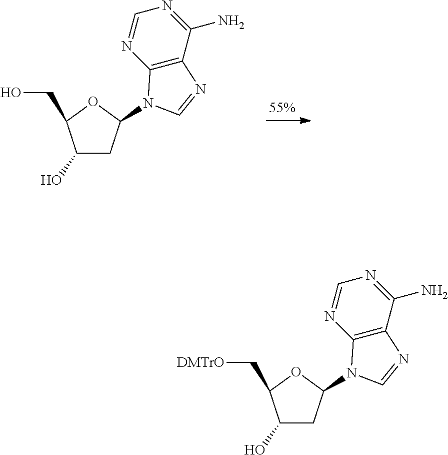

### (2R,3S,5R)-5-(6-amino-9H-purin-9-yl)-2-((bis(4-methoxyphenyl)(phenyl)methoxy)methyl)tetrahydrofuran-3-ol

(2R,3S,5R)-5-(6-amino-9H-purin-9-yl)-2-(hydroxymethyl)tetrahydrofuran-3-ol (3.0 g, 11.9 mmol) was dried by coevaporation with MeCN twice and pyridine once before it was suspended in pyridine (70 mL). To the mixture was added 4,4′-dimethoxytrityl chloride (4.45 g, 13.1 mmol) at rt. The reaction mixture was stirred at rt for 2 h before it was quenched with NaHCO₃ aqueous solution (150 mL, 5%). It was extracted with EtOAc (500 mL×2). The combined organic layers were washed with sat. brine, dried over Na₂SO₄, and concentrated. The crude material was purified by silica gel column chromatography, eluting with 13% to 15% MeOH in EtOAc to afford the title product (3.62 g, 55%).

¹H NMR (400 MHz, CD₃OD) δ 8.21 (s, 1H), 8.13 (s, 1H), 7.38-7.33 (m, 2H), 7.26-7.13 (m, 7H), 6.80-6.73 (m, 4H), 6.43 (dd, J=6.4, 6.0 Hz, 1H), 4.64 (m, 1H), 4.57 (s, 1H), 4.12 (m, 1H), 3.75 (s, 6H), 3.33 (d, J=4.4 Hz, 2H), 2.92 (m, 1H), 2.50 (m, 1H); ESI-MS m/z: Calculated for [C₃₁H₃₁N₅O₅+H]⁺ 554.2; Found 554.0.

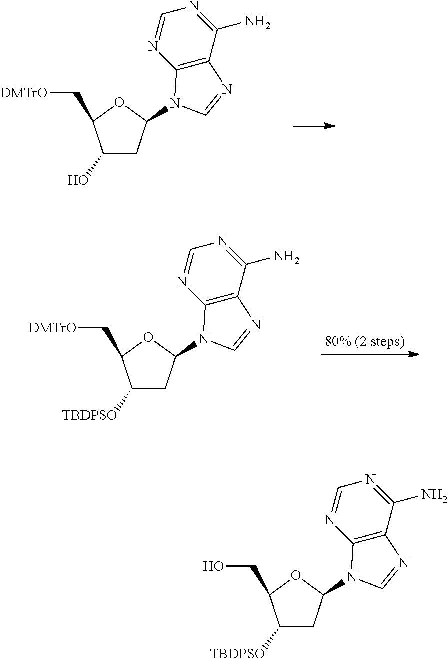

### ((2R,3S,5R)-5-(6-amino-9H-purin-9-yl)-3-((tert-butyldiphenylsilyl)oxy)tetrahydrofuran-2-yl)methanol

(2R,3S,5R)-5-(6-amino-9H-purin-9-yl)-2-((bis(4-methoxyphenyl)(phenyl)methoxy)methyl) tetrahydrofuran-3-ol (3.62 g, 6.54 mmol) was coevaporated with MeCN once before it was dissolved in DMF (37.5 mL) in a flask with a room temp water bath. To the solution was added imidazole (3.56 g, 52.3 mmol), Et₃N (3.65 mL, 26.2 mmol), and TBDPS-Cl (3.36 mL, 13.1 mmol) at rt, and the reaction mixture was stirred at rt for 1 h before additional imidazole (1.78 g, 26.2 mmol), TBDPS-Cl (3.36 mL, 13.1 mmol), and DMAP (0.799 g, 6.54 mmol) was added, and it was kept stirring at rt overnight. The reaction was quenched with water (75 mL), and extracted with MTBE (250 mL×2). The combined organic layers were washed with sat. NaHCO₃(aq.), sat. brine, dried over Na₂SO₄, and concentrated. The crude material was purified by silica gel column chromatography, eluting with 90% to 100% EtOAc in n-heptane to afford desired product, which was used directly for next step, assuming 100% yield.

To a flask charged with 9-((2R,4S,5R)-5-((bis(4-methoxyphenyl)(phenyl)methoxy)methyl)-4-((tert-butyldiphenylsilyl)oxy)tetrahydrofuran-2-yl)-9H-purin-6-amine (5.18 g, 6.54 mmol) was added a mixture of 1,1,1,3,3,3-Hexafluoro-2-propanol (21.4 mL), 2,2,2-Trifluoroethanol (10.6 mL), DCM (40.0 mL) and Triethylsilane (17.8 mL, 111 mmol) at rt. it was stirred at rt for 30 min before it was concentrated on rotavap, and coevaporated with with MeOH once. The crude material was suspended in methanol (52.9 mL) and DCM (53.0 mL), into which was added p-toluenesulfonic acid monohydrate (0.124 g, 0.654 mmol) at rt, it was kept stirring at rt for 30 min before triethylamine (0.182 mL, 1.31 mmol) was added, and then concentrated on rotavap. The residue was coevaporated with THF once before it was redissolved in a mixture of THF (37.0 mL), water (9.31 mL) and TFA (1.51 mL, 19.6 mmol) at 0° C. The reaction mixture was stirred with ice bath for 1 h before it diluted with EtOAc (250 mL), and quenched with saturated sodium bicarbonate aq. solution (54.9 mL). The solid suspended in EtOAc layer was collected by filtration, and rinsed with EtOAc to give 1^{st} crop of product. After phase separation of the filtrate, the aqueous layer was extracted with a mixture of EtOAc (250 mL) and 2-MeTHF (200 mL). Combined organic layers were washed with sat. brine, dried over Na₂SO₄, and concentrated. To the resulted residue was added a mixture of DCM (30 mL), MTBE (30 mL), and n-heptane (30 mL), and the solid was collected by filtration, rinsed with MTBE to give 2^{nd} crop of product. The combined solid was dried over vacuum with a stream of N₂ to give the title product (2.55 g, 80% over two steps).

¹H NMR (400 MHz, CD₃OD) δ 8.23 (s, 1H), 8.11 (s, 1H), 7.79 (s, 2H), 7.70-7.65 (m, 4H), 7.48-7.37 (m, 6H), 6.51 (dd, J=8.8, 5.6 Hz, 1H), 4.64 (m, 1H), 4.52 (brs, 1H), 4.08 (m, 1H), 3.57 (dd, J=12.4, 2.4 Hz, 1H), 3.16 (dd, J=12.4, 2.8 Hz, 1H), 2.66 (m, 1H), 2.42 (m, 1H); ESI-MS m/z: Calculated for [C₂₆H₃₁N₅O₃Si+H]⁺ 490.2; Found 490.0.

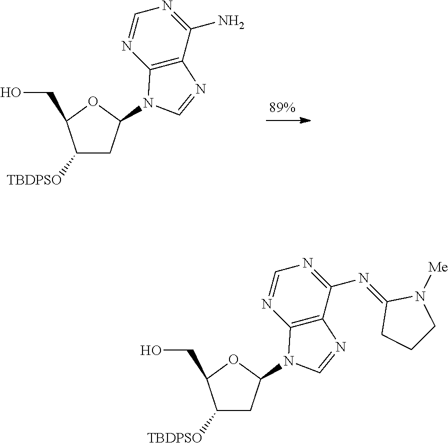

### ((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methanol

((2R,3S,5R)-5-(6-amino-9H-purin-9-yl)-3-((tert-butyldiphenylsilyl)oxy)tetrahydrofuran-2-yl)methanol (2.43 g, 4.96 mmol) was coevaporated with anhydrous MeCN once before it was suspended in DMF (89 mL), which was then treated with 2,2-diethoxy-1-methylpyrrolidine (3.82 mL, 19.9 mmol) at RT for 1 h before water (0.89 mL, 50 mmol) was added and it was kept stirring at rt for 30 min. The mixture was then diluted with water (180 mL) and extracted with EtOAc (300 mL×2). The combined EtOAc layers were washed with half saturated brine, dried over Na₂SO₄, and concentrated. The crude material was purified by silica gel column chromatography, eluting with 5% to 15% MeOH in EtOAc to afford the title product (2.52 g, 89%).

¹H NMR (400 MHz, CD₃OD) δ 8.39 (s, 2H), 7.69 (m, 4H), 7.50-7.36 (m, 6H), 6.57 (dd, J=12.4, 5.6 Hz, 1H), 4.66 (m, 1H), 4.09 (m, 1H), 3.56 (m, 3H), 3.21 (dd, J=12.4, 3.2 Hz, 1H), 3.11 (s, 3H), 2.81 (m, 2H), 2.69 (m, 1H), 2.46 (m, 1H), 2.04 (m, 2H), 1.12 (s, 9H); ESI-MS m/z: Calculated for [C₃₁H₃₈N₆₀₃Si+H]⁺ 571.3; Found 571.1.

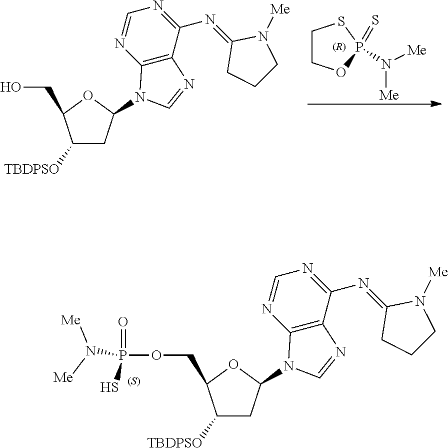

### O-(((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methyl)S-hydrogen (S)-dimethylphosphoramidothioate

A mixture of ((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methanol (157 mg, 0.275 mmol) and (R)-2-(dimethylamino)-1,3,2-oxathiaphospholane 2-sulfide (76 mg, 0.41 mmol) was dried by coevaporation with anhydrous MeCN once before it was dissolved in THF (1.57 mL). To the solution was then added sodium-tert-amoxide solution in toluene (4 M, 247 μl, 0.825 mmol) dropwise at 0° C. The reaction mixture was stirred at 0° C. for 2 h before it was quenched with acetic acid (94 μL, 1.6 mmol) at 0° C. It was then diluted with EtOAc (10 mL), and sodium dihydrogen phosphate aq. solution (9.9 mL, 10%) was added. The mixture was extracted with EtOAc (40 mL×2). Combined EtOAc layers were washed with half sat. brine, dried over Na₂SO₄, and concentrated. The residue was then redissolved in a mixture of THF (5 mL) and EtOAc (5 mL), into which was added n-heptane (50 mL). The solid was collected the solid by filtration, rinsed with n-heptane-EA (30 mL, 5:1 v/v), and dried over vacuum with a stream of N₂ overnight to give the title product (161 mg, 84%).

¹H NMR (400 MHz, CDCl₃) δ 9.08 (brs, 1H), 8.12 (brs, 1H), 7.72-7.62 (m, 4H), 7.52-7.36 (m, 6H), 6.55 (dd, J=9.6, 4.8 Hz, 1H), 4.60 (d, J=5.2 Hz, 1H), 4.21 (s, 1H), 4.18 (m, 1H), 3.98 (m, 1H), 3.73-3.55 (m, 2H), 3.44 (s, 3H), 3.34 (m, 1H), 2.82 (m, 1H), 2.76-2.62 (m, 1H), 2.54 (s, 3H), 2.51 (s, 3H), 2.36-2.22 (m, 2H), 2.10 (m, 1H), 1.12 (s, 9H); ³¹P NMR (162 MHz, CDCl₃) δ 65.7; ESI-MS m/z: Calculated for [C₃₃H₄₄N₇₀₄PSSi+H]⁺ 694.3; Found 694.0.

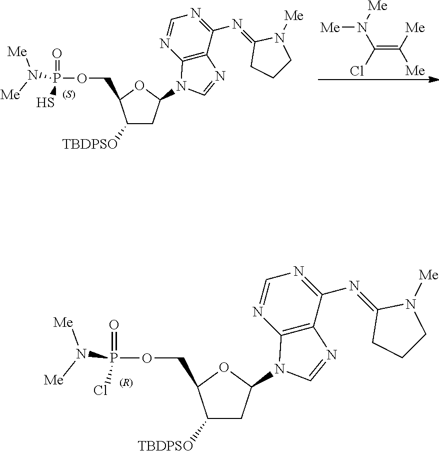

### ((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methyl (R)-dimethylphosphoramidochloridate

O-(((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methyl)S-hydrogen (S)-dimethylphosphoramidothioate (100 mg, 0.143 mmol) was dried by coevaporation with anhydrous MeCN once before it was dissolved in DCM (1.29 mL), into which was added 1-Chloro-N,N,2-trimethylpropenylamine (76 μL, 0.57 mmol) at 0° C.. The reaction mixture was stirred at 0° C. for 2.5 h before additional 1-Chloro-N,N,2-trimethylpropenylamine (19 μL, 0.14 mmol) was added. It was stirred with ice bath for 30 min before it was diluted with EtOAc, and quenched with saturated sodium bicarbonate aqueous solution (9.6 mL) at 0° C. After phase separation, it was back extracted once with EtOAc. The combined layers (100 mL) were washed with half saturated brine, dried over Na₂SO₄, and concentrated. The crude material was purified by silica gel column chromatography, eluting with 0% to 100% THF in EtOAc to afford the title product (132 mg). The diastereomeric ratio was determined to be 95.5:4.5 by HPLC.

¹H NMR (400 MHz, CDCl₃) δ 8.44 (s, 1H), 7.91 (s, 1H), 7.64-7.56 (m, 4H), 7.44-7.30 (m, 6H), 6.46 (dd, J=6.8, 6.4 Hz, 1H), 4.58 (m, 1H), 4.18 (m, 1H), 4.10-3.75 (m, 2H), 3.48-3.41 (m, 2H), 3.08 (s, 3H), 2.92-2.82 (m, 2H), 2.51 (s, 3H), 2.48 (s, 3H), 2.44 (m, 1H), 2.10-1.70 (m, 3H), 1.05 (s, 9H); ³¹P NMR (162 MHz, CDCl₃) δ 18.5; ESI-MS m/z: Calculated for [C₃₃H₄₃ClN₇₀₄PSi+H]⁺ 696.3; Found 696.1.

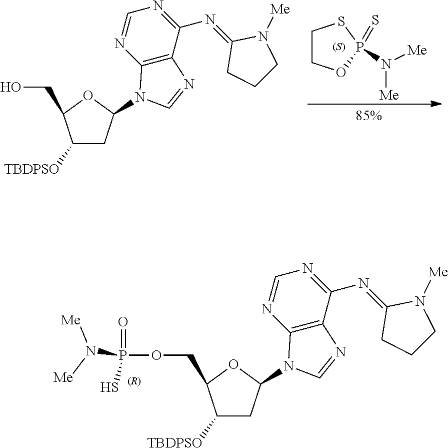

### O-(((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methyl)S-hydrogen (R)-dimethylphosphoramidothioate

A mixture of ((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methanol (210 mg, 0.368 mmol) and (S)-2-(dimethylamino)-1,3,2-oxathiaphospholane 2-sulfide (101 mg, 0.553 mmol) was dried by coevaporation with anhydrous MeCN once before it was dissolved in THF (2.1 mL), into which was then added a solution of sodium-tert-amoxide in toluene (0.33 mL, 1.1 mmol) dropwise at 0° C. The reaction mixture was stirred at 0° C. for 1 h before it was quenched with acetic acid (126 μL, 2.21 mmol). The mixture was diluted with EtOAc, and then aqueous NaH₂PO₄ solution (25 mL, 10%) was added. The mixture was extracted with EtOAc (40 mL×2). Th5%)e combined organic layers were washed with half saturated brine (30 mL), dried over Na₂SO₄, and concentrated. The crude material was redissolved in a mixture of THF (6.5 mL) and EtOAc (6.5 mL), into the solution was then added n-heptane (65 mL). The solid was collected by filtration, rinsed with a mixture of n-heptane-EA (40 mL, 5:1 v/v), and dried under vacuum with a stream of N₂ overnight to afford the title product (218 mg, 85%).

¹H NMR (400 MHz, CDCl₃) δ 8.48 (brs, 1H), 8.38 (brs, 1H), 7.62-7.56 (m, 4H), 7.42-7.28 (m, 6H), 6.45 (dd, J=8.8, 6.0 Hz, 1H), 4.53 (d, J=4.2 Hz, 1H), 4.18 (s, 1H), 4.12 (m, 1H), 3.60-3.42 (m, 2H), 3.13 (m, 1H), 3.09 (s, 3H), 2.80 (m, 1H), 2.53 (s, 3H), 2.50 (s, 3H), 2.48 (m, 1H), 2.30 (m, 1H), 2.15-1.95 (m, 2H), 1.04 (s, 9H); ³¹P NMR (162 MHz, CDCl₃) δ 65.8; ESI-MS m/z: Calculated for [C₃₃H₄₄N₇O₄PSSi+H]⁺ 694.3; Found 693.8.

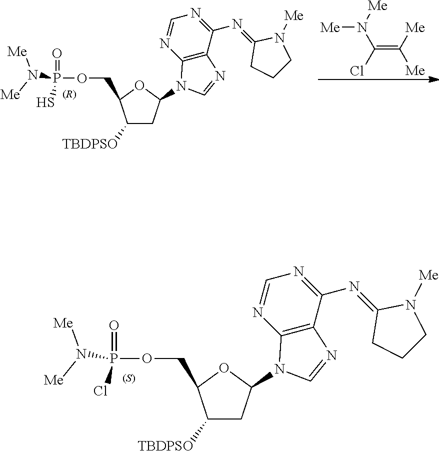

### ((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methyl (S)-dimethylphosphoramidochloridate

O-(((2R,3S,5R)-3-((tert-butyldiphenylsilyl)oxy)-5-(6-(((E)-1-methylpyrrolidin-2-ylidene)amino)-9H-purin-9-yl)tetrahydrofuran-2-yl)methyl)S-hydrogen (R)-dimethylphosphoramidothioate (93 mg, 0.13 mmol) was dried by coevaporation with anhydrous MeCN once before it was dissolved in DCM (1.2 mL), into which was added at 0° C. 1-Chloro-N,N,2-trimethylpropenylamine (71 mL, 0.54 mmol). The reaction mixture was stirred at 0° C. for 2.5 h before additional 1-Chloro-N,N,2-trimethylpropenylamine (18 μL, 0.13 mmol) was added.

It was stirred for 30 min before it was diluted with with EtOAc, and then quenched with saturated sodium bicarbonate aqueous solution (9.0 mL) at 0° C. After phase separation, it was back extracted once with EtOAc. The combined organic layers (80 mL) were washed with half sat. brine, dried over Na₂SO₄, concentrated. The crude material was purified by silica gel column chromatography, eluting with 0% to 100% THF in EtOAc to afford the title product (124 mg). The diastereomeric ratio was determined to be 96.5:3.5 by HPLC.

¹H NMR (400 MHz, CDCl₃) δ 8.44 (s, 1H), 7.89 (s, 1H), 7.64-7.56 (m, 4H), 7.44-7.30 (m, 6H), 6.44 (dd, J=7.2, 6.4 Hz, 1H), 4.58 (m, 1H), 4.17 (m, 1H), 4.10-3.75 (m, 2H), 3.49-3.41 (m, 2H), 3.08 (s, 3H), 2.94-2.86 (m, 2H), 2.50 (s, 3H), 2.47 (s, 3H), 2.44 (m, 1H), 2.10-1.70 (m, 3H), 1.05 (s, 9H); ³¹P NMR (162 MHz, CDCl₃) δ 18.5; ESI-MS m/z: Calculated for [C₃₃H₄₃ClN₇₀₄PSi+H]⁺ 696.3; Found 696.1.

### Example 15—Solution Phase Preparation of Oligomers Using Both Morpholino Nucleoside Activated Monomers and 2′-Deoxyribonucleoside Activated Monomers

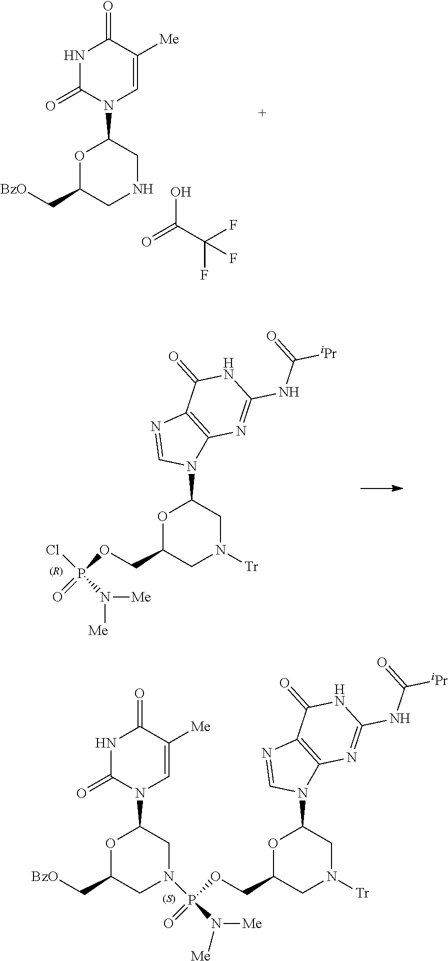

### ((2S,6R)-4-((S)-(dimethylamino)(((2S,6R)-6-(2-isobutyramido-6-oxo-1,6-dihydro-9H-purin-9-yl)-4-tritylmorpholin-2-yl)methoxy)phosphoryl)-6-(5-methyl-2,4-dioxo-3,4-dihydropyrimidin-1(2H)-yl)morpholin-2-yl)methyl benzoate

To a solution of ((2S,6R)-6-(5-methyl-2,4-dioxo-3,4-dihydropyrimidin-1(2H)-yl)morpholin-2-yl)methyl benzoate 2,2,2-trifluoroacetate (1.40 g, 3.05 mmol) in 1,3-dimethyl-2-imidazolidinone (15.8 mL) and THF (21.0 mL) was added 1,2,2,6,6-pentamethylpiperidine (2.16 mL, 11.9 mmol). The mixture was cooled below 10° C. before ((2S,6R)-6-(2-isobutyramido-6-oxo-1,6-dihydro-9H-purin-9-yl)-4-tritylmorpholin-2-yl)methyl (R)-dimethylphosphoramidochloridate (2.10 g, 2.98 mmol) was added. After addition, the reaction mixture was then stirred at rt for 5 h. To the mixture was added MTBE (~20 mL) and n-heptane (60 mL). The supernatant was decanted, and the residue was purified by silica gel column chromatography, eluting with 5% to 15% MeOH in DCM to give product (2.71 g) as white foam solid.

MS (ESI) m/z: [M+H]⁺ Calcd for C52H57N10O10P 1013.4; Found 1013.3

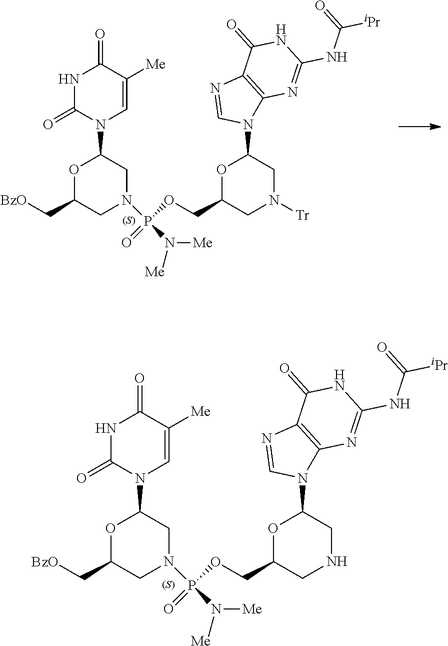

### ((2S,6R)-4-((S)-(dimethylamino)(((2S,6R)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate

To a solution of ((2S,6R)-4-((S)-(dimethylamino)(((2S,6R)-6-(2-isobutyramido-6-oxo-1,6-dihydro-9H-purin-9-yl)-4-tritylmorpholin-2-yl)methoxy)phosphoryl)-6-(5-methyl-2,4-dioxo-3,4-dihydropyrimidin-1(2H)-yl)morpholin-2-yl)methyl benzoate (2.71 g, 2.68 mmol) in dichloromethane (32.5 mL) was added ethanol (1.56 mL, 26.8 mmol) and TFA (1.24 mL, 16.0 mmol) at rt. The reaction mixture was stirred at rt for 1 h before MTBE (100 mL) was added. The suspension was stirred for 5 min, and the solid was collected by filtration, rinsed with MTBE, and dried in vacuo.

The resulted solid was redissolved in DCM (30 mL), and treated with 1,2,2,6,6-pentamethylpiperidine (1.45 mL, 8.03 mmol) at rt for about 10 min before MTBE (100 mL) was added. The solid was collected by filtration, and dried in vacuo for 30 min to afford product (2.30 g).

MS (ESI) m/z: [M+H]⁺ Calcd for C33H43N10O10P 771.3; Found 771.3

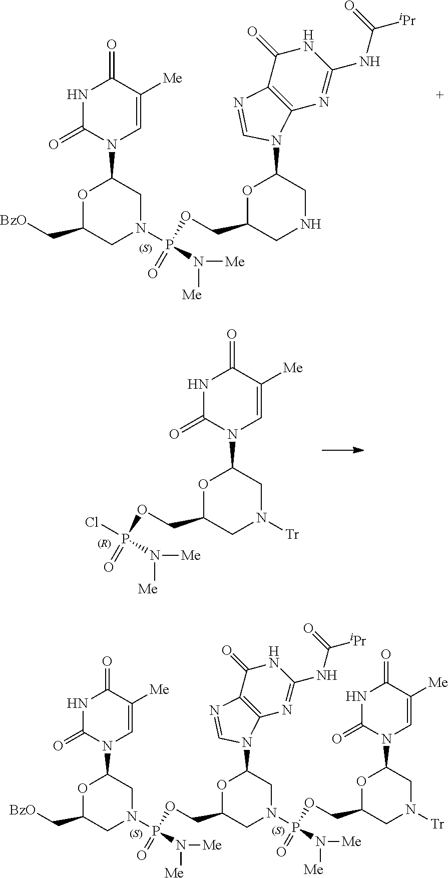

### ((2S,6R)-4-((S)-(dimethylamino)(((2S,6R)-4-((S)-(dimethylamino)(((2S,6R)-6-(5-methyl-2,4-dioxo-3,4-dihydropyrimidin-1(2H)-yl)-4-tritylmorpholin-2-yl)methoxy)phosphoryl)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate

To a solution of ((2S,6R)-4-((S)-(dimethylamino)(((2S,6R)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate (2.30 g, 2.69 mmol) in 1,3-dimethyl-2-imidazolidinone (24.0 mL) was added 1,2,2,6,6-pentamethylpiperidine (1.22 mL, 6.71 mmol) and ((2S,6R)-6-(5-methyl-2,4-dioxo-3,4-dihydropyrimidin-1(2H)-yl)-4-tritylmorpholin-2-yl)methyl (R)-dimethylphosphoramidochloridate (1.88 g, 3.09 mmol). The reaction mixture was then stirred at rt overnight before MTBE (150 mL) was added. The supernatant was decanted, and the resulted sticky solid was rinsed with MTBE, followed by trituration with a mixture of EtOAc (20 mL) and MTBE (60 mL) to give product (3.82 g).

MS (ESI) m/z: [M+H]⁺ Calcd for C64H76N14O15P2 1343.5; Found 1343.5

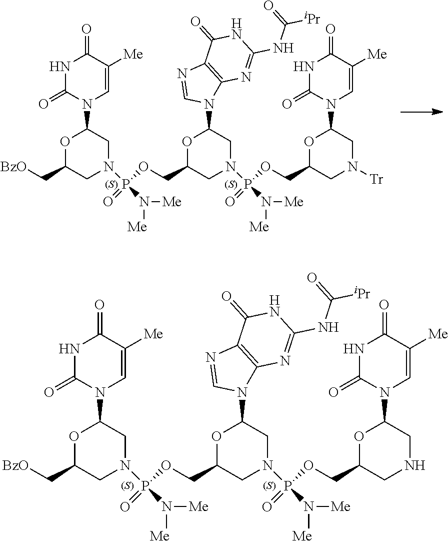

### ((2S,6R)-4-((S)-(dimethylamino)(((2S,6R)-4-((S)-(dimethylamino)(((2S,6R)-6-(5-methyl-2,4-dioxo-3,4-dihydropyrimidin-1(2H)-yl)morpholin-2-yl)methoxy)phosphoryl)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate

To a solution of ((2S,6R)-4-((S)-(dimethylamino)(((2S,6R)-4-((S)-(dimethylamino)(((2S,6R)-6-(5-methyl-2,4-dioxo-3,4-dihydropyrimidin-1(2H)-yl)-4-tritylmorpholin-2-yl)methoxy)phosphoryl)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate (3.82 g, 2.67 mmol) in dichloromethane (39.5 mL) was added ethanol (1.56 mL, 26.7 mmol) and TFA (1.24 mL, 16.0 mmol) at rt. The reaction mixture was stirred at rt for 45 min before MTBE (120 mL) was added. The suspension was stirred at rt for 15 min before the solid was collected by filtration, rinsed with MTBE, and dried in vacuo.

The resulted solid was then redissolved in DCM (39.5 mL), and treated with 1,2,2,6,6-pentamethylpiperidine (1.94 mL, 10.7 mmol) at rt for 10 min before MTBE (120 mL) was added. The solid was collected by filtration, and dried in vacuo for 30 min to give product (2.98 g).

MS (ESI) m/z: [M+H]⁺ Calcd for C45H62N14O15P2 1101.4; Found 1101.4

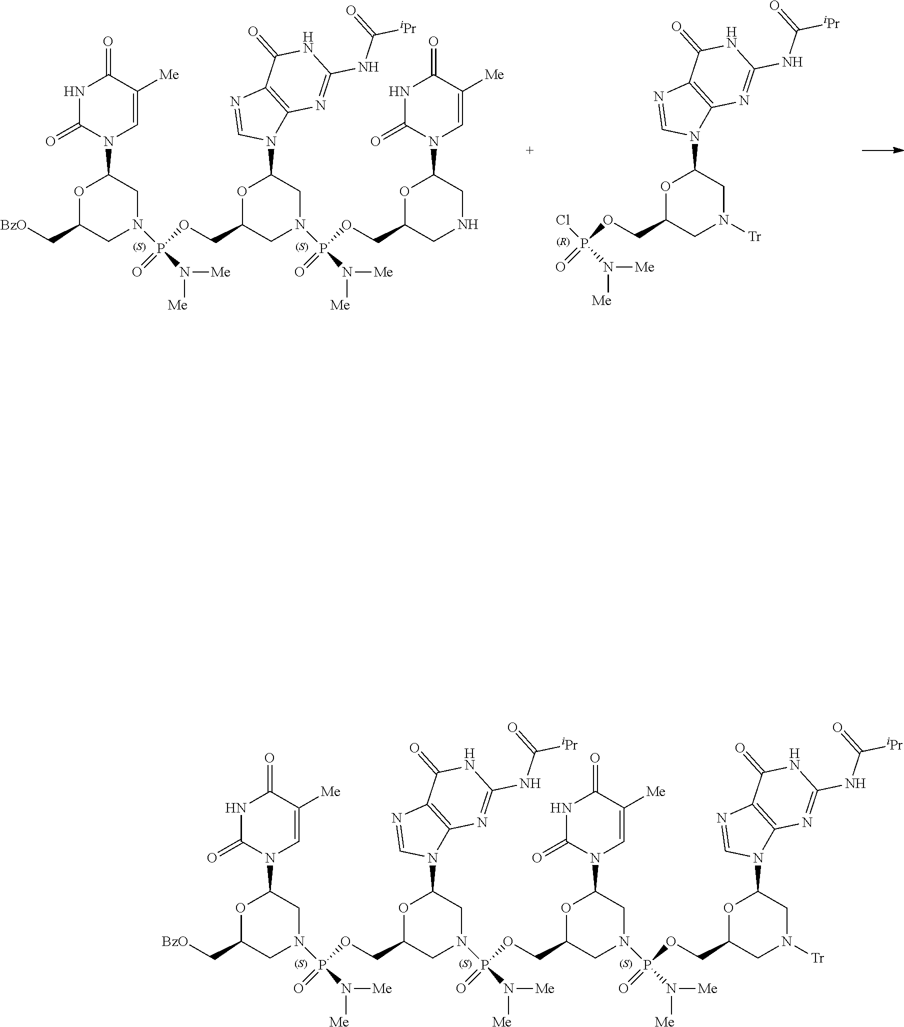

### ((2S,6R)-4-((S)-(dimethylamino)(((2S,6R)-4-((S)-(dimethylamino)(((2S,6R)-4-((S)-(dimethylamino)(((2S,6R)-6-(2-isobutyramido-6-oxo-1,6-dihydro-9H-purin-9-yl)-4-tritylmorpholin-2-yl)methoxy)phosphoryl)-6-(5-methyl-2,4-dioxo-3,4-dihydropyrimidin-1(2H)-yl)morpholin-2-yl)methoxy)phosphoryl)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate

To a solution of ((2S,6R)-4-((S)-(dimethylamino)(((2S,6R)-4-((S)-(dimethylamino)(((2S,6R)-6-(5-methyl-2,4-dioxo-3,4-dihydropyrimidin-1(2H)-yl)morpholin-2-yl)methoxy)phosphoryl)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate (2.98 g, 2.71 mmol) in 1,3-dimethyl-2-imidazolidinone (20.0 mL) was added 1,2,2,6,6-pentamethylpiperidine (1.47 mL, 8.12 mmol) and ((2S,6R)-6-(2-isobutyramido-6-oxo-1,6-dihydro-9H-purin-9-yl)-4-tritylmorpholin-2-yl)methyl (R)-dimethylphosphoramidochloridate (2.29 g, 3.25 mmol) at rt. The reaction mixture was stirred at rt overnight before morpholine (0.236 mL, 2.71 mmol) was added. The mixture was stirred for about 20 min before EtOAc (60 mL) was added, followed by addition of MTBE (120 mL). The solid was collected by filtration, rinsed with with EtOAc-MTBE (1:1, v/v). The second crop was also collected from the filtrate, and rinsed with MTBE-EtOAc (2:1, v/v). The combined solid was slurried with EtOAc (20 mL) for 5 min, and then into the slurry was added MTBE (20 mL). The solid was collected by filtration, rinsed with EtOAc-MTBE (1:1, v/v), dried in vacuo for 30 min to product (4.72 g).

MS (ESI) m/z: [M+H]⁺ Calcd for C80H100N21O20P3 1768.7; Found 1768.4

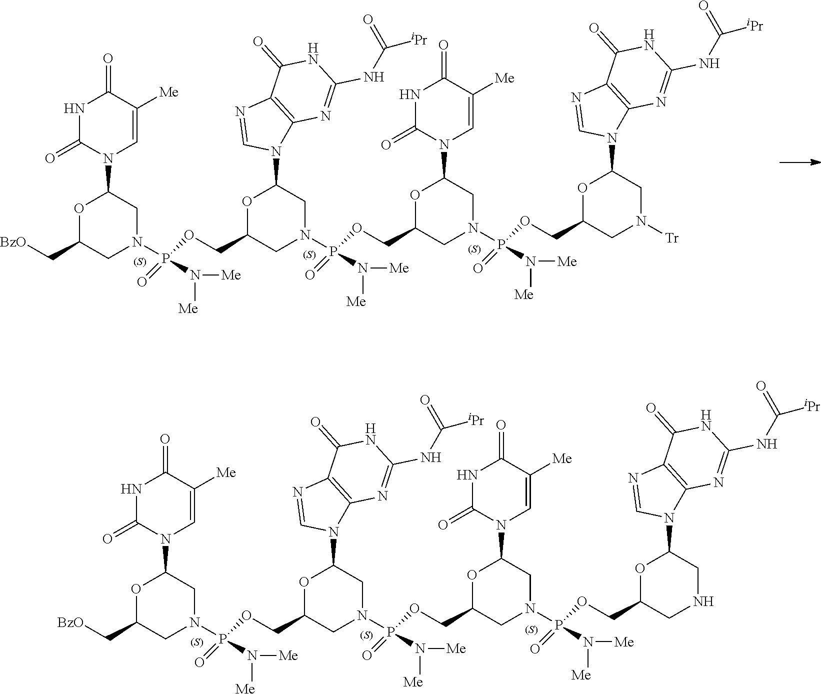

### ((2S,6R)-4-((S)-(dimethylamino)(((2S,6R)-4-((S)-(dimethylamino)(((2S,6R)-4-((S)-(dimethylamino)(((2S,6R)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methoxy)phosphoryl)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate

To a solution of ((2S,6R)-4-((S)-(dimethylamino)(((2S,6R)-4-((S)-(dimethylamino)(((2S,6R)-4-((S)-(dimethylamino)(((2S,6R)-6-(2-isobutyramido-6-oxo-1,6-dihydro-9H-purin-9-yl)-4-tritylmorpholin-2-yl)methoxy)phosphoryl)-6-(5-methyl-2,4-dioxo-3,4-dihydropyrimidin-1(2H)-yl)morpholin-2-yl)methoxy)phosphoryl)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate (4.72 g, 2.67 mmol) in dichloromethane (47.2 mL) was added ethanol (1.56 mL, 26.7 mmol) and TFA (1.23 mL, 16.0 mmol) at rt. The reaction mixture was stirred at rt for 20 min before EtOAc (80 mL) was added, followed by addition of MTBE (80 mL). The suspension was stirred for 5 min, and the solid was then collected by filtration, rinsed with EtOAc-MTBE (1:1, v/v), and dried in vacuo.

The resulted solid was redissolved in dichloromethane (47.2 mL), and then treated with 1,2,2,6,6-pentamethylpiperidine (1.93 mL, 10.7 mmol) at rt for 10 min before EtOAc (60 mL) was added, followed by addition of MTBE (100 mL). The solid was collected by filtration, and dried in vacuo for 30 min. The resulting solid was dissolved in a mixture of DCM (40 mL) and MeOH (2 mL), and to the solution was added EtOAc (40 mL) and then MTBE (80 mL). The solid was collected by filtration, and dried in vacuo overnight to give product (3.46 g) as white solid.

MS (ESI) m/z: [M+H]⁺ Calcd for C61H86N21O20P3 1526.6; Found 1526.6

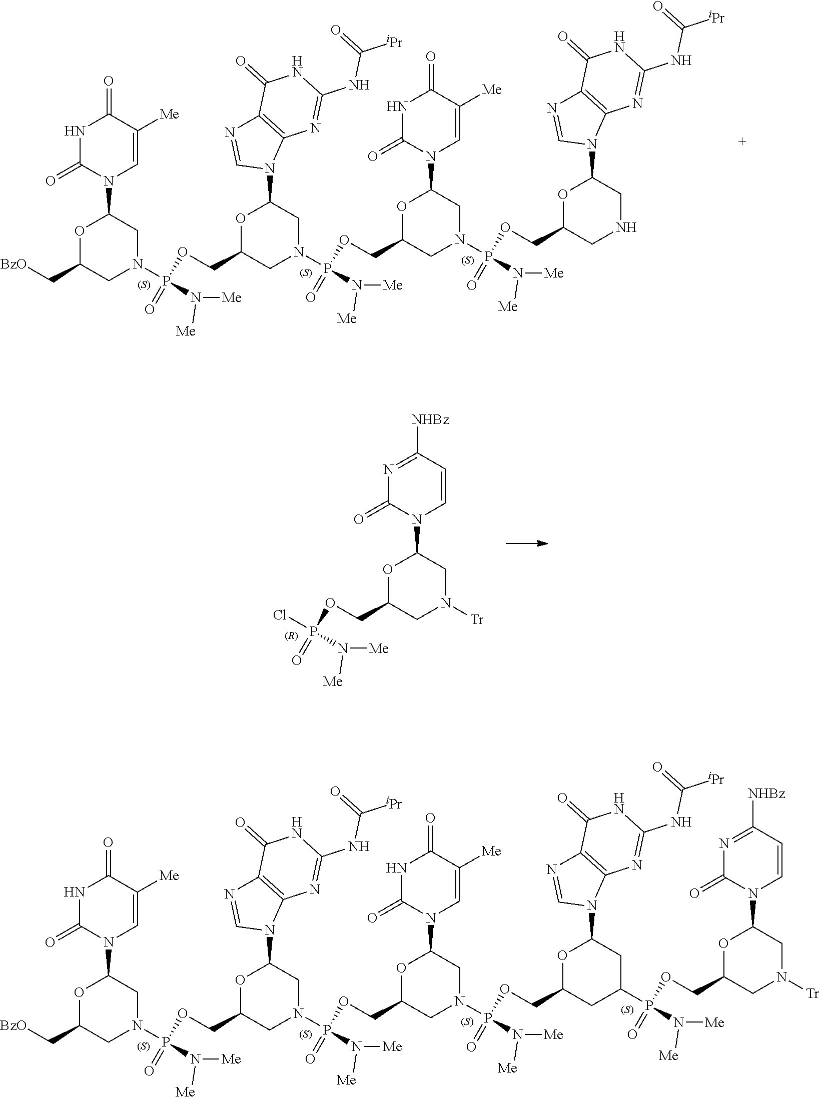

### ((2S,6R)-4-((S)-(((2S,6R)-4-((S)-(((2S,6R)-4-((S)-(((2S,6R)-4-((S)-(((2S,6R)-6-(4-benzamido-2-oxopyrimidin-1(2H)-yl)-4-tritylmorpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methyl benzoate

To a solution of ((2S,6R)-4-((S)-(dimethylamino)(((2S,6R)-4-((S)-(dimethylamino)(((2S,6R)-4-((S)-(dimethylamino)(((2S,6R)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methoxy)phosphoryl)-6-(2-isobutyramido-6-oxo-1,6-dihydro-9H-purin-9-yl)morpholin-2-yl)methoxy)phosphoryl)-6-(5-methyl-2,4-dioxo-3,4-dihydropyrimidin-1(2H)-yl)morpholin-2-yl)methyl benzoate (302 mg, 0.198 mmol) in 1,3-dimethyl-2-imidazolidinone (3.01 mL) was added 1,2,2,6,6-pentamethylpiperidine (107 μL, 0.594 mmol) followed by addition of ((2S,6R)-6-(4-benzamido-2-oxopyrimidin-1(2H)-yl)-4-tritylmorpholin-2-yl)methyl (R)-dimethylphosphoramidochloridate (166 mg, 0.237 mmol) at rt. The reaction mixture was stirred at rt overnight. To the mixture was added EtOAc (30 mL), followed by addition of MTBE (45 mL). The solid was collected by filtration, rinsed with EtOAc-MTBE (2:3, v/v) to give product (454 mg).

³¹P NMR (162 MHz, CD3OD) δ ppm 17.25 (brs, 2 P), 16.97 (s, 1 P), 16.91 (s, 1 P)

MS (ESI) m/z: [M+2H]²⁺ Calcd for C98H122N26O25P4 1094.9; Found 1094.7

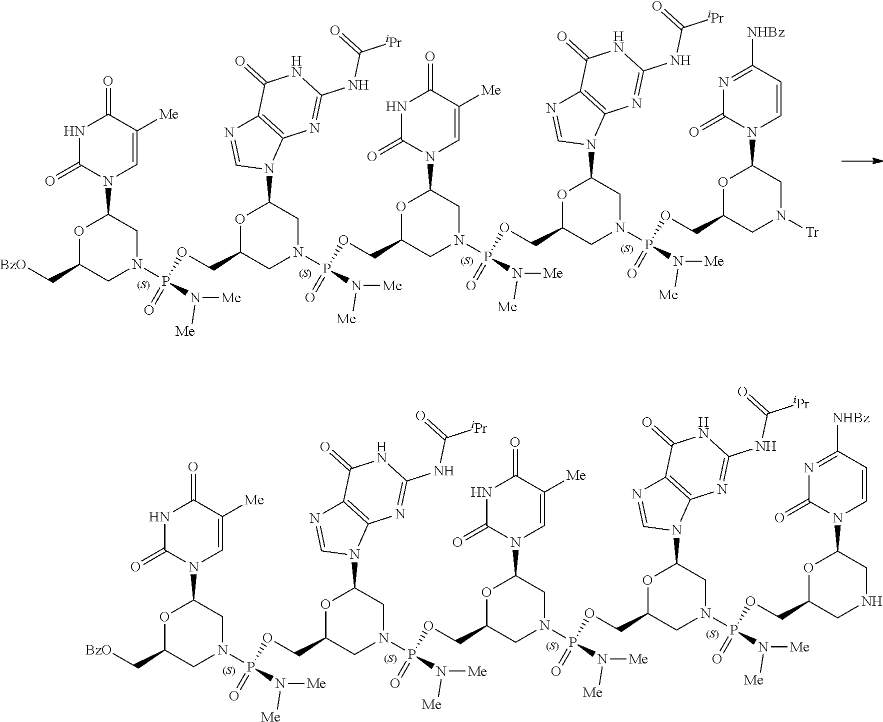

### ((2S,6R)-4-((S)-(((2S,6R)-4-((S)-(((2S,6R)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methyl benzoate

To a flask charged with ((2S,6R)-4-((S)-(((2S,6R)-4-((S)-(((2S,6R)-4-((S)-(((2S,6R)-4-((S)-(((2S,6R)-6-(4-benzamido-2-oxopyrimidin-1(2H)-yl)-4-tritylmorpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methyl benzoate (433 mg, 0.198 mmol) was added a mixture of TFA (76 μL, 0.99 mmol), ethanol (116 μL, 1.98 mmol) and DCM (4.33 mL) at rt. The reaction mixture was stirred at rt for about 1 h before EtOAc (26 mL) was added. The suspension was centrifuged, and the supernatant was decanted. The resulted solid was sonicated in a mixture of DCM-EtOAc (1:6, v/v, 35 mL) for 2 min before centrifugation. Decantation of the supernatant afforded TFA salt, which was then treated with a solution of 1,2,2,6,6-pentamethylpiperidine (362 μL, 1.98 mmol) in DCM (8.53 mL) at rt for 5 min before addition of EtOAc (45 mL) and MTBE (38 mL) to the solution. The solid was collected by filtration, rinsed with a mixture of EtOAc (30 mL) and MTBE (26 mL) to give product (320 mg).

MS (ESI) m/z: [M+2H]²⁺ Calcd for C79H108N26O25P4 973.4; Found 973.4

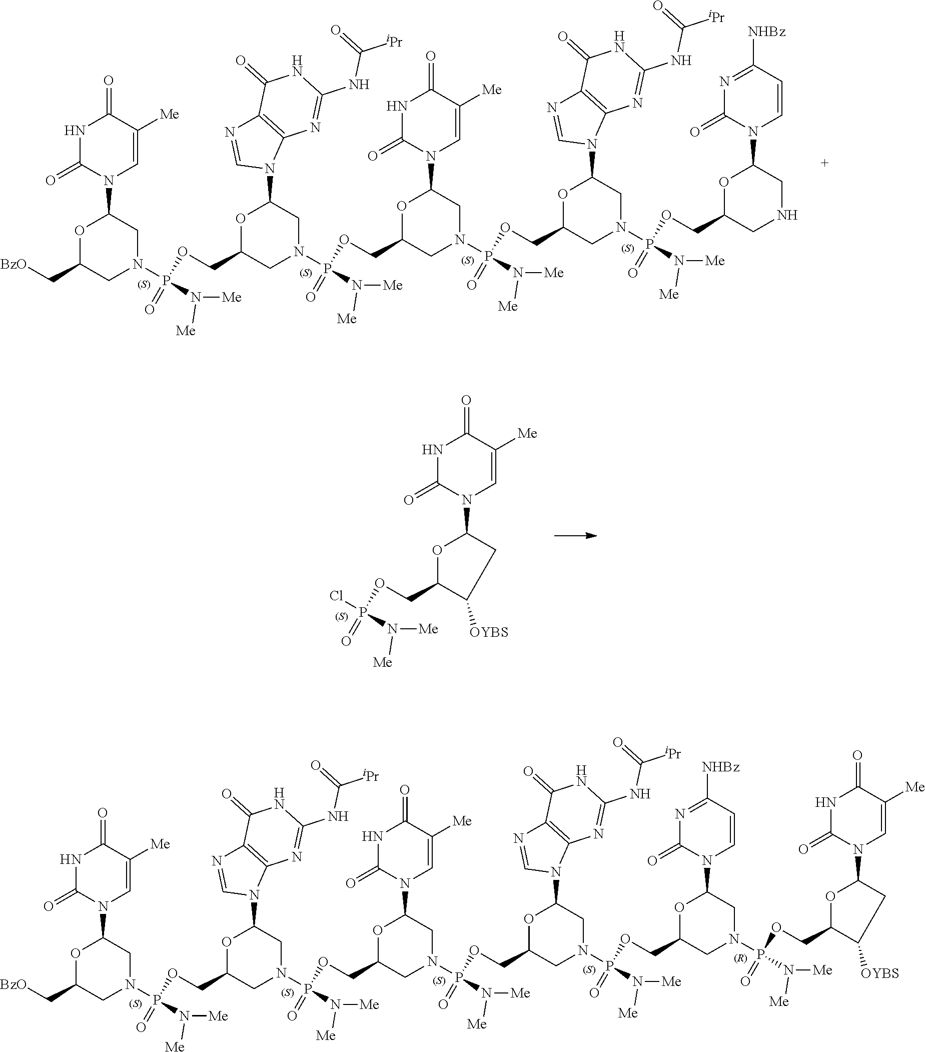

### ((2S,6R)-4-((S)-(((2S,6R)-4-((S)-(((2S,6R)-4-((S)-(((2S,6R)-4-((S)-(((2S,6R)-6-(4-benzamido-2-oxopyrimidin-1(2H)-yl)-4-((R)-(((2R,3S,5R)-3-((tert-butyldimethylsilyl)oxy)-5-(5-methyl-2,4-dioxo-3,4-dihydropyrimidin-1(2H)-yl)tetrahydrofuran-2-yl)methoxy)(dimethylamino)phosphoryl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methyl benzoate

To a solution of ((2S,6R)-4-((S)-(((2S,6R)-4-((S)-(((2S,6R)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methyl benzoate (320 mg, 0.164 mmol) in 1,3-dimethyl-2-imidazolidinone (3.20 mL) was added 1,2,2,6,6-pentamethylpiperidine (89 μL, 0.49 mmol) followed by addition of ((2R,3S,5R)-3-((tert-butyldimethylsilyl)oxy)-5-(5-methyl-2,4-dioxo-3,4-dihydropyrimidin-1(2H)-yl)tetrahydrofuran-2-yl)methyl (S)-dimethylphosphoramidochloridate (95 mg, 0.20 mmol) at rt. The reaction mixture was stirred at rt overnight before EtOAc (16 mL) was added, followed by addition of MTBE (48 mL). The solid was collected by filtration, rinsed with a mixture of EtOAc-MTBE (1:3, v/v) to give product (466 mg).

³¹P NMR (162 MHz, CD3OD) δ ppm 17.25 (brs, 2 P), 17.03 (s, 1 P), 16.97 (s, 1 P), 16.92 (s, 1 P)

MS (ESI) m/z: [M+2H]²⁺ Calcd for C97H140N29O31P5Si 1196.4; Found 1196.2

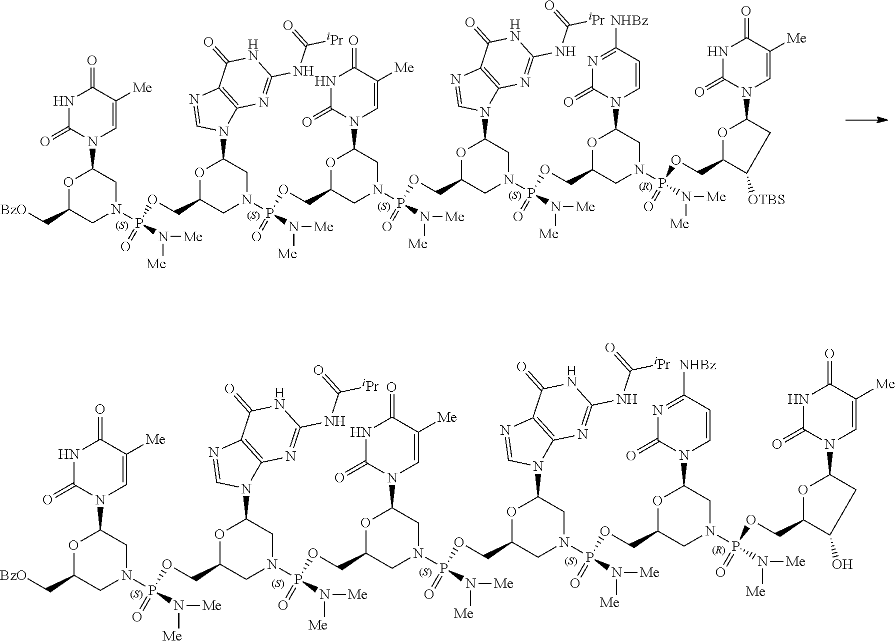

### ((2S,6R)-4-((S)-(((2S,6R)-4-((S)-(((2S,6R)-4-((S)-(((2S,6R)-4-((S)-(((2S,6R)-6-(4-benzamido-2-oxopyrimidin-1(2H)-yl)-4-((R)-(dimethylamino)(((2R,3S,5R)-3-hydroxy-5-(5-methyl-2,4-dioxo-3,4-dihydropyrimidin-1(2H)-yl)tetrahydrofuran-2-yl)methoxy)phosphoryl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methyl benzoate

To a solution of ((2S,6R)-4-((S)-(((2S,6R)-4-((S)-(((2S,6R)-4-((S)-(((2S,6R)-4-((S)-(((2S,6R)-6-(4-benzamido-2-oxopyrimidin-1(2H)-yl)-4-((R)-(((2R,3S,5R)-3-((tert-butyldimethylsilyl)oxy)-5-(5-methyl-2,4-dioxo-3,4-dihydropyrimidin-1(2H)-yl)tetrahydrofuran-2-yl)methoxy)(dimethylamino)phosphoryl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)-6-(2-isobutyramido-6-oxo-1,6-dihydro-9H-purin-9-yl)morpholin-2-yl)methoxy)(dimethylamino)phosphoryl)-6-(5-methyl-2,4-dioxo-3,4-dihydropyrimidin-1(2H)-yl)morpholin-2-yl)methyl benzoate (466 mg, 0.195 mmol) in a mixture of pyridine (3.70 mL, 45.8 mmol), TEA (3.72 mL, 26.7 mmol), and DCM (3.72 mL) with a room temperature water bath was added triethylamine trihydrofluoride (476 μL, 2.92 mmol). After addition, the water bath was removed, and the reaction mixture was stirred at ambient temperature overnight. To the reaction mixture was then added methoxytrimethylsilane (1.85 mL, 13.4 mmol) at 0° C. After addition, the mixture was stirred at ambient temperature for 3 h before EtOAc (30 mL) was added, followed by addition of MTBE (15 mL). The solid was collected by filtration, rinsed with a mixture of EtOAc (36 mL) and MTBE (18 mL) to give product (399 mg).

MS (ESI) m/z: [M+2H]²⁺ Calcd for C91H126N29O31P5 1138.9; Found 1139.1

### Example 16—Another Example to Prepare Chiral 2-(dimethylamino)-1,3,2-oxathiaphospholane 2-sulfide

### Example 16A: Preparation of chiral 2-((4-bromophenyl)thio)-L 3,2-oxathiaphospholane 2-sulfide and (R)-2-((4-bromophenyl)thio)-1,3,2-oxathiaphospholane 2-sulfide

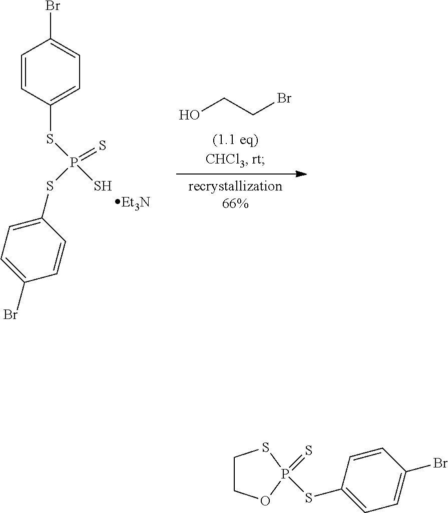

2-((4-Bromophenyl)thio)-1,3,2-oxathiaphospholane 2-sulfide. A mixture of triethylamine bis(4-bromophenyl)phosphorotetrathioate (20.0 g, 34.9 mmol) and 2-bromoethanol (2.60 mL, 36.6 mmol) in chloroform (160 mL) was stirred at rt for 22 h. Additional 2-bromoethanol (0.12 mL, 0.05 eq.) was added and stirring was continued at rt for additional 3 h. The reaction mixture was concentrated in vacuo until tubid (ca. 20 mL), and treated with water (50 mL) while stirring and then with n-heptane (50 mL). The mixture was cooled with an ice bath and stirred at 0° C. for 1 h. The precipitate was filtered, and washed with water (40 mL) and n-heptane (50 mL) to give a crude product (9.8 g).

The filter cake was dissolved in CH2CH2 (80 mL) and the aqueous layer was removed. The organic layer was concentrated in vacuo to ca. 40 mL and treated with n-heptane (50 mL). After stirring at rt for 20 min, the mixture was concentrated in vacuo to a half volume (ca. 40 mL). The resulting precipitate was filtered, washed with n-heptane (5 mL), and dried over N2 purge for 2 h to give the title compound (8.7 g).

For the recrystallization, the filter cake was dissolved in MTBE (160 mL) with heating to 60° C. The insoluble residue (triethylammonium salt by NMR) was filtered off and the filtrate was concentrated in vacuo until precipitate. The suspension (ca. 130 mL) was heated to 50° C. and treated with n-heptane (50 mL). The turbid mixture was stirred at 60° C. for 30 min and slowly cooled to rt over 12 h. The flask wall was scratched to release the solid, and stirred at rt for another 1 h. The precipitate was filtered, washed with n-heptane and dried over N₂ purge to give the title compound (7.48 g, 66%) as an off-white solid.

1H NMR (400 MHz, CHLOROFORM-d) δ=7.57-7.52 (m, 2H), 7.52-7.47 (m, 2H), 4.46-4.34 (m, 1H), 4.28-4.13 (m, 1H), 3.34-3.20 (m, 1H), 2.69 (qd, J=6.0, 13.9 Hz, 1H). 31P NMR (162 MHz, CHLOROFORM-d) δ=111.81 (s, 1P).

LRMS (ESI) m/z: [M+H]+ Calcd for C8H9BrOPS3 328.9; Found 328.8

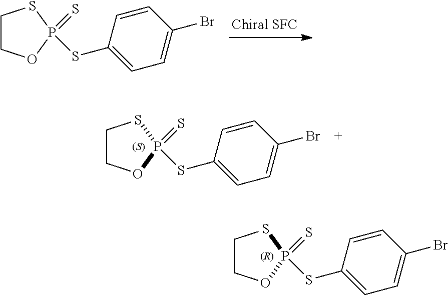

### (S)-2-((4-bromophenyl)thio)-1,3,2-oxathiaphospholane 2-sulfide and (R)-2-((4-bromophenyl)thio)-1,3,2-oxathiaphospholane 2-sulfide

2-((4-bromophenyl)thio)-1,3,2-oxathiaphospholane 2-sulfide (10.4 g) was subjected to preparative chiral SFC separation to give (S)-2-((4-bromophenyl)thio)-1,3,2-oxathiaphospholane 2-sulfide (4.70 g, 100% ee, retention time: 3.30 min) and (R)-2-((4-bromophenyl)thio)-1,3,2-oxathiaphospholane 2-sulfide (4.32 g, 99.8% ee, retention time: 3.66 min).

For (S)-isomer:

1H NMR (400 MHz, CHLOROFORM-d) δ=7.55 (d, J=8.3 Hz, 2H), 7.49 (br d, J=7.5 Hz, 2H), 4.40 (dtd, J=4.9, 9.1, 13.9 Hz, 1H), 4.27-4.14 (m, 1H), 3.35-3.21 (m, 1H), 2.74-2.63 (m, 1H).

31P NMR (162 MHz, CHLOROFORM-d) δ=111.81 (s, 1P).

LRMS (ESI) m/z: [M+HCO2]− Calcd for C9H9BrO3PS3 372.9; Found 373.0

For (R)-isomer:

1H NMR (400 MHz, CHLOROFORM-d) δ=7.55 (d, J=8.3 Hz, 2H), 7.49 (br d, J=8.5 Hz, 2H), 4.45-4.35 (m, 1H), 4.27-4.14 (m, 1H), 3.37-3.19 (m, 1H), 2.77-2.61 (m, 1H).

31P NMR (162 MHz, CHLOROFORM-d) δ=111.81 (s, 1P).

LRMS (ESI) m/z: [M+HCO2]− Calcd for C9H9BrO3PS3 372.9; Found 372.8
Resolution by chiral SFC: purification method
Column: Chiralpak IC—H, 21×250 mm, 5μ
Flowrate: 70 mL/min
Mobile Phase: 20% isopropanol in CO2
Gradient: Isocratic
Injection Volume: 1 mL (500 mg of racemic compound was dissolved in a mixture of 15 mL
isopropanol and 15 mL CH2Cl2)
Detection: 220 nm
   Analytical chiral SFC
Column: Chiralpak IC—H, 4.6×100 mm, 5μ
Flowrate: 2.5 mL/min
Mobile Phase: 20% isopropanol in CO2
Gradient: Isocratic
Injection Volume: 5 uL (1 mg/mL)
Detection: 220 nm

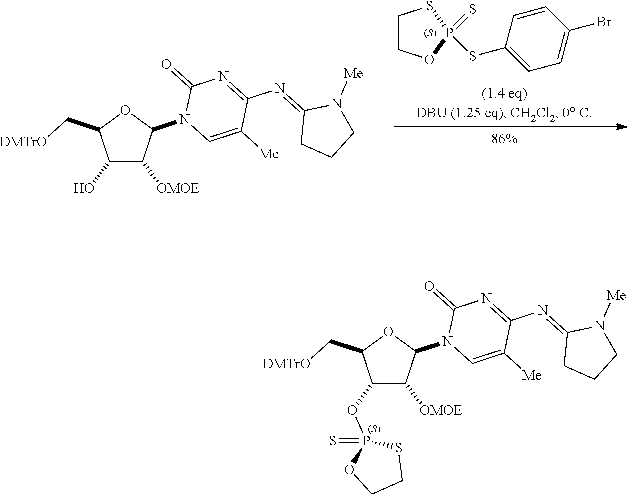

### 1-((2R,3R,4R,5R)-5-((bis(4-methoxyphenyl)(phenyl)methoxy)methyl)-3-(2-methoxy ethoxy)-4-(((S)-2-sulfido-1,3,2-oxathiaphospholan-2-yl)oxy)tetrahydrofuran-2-yl)-5-methyl-4-(((E)-1-methylpyrrolidin-2-ylidene)amino)pyrimidin-2(1H)-one

1-((2R,3R,4R,5R)-5-((bis(4-methoxyphenyl)(phenyl)methoxy)methyl)-4-hydroxy-3-(2-methoxy ethoxy)tetrahydrofuran-2-yl)-5-methyl-4-(((E)-1-methylpyrrolidin-2-ylidene)amino) pyrimidin-2(1H)-one (200 mg, 0.286 mmol) was azeotroped twice with acetonitrile (6 mL). The residue was dissolved in CH₂Cl₂ (3 mL), treated with (S)-2-((4-bromophenyl)thio)-1,3,2-oxathiaphospholane 2-sulfide (131 mg, 0.401 mmol), cooled to 0° C., and treated with DBU (0.056 mL, 0.37 mmol). After stirring at 0° C. for 3 h, the reaction was quenched with 10% sodium dihydrogen phosphate (3 mL) and diluted with CH₂Cl₂ (3 mL). The organic layer was separated, and the aqueous layer was extracted twice with ethyl acetate (5 mL). The organic layers were combined, washed with sat. NaHCO₃ (5 mL) and brine (5 mL), dried over MgSO₄, and concentrated in vacuo. The residue was purified by column chromatography (EA in hep=20% and 100% and then THF in EA=0% to 100%) to give the title compound (205 mg, 86%).

¹H NMR (400 MHz, CHLOROFORM-d) δ=7.76 (s, 1H), 7.47 (br d, J=7.8 Hz, 2H), 7.37 (br d, J=6.3 Hz, 4H), 7.33-7.28 (m, 2H), 7.25-7.18 (m, 1H), 6.84 (br d, J=8.5 Hz, 4H), 6.14 (s, 1H), 5.37-5.26 (m, 1H), 4.60-4.47 (m, 1H), 4.45-4.31 (m, 2H), 4.26 (br s, 1H), 4.17-4.04 (m, 1H), 3.87 (br s, 1H), 3.79 (s, 6H), 3.63-3.53 (m, 3H), 3.48-3.35 (m, 5H), 3.34 (s, 3H), 3.29-3.09 (m, 2H), 3.03 (s, 3H), 2.11-1.98 (m, 2H), 1.47 (s, 3H).

³¹P NMR (162 MHz, CHLOROFORM-d) δ=104.79 (s, 1P).

LRMS (ESI) m/z: [M+H]+ Calcd for C41H50N4O9PS2 837.3; Found 837.1

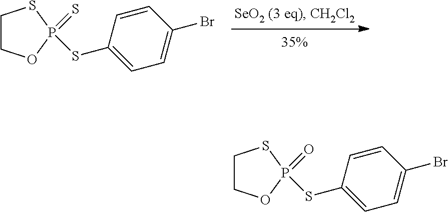 [SMILES: S=P1(Sc2ccc(Br)cc2)OCCS1 ; O=P1(Sc2ccc(Br)cc2)OCCS1]

2-((4-Bromophenyl)thio)-1,3,2-oxathiaphospholane 2-oxide. A solution of 2-((4-bromophenyl)thio)-1,3,2-oxathiaphospholane 2-sulfide (1.17 g, 3.56 mmol) in CH₂Cl₂ (23 mL) was treated with selenium dioxide (0.395 g, 3.56 mmol) and stirred at rt for 2.5 h. Two additional selenium dioxide (0.395 g, 3.56 mmol) was added and stirring was continued at rt for 25 h. The reaction mixture was filtered though a dry silica gel (5 g) and washed with CH₂Cl₂. The filtrate was washed with 10% NaH₂PO₄ (12 mL), dried over MgSO₄, and concentrated in vacuo. The residue was treated with n-heptane (13 mL) and MTBE (2 mL). The sticky solid was scratched and the resulting slurry was stirred at rt for 20 min. The precipitate was filtered, washed with n-heptane, and derived over N₂ purge to give the title compound (388 mg, 35%) as a yellow solid.

¹H NMR (400 MHz, CHLOROFORM-d) δ=7.53 (s, 4H), 7.38 (s, 1H), 4.49-4.32 (m, 1H), 4.11-3.95 (m, 1H), 3.45-3.23 (m, 1H), 2.88-2.70 (m, 1H), 1.25 (s, 1H), 0.88 (br s, 1H).

³¹P NMR (162 MHz, CHLOROFORM-d) δ=66.26 (s, 1P).

LRMS (ESI) m/z: [M+H]+ Calcd for C8H9BrO2PS2 310.9; Found 310.8

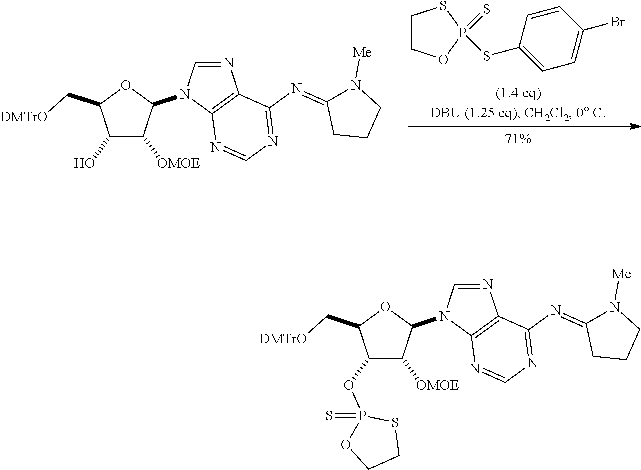

### 2-(((2R,3R,4R,5R)-2-((bis(4-methoxyphenyl)(phenyl)methoxy)methyl)-4-(2-methoxyethoxy)-5-(4-(((E)-1-methylpyrrolidin-2-ylidene)amino)-7H-pyrrolo[2,3-d]pyrimidin-7-yl)tetrahydrofuran-3-yl)oxy)-1,3,2-oxathiaphospholane 2-oxide

(2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-ol (3.0 g, 4.2 mmol) was azeotroped twice with acetonitrile (28 mL). The residue was dissolved in CH₂Cl₂ (60 mL), treated with 2-((4-bromophenyl)thio)-1,3,2-oxathiaphospholane 2-oxide (1.84 g, 5.93 mmol), cooled to 0° C. and treated with DBU (0.797 mL, 5.291 mmol). After stirring at 0° C. for 3 h, the reaction was quenched with 10% sodium dihydrogen phosphate (45 mL) and diluted with ethyl acetate (45 mL). The organic layer was separated, and the aqueous layer was extracted with ethyl acetate (45 mL). The combined organic layer was washed with sat. NaHCO₃ (45 mL) and brine (20 mL), dried over MgSO₄, and concentrated in vacuo. The residue was purified by column chromatography (EA in hep=20% to 100%, and then THF in EA=0% to 100%) to give the title compound (2.506 g, 71.3%) as a off-white foam.

¹H NMR (400 MHz, CHLOROFORM-d) δ=8.48 (s, 1H), 8.06 (two s, 1H), 7.41 (br d, J=7.5 Hz, 2H), 7.31 (br d, J=8.5 Hz, 4H), 7.28-7.17 (m, 3H), 6.79 (two d, 4H), 6.17 (two d, J=6.5, 7.0 Hz, 1H), 5.36-5.24 (m, 1.5H), 5.13 (t, J=5.63 Hz, 0.5H), 4.47-4.41 (m, 1H), 4.41-4.27 (m, 2H), 3.92-3.84 (m, 1H), 3.78 (s, 6H), 3.75-3.67 (m, 1H), 3.57-3.41 (m, 7H), 3.41-3.32 (m, 1H), 3.22 (s, 1.5H), 3.15 (s, 4.5H), 3.03-2.89 (m, 2H), 2.07 (m, 2H).

³¹P NMR (162 MHz, CHLOROFORM-d) δ=45.49 (0.16P), 45.46 (s, 1P), 45.05 (0.07P), 44.94 (s, 1P).

LRMS (ESI) m/z: [M+H]+ Calcd for C41H₄₈N₆₀₉PS 831.3; Found 831.2 Example 16B: Preparation of (R) and (S)-2-((4-nitrophenyl)thio)-1,3,2-oxathiaphospholane 2-sulfide

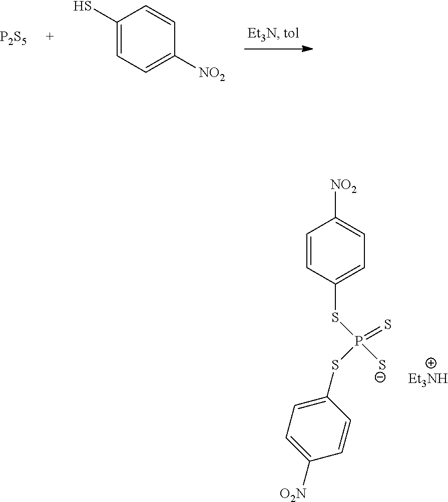 [SMILES: "P2S5" ; "Et3NH" ; O=[N+]([O-])c1ccc(S)cc1 ; O=[N+]([O-])c1ccc(SP(=S)([S-])Sc2ccc([N+](=O)[O-])cc2)cc1]

Triethylammonium bis(4-nitrophenyl) phosphorotetrathioate. A mixture of phosphorus(V) sulfide (20.0 g, 90.0 mmol) and 4-nitrothiophenol (27.9 g, 180 mmol) in toluene (160 mL) was purged with nitrogen gas for 5 min and treated with triethylamine (26.3 mL, 189 mmol) over 30 min maintaining the internal temperature below 43° C. After stirring at rt for 23 h, the solution was removed by decantation and the sticky solid residue was dissolved in methanol (120 mL).

The mixture was treated with n-heptane (120 mL), stirred at rt for 20 min, and treated with water (100 mL). After stirring at rt for 1 h, the precipitate was transferred to a glass filter with a 2:3 mixture of methanol and water (total 120 mL), washed twice with water (60.0 mL) and twice with n-heptane (30.0 mL). The filter cake was dried under vacuum at 50° C. for 1 d to give the title compound (40 g, 88%) as an off-white solid.

¹H NMR (400 MHz, CHLOROFORM-d) δ=9.06 (br s, 1H), 8.21 (d, J=8.8 Hz, 4H), 7.88-7.84 (m, 4H), 3.32-3.22 (m, 6H), 1.41 (t, J=7.3 Hz, 9H).

³¹P NMR (162 MHz, CHLOROFORM-d) δ=96.38 (s, 1P).

LRMS (ESI) m/z: [M−H]− Calcd for C12H8N2O4PS4 402.9; Found 402.9

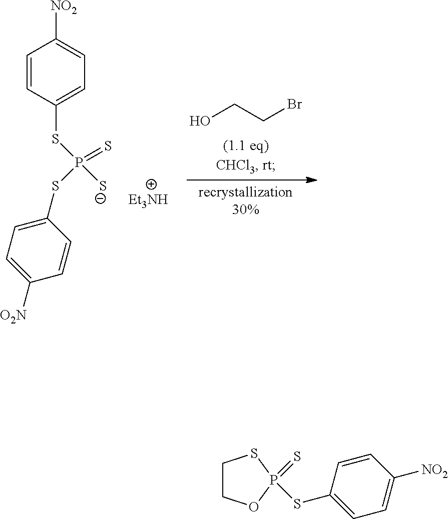

2-((4-Nitrophenyl)thio)-1,3,2-oxathiaphospholane 2-sulfide. A solution of triethylamine bis(4-nitrophenyl) phosphorotetrathioate (20.0 g, 40.0 mmol) and 2-bromoethan-1-ol (3.09 mL, 43.5 mmol) in chloroform (180 mL) was stirred at rt for 21 h. The solution was decanted and concentrated in vacuo to ca. 60 mL. While stirring, the residue was treated with water (56 mL) and then with n-heptane (56 mL). The mixture was cooled with an ice bath and stirred at 0° C. for 1 h. The precipitate was filtered, and washed with water (44 mL) and n-heptane (44 mL). The filter cake (17.7 g) was dissolved with CH₂Cl₂ (64 mL) and the insoluble solid was filtered off. The filtrate was treated with n-heptane (100 mL), stirred at rt for 20 min, and concentrated in vacuo to a half volume. The resulting precipitate was filtered, washed with n-heptane (5 mL), and dried over N₂ purge for 2 h to give a crude product (10.7 g) as a yellow solid.

The crude product was dissolved in a mixture of ethyl acetate (100 mL) and CH₂Cl₂ (20 mL) with heating and filtered through a glass filter to remove the insoluble solid. The filtrate was heated to 60° C., and treated with n-heptane (20 mL). The resulting solution was stirred at 60° C. for 20 min, slowly cooled to 45° C. over 2 h and stirred at rt for 3 h. The resulting precipitate was filtered, washed with a 2:1 mixture of n-heptane/EtOAc, and dried over N₂ purge to give the title compound (4.8 g, 72 wt %, 29.8%) as an off-white solid.

¹H NMR (400 MHz, CHLOROFORM-d) δ=8.26 (d, J=8.5 Hz, 2H), 7.80 (dd, J=2.0, 8.8 Hz, 2H), 4.57-4.45 (m, 1H), 4.41-4.27 (m, 1H), 3.49-3.37 (m, 1H), 3.04-2.93 (m, 1H).

³¹P NMR (162 MHz, CHLOROFORM-d) δ=109.53 (s, 1P).

LRMS (ESI) m/z: [M+H]+ Calcd for C8H9NO3PS3 293.9; Found 294.0

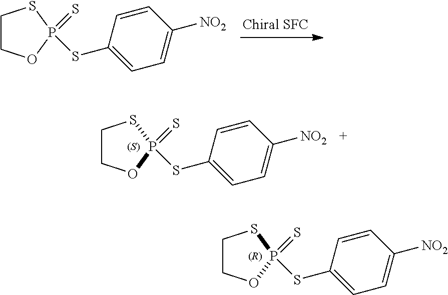

(R)-2-((4-nitrophenyl)thio)-1,3,2-oxathiaphospholane 2-sulfide and (S)-2-((4-nitrophenyl)thio)-1,3,2-oxathiaphospholane 2-sulfide. 2-((4-nitrophenyl)thio)-1,3,2-oxathiaphospholane 2-sulfide (303.9 mg) was subjected to a preparative chiral SFC separation to give (S)-2-((4-nitrophenyl)thio)-1,3,2-oxathiaphospholane 2-sulfide (99.1 mg, 100% ee, retention time: 2.27 min) and (R)-2-((4-nitrophenyl)thio)-1,3,2-oxathiaphospholane 2-sulfide (96.1 mg, 94.0% ee, retention time: 2.45 min).
For (S)-isomer:

¹H NMR (400 MHz, CHLOROFORM-d) δ=8.26 (d, J=8.5 Hz, 2H), 7.80 (dd, J=2.0, 8.8 Hz, 2H), 4.57-4.45 (m, 1H), 4.41-4.27 (m, 1H), 3.49-3.37 (m, 1H), 3.04-2.93 (m, 1H).

³¹P NMR (162 MHz, CHLOROFORM-d) δ=109.50 (s, 1P).

For (R)-isomer:

¹H NMR (400 MHz, CHLOROFORM-d) δ=8.26 (d, J=8.5 Hz, 2H), 7.80 (dd, J=2.0, 8.8 Hz, 2H), 4.57-4.45 (m, 1H), 4.41-4.27 (m, 1H), 3.49-3.37 (m, 1H), 3.04-2.93 (m, 1H).

³¹P NMR (162 MHz, CHLOROFORM-d) δ=109.50 (s, 1P).

### Resolution by Chiral SFC: Purification Method

Column: ChiralCel OD-H, 21×250 mm
Flowrate: 70 mL/min
Mobile Phase: 25% methanol in CO₂
Gradient: Isocratic
Injection Volume: 0.5 mL (304 mg of racemic compound was dissolved in a mixture of 10 mL
methanol and 10 mL CH₂Cl₂)
Detection: 220 nm

### Analytical Chiral SFC

Column: ChiralCel OD-H, 4.6×100 mm
Flowrate: 2.5 mL/min
Mobile Phase: 25% methanol in CO₂
Gradient: Isocratic
Injection Volume: 5 uL
Detection: 220 nm

### Example 16C: Preparation of (R) and (S)-2-((perfluorophenyl)thio)-1,3,2-oxathiaphospholane 2-sulfide

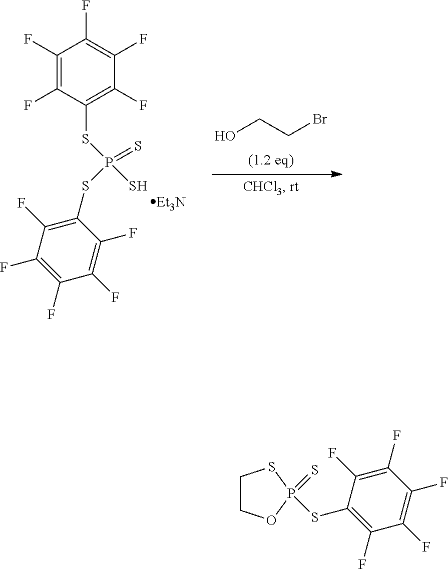

2-((perfluorophenyl)thio)-1,3,2-oxathiaphospholane 2-sulfide. A solution of triethylamine bis(perfluorophenyl) phosphorotetrathioate (15.0 g, 25.2 mmol; prepared using literature procedure reported in Science, 2018, 361, 1234) and 2-bromoethan-1-ol (2.1 mL, 30.2 mmol) in chloroform (120 mL) was stirred at rt for 21 h. n-Heptane (120 mL) was added to the reaction mixture, stirred at rt for 1 h, filtered the solids through a pad of Celite. The filtrate was dried over Na₂SO₄, filtered, concentrated and purified by using silica gel column chromatography (0 to 100% EtOAc/n-heptane) to give 2-((perfluorophenyl)thio)-1,3,2-oxathiaphospholane 2-sulfide (3.8 g, 11.2 mmol, 44%) as viscous oil (solidified at low temperature).

¹H NMR (400 MHz, AETRONITRILE-d₃) δ=4.64-4.51 (m, 1H), 4.48-4.34 (m, 1H), 3.68-3.54 (m, 1H), 3.33-3.23 (m, 1H).

³¹P NMR (162 MHz, AETRONITRILE-d₃) δ=109.6 (m, 1P).

LRMS (ESI) m/z: [M+H]+ Calcd for C8H5F50PS3 338.9; Found 338.8

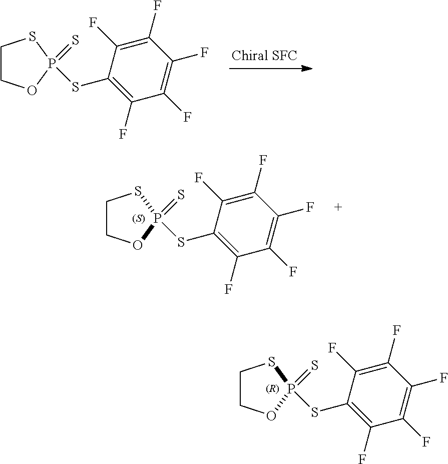

### (S)-2-((perfluorophenyl)thio)-1,3,2-oxathiaphospholane 2-sulfide and (R)-2-((perfluorophenyl)thio)-1,3,2-oxathiaphospholane 2-sulfide

2-((perfluorophenyl)thio)-1,3,2-oxathiaphospholane 2-sulfide (531.5 mg) was subjected to a preparative chiral SFC separation to give (S)-2-((perfluorophenyl)thio)-1,3,2-oxathiaphospholane 2-sulfide (173.3 mg, 100% ee, retention time: 1.86 min) and (R)-2-((perfluorophenyl)thio)-1,3,2-oxathiaphospholane 2-sulfide (174.6 mg, 94.1% ee, retention time: 2.16 min).

For (S)-isomer:

¹H NMR (400 MHz, AETRONITRILE-d₃) δ=4.64-4.50 (m, 1H), 4.49-4.35 (m, 1H), 3.68-3.52 (m, 1H), 3.37-3.21 (m, 1H).

³¹P NMR (162 MHz, AETRONITRILE-d₃) δ=109.6 (m, 1P).

For (R)-isomer:

¹H NMR (400 MHz, AETRONITRILE-d₃) δ=4.65-4.51 (m, 1H), 4.48-4.35 (m, 1H), 3.66-3.54 (m, 1H), 3.34-3.22 (m, 1H).

³¹P NMR (162 MHz, AETRONITRILE-d₃) δ=109.6 (m, 1P).

### Resolution by Chiral SFC: Purification Method

Column: ChiralCel OD-H, 21×250 mm
Flowrate: 60 mL/min
Mobile Phase: 10% Isopropanol in C02
Gradient: Isocratic
Injection Volume: 0.5 mL (531.5 mg of racemic compound was dissolved in 20 mL Isopropanol)
Detection: 220 nm

### Analytical Chiral SFC

Column: ChiralCel OD-H, 4.6×100 mm
Flowrate: 2.5 mL/min
Mobile Phase: 10% Isopropanol in CO₂
Gradient: Isocratic
Injection Volume: 5 uL
Detection: 220 nm

### Example 16D: Preparation of 2-(dimethylamino)-1,3,2-oxathiaphospholane 2-sulfide (2-(dimethylamino)-1,3,2-oxathiaphospholane 2-sulfide) from 2-((perfluorophenyl)thio)-1,3,2-oxathiaphospholane 2-sulfide

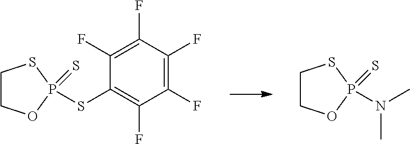

A solution of 2-((perfluorophenyl)thio)-1,3,2-oxathiaphospholane 2-sulfide (2.6 g, 7.68 mmol) in dimethylamine (ca 8% in acetonitrile solution) (15 mL) was stirred at 0° C. for 2 h. The reaction mixture was concentrated under reduced pressure and the crude residue was purified by silica gel column chromatography (0 to 100% EtOAc/n-heptane) to give 2-(dimethylamino)-1,3,2-oxathiaphospholane 2-sulfide (0.83 g, 4.53 mmol, 59% yield) as clear oil (solidified at lower temperature).

¹H NMR (400 MHz, CHLOROFORM-d) δ=4.55-4.42 (m, 1H), 4.38-4.30 (m, 1H), 3.56-3.40 (m, 2H), 2.90 (s, 3H), 2.86 (s, 3H).

³¹P NMR (162 MHz, CHLOROFORM-d) 06=102.1 (s, 1P).

### Example 16E: Preparation of (R)-2-(dimethylamino)-1,3,2-oxathiaphospholane 2-sulfide from (S)-2-((perfluorophenyl)thio)-1,3,2-oxathiaphospholane 2-sulfide from Example 14B

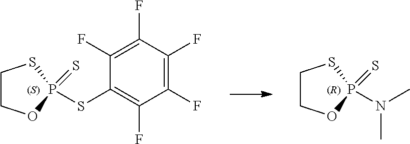

(R)-2-(dimethylamino)-1,3,2-oxathiaphospholane 2-sulfide. A solution of (S)-2-((perfluorophenyl)thio)-1,3,2-oxathiaphospholane 2-sulfide (171 mg, 0.51 mmol, 100% ee) in dimethylamine (ca 8% in acetonitrile solution) (2 mL, 39.48 mmol) was stirred at 0° C. for 2 h. The reaction mixture was directly concentrated under reduced pressure, and the crude residue was purified by silica gel column chromatography (0 to 100% EtOAc/n-heptane) to give (R)-2-(dimethylamino)-1,3,2-oxathiaphospholane 2-sulfide (48 mg, 0.26 mmol, 52% yield, 98% ee) as clear oil.

¹H NMR (400 MHz, CHLOROFORM-d) δ=4.55-4.42 (m, 1H), 4.38-4.30 (m, 1H), 3.56-3.40 (m, 2H), 2.90 (s, 3H), 2.87 (s, 3H).

³¹P NMR (162 MHz, CHLOROFORM-d) δ=102.1 (s, 1P).

The foregoing are illustrative of embodiments presented herein, and is not to be construed as limiting thereof. The invention is defined by the following claims, with equivalents of the claims to be included therein.

Documents mentioned in this disclosure are incorporated by reference as if fully rewritten herein. If there is any conflict between an incorporated document and something written in this disclosure, this disclosure controls.

1. A stereo-encoded compound that is a morpholino monomer of Formula (I) 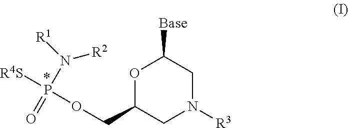
or a diastereomer or enantiomer thereof, or a salt thereof, wherein
P* represents a stereocenter that is either in the (R) or (S) configuration;
R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle;
R³ is a protecting group for morpholino nitrogen;
R⁴ is hydrogen or C1-C6 alkyl,
Base is selected from the group consisting of: 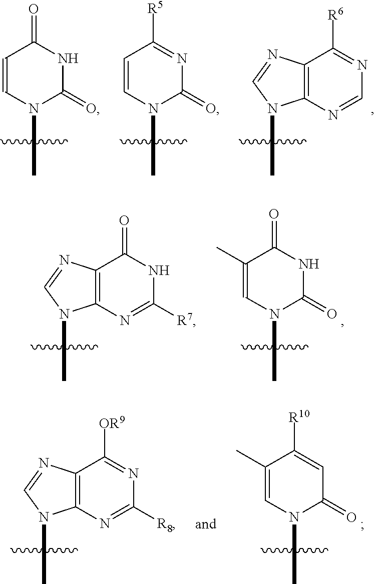
R⁵, R⁶, R⁷, R⁸, and R¹⁰ are independently —NR²⁰R²¹ or —N═CR¹²—NR¹³R¹⁴;
where any of R⁵, R⁶, R⁷, R⁸, and R¹⁰ is independently —NR²⁰R²¹, R²⁰ and R²¹ are independently selected from —H, —C(O)R¹¹ and —C(O)OR¹¹;
R¹¹ is optionally substituted C1-C6 alkyl, optionally substituted benzyl, or aryl, optionally substituted with one or more substituents selected from halogen, nitro and alkoxyl;
when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl;
when any of R⁵, R⁶, R⁷, R⁸, and R¹⁰ is independently —NHC(O)OR¹¹, R¹¹ is optionally substituted C1-C6 alkyl, optionally substituted benzyl, 2,2,2-trichloroethyl, or optionally substituted aryl;
R¹², R¹³, and R¹⁴ are independently C1-C6 alkyl, or R¹² and R¹³ together form a 5 or 6-membered heterocycle with the adjacent C and N; and
R⁹ is H, optionally substituted C1-C6 alkyl, optionally substituted benzyl, acyl, carbonate, carbamate, or silyl.

2. The compound of claim 1, wherein the compound is crystalline.

3. The compound of claim 1, wherein R¹ and R² are independently optionally substituted C1-C6 alkyl.

4. The compound of claim 1, wherein R¹ and R² are methyl.

5. The compound of claim 1, wherein R¹ and R² together with the nitrogen they connect to form a heterocycle selected from morpholinyl, piperazinyl, pyrrolidinyl, and azetidinyl.

6. The compound of claim 1, wherein R³ is selected from optionally substituted C1-C6 alkyl, trityl, benzyl, and sulfonyl.

7. The compound of claim 1, wherein R³ is selected from the group consisting of trityl, p-methoxyphenyldiphenylmethyl, benzyl, methoxybenzyl, dimethoxybenzyl, diphenylmethyl, nitrobenzenesulfonyl, and dinitrobenzenesulfonyl.

8. The compound of claim 1, wherein R⁴ is H or methyl.

9. The compound of claim 1, wherein R¹ and R² are methyl, R³ is trityl, and R⁴ is H or methyl.

10. The compound of claim 1, wherein any of R⁵, R⁶, R⁷, R³ and R¹⁰ is independently —NHBz or —NHC(O)iPr.

11. The compound of claim 1, wherein any of R⁵, R⁶, R⁷, R³ and R¹⁰ is independently

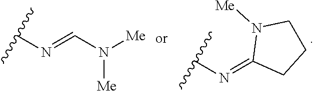 [SMILES: */N=C/N(C)C ; */N=C1/CCCN1C]

12. The compound of claim 1, wherein R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-dimethylbenzyl, 3,4-dimethylbenzyl,2,6-dimethylbenzyl, 4-methoxybenzyl, and 4-pivaloyloxy benzyl.

13. The compound of claim 1, selected from the following compounds: 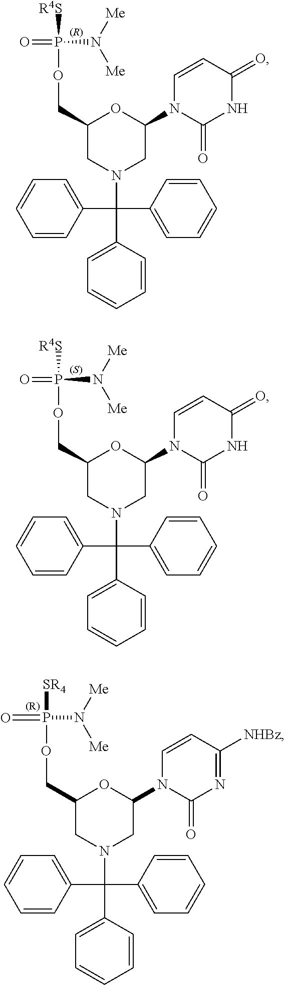 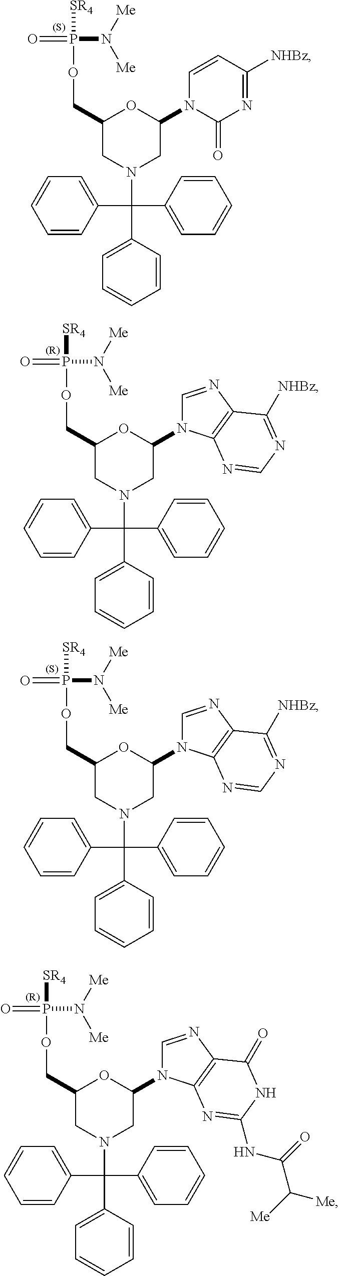 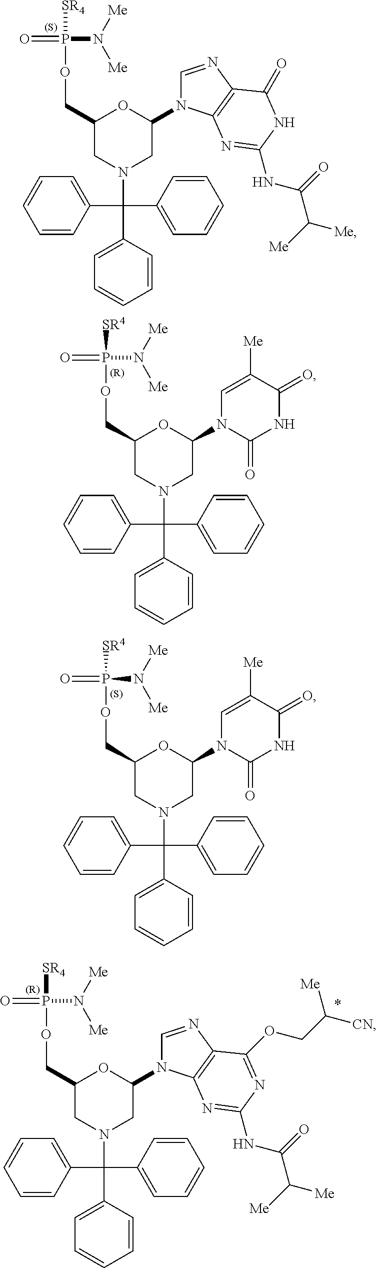 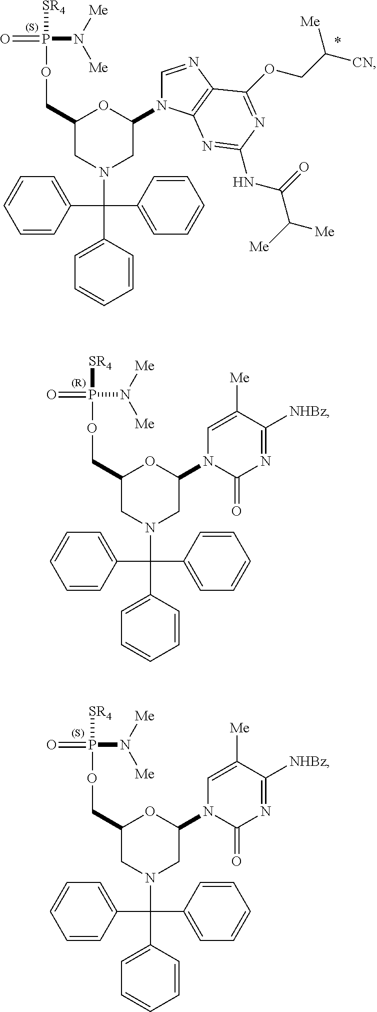 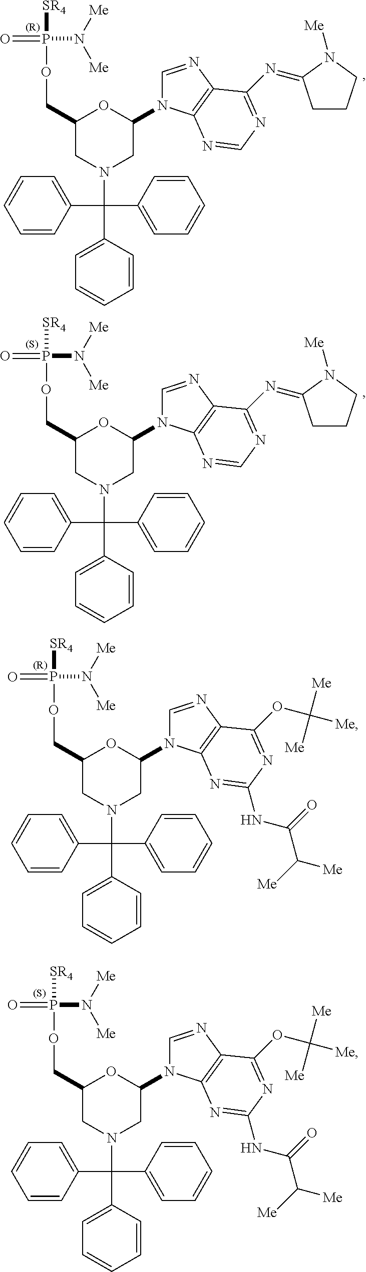 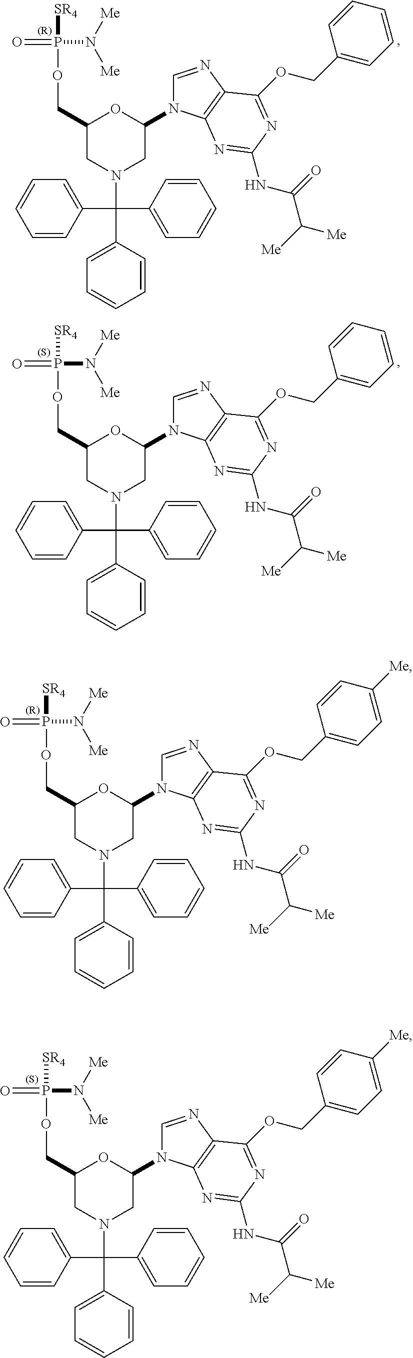 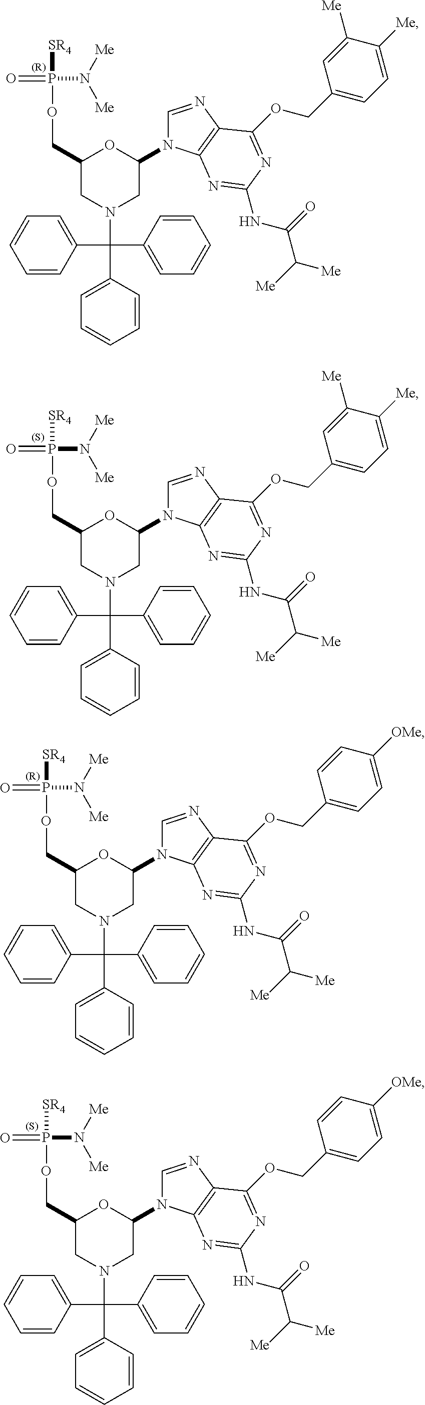 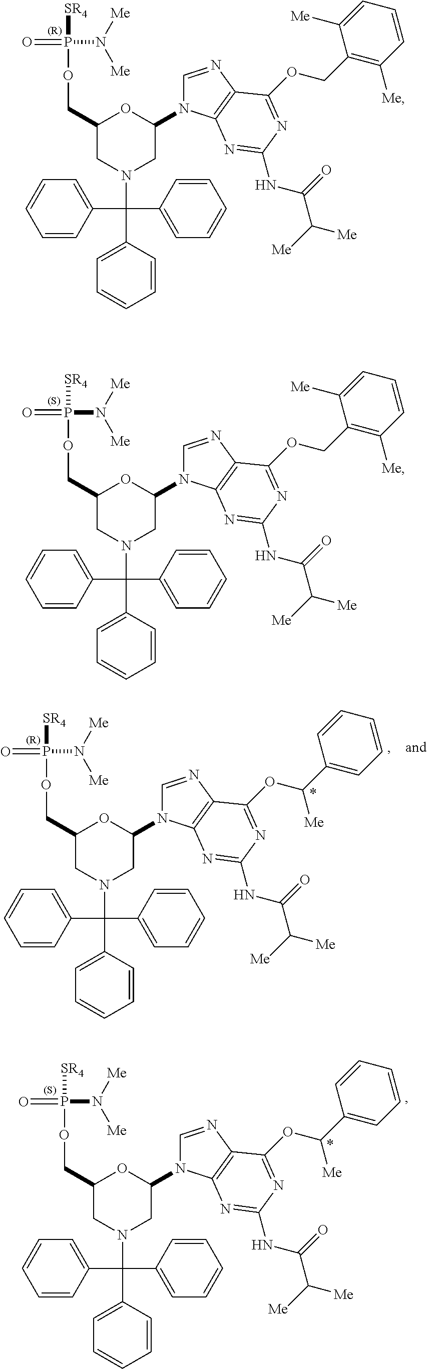
wherein * indicates either (R) or (S) stereochemistry, or a mixture thereof, and
R⁴ is H or methyl.

14. A method of preparing a stereo-encoded activated monomer of Formula (II) 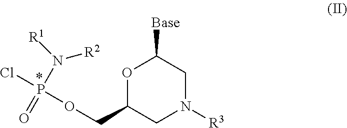
or a diastereomer or enantiomer thereof, or a salt thereof, wherein
P* represents a stereocenter that is either in the (R) or (S) configuration;
R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle;
R³ is a protecting group for morpholino nitrogen, selected from optionally substituted C1-C6 alkyl, trityl, benzyl, or sulfonyl;
Base is selected from the group consisting of: 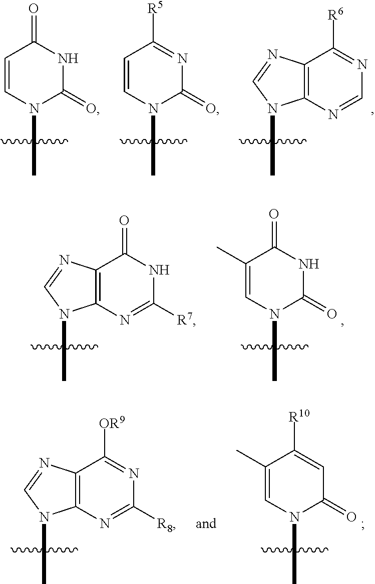
R⁵, R⁶, R⁷, R⁸, and R¹⁰ are independently —NR²⁰R²¹ or —N═CR¹²—NR¹³R¹⁴;
where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹,
when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl;
when any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl;
R¹², R¹³, and R¹⁴ are independently C1-C6 alkyl, or R¹² and R¹³ together form a 5 or 6-membered heterocycle with the adjacent C and N;
R⁹ is H, optionally substituted C1-C6 alkyl, optionally substituted benzyl, acyl, carbonate, carbamate, or silyl;
comprising the following steps:
(a) providing a stereo-encoded morpholino monomer of Formula (Ia) 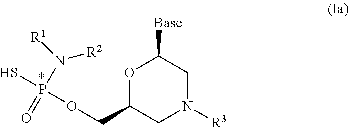
(b) optionally alkylating the sulfur of the stereo-encoded morpholino monomer of Formula (Ia) in step (a) to afford a stereo-encoded morpholino monomer of Formula (I) 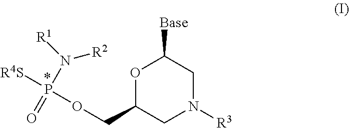
wherein R⁴ is C1-C6 alkyl;
(c) reacting the stereo-encoded morpholino monomer of Formula (Ia) from step (a) or the stereo-encoded morpholino monomer of Formula (I) from step (b) with a chlorinating agent to afford the stereo-encoded activated monomer of Formula (II).

15. The method of claim 14, wherein R⁴ in step (b) is methyl.

16. The method claim 14, wherein R¹ and R² are independently C1-C6 alkyl.

17. The method of claim 14, wherein R¹ and R² are methyl.

18. The method of claim 14, wherein R¹ and R², together with the nitrogen to which they connect, form a heterocycle selected from morpholinyl, piperazinyl, pyrrolidinyl, and azetidinyl.

19. The method of claim 14, wherein R³ is selected from the group consisting of trityl, p-methoxyphenyldiphenylmethyl, benzyl, methoxybenzyl, dimethoxybenzyl, diphenylmethyl, nitrobenzenesulfonyl, and dinitrobenzenesulfonyl.

20. The method of claim 14, wherein R³ is trityl.

21. The method of claim 14, wherein R¹ and R² are methyl, R³ is trityl and step (b) is absent, or wherein and R¹ and R² are methyl, R³ is trityl, and R⁴ is methyl in step (b).

22. The method of claim 14, wherein any of R⁵, R⁶, R⁷ R⁸ and R¹⁰ is independently —NHBz or —NHC(O)iPr.

23. The method of claim 14, wherein any of R⁵, R⁶, R⁷ R⁸ and R¹⁰ is independently

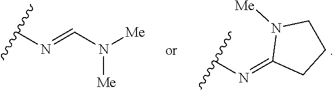 [SMILES: */N=C/N(C)C ; */N=C1/CCCN1C]

24. The method of claim 14, wherein R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl.

25. The method of claim 14, wherein the chlorinating agent is SO₂Cl₂ or tetramethyl chloroenamine.

26. The method of claim 25, wherein step (c) is conducted in the presence of a base.

27. The method of claim 14 wherein step (b) is absent and the chlorinating agent is tetramethyl chloroenamine.

28. A stereo-encoded activated monomer prepared by the method according to claim 14.

29. A method for preparing a stereo-encoded morpholino monomer of Formula (I) 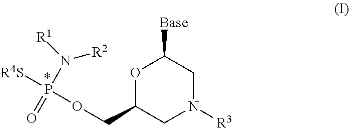
or a diastereomer or enantiomer thereof, or a salt thereof, wherein
P* represents a stereocenter that is either in the (R) or (S) configuration;
R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle;
R³ is a protecting group for morpholino nitrogen, selected from optionally substituted C1-C6 alkyl, trityl, benzyl, or sulfonyl;
R⁴ is hydrogen or C1-C6 alkyl,
Base is selected from the group consisting of: 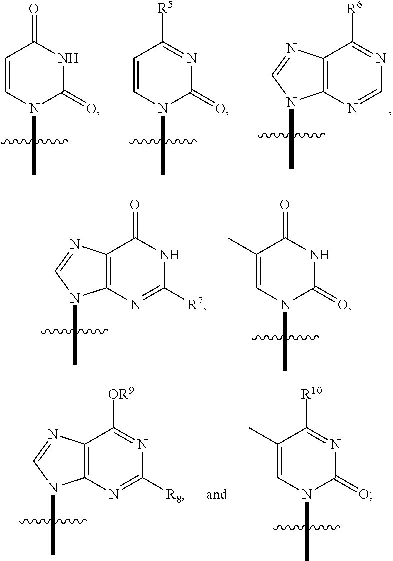
R⁵, R⁶, R⁷, R⁸, and R¹⁰ are independently —NR²⁰R²¹ or —N═CR¹²—NR¹³R¹⁴;
where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹;
when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl;
when any of R⁵, R⁶, R⁷, R⁸, and R¹⁰ is independently-NHC(O)OR¹¹, R¹¹ is C1-C6 alkyl, benzyl, 2,2,2-trichloroethyl, or optionally substituted aryl;
R¹², R¹³, and R¹⁴ are independently C1-C6 alkyl, or R¹² and R¹³ together form a 5 or 6-membered heterocycle with the adjacent C and N;
R⁹ is H, optionally substituted C1-C6 alkyl, optionally substituted benzyl, acyl, carbonate, carbamate, cyanoethyl, α-methylcyanoethyl, β-methylcyanoethyl, pivaloyloxy benzyl, or silyl;
comprising the step of
reacting a compound of Formula (III) 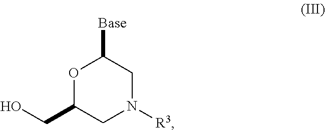
wherein R³ and Base are as defined above,
with a chiral reagent of Formula (IV) 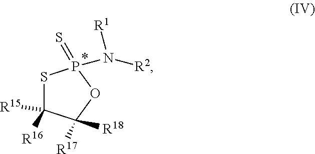
wherein R¹ and R² are as defined above;
R¹⁵, R¹⁶, R¹⁷ and R¹⁸ are independently H, C1-C10 alkyl, C2-C12 alkenyl, aryl
or any two of R¹⁵, R¹⁶, R¹⁷ and R¹⁸ together with the carbons they are attached form a cycloalkyl or heteroalkyl ring which is optionally substituted with one or more C1-C10 alkyl or C2-C12 alkenyl groups,
in the presence of a base to obtain the stereo-encoded morpholino monomer of Formula (I).

30. The method of claim 29, wherein R¹ and R² are methyl.

31. The method of claim 29, wherein R¹⁵, R¹⁶, R¹⁷ and R¹⁸ are independently H, C1-C10 alkyl, or aryl.

32. The method of claim 29, wherein
R¹⁵, R¹⁶, R¹⁷ and R¹⁸ are H, or
R¹⁵ is C1-C10 alkyl or aryl, and R¹⁶, R¹⁷, R¹⁸ are H, or
R¹⁶ is C1-C10 alkyl or aryl, and R¹⁵, R¹⁷, R¹⁸ are H; or
R¹⁵ is phenyl and R¹⁶, R¹⁷, R¹⁸ are H, or
R¹⁶ is phenyl, and R¹⁵, R¹⁷, R¹⁸ are H.

33. The method of claim 29, wherein the chiral agent of Formula (IV) is selected from

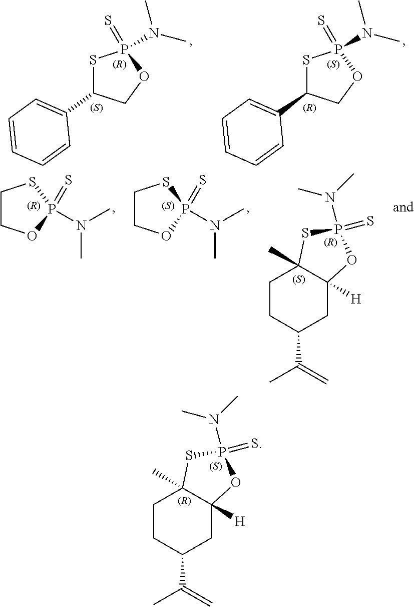

34. The method of claim 29, wherein the base is selected from the group consisting of NaH, DBU, sodium tert-amyulate, sodium tert-pentoxide, NaOtBu, KOtBu, potassium tert-pentoxide, and NaHMDS.

35. The method of claim 29, wherein the reaction is conducted at room temperature in a polar solvent selected from the group consisting of THF, acetonitrile, 2-MeTHF, 1,6-dioxane, and DME.

36. The method of claim 29, wherein the stereo-encoded morpholino monomer of Formula (I) is crystalline.

37. The method of claim 29, wherein the de/ee ratio/stereoselectivity is at least 90%, 95% or 98%.

38. A stereo-encoded morpholino monomer of Formula (I) made by the method according to claim 29.

39. A chiral reagent of Formula (IV) 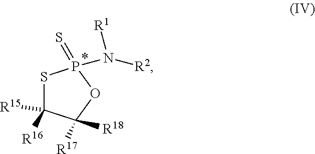
or a diastereomer or enantiomer thereof, or a salt thereof, wherein
P* represents a stereocenter that is either in the (R) or (S) configuration;
R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle;
R¹⁵, R¹⁶, R¹⁷ and R¹⁸ are independently —H, C1-C10 alkyl, C2-C12 alkenyl, aryl, or any two of R¹⁵, R¹⁶, R¹⁷ and R¹⁸ together with the carbons they are attached form a cycloalkyl or heteroalkyl ring which is optionally substituted with one or more C1-C10 alkyl or C2-C12 alkenyl groups.

40. The chiral reagent of claim 39, wherein R¹ and R² are C1-C6 alkyl.

41. The chiral reagent of claim 39, wherein R¹ and R² are methyl.

42. The chiral reagent of claim 39, wherein R¹ and R² together with the nitrogen they connect to form a heterocycle selected from morpholinyl, piperazinyl, pyrrolidinyl, and azetidinyl.

43. The chiral reagent of claim 39, wherein R¹⁵, R¹⁶, R¹⁷ and R¹⁸ are independently H, C1-C10 alkyl or aryl.

44. The chiral reagent of claim 39, wherein
R¹⁵, R¹⁶, R¹⁷ and R¹⁸ are H, or
R¹⁵ is C1-C10 alkyl or aryl, and R¹⁶, R¹⁷, R¹⁸ are H, or
R¹⁶ is C1-C10 alkyl or aryl, and R¹⁵, R¹⁷, R¹⁸ are H.

45. The chiral reagent of claim 39, wherein
R¹⁵ is phenyl and R¹⁶, R¹⁷, R¹⁸ are H, or
R¹⁶ is phenyl, and R¹⁵, R¹⁷, R¹⁸ are H.

46. The chiral reagent of claim 39, wherein R¹⁵, R¹⁶, R¹⁷ and R¹⁸ are H.

47. The chiral reagent of claim 39, selected from the following structures

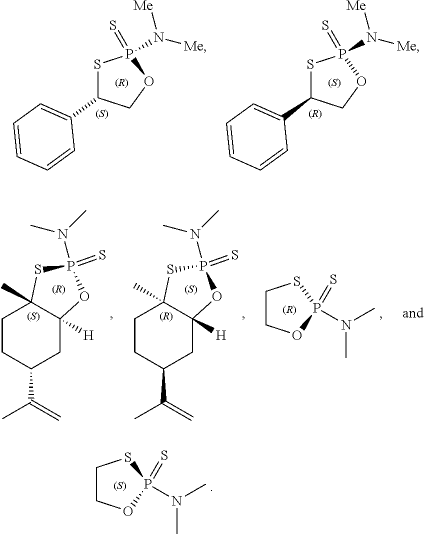

48. The chiral reagent of claim 39, wherein
(a) R¹⁵ is methyl;
R¹⁶ and R¹⁷ together form 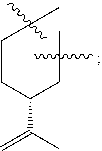 [SMILES: *CC[C@H](C*)C(=C)C]
and
R¹⁸ is H, or
(b) R¹⁶ is methyl; R¹⁵ and R¹⁸ together form 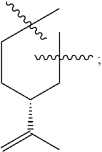 [SMILES: *CC[C@H](C*)C(=C)C]
and R¹⁷ is H.

49. The chiral reagent of claim 39, selected from

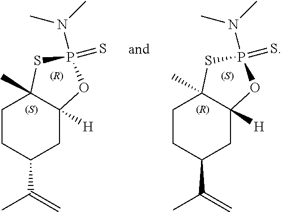

50. A method of preparing 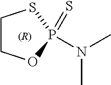
((R)-2-(dimethylamino)-1,3,2-oxathiaphospholane 2-sulfide), and/or 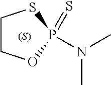
((S)-2-(dimethylamino)-1,3,2-oxathiaphospholane 2-sulfide), comprising
separating racemic 2-(dimethylamino)-1,3,2-oxathiaphospholane 2-sulfide by supercritical fluid chromatography (SFC) or chiral high-performance liquid chromatography (HPLC).

51. A method of making a chiral reagent of formula (IV) 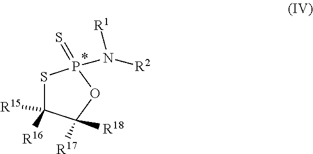
or a diastereomer or enantiomer thereof, or a salt thereof, wherein
P* represents a stereocenter that is either in the (R) or (S) configuration;
R¹ and R² are independently H or C1-C6 alkyl, or R¹ and R² together with the nitrogen they connect to form an optionally substituted heterocycle selected from morpholine, piperazine, pyrrolidine, and azetidine;
R¹⁵, R¹⁶, R¹⁷ and R¹⁸ are independently H, C1-C10 alkyl, C2-C12 alkenyl, aryl,
or any two of R¹⁵, R¹⁶, R¹⁷ and R¹⁸ together with the carbons they are attached form a cycloalkyl or heteroalkyl ring which is optionally substituted with one or more C1-C10 alkyl or C2-C12 alkenyl groups,
comprising reacting a chiral compound of Formula (V) 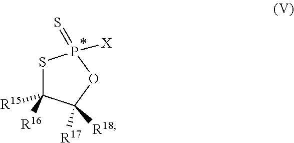
wherein P* indicates either (R) or (S) configuration, X is a leaving group selected from —S—Ar, —O—Ar or halide, wherein Ar is phenyl, pyridinyl, naphthyl, optionally substituted with one or more of C1-C6 alkyl, cyano, nitro or halo,
with NHR¹R² in the presence of an organic base to obtain the chiral reagent of Formula (IV),
wherein R¹ and R² are as defined above.

52. The method of claim 51, wherein Ar is selected from phenyl, pentafluorophenyl, 4-bromophenyl, mono- or di-nitrophenyl, and 2,3,5,6-tetrafluoropyridin-4-yl.

53. The method of claim 51, wherein the organic base is selected from the group consisting of 1,8-diazabicyclo[5.4.0]undec-7-ene, imidazole, triethylamine, Hunig's base, lutidine, pyridine and combinations thereof.

54. The method of claim 51, wherein the chiral reagent of Formula (IV) is selected from

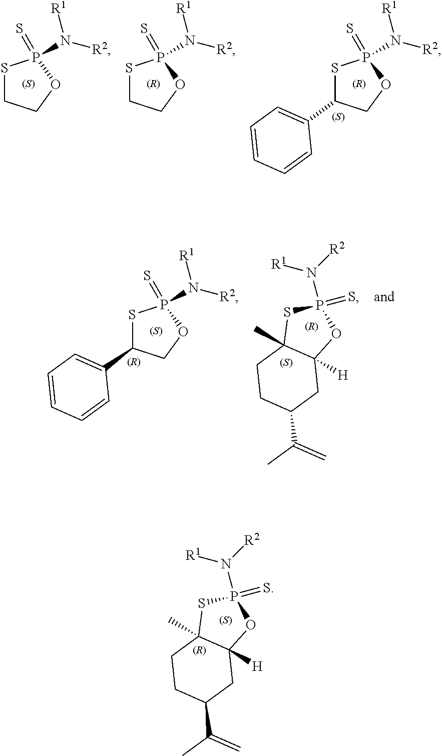

55. The method of claim 50, wherein R¹ and R² are methyl.

56. The method of claim 51, wherein the chiral reagent of Formula (IV) is 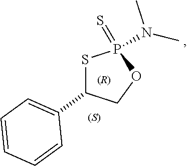
and
the compound of formula (V) is

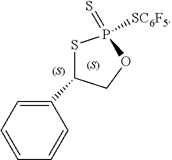

57. The method of claim 51, wherein the chiral reagent of Formula (IV) is 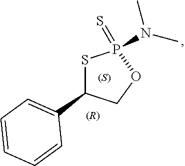
and
the compound of Formula (V) is

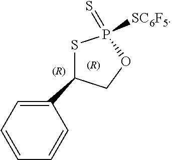

58. The method of claim 51, wherein the chiral reagent of Formula (IV) is 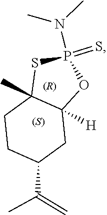
and
the compound of Formula (V) is

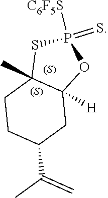

59. The method of claim 51, wherein the chiral reagent of Formula (IV) is 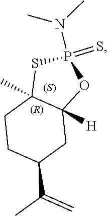
and
the compound of Formula (V) is

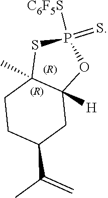

60. The method of claim 51, wherein the de/stereoselectvitiy is at least 90%, 95%, or 98%.

61. A chiral reagent of Formula (IV) made by the method according to claim 51.

62. A method of making chiral agent of Formula (IVa) 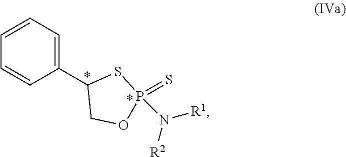
wherein * and P* each represents a stereocenter that is independently either in the (R) or (S) configuration;
R¹ and R² are independently H or C1-C6 alkyl, or R¹ and R² together with the nitrogen they connect to form an optionally substituted heterocycle selected from morpholinyl, piperazinyl, pyrrolidinyl, and azetidinyl;
comprising the step of reacting a compound of Formula (VI) 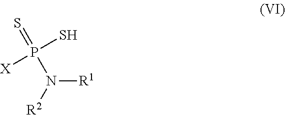
or a salt thereof, wherein X is a leaving group selected from-S—Ar, —O—Ar or halide, wherein Ar is phenyl, pyridinyl, naphthyl, optionally substituted with one or more of C1-C6 alkyl, cyano, nitro or halo,
with a chiral styrene oxide in the presence of chloroacetic acid to obtain the chiral reagent of Formula (IVa).

63. The method of claim 62, wherein R¹ and R² are methyl.

64. The method of claim 62, wherein Ar is selected from phenyl, pentafluorophenyl, 4-bromophenyl, mono- or di-nitrophenyl, and 2,3,5,6-tetrafluoropyridin-4-yl.

65. The method of claim 62, wherein the chiral reagent of Formula (IVa) has the structure: 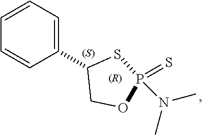
and the chiral styrene oxide is (S)-styrene oxide.

66. The method of claim 62, wherein the chiral reagent of Formula (IVa) has the structure: 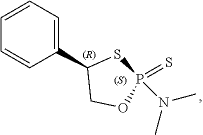
and the chiral styrene oxide is (R)-styrene oxide.

67. The method of claim 62, wherein the salt of Formula (VI) is a triethylamine salt.

68. A chiral reagent of Formula (IVa) made by the method according to claim 62.

69. A method of making a compound of Formula (VIa) 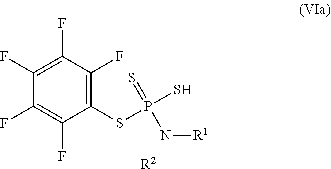
or a salt thereof, comprising the step of reacting a compound of Formula (VII) 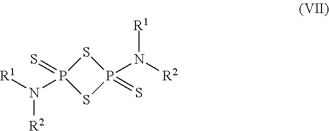
with 2,3,4,5,6-pentafluorobenzenethiol to obtain the compound of Formula (VIa) or a salt thereof.

70. The method of claim 69, wherein R¹ and R² are methyl.

71. The method of claim 69, wherein the salt of Formula (VIa) is triethylamine salt.

72. A chiral compound of Formula (V) 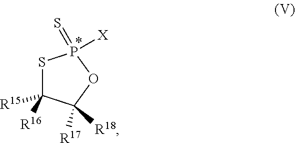
with at least 90% ee, wherein
P* indicates either (R) or (S) configuration,
R¹⁵, R¹⁶, R¹⁷ and R¹⁸ are H,
X is a leaving group selected from-S—Ar, —O—Ar or halide, wherein Ar is phenyl, pyridinyl, naphthyl, optionally substituted with one or more of C1-C6 alkyl, cyano, nitro or halo.

73. The chiral compound of Formula (V) according to claim 72, wherein X is —S—Ar, and Ar is phenyl, 4-bromophenyl, 4-nitrophenyl, pentafluorophenyl, dinitrophenyl, or 2,3,5,6-tetrafluoropyridin-4-yl.

74. The chiral compound of Formula (V) according to claim 72, wherein the compound of Formula (V) is:

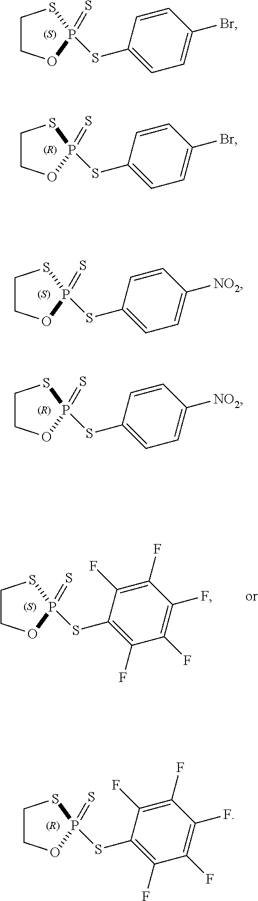

75. The chiral compound of Formula (V) according to claim 72, wherein the ee thereof is at least 92%, 95%, 98%, or 99%.

76. A method for preparing a chiral compound of Formula (V) 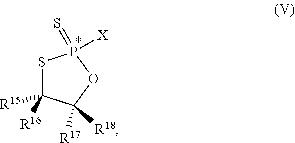
with at least 90% ee, wherein
P* indicates either (R) or (S) configuration,
R¹⁵, R¹⁶, R¹⁷ and R¹⁸ are H,
X is a leaving group selected from-S—Ar, —O—Ar or halide, wherein Ar is phenyl, pyridinyl, naphthyl, optionally substituted with one or more of C1-C6 alkyl, cyano, nitro or halo comprising the step of separating a racemic mixture of 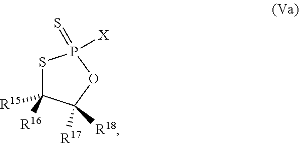
by chiral SFC or chiral HPLC,
wherein R¹⁵, R¹⁶, R¹⁷ and R¹⁸ and X are as defined above.

77. The method according to claim 51, wherein, for the chiral compound of Formula (V), X is —S—Ar, and Ar is phenyl, 4-bromophenyl, 4-nitrophenyl, pentafluorophenyl, dinitrophenyl, or 2,3,5,6-tetrafluoropyridin-4-yl.

78. The method according to claim 51, wherein the chiral compound of Formula (V) is:

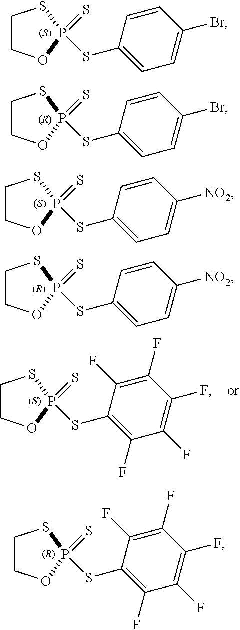

79. The method according to claim 51, wherein the ee of the chiral compound of Formula (V) is at least 92%, 95%, 98%, or 99%.

80. A method of preparing a morpholino dimer of Formula (Vill), 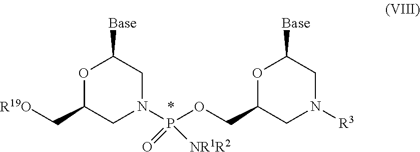
or a diastereomer or enantiomer thereof, or a salt thereof, wherein
P* represents a stereocenter that is either in the (R) or (S) configuration;
R¹ and R² are independently H or C1-C6 alkyl, or R¹ and R² together with the nitrogen they connect to form an optionally substituted heterocycle selected from morpholine, piperazine, pyrrolidine, and azetidine;
R³ is optionally substituted alkyl, trityl, benzyl, or sulfonyl;
Base is selected from the group consisting of: 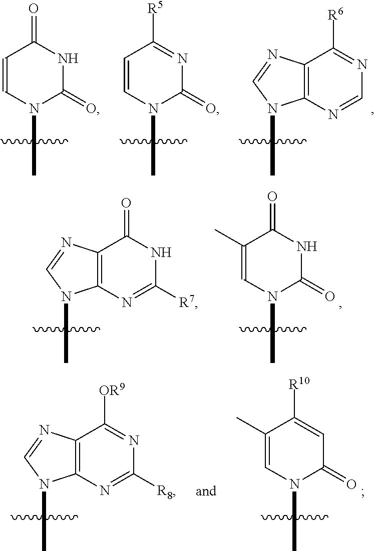
R⁵, R⁶, R⁷, R⁸, and R¹⁰ are independently —NR²⁰R²¹ or —N═CR¹²—NR¹³R¹⁴;
when any of R⁵, R⁶, R⁷, R⁸, and R¹⁰ is independently —NR²⁰R²¹, R²⁰ and R²¹ are independently selected from —H, —C(O)R¹¹, and —C(O)OR¹¹;
when any of R⁵, R⁶, R⁷, R⁸, and R¹⁰ is —NHC(O)R¹¹, R¹¹ is selected from methyl, ethyl, isopropyl, isobutyl, phenyl, 4-methoxyphenyl, 4-bromophenyl, and 4-nitrophenyl;
when any of R⁵, R⁶, R⁷, R⁸, and R¹⁰ is —NHC(O)OR¹¹, R¹¹ is C1-C6 alkyl, benzyl, 2,2,2-trichloroethyl, or optionally substituted aryl;
R¹², R¹³, and R¹⁴ are independently C1-C6 alkyl, or R¹² and R¹³ together form a 5 or 6-membered heterocycle with the adjacent C and N;
R⁹ is selected from the group consisting of H, cyanoethyl, (R) and/or (S) α-methylcyanoethyl, (R) and/or (S) p-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl;
R¹⁹ is H or a protecting group for a hydroxyl group, or a linker to solid support;
comprising the steps of
(a) providing a stereo-encoded morpholino monomer of Formula (Ia) 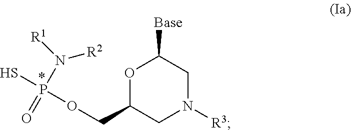
(b) optionally alkylating the sulfur of the stereo-encoded morpholino monomer of Formula (Ia) in step (a) to afford a stereo-encoded morpholino monomer of Formula (I) 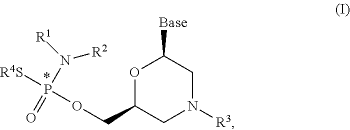
wherein R⁴ is C1-C6 alkyl;
(c) reacting the stereo-encoded morpholino monomer of Formula (Ia) from step (a) or the stereo-encoded morpholino monomer of Formula (I) from step (b) with a chlorinating agent to afford a stereo-encoded activated monomer of Formula (II) 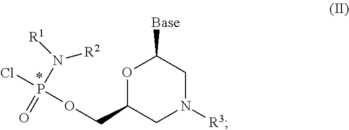
(d) optionally isolating the stereo-encoded activated compound of Formula (II) from step (c);
(e) coupling the stereo-encoded activated compound of Formula (II) from step (c) or (d) with a compound of Formula (IX) 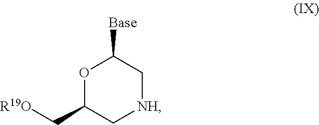
to obtain the morpholino dimer of Formula (VIII).

81. The method of claim 80, wherein R¹ and R² are methyl.

82. The method of claim 80, wherein R³ is trityl.

83. The method of claim 80, wherein R¹⁹ is H, TBS, TBDPS, benzoyl or DMTr, or a linker to solid support.

84. A morpholino dimer of Formula (VIII) prepared by the method according to claim 80.

85. A method of making a phosphorodiamidate morpholino oligomer of formula (X) 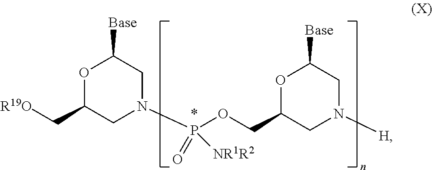
or a diastereomer or enantiomer thereof, or a salt thereof, wherein
P* represents a stereocenter that is either in the (R) or (S) configuration,
R¹ and R² are independently H, optionally substituted C1-C6 alkyl, optionally substituted phenyl, optionally substituted naphthyl, or R¹ and R² together with the nitrogen to which they connect to form an optionally substituted heterocycle,
wherein “optionally substituted” indicates that a moiety may be substituted with one or more independently selected substitutents selected from C1-C6 alkyl, C1-C6 alkenyl, C1-C6 alkynyl, C1-C6 alkoxy, —OH, —NH₂, —NO₂, —CH₂—NH—CH₃, —OCH₂CH₂CH₃, or OCH(CH₃)₂;
R¹⁹ is H, a protecting group for a hydroxyl group, or a linker to solid support, comprising the following steps:
(a) providing a stereo-encoded morpholino monomer of Formula (Ia) 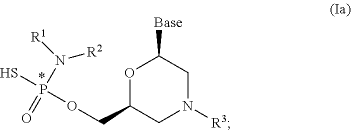
wherein R³ is optionally substituted alkyl, trityl, benzyl, or sulfonyl;
(b) optionally sulfur-alkylating the stereo-encoded morpholino monomer of Formula (Ia) in step (a) to afford a stereo-encoded morpholino monomer of Formula (I) 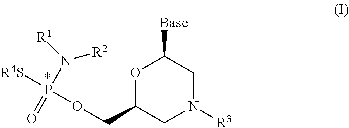
wherein R⁴ is C1-C6 alkyl;
(c) reacting the stereo-encoded morpholino monomer of Formula (Ia) from step (a) or the stereo-encoded morpholino monomer of Formula (I) from step (b) with a chlorinating agent to afford a stereo-encoded activated monomer of Formula (II) 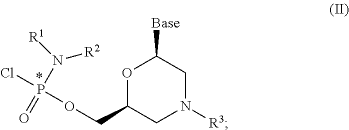
(d) optionally isolating the stereo-encoded activated compound of Formula (II) from step (c);
(e) coupling the compound of Formula (II) from step (c) or (d) with a compound of Formula (IX) 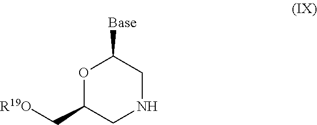
to obtain a morpholino dimer of Formula (VIII) 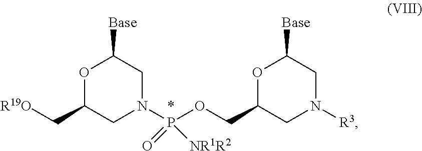
wherein R¹⁹ is H, a suitable protecting group for a hydroxyl group, or a linker to solid support;
(f) deprotecting the morpholino dimer of Formula (VIII) to obtain an intermediate oligomer of Formula (X) 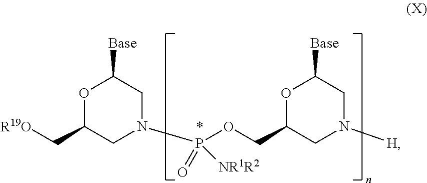
wherein n is 1;
(g) repeating step (i) and step (ii) below m times, wherein m is an integer from 0 to 28 inclusive:
(i) reacting the intermediate oligomer of Formula (X) 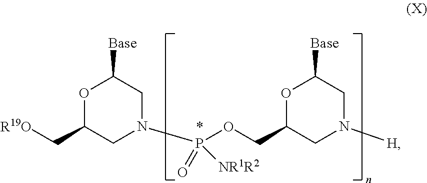
wherein n is an integer from 1 to 28 inclusive,
with a stereo-encoded morpholino monomer of Formula (I) 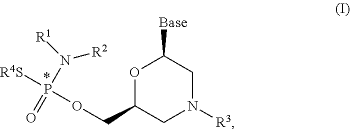
wherein R⁴ is H or C1-C6 alkyl,
to provide an intermediate of formula (XI): 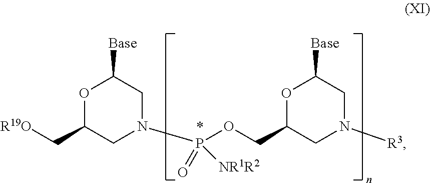
wherein n is an integer from 1 to 29 inclusive; and
(ii) deprotecting the intermediate of Formula (XI) from step (i) to provide phosphorodiamidate morpholino oligomer of Formula (X): 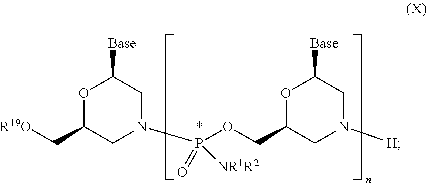
wherein n is an integer from 1 to 29 inclusive;
(h) optionally removing R¹⁹ group in the intermediate oligomer of formula (X) from step (ii) of step (g), when R¹⁹ is a suitable protecting group for a hydroxyl group or a linker to solid support, to provide the phosphorodiamidate morpholino oligomer of Formula (X): 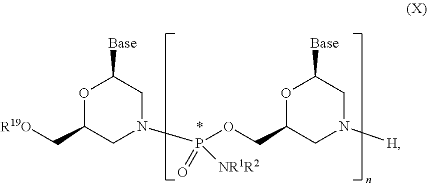
wherein R¹⁹ is H.

86. The method of claim 85, wherein R¹ and R² are methyl.

87. The method of claim 85 or 86, wherein R³ is trityl.

88. The method of claim 85, wherein R¹⁹, as a hydroxyl protecting group in Formulas (IX), (VIII), (IX) and (X), is selected from H, TBS, TBDPS, benzoyl, or DMTr.

89. A phosphorodiamidate morpholino oligomer made by the method according to claim 85.

90. Use of a stereo-encoded morpholino monomer of Formula (I) 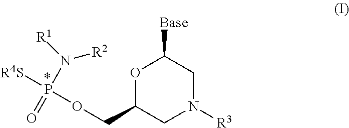
or a diastereomer or enantiomer thereof, or a salt thereof, wherein
P* represents a stereocenter that is either in the (R) or (S) configuration;
R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle;
R³ is a protecting group for morpholino nitrogen, selected from optionally substituted C1—C6 alkyl, trityl, benzyl, or sulfonyl;
R⁴ is hydrogen or C1-C6 alkyl,
wherein “optionally substituted” indicates that a moiety may be substituted with one or more independently selected substitutents selected from C1-C6 alkyl, C1-C6 alkenyl, C1-C6 alkynyl, C1-C6 alkoxy, —OH, —NH₂, —NO₂, —CH₂—NH—CH₃, —OCH₂CH₂CH₃, or OCH(CH₃)₂;
in the preparation of a morpholino dimer of Formula (VIII), 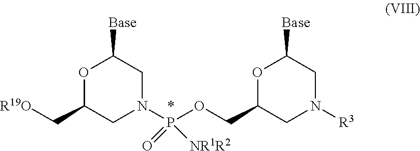
or a diastereomer or enantiomer thereof, or a salt thereof, wherein
P* represents a stereocenter that is either in the (R) or (S) configuration;
R¹ and R² are independently H, optionally substituted C1-C6 alkyl, optionally substituted phenyl, optionally substituted naphthyl, or R¹ and R² together with the nitrogen they connect to form an optionally substituted heterocycle;
R³ is optionally substituted alkyl, trityl, benzyl, or sulfonyl;
R¹⁹ is H, a protecting group for a hydroxyl group selected from silyl, acyl, optionally substituted trityl, or a linker to solid support,
wherein the stereoselectivity or diastereoselectivity is at least 90%, 95%, or 98%.

91. The use of claim 90, wherein the stereo-encoded morpholino monomer of formula (I) is selected from: 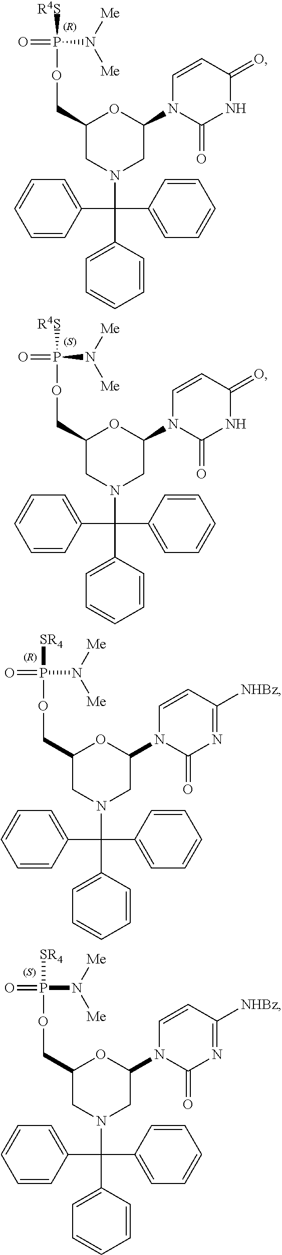 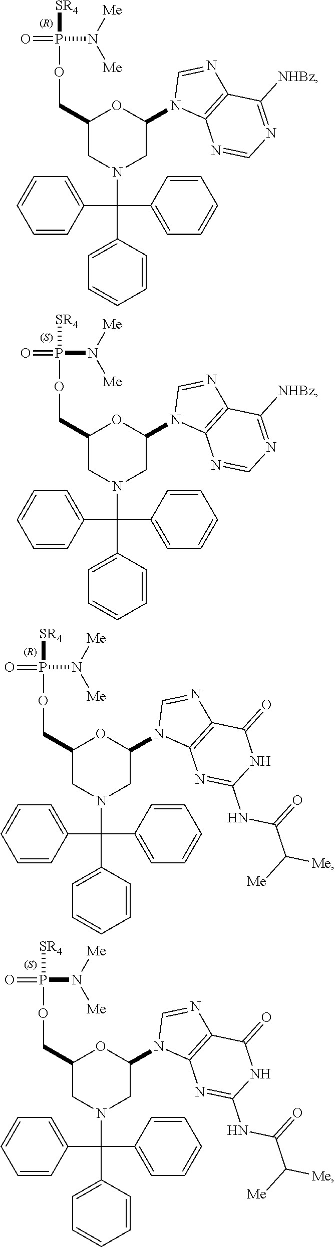 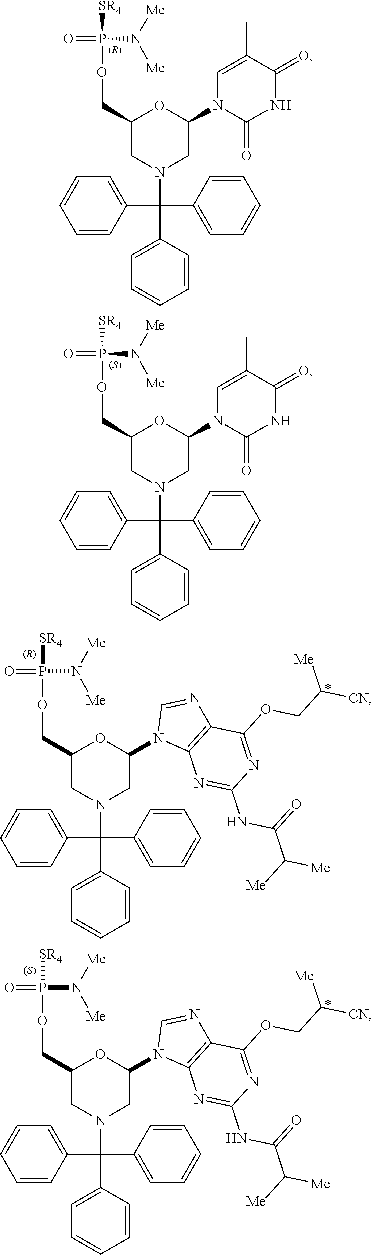 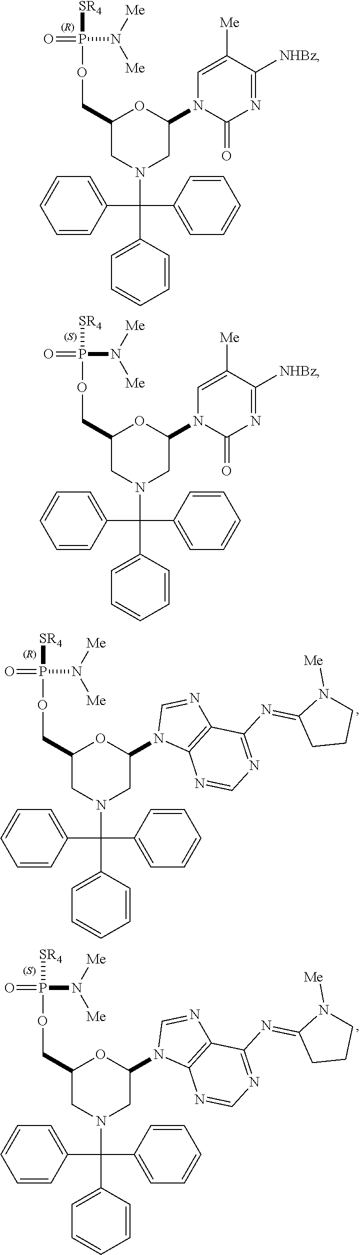 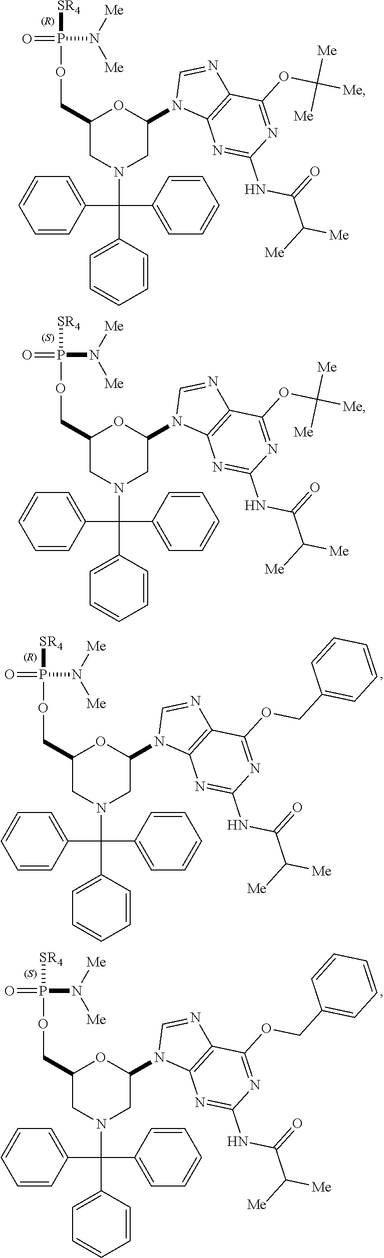 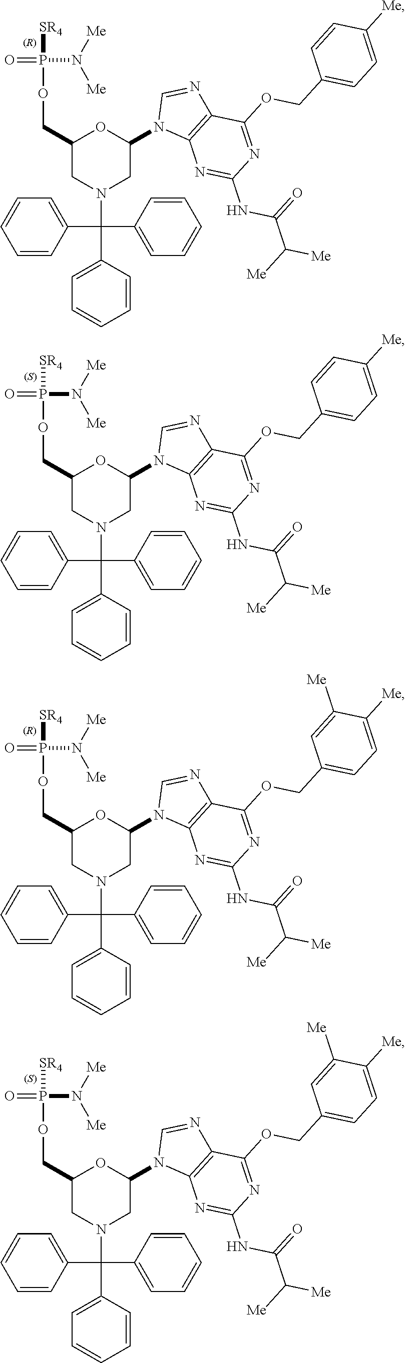 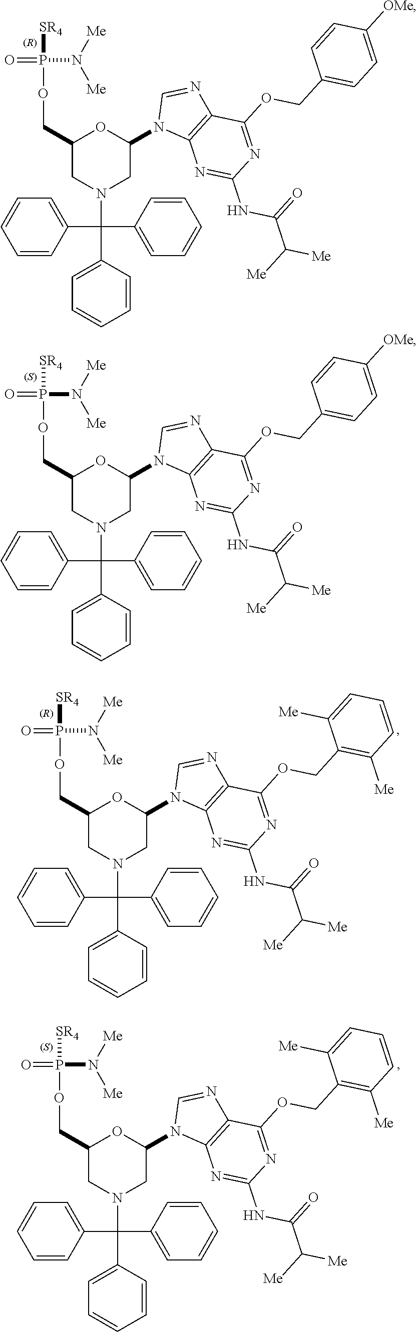 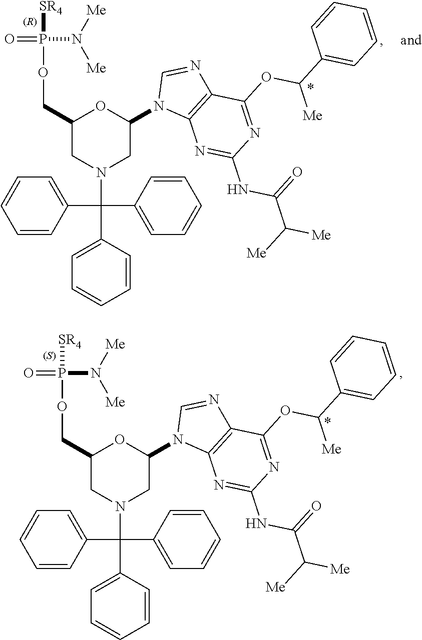
wherein * indicates either (R) or (S) stereochemistry, or a mixture thereof, and
R⁴ is H or methyl.

92. Use of a chiral reagent of Formula (IV) 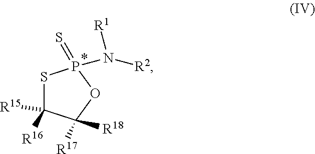
P* represents a stereocenter that is either in the (R) or (S) configuration;
R¹ and R² are independently H, optionally substituted C1-C6 alkyl, optionally substituted phenyl, optionally substituted naphthyl, or R¹ and R² together with the nitrogen they connect to form an optionally substituted heterocycle;
R¹⁵, R¹⁶, R¹⁷ and R¹⁸ are independently H, C1-C10 alkyl, C2-C12 alkenyl, aryl,
or any two of R¹⁵, R¹⁶, R¹⁷ and R¹⁸ together with the carbons they are attached form a cycloalkyl or heteroalkyl ring that is optionally substituted with one or more C1-C10 alkyl or C2-C12 alkenyl groups,
in the preparation of a stereo-encoded morpholino monomer of formula (I), 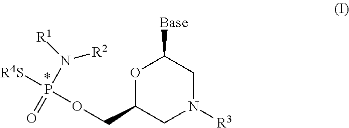
or a diastereomer or enantiomer thereof, or a salt thereof, wherein
P* represents a stereocenter that is either in the (R) or (S) configuration;
R¹ and R² are as defined above;
R³ is a protecting group for morpholino nitrogen, selected from optionally substituted C1-C6 alkyl, trityl, benzyl, or sulfonyl;
R⁴ is hydrogen or C1-C6 alkyl,
Base is selected from the group consisting of: 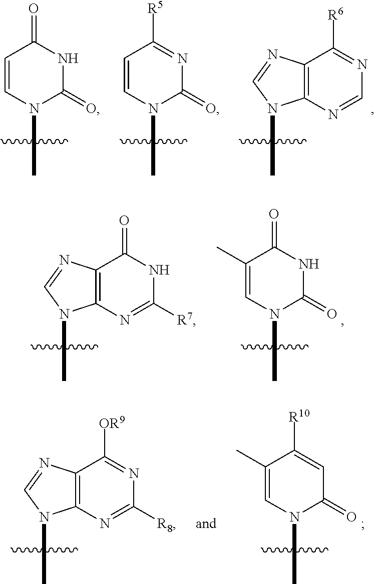
R⁵, R⁶, R⁷, R⁸, and R¹⁰ are independently —NR²⁰R²¹ or —N═CR¹²—NR¹³R¹⁴
when R⁵, R⁶, R⁷, R⁸, and R¹⁰ are independently —NR²⁰R²¹, R²⁰ and R²¹ are independently selected from —H, —C(O)R¹¹, and —C(O)OR¹¹;
when R⁵, R⁶, R⁷, R⁸, and R¹⁰ are —NHC(O)R¹¹, R¹¹ is selected from methyl, ethyl, isopropyl, isobutyl, phenyl, 4-methoxyphenyl, 4-bromophenyl, and 4-nitrophenyl;
when R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl;
R¹², R¹³, and R¹⁴ are independently C1-C6 alkyl, or R¹² and R¹³ together form a 5 or 6-membered heterocycle with the adjacent C and N;
R⁹ is H, optionally substituted C1-C6 alkyl, optionally substituted benzyl, acyl, carbonate, carbamate, cyanoethyl, α-methylcyanoethyl, s-methylcyanoethyl, pivaloyloxy benzyl, or silyl;
wherein the stereoselectivity or diastereoselectivity is at least 90%, 95%, or 98%.

93. The use of claim 92, wherein the chiral reagent is selected from

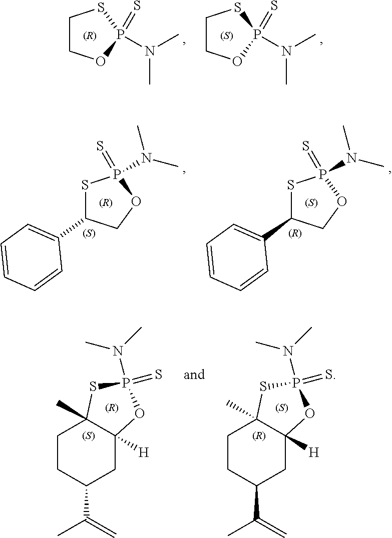

94. A stereo-encoded compound that is a monomer of Formula (Id) 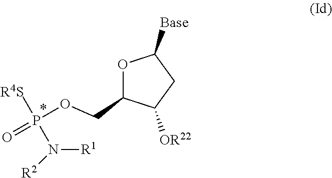
or a diastereomer or enantiomer thereof, or a salt thereof, wherein
P* represents a stereocenter that is either in the (R) or (S) configuration;
R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle;
R²² is H or a protecting group for hydroxyl group selected from silyl, acyl, optionally substituted trityl, benzyl, 2-tetrahydropyranyl, and ethoxyethyl, wherein silyl is trialkylsilyl, where the alkyl are the same or different and are C1-C6 alkyl;
R⁴ is H or C1-C6 alkyl,
Base is selected from the group consisting of: 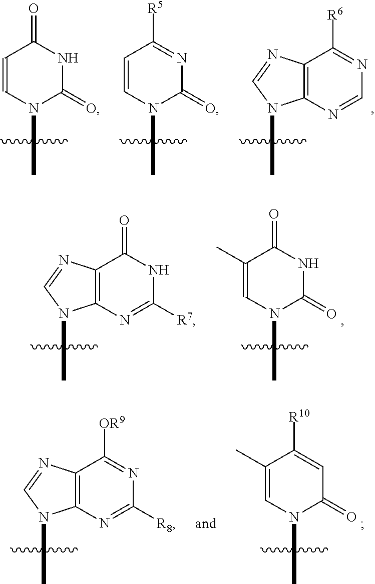
R⁵, R⁶, R⁷, R⁸, and R¹⁰ are independently —NR²⁰R²¹ or —N═CR¹²—NR¹³R¹⁴;
R⁵, R⁶, R⁷, R⁸, and R¹⁰ are independently —NR²⁰R²¹, R²⁰ and R²¹ are independently selected from —H, —C(O)R¹¹, and —C(O)OR¹¹;
when R⁵, R⁶, R⁷, R⁸, and R¹⁰ are —NHC(O)R¹¹, R¹¹ is selected from methyl, ethyl, isopropyl, isobutyl, phenyl, 4-methoxyphenyl, 4-bromophenyl, and 4-nitrophenyl;
when R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl;
R¹², R¹³, and R¹⁴ are independently C1-C6 alkyl, or R¹² and R¹³ together form a 5 or 6-membered heterocycle with the adjacent C and N;
R⁹ is H, optionally substituted C1-C6 alkyl, optionally substituted benzyl, acyl, carbonate, carbamate, cyanoethyl, α-methylcyanoethyl, β-methylcyanoethyl, pivaloyloxy benzyl, or silyl;
wherein “optionally substituted” indicates that a moiety may be substituted with one or more independently selected substitutents selected from C1-C6 alkyl, C1-C6 alkenyl, C1-C6 alkynyl, C1-C6 alkoxy, —OH, —NH₂, —NO₂, —CH₂—NH—CH₃, —OCH₂CH₂CH₃, or OCH(CH₃)₂.

95. The compound of claim 94, wherein the compound is crystalline.

96. The compound of claim 94, wherein R¹ and R² are independently optionally substituted C1-C6 alkyl.

97. The compound of claim 94, wherein R¹ and R² are methyl.

98. The compound of claim 94, wherein R¹ and R² together with the nitrogen they connect to form a heterocycle selected from morpholinyl, piperazinyl, pyrrolidinyl, and azetidinyl.

99. The compound of claim 94, wherein R²² is selected from the group consisting of H, TBS, TBDPS, benzoyl, trityl, DMTr, p-methoxyphenyldiphenylmethyl, benzyl, 4-methoxybenzyl, 2-tetrahydropyranyl, and ethoxyethyl.

100. The compound of claim 94, wherein R²² is TBS or TBDPS.

101. The compound of claim 94, wherein R⁴ is H or methyl.

102. The compound of claim 94, wherein R¹ and R² are methyl, R²² is TBS or TBDPS, and R⁴ is H or methyl.

103. The compound of claim 94, wherein any of R⁵, R⁶, R⁷ R³ and R¹⁰ is independently —NHBz or —NHC(O)iPr.

104. The compound of claim 94, wherein any of R⁵, R⁶, R⁷ R³ and R¹⁰ is independently

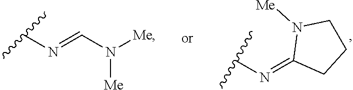 [SMILES: */N=C/N(C)C ; */N=C1/CCCN1C]

105. The compound of claim 94, wherein R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-, 3,4-, and 2,6-dimethylbenzyl, 4-methoxybenzyl, and 4-pivaloyloxy benzyl.

106. The compound of claim 94, selected from the following compounds: 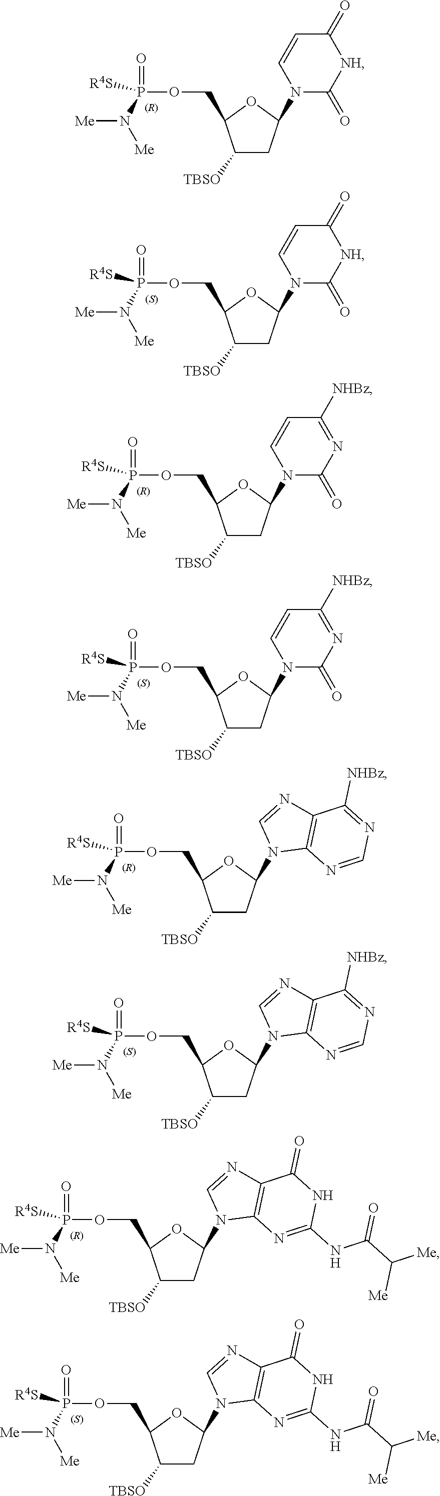 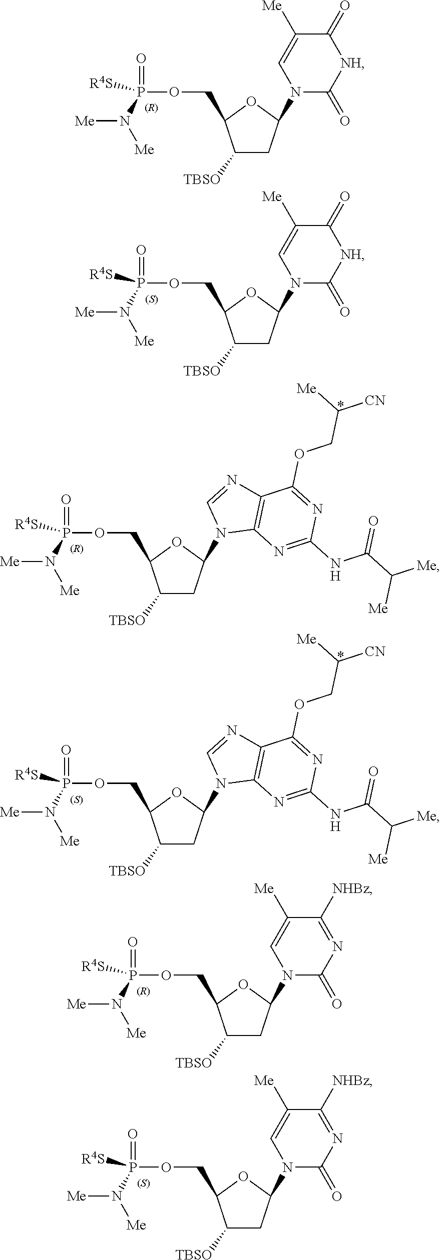 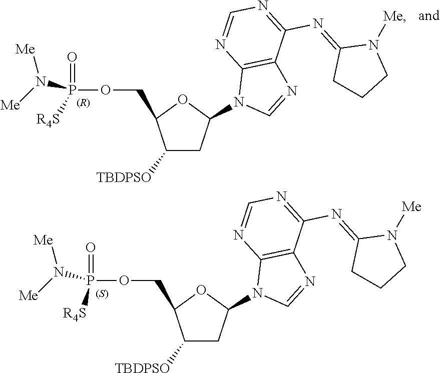
wherein * indicates either (R) or (S) stereochemistry, or a mixture thereof, and
R⁴ is H or methyl.

107. A method of preparing a stereo-encoded activated monomer of Formula (IId) 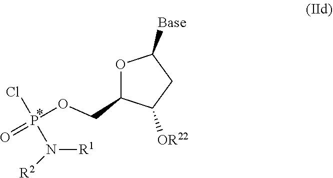
or a diastereomer or enantiomer thereof, or a salt thereof, wherein
P* represents a stereocenter that is either in the (R) or (S) configuration;
R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle;
R²² is a protecting group for hydroxyl oxygen;
Base is selected from the group consisting of: 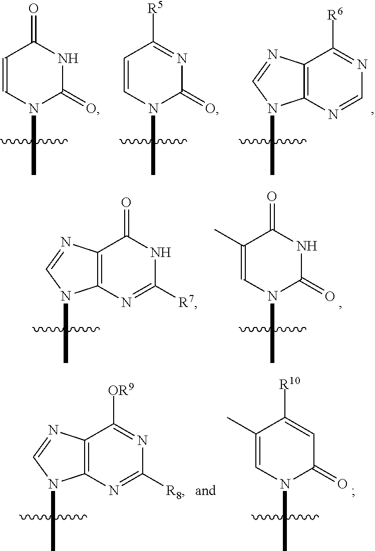
R⁵, R⁶, R⁷, R⁸, and R¹⁰ are independently —NR²⁰R²¹ or —N═CR¹²—NR¹³R¹⁴;
R⁵, R⁶, R⁷, R⁸, and R¹⁰ are independently —NR²⁰R²¹, R²⁰ and R²¹ are independently selected from —H, —C(O)R¹¹, and —C(O)OR¹¹;
when R⁵, R⁶, R⁷, R⁸, and R¹⁰ are —NHC(O)R¹¹, R¹¹ is selected from methyl, ethyl, isopropyl, isobutyl, phenyl, 4-methoxyphenyl, 4-bromophenyl, and 4-nitrophenyl;
when R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl;
R¹², R¹³, and R¹⁴ are independently C1-C6 alkyl, or R¹² and R¹³ together form a 5 or 6-membered heterocycle with the adjacent C and N;
R⁹ is H, optionally substituted C1-C6 alkyl, optionally substituted benzyl, acyl, carbonate, carbamate, cyanoethyl, α-methylcyanoethyl, s-methylcyanoethyl, pivaloyloxy benzyl, or silyl;
wherein “optionally substituted” indicates that a moiety may be substituted with one or more independently selected substitutents selected from C1-C6 alkyl, C1-C6 alkenyl, C1-C6 alkynyl, C1-C6 alkoxy, —OH, —NH₂, —NO₂, —CH₂—NH—CH₃, —OCH₂CH₂CH₃, or OCH(CH₃)₂;
comprising the following steps:
(a) providing a stereo-encoded monomer of Formula (Iad) 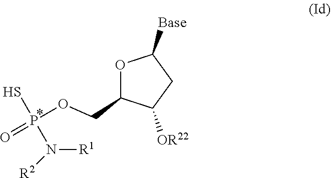
(b) optionally alkylating the sulfur of the stereo-encoded monomer of Formula (Iad) in step (a) to afford a stereo-encoded monomer of Formula (Id) 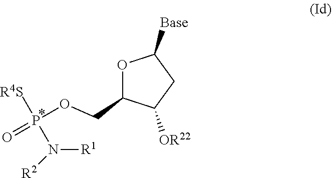
wherein R⁴ is C1-C6 alkyl;
(c) reacting the stereo-encoded monomer of Formula (Ia) from step (a) or the stereo-encoded monomer of Formula (I) from step (b) with a chlorinating agent to afford the stereo-encoded activated monomer of Formula (IId).

108. The method of claim 107, wherein R⁴ is methyl.

109. The method of claim 107, wherein R¹ and R² are independently C1-C6 alkyl.

110. The method of claim 107, wherein R¹ and R² are methyl.

111. The method of 107, wherein R¹ and R², together with the nitrogen to which they connect, form a heterocycle selected from morpholinyl, piperazinyl, pyrrolidinyl, and azetidinyl.

112. The method of claim 107, wherein R²² is selected from the group consisting of H, TBS, TBDPS, benzoyl, trityl, DMTr, p-methoxyphenyldiphenylmethyl, benzyl, 4-methoxybenzyl, 2-tetrahydropyranyl, and ethoxyethyl.

113. The method of claim 107, wherein R²² is TBS or TBDMS.

114. The method of claim 107, wherein R¹ and R² are methyl, R²² is TBS, and R⁴ is H or methyl.

115. The method of claim 107, wherein any of R⁵, R⁶, R⁷ R³ and R¹⁰ is independently —NHBz or —NHC(O)iPr.

116. The method of claim 107, wherein any of R⁵, R⁶, R⁷ R⁸ and R¹⁰ are independently

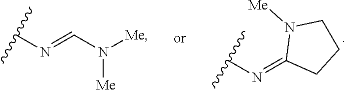 [SMILES: */N=C/N(C)C ; */N=C1/CCCN1C]

117. The method of claim 107, wherein R⁹ is selected from the group consisting of H, cyanoethyl, (R) and/or (S) α-methylcyanoethyl, (R) and/or (S) β-methylcyanoethyl, isobutyl, t-butyl, benzyl, α-methylbenzyl, 4-methylbenzyl, 2,4-dimethylbenzyl, 3,4-dimethylbenzyl, 2,6-dimethylbenzyl, 4-methoxybenzyl, and 4-pivaloyloxy benzyl.

118. The method of claim 107, wherein the chlorinating agent is SO₂Cl₂.

119. The method of claim 112, wherein step (b) further includes reacting with 2,4,6-collidine.

120. The method of claim 107, wherein R⁴ is H and the chlorinating agent is tetramethyl chloroenamine.

121. A stereo-encoded activated monomer prepared by the method according to claim 107.

122. A method for preparing a stereo-encoded monomer of Formula (Id) 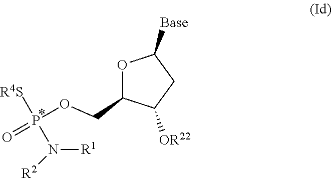
or a diastereomer or enantiomer thereof, or a salt thereof, wherein
P* represents a stereocenter that is either in the (R) or (S) configuration;
R¹ and R² are independently selected from —H, optionally substituted C1-C6 alkyl, optionally substituted phenyl, and optionally substituted naphthyl, or R¹ and R² together with the nitrogen to which they connect form an optionally substituted heterocycle;
R²² is a protecting group for hydroxyl oxygen;
R⁴ is hydrogen or C1-C6 alkyl,
Base is selected from the group consisting of: 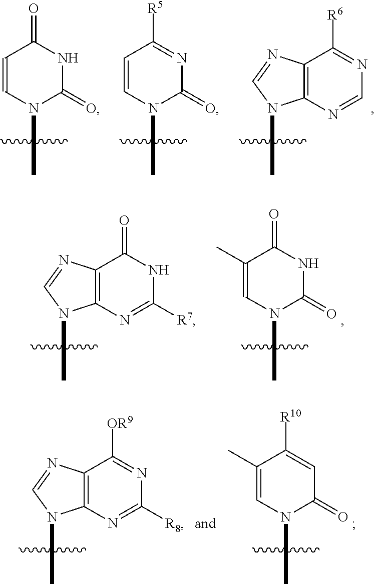
R⁵, R⁶, R⁷, R⁸, and R¹⁰ are independently —NR²⁰R²¹ or —N═CR¹²—NR¹³R¹⁴;
where any of R⁵, R⁶, R⁷, R⁸, and R¹⁰ is independently —NR²⁰R²¹, R²⁰ and R²¹ are independently selected from —H, —C(O)R¹¹, and —C(O)OR¹¹;
when any of R⁵, R⁶, R⁷, R⁸, and R¹⁰ is independently —NHC(O)R¹¹, R¹¹ is selected from methyl, ethyl, isopropyl, isobutyl, phenyl, 4-methoxyphenyl, 4-bromophenyl, and 4-nitrophenyl;
when any of R⁵, R⁶, R⁷, R⁸, and R¹⁰ is independently —NHC(O)OR¹¹, R¹¹ is C1-C6 alkyl, benzyl, 2,2,2-trichloroethyl, or optionally substituted aryl;
R¹², R¹³, and R¹⁴ are independently C1-C6 alkyl, or R¹² and R¹³ together form a 5 or 6-membered heterocycle with the adjacent C and N;
R⁹ is H, optionally substituted C1-C6 alkyl, optionally substituted benzyl, acyl, carbonate, carbamate, cyanoethyl, α-methylcyanoethyl, s-methylcyanoethyl, pivaloyloxy benzyl, or silyl;
comprising the step of
reacting a compound of Formula (IIId) 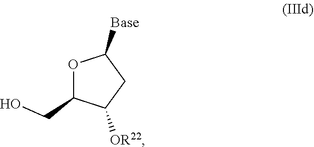
wherein R²² and Base are as defined above,
with a chiral reagent of Formula (IV) 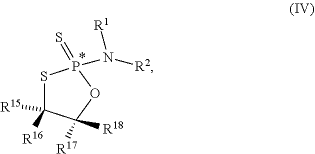
wherein R¹ and R² are as defined above;
R¹⁵, R¹⁶, R¹⁷ and R¹⁸ are independently H, C1-C10 alkyl, C2-C12 alkenyl, aryl,
or any two of R¹⁵, R¹⁶, R¹⁷ and R¹⁸ together with the carbons they are attached form a cycloalkyl or heteroalkyl ring which is optionally substituted with one or more C1-C10 alkyl or C2-C12 alkenyl groups,
in the presence of a base to obtain the stereo-encoded monomer of Formula (Id).

123. The method of claim 122, wherein R¹ and R² are methyl.

124. The method of claim 122, wherein R¹⁵, R¹⁶, R¹⁷ and R¹⁸ are independently H, C1-C10 alkyl, or aryl.

125. The method of claim 122, wherein
R¹⁵, R¹⁶, R¹⁷ and R¹⁸ are H, or
R¹⁵ is C1-C10 alkyl or aryl, and R¹⁶, R¹⁷, R¹⁸ are H, or
R¹⁶ is C1-C10 alkyl or aryl, and R¹⁵, R¹⁷, R¹⁸ are H; or
R¹⁵ is phenyl and R¹⁶, R¹⁷, R¹⁸ are H, or
R¹⁶ is phenyl, and R¹⁵, R¹⁷, R¹⁸ are H.

126. The method of claim 122, wherein the chiral agent of Formula (IV) is selected from

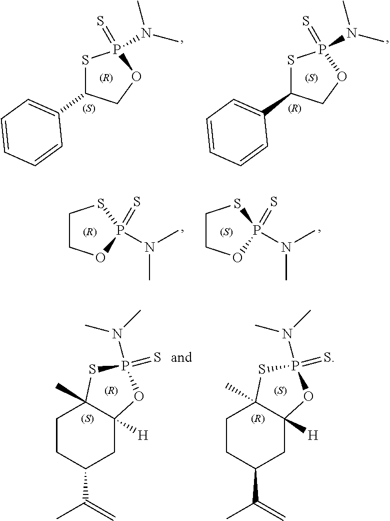

127. The method of claim 122, wherein the base is selected from the group consisting of NaH, DBU, sodium tert-amylate, sodium tert-pentoxide, NaOtBu, KOtBu, potassium tert-pentoxide, and NaHMDS.

128. The method of claim 122, wherein the reaction is conducted at room temperature in a polar solvent selected from the group consisting of THF, acetonitrile, 2-MeTHF, 1,6-dioxane, and DME.

129. The method of claim 122, wherein the stereo-encoded monomer of Formula (Id) is crystalline.

130. The method of claim 122, wherein the de/ee ratio/stereoselectivity is at least 90%, 95% or 98%.

131. A stereo-encoded monomer of Formula (Id) made by the method according to claim 122.

132. A method of making a phosphorodiamidate morpholino-DNA oligomer of formula (Xd) 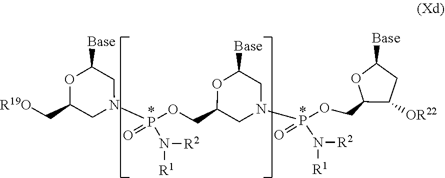
or a diastereomer or enantiomer thereof, or a salt thereof, wherein
each P* represents a stereocenter that is either in the (R) or (S) configuration,
n is an integer between 1 and 7 inclusive,
R¹ and R² are independently H, optionally substituted C1-C6 alkyl, optionally substituted phenyl, optionally substituted naphthyl, or R¹ and R² together with the nitrogen to which they connect to form an optionally substituted heterocycle, and wherein
each Base may be the same or different and is selected from the group consisting of: 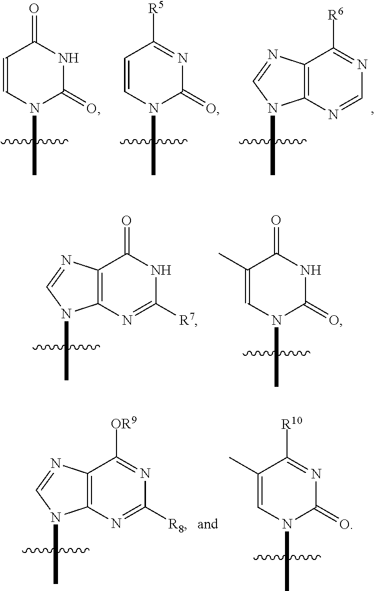
wherein R⁵, R⁶, R⁷, R⁸, and R¹⁰ are independently —NR²⁰R²¹ or —N═CR¹²—NR¹³R¹⁴;
wherein any of R⁵, R⁶, R⁷, R⁸, and R¹⁰ is independently —NR²⁰R²¹, R²⁰ and R²¹ are independently selected from —H, —C(O)R¹¹, and —C(O)OR¹¹; where R⁵, R⁶, R⁷, R⁸, and R¹⁰ are —NHC(O)R¹¹, R¹¹ is methyl, ethyl, isopropyl, isobutyl, phenyl, 4-methoxyphenyl, 4-bromophenyl, or 4-nitrophenyl; or where R⁵, R⁶, R⁷, R⁸, and R¹⁰ are —NHC(O)OR¹¹, R¹¹ is C1-C6 alkyl, benzyl, 2,2,2-trichloroethyl, or optionally substituted aryl;
or wherein any of R⁵, R⁶, R⁷, R⁸, and R¹⁰ is independently —N═CR¹²—NR¹³R¹⁴; or wherein R¹², R¹³, and R¹⁴ are independently C1-C6 alkyl, or R¹² and R¹³ together form a 5 or 6-membered heterocycle with the adjacent C and N; and wherein
R⁹ is selected from the group consisting of H, optionally substituted C1-C6 alkyl, optionally substituted benzyl, acyl, carbonate, carbamate, and silyl;
R¹⁹ and R²² are the same or different and are independently H or a protecting group for a hydroxyl group, or a linker to solid support,
comprising the following steps:
(a) providing a stereo-encoded morpholino monomer of Formula (Ia) 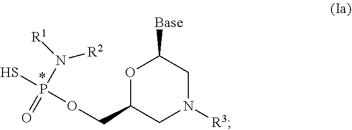
wherein R³ is optionally substituted alkyl, trityl, benzyl, or sulfonyl;
(b) optionally sulfur-alkylating the stereo-encoded morpholino monomer of Formula (Ia) in step (a) to afford a stereo-encoded morpholino monomer of Formula (I) 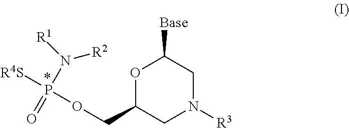
wherein R⁴ is C1-C6 alkyl;
(c) reacting the stereo-encoded morpholino monomer of Formula (Ia) from step (a) or the stereo-encoded morpholino monomer of Formula (I) from (b) with a chlorinating agent to afford a stereo-encoded activated monomer of Formula (II) 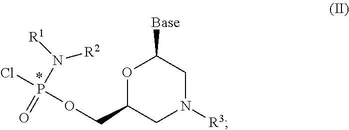
(d) optionally isolating the stereo-encoded activated compound of Formula (II) from step (c);
(e) coupling the compound of Formula (II) from step (c) or (d) with a compound of Formula (IX) 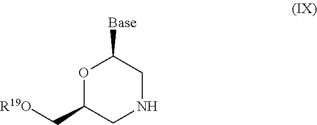
to obtain a morpholino dimer of Formula (VIII) 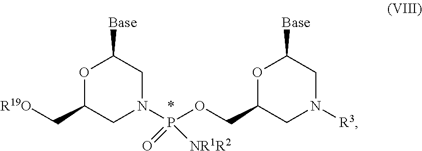
wherein R¹⁹ is H, a suitable protecting group for a hydroxyl group, or a linker to solid support;
(f) deprotecting the morpholino dimer of Formula (VIII) to obtain an intermediate oligomer of Formula (X) 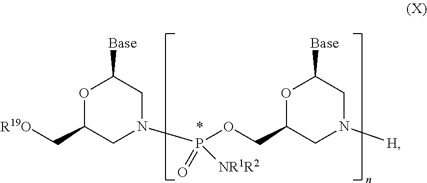
wherein n is 1;
(g) repeating step (i) and step (ii) below m times, wherein m is an integer from 0 to 6 inclusive:
(i) reacting the intermediate oligomer of Formula (X) 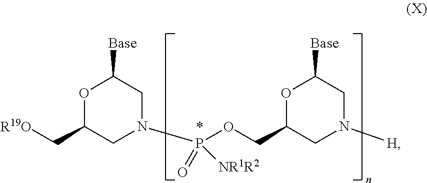
wherein n is an integer from 1 to 7 inclusive,
with a stereo-encoded activated compound of Formula (II) prepared from a stereo-encoded morpholino monomer of Formula (I) by steps (a) to (d) 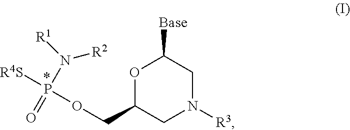
wherein R⁴ is H or C1-C6 alkyl,
to provide an intermediate of formula (XI): 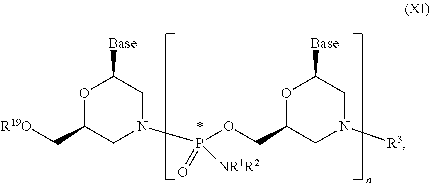
wherein n is an integer from 1 to 7 inclusive; and
(ii) deprotecting the intermediate of Formula (XI) from step (i) to provide a phosphorodiamidate morpholino oligomer of Formula (X): 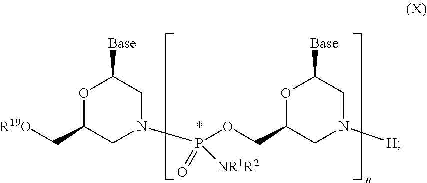
wherein n is an integer from 1 to 7 inclusive;
(h) providing a stereo-encoded DNA monomer of Formula (Iad) 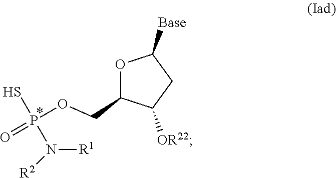
wherein R²² is H or a protecting group for hydroxyl group;
(j) optionally sulfur-alkylating the stereo-encoded DNA monomer of Formula (Iad) in step (h) to afford a stereo-encoded DNA monomer of Formula (Id) 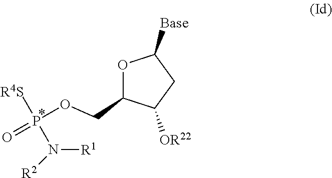
wherein R⁴ is C1-C6 alkyl;
(k) reacting the stereo-encoded DNA monomer of Formula (Iad) from step (h) or the stereo-encoded DNA monomer of Formula (Id) from (j) with a chlorinating agent to afford a stereo-encoded activated monomer of Formula (IId) 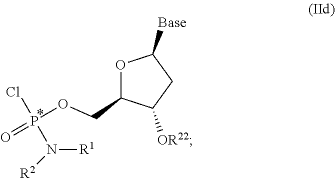
(l) optionally isolating the stereo-encoded activated compound of Formula (IId) from step (k);
(m) coupling the compound of Formula (IId) from step (k) or (l) with a compound of Formula (X) from step (g): 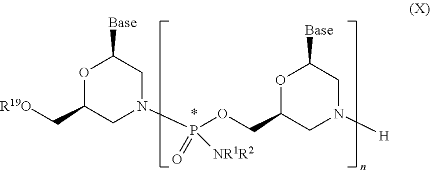
wherein n is an integer from 1 to 7 inclusive;
to provide a phosphorodiamidate morpholino-DNA oligomer of formula (Xd): 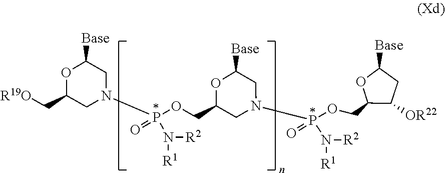
(n) optionally, removing the R²² group and/or the R¹⁹ group in the phosphorodiamidate morpholino-DNA oligomer of formula (Xd), to provide a PMO-DNA hetero-oligomer of Formula (Xd): 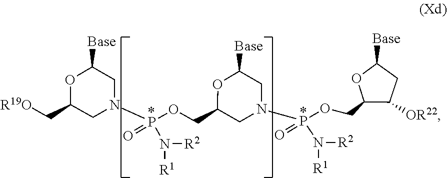
wherein n is from 1 to 7 inclusive, and R¹⁹ and R²² are independently H or a hydroxyl protecting groups that may be the same or different, or a linker to solid support.

133. The method of claim 132, wherein R¹ and R² are methyl.

134. The method of claim 132, wherein R³ is trityl.

135. The method of claim 132, wherein R¹⁹ and R²² are independently selected from H, TBS, TBDPS, benzoyl, trityl, DMTr, p-methoxyphenyldiphenylmethyl, benzyl, 4-methoxybenzyl, 2-tetrahydropyranyl, and ethoxyethyl, or a linker to solid support.

136. A phosphorodiamidate morpholino-DNA hetero-oligomer made by the method according to claim 132.

137. A stereo-encoded activated monomer of Formula (II) according to claim 14: 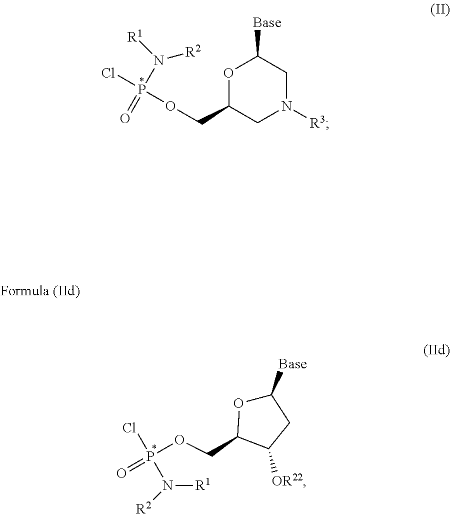
selected from:

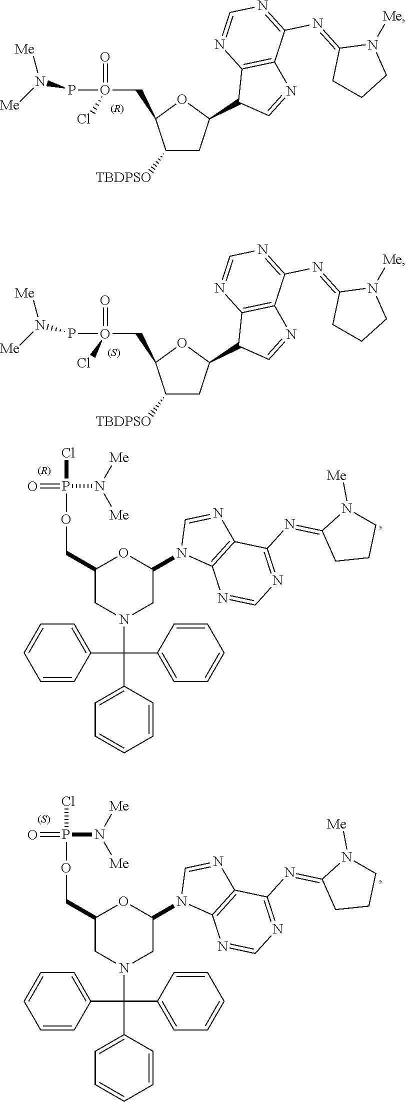

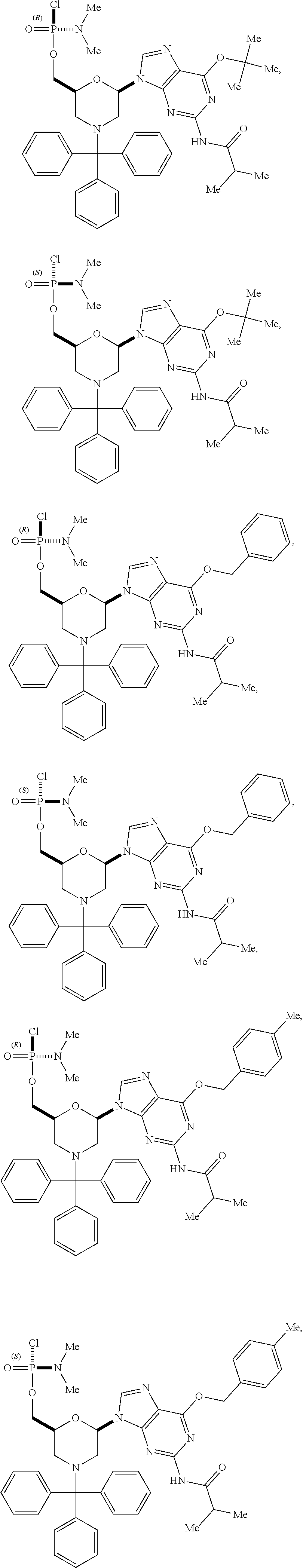

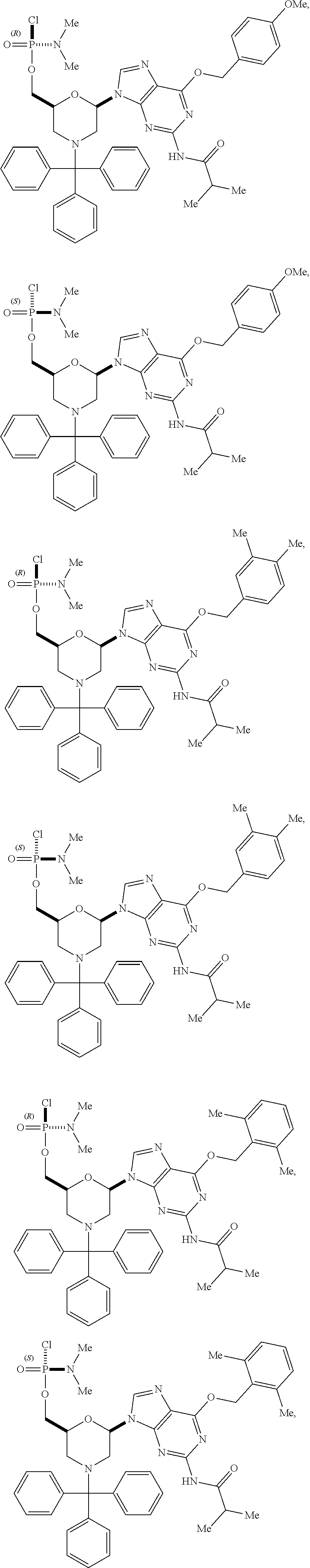

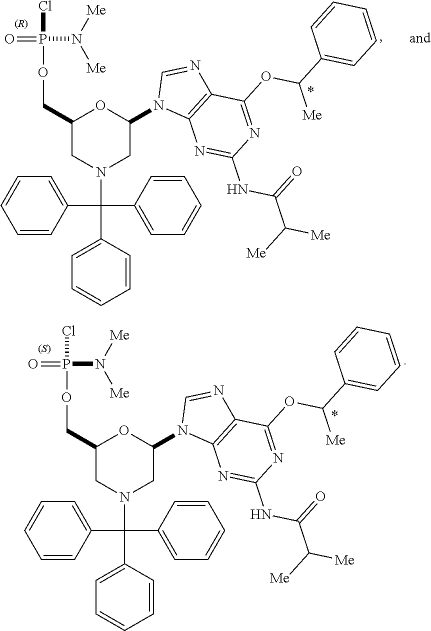

138. A stereo-encoded activated monomer selected from the group consisting of: 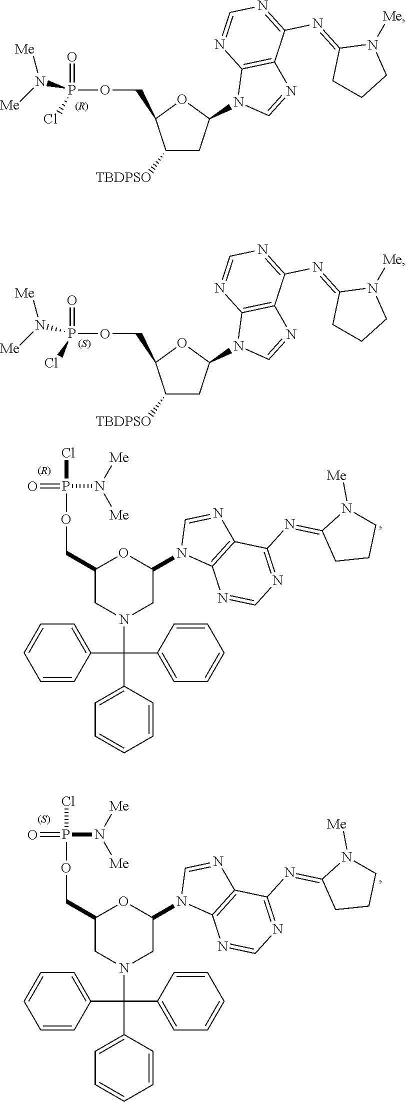 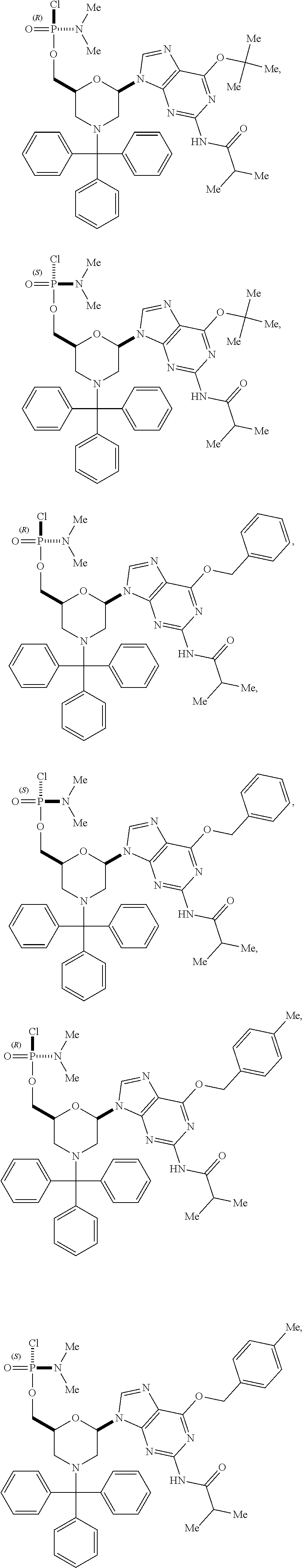 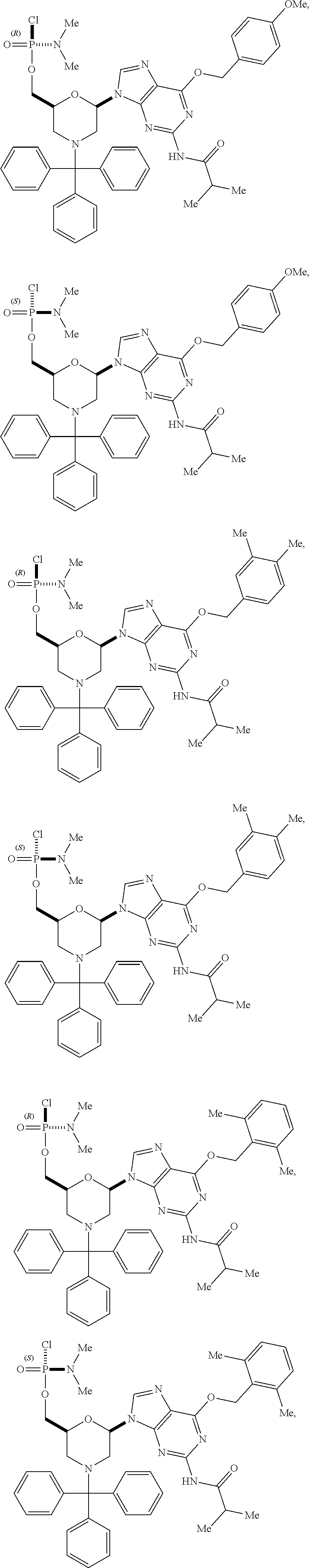 [chemical structure]
